(19) United States
(12) Patent Application Publication
(10) Pub. No.: US 2024/0285803 A1
(43) Pub. Date: Aug. 29, 2024

(54) COMPOSITIONS AND METHODS FOR CELL TYPE-SPECIFIC GENE EXPRESSION IN THE INNER EAR

(71) Applicant: Decibel Therapeutics, Inc., Boston, MA (US)

(72) Inventors: Theresa ABELL, Medford, MA (US); Kathryn ELLIS, Arlington, MA (US)

(21) Appl. No.: 18/568,077

(22) PCT Filed: Jun. 10, 2022
(86) PCT No.: PCT/US2022/033079

§ 371 Date: Dec. 7, 2023

Related U.S. Application Data
(60) Provisional application No. 63/209,562, filed on Jun. 11, 2021.

(51) Int. Cl. A61K 48/00; A61P 27/16; C12N 15/113; C12N 15/86

(52) U.S. Cl. CPC A61K 48/0058; A61P 27/16; C12N 15/113; C12N 15/86; C12N 2310/141; C12N 2750/14143; C12N 2830/48; C12N 2830/50

(57) ABSTRACT

The disclosure provides nucleic acid vectors containing a promoter operably linked to a polynucleotide and to a microRNA target sequence for a microRNA that is differentially expressed between different inner ear cell types. Such vectors and compositions containing the same can be used to prevent or reduce off-target expression of the polynucleotide, and, therefore, to achieve cell type-specific expression of the polynucleotide in the inner ear. Accordingly, the nucleic acid vectors and compositions described herein can be used to treat subjects having or at risk of developing hearing loss or vestibular dysfunction.

### SEQUENCE LISTING

The instant application contains a Sequence Listing which has been submitted electronically in ASCII format and is hereby incorporated by reference in its entirety. The ASCII copy, created on Jun. 10, 2022, is named 51124-090WO2_Sequence_Listing_6_10_22_ST25 and is 239,852 bytes in size.

### BACKGROUND

Hearing loss is a major public health issue that is estimated to affect nearly 15% of school-age children and one out of three people by age sixty-five. The most common type of hearing loss is sensorineural hearing loss, a type of hearing loss caused by defects in the cells of the inner ear, such as cochlear hair cells, or the neural pathways that project from the inner ear to the brain. Sensorineural hearing loss is often acquired, and has a variety of causes, including acoustic trauma, disease or infection, head trauma, ototoxic drugs, and aging. There are also genetic causes of sensorineural hearing loss, such as mutations in genes involved in the development and function of cells of the inner ear. Mutations in over 90 such genes have been identified, including mutations inherited in an autosomal recessive, autosomal dominant, or X-linked pattern.

Factors that disrupt the development, survival, or integrity of cells in the cochlea, such as genetic mutations, disease or infection, ototoxic drugs, head trauma, and aging, may similarly affect cells in the vestibule and are, therefore, also implicated in vestibular dysfunction. Indeed, patients carrying mutations that disrupt hair cell development or function can present with both hearing loss and vestibular dysfunction, or either disorder alone. Extensive loss of vestibular sensory cells is highly debilitating and can elicit nauseating bouts of dizziness, imbalance, and incapacitation. Approximately 35% of US adults age 40 years and older exhibit balance disorders and this proportion dramatically increases with age, leading to disruption of daily activities, decline in mood and cognition, and an increased prevalence of falls among the elderly.

Accordingly, there is a need for therapies that can be used to treat of hearing loss or vestibular dysfunction.

### SUMMARY OF THE INVENTION

The present invention provides nucleic acid vectors designed to express a polynucleotide of interest (e.g., a transgene encoding a protein or a polynucleotide that can be transcribed to produce an inhibitory RNA) in a cell type-specific manner in the inner ear. These vectors contain a promoter operably linked to the polynucleotide of interest and to a polynucleotide that can be transcribed to produce a microRNA (miRNA) target sequence that is recognized by a miRNA that is differentially expressed in different inner ear cell types (e.g., a miRNA that is not expressed in a cell type in which the polynucleotide of interest is suitable for expression and that is expressed in an inner ear cell type in which it is desired to prevent or reduce expression of the polynucleotide of interest). The vectors can contain one or more different polynucleotides of interest and one or more polynucleotides that can be transcribed to produce a miRNA target sequence (e.g., one or more copies of a polynucleotide that can be transcribed to produce the same miRNA target sequence or one or more copies of each of multiple, different polynucleotides, each of which can be transcribed to produce a different miRNA target sequence). The invention also provides methods of using the nucleic acid vectors to treat hearing loss (e.g., sensorineural hearing loss), tinnitus, or vestibular dysfunction (e.g., vertigo, dizziness, imbalance, bilateral vestibulopathy, oscillopsia, or a balance disorder) in a subject, such as a human subject.

In a first aspect, the invention provides a nucleic acid vector containing a first promoter operably linked to: (i) a first polynucleotide that can be transcribed to produce an expression product (e.g., a polynucleotide that can be transcribed to produce a protein or an inhibitory RNA molecule); and (ii) at least one polynucleotide that can be transcribed to produce a microRNA (miRNA) target sequence (e.g., 1, 2, 3, 4, 5, 6, 7, 8, 9, 10, or more polynucleotides that can be transcribed to produce miRNA target sequences), in which: the first polynucleotide is suitable for expression in a first inner ear cell type, but not in a different, second inner ear cell type; and the miRNA target sequence transcribed from the at least one polynucleotide operably linked to the first promoter is recognized by a miRNA expressed in the second inner ear cell type but not in the first inner ear cell type. In some embodiments, the expression product transcribed from the first polynucleotide promotes conversion of the first inner ear cell type to the second inner ear cell type. In some embodiments, the first polynucleotide is expressed in the first inner ear cell type but not in the second inner ear cell type.

In some embodiments, the vector contains at least two (e.g., 2, 3, 4, 5, 6, 7, 8, 9, 10, or more) polynucleotides that can be transcribed to produce miRNA target sequences. In some embodiments, the vector contains a polynucleotide that can be transcribed to produce a first miRNA target sequence and a polynucleotide that can be transcribed to produce a second miRNA target sequence, in which each miRNA target sequence is recognized by a different miRNA. In some embodiments, the vector further includes a polynucleotide that can be transcribed to produce a third miRNA target sequence, in which each of the first, second, and third miRNA target sequences are recognized by different miRNAs. In some embodiments, the vector includes at least two copies (e.g., 2, 3, 4, 5, 6, 7, 8, 9, 10, or more copies) of a polynucleotide that can be transcribed to produce the same miRNA target sequence. In some embodiments, the vector includes at least three copies (e.g., 3, 4, 5, 6, 7, 8, 9, 10, or more copies) of the polynucleotide that can be transcribed to produce the same miRNA target sequence. In some embodiments, each polynucleotide that can be transcribed to produce a miRNA target sequence that is operably linked to the first promoter is the same. In some embodiments, each polynucleotide that can be transcribed to produce a miRNA target sequence is located 3′ of the first polynucleotide.

In some embodiments, the vector further includes a WPRE sequence located 3′ of the first polynucleotide, and each polynucleotide that can be transcribed to produce a miRNA target sequence is located between the first polynucleotide and the WPRE sequence.

In some embodiments, each polynucleotide that can be transcribed to produce a miRNA target sequence is in the 3′ UTR of the first polynucleotide. In some embodiments, each polynucleotide that can be transcribed to produce a miRNA target sequence is in the 5′ UTR of the first polynucleotide.

In some embodiments, each polynucleotide that can be transcribed to produce a miRNA target sequence that is operably linked to the first promoter is independently targeted by a miRNA listed in Table 2. In some embodiments, each polynucleotide that can be transcribed to produce a miRNA target sequence that is operably linked to the first promoter is independently targeted by one of: miR-183, miR-96, miR-182, miR-18a, miR-100, miR-124a, miR-140, miR-194, miR-135, or miR-135b.

In some embodiments, the first inner ear cell type is a cochlear supporting cell and the second inner ear cell type is a cochlear hair cell or a spiral ganglion neuron. In some embodiments, the second inner ear cell type is a cochlear hair cell. In some embodiments, the second inner ear cell type is a spiral ganglion neuron.

In some embodiments, the first inner ear cell type is a vestibular supporting cell and the second inner ear cell type is a vestibular hair cell or a vestibular ganglion neuron. In some embodiments, the second inner ear cell type is a vestibular hair cell. In some embodiments, the second inner ear cell type is a vestibular type I hair cell. In some embodiments, the second inner ear cell type is a vestibular ganglion neuron.

In some embodiments, the first inner ear cell type is a vestibular type II hair cell and the second inner ear cell type is a vestibular type I hair cell.

In some embodiments, the first inner ear cell type is a vestibular type II hair cell and the second inner ear cell type is a vestibular ganglion neuron.

In some embodiments, the first polynucleotide is a transgene encoding a protein, is a polynucleotide that can be transcribed to produce an inhibitory RNA, or encodes a component of a gene editing system. In some embodiments, the first polynucleotide is a transgene encoding a protein. In some embodiments, the transgene is a wild-type version of a gene listed in Table 4. In some embodiments, the transgene is a polynucleotide listed in Table 5. In some embodiments, the first polynucleotide can be transcribed to produce an inhibitory RNA. In some embodiments, the inhibitory RNA is an siRNA, shRNA, or shRNA-mir. In some embodiments, the inhibitory RNA is an inhibitory RNA targeting Sox2 (e.g., an inhibitory RNA described herein). In some embodiments, the first polynucleotide encodes a component of a gene editing system. In some embodiments, the first polynucleotide can be transcribed to produce a guide RNA. In some embodiments, the first polynucleotide encodes a nuclease. In some embodiments, the first polynucleotide encodes Atoh1, Gfi1, Pou4f3, Ikzf2, dnSox2, or Gjb2.

In some embodiments, the first promoter is supporting cell-specific promoter, a hair cell-specific promoter, or a ubiquitous promoter. In some embodiments, the first promoter is a CMV promoter, a MYO15 promoter, an LFNG promoter, an FGFR3 promoter, a SLC1A3 promoter, a GFAP promoter, or a SLC6A14 promoter. In some embodiments, the first promoter is an inner ear cell type-specific promoter listed in Table 12 (e.g., a supporting cell- or hair cell-specific promoter listed in Table 12).

In some embodiments, the vector further includes a second polynucleotide that can be transcribed to produce an expression product, in which the second polynucleotide is different from the first polynucleotide.

In some embodiments, the vector includes in 5′ to 3′ order: the first promoter, the first polynucleotide, the second polynucleotide, and the at least one polynucleotide that can be transcribed to produce a miRNA target sequence, in which the second polynucleotide is suitable for expression in the first inner ear cell type, but not in the second inner ear cell type. In some embodiments, the vector further includes a WPRE sequence located 3′ of the second polynucleotide, and each polynucleotide that can be transcribed to produce a miRNA target sequence is located between the second polynucleotide and the WPRE sequence. In some embodiments, each polynucleotide that can be transcribed to produce a miRNA target sequence is in the 3′ UTR of the second polynucleotide. In some embodiments, each polynucleotide that can be transcribed to produce a miRNA target sequence is in the 5′ UTR of the first polynucleotide.

In some embodiments, the second polynucleotide is operably linked to a second promoter. In some embodiments, the vector includes in 5′ to 3′ order: the first promoter, the first polynucleotide, the at least one polynucleotide that can be transcribed to produce a miRNA target sequence, the second promoter, and the second polynucleotide. In some embodiments, expression of the second polynucleotide is not regulated by a miRNA target sequence. In some embodiments, the vector further includes at least one polynucleotide that can be transcribed to produce a miRNA target sequence 3′ of the second polynucleotide that is operably linked to the second promoter, in which the second polynucleotide is suitable for expression in a third inner ear cell type, but not in a different, fourth inner ear cell type, and in which the miRNA target sequence transcribed from the at least one polynucleotide operably linked to the second promoter is recognized by a miRNA expressed in the fourth inner ear cell type, but not in the third inner ear cell type. In some embodiments, the vector further includes a WPRE sequence located 3′ of the second polynucleotide, and each polynucleotide that can be transcribed to produce a miRNA target sequence that is operably linked to the second polynucleotide is located between the second polynucleotide and the WPRE sequence. In some embodiments, each polynucleotide that can be transcribed to produce a miRNA target sequence that is operably linked to the second promoter is in the 3′ UTR of the second polynucleotide. In some embodiments, each polynucleotide that can be transcribed to produce a miRNA target sequence that is operably linked to the second promoter is in the 5′ UTR of the second polynucleotide.

In some embodiments, the vector further includes a third polynucleotide that can be transcribed to produce an expression product, in which the third polynucleotide is different from the first polynucleotide and the second polynucleotide.

In some embodiments, the vector includes in 5′ to 3′ order: the first promoter, the first polynucleotide, the second polynucleotide, the third polynucleotide, and the at least one polynucleotide that can be transcribed to produce a miRNA target sequence, in which the third polynucleotide is suitable for expression in the first inner ear cell type, but not in the second inner ear cell type. In some embodiments, the vector further includes a WPRE sequence located 3′ of the third polynucleotide, and each polynucleotide that can be transcribed to produce a miRNA target sequence is located between the third polynucleotide and the WPRE sequence. In some embodiments, each polynucleotide that can be transcribed to produce a miRNA target sequence is in the 3′ UTR of the third polynucleotide. In some embodiments, each polynucleotide that can be transcribed to produce a miRNA target sequence is in the 5′ UTR of the first polynucleotide.

In some embodiments, the first polynucleotide is operably linked to the first promoter and the second and third polynucleotides are operably linked to the second promoter. In some embodiments, the vector includes in 5′ to 3′ order: the first promoter, the first polynucleotide, the at least one polynucleotide that can be transcribed to produce a miRNA target sequence, the second promoter, the second polynucleotide, and the third polynucleotide. In some embodiments, expression of the second and third polynucleotides is not regulated by a miRNA target sequence. In some embodiments, the vector further includes at least one polynucleotide that can be transcribed to produce a miRNA target sequence 3′ of the third polynucleotide that is operably linked to the second promoter, wherein the second and third polynucleotides are suitable for expression in a third inner ear cell type, but not in a different, fourth inner ear cell type, and wherein miRNA target sequence transcribed from the at least one polynucleotide operably linked to the second promoter is recognized by a miRNA expressed in the fourth inner ear cell type, but not in the third inner ear cell type. In some embodiments, the vector further includes a WPRE sequence located 3′ of the third polynucleotide, and each polynucleotide that can be transcribed to produce a miRNA target sequence that is operably linked to the second promoter is located between the third polynucleotide and the WPRE sequence. In some embodiments, each polynucleotide that can be transcribed to produce a miRNA target sequence that is operably linked to the second promoter is in the 3′ UTR of the third polynucleotide. In some embodiments, each polynucleotide that can be transcribed to produce a miRNA target sequence that is operably linked to the second promoter is in the 5′ UTR of the second polynucleotide.

In some embodiments, the first polynucleotide and the second polynucleotide are operably linked to the first promoter and the third nucleic acid is operably linked to a second promoter. In some embodiments, the vector includes in 5′ to 3′ order: the first promoter, the first polynucleotide, the second polynucleotide, the at least one polynucleotide that can be transcribed to produce a miRNA target sequence, the second promoter, and the third polynucleotide. In some embodiments, expression of the third polynucleotide is not regulated by a miRNA target sequence. In some embodiments, the vector further includes at least one polynucleotide that can be transcribed to produce a miRNA target sequence 3′ of the third polynucleotide that is operably linked to the second promoter, in which the third polynucleotide is suitable for expression in a third inner ear cell type, but not in a different, fourth inner ear cell type, and in which the miRNA target sequence transcribed from the at least one polynucleotide operably linked to the second promoter is recognized by a miRNA expressed in the fourth inner ear cell type, but not in the third inner ear cell type. In some embodiments, the vector further includes a WPRE sequence located 3′ of the second polynucleotide, and each polynucleotide that can be transcribed to produce a miRNA target sequence that is operably linked to the first promoter is located between the second polynucleotide and the WPRE sequence. In some embodiments, each polynucleotide that can be transcribed to produce a miRNA target sequence that is operably linked to the first promoter is in the 3′ UTR of the second polynucleotide. In some embodiments, each polynucleotide that can be transcribed to produce a miRNA target sequence that is operably linked to the first promoter is in the 5′ UTR of the first polynucleotide. In some embodiments, the vector further includes a WPRE sequence located 3′ of the third polynucleotide, and each polynucleotide that can be transcribed to produce a miRNA target sequence that is operably linked to the second promoter is located between the third polynucleotide and the WPRE sequence. In some embodiments, each polynucleotide that can be transcribed to produce a miRNA target sequence that is operably linked to the second promoter is in the 3′ UTR of the third polynucleotide. In some embodiments, each polynucleotide that can be transcribed to produce a miRNA target sequence that is operably linked to the second promoter is in the 5′ UTR of the third polynucleotide.

In some embodiments, the first polynucleotide is operably linked to the first promoter, the second polynucleotide is operably linked to the second promoter, and the third polynucleotide is operably linked to a third promoter.

In some embodiments, the vector includes in 5′ to 3′ order: the first promoter, the first polynucleotide, at least one polynucleotide that can be transcribed to produce a miRNA target sequence, the second promoter, the second polynucleotide, the third promoter, and the third polynucleotide. In some embodiments, expression of the second and third polynucleotides is not regulated by a miRNA target sequence. In some embodiments, the vector includes in 5′ to 3′ order: the first promoter, the first polynucleotide, at least one polynucleotide that can be transcribed to produce a miRNA target sequence, the second promoter, the second polynucleotide, at least one polynucleotide that can be transcribed to produce a miRNA target sequence, the third promoter, and the third polynucleotide. In some embodiments, expression of the third polynucleotide is not regulated by a miRNA target sequence. In some embodiments, the vector further includes at least one polynucleotide that can be transcribed to produce a miRNA target sequence 3′ of the third polynucleotide that is operably linked to the third promoter, in which the third polynucleotide is suitable for expression in a fifth inner ear cell type, but not in a different, sixth inner ear cell type, and in which the miRNA target sequence transcribed from the at least one polynucleotide operably linked to the third promoter is recognized by a miRNA expressed in the sixth inner ear cell type, but not in the fifth inner ear cell type. In some embodiments, the vector further includes a WPRE sequence located 3′ of the second polynucleotide, and each polynucleotide that can be transcribed to produce a miRNA target sequence that is operably linked to the second promoter is located between the second polynucleotide and the WPRE sequence. In some embodiments, each polynucleotide that can be transcribed to produce a miRNA target sequence that is operably linked to the second promoter is in the 3′ UTR of the second polynucleotide. In some embodiments, each polynucleotide that can be transcribed to produce a miRNA target sequence that is operably linked to the second promoter is in the 5′ UTR of the second polynucleotide. In some embodiments, the vector further includes a WPRE sequence located 3′ of the third polynucleotide, and each polynucleotide that can be transcribed to produce a miRNA target sequence that is operably linked to the third promoter is located between the third polynucleotide and the WPRE sequence. In some embodiments, each polynucleotide that can be transcribed to produce a miRNA target sequence that is operably linked to the third promoter is in the 3′ UTR of the third polynucleotide. In some embodiments, each polynucleotide that can be transcribed to produce a miRNA target sequence that is operably linked to the third promoter is in the 5′ UTR of the third polynucleotide.

In some embodiments, the fourth inner ear cell type is different from the second inner ear cell type. In some embodiments, the first inner ear cell type is the same as the fourth inner ear cell type. In some embodiments, the first inner ear cell type is different than the fourth inner ear cell type.

In some embodiments, the fourth inner ear cell type is the same as the second inner ear cell type. In some embodiments, the third inner ear cell type is different from the first inner ear cell type.

In some embodiments, the third inner ear cell type is the same as the second inner ear cell type. In some embodiments, the third inner ear cell type is different from the second inner ear cell type.

In some embodiments, the third inner ear cell type is the same as the first inner ear cell type.

In some embodiments, the sixth inner ear cell type is different from the fourth and the second inner ear cell types. In some embodiments, the sixth inner ear cell type is the same as either the fourth inner ear cell type or the second inner ear cell type. In some embodiments, the sixth inner ear cell type is the same as the fourth and the second inner ear cell types.

In some embodiments, the fifth inner ear cell type is different from the first and third inner ear cell types. In some embodiments, the fifth inner ear cell type is the same as either the first inner ear cell type or the third inner ear cell type. In some embodiments, the fifth inner ear cell type is the same as the first and the third inner ear cell types.

In some embodiments, the second promoter is a supporting cell-specific promoter, a hair cell-specific promoter, or a ubiquitous promoter. In some embodiments, the second promoter is a CMV promoter, a MYO15 promoter, an LFNG promoter, an FGFR3 promoter, a SLC1A3 promoter, a GFAP promoter, or a SLC6A14 promoter. In some embodiments, the second promoter is an inner ear cell type-specific promoter listed in Table 12 (e.g., a supporting cell- or hair cell-specific promoter listed in Table 12). In some embodiments, the second polynucleotide is a transgene encoding a protein, is a polynucleotide that can be transcribed to produce an inhibitory RNA, or encodes a component of a gene editing system. In some embodiments, the second polynucleotide is a transgene encoding a protein. In some embodiments, the transgene is a wild-type version of a gene listed in Table 4. In some embodiments, the transgene is a polynucleotide listed in Table 5. In some embodiments, the second polynucleotide can be transcribed to produce an inhibitory RNA. In some embodiments, the inhibitory RNA is an siRNA, shRNA, or shRNA-mir. In some embodiments, the inhibitory RNA is an inhibitory RNA targeting Sox2 (e.g., an inhibitory RNA described herein). In some embodiments, the second polynucleotide encodes a component of a gene editing system. In some embodiments, the second polynucleotide can be transcribed to produce a guide RNA. In some embodiments, the second polynucleotide encodes a nuclease. In some embodiments, the second polynucleotide encodes Atoh1, Gfi1, Pou4f3, Ikzf2, dnSox2, or Gjb2. In some embodiments, one or more (e.g., 1, 2, 3, 4, 5, 6, 7, 8, 9, 10, or more) polynucleotides that can be transcribed to produce a miRNA target sequence are operably linked to the second promoter. In some embodiments, each polynucleotide that can be transcribed to produce a miRNA target sequence that is operably linked to the second promoter is independently targeted by a miRNA listed in Table 2. In some embodiments, each polynucleotide that can be transcribed to produce a miRNA target sequence that is operably linked to the second promoter is independently targeted by one of: miR-183, miR-96, miR-182, miR-18a, miR-100, miR-124a, miR-140, miR-194, miR-135, or miR-135b.

In some embodiments, the third promoter is a supporting cell-specific promoter, a hair cell-specific promoter, or a ubiquitous promoter. In some embodiments, the third promoter is a CMV promoter, a MYO15 promoter, a LFNG promoter, a FGFR3 promoter, a SLC1A3 promoter, a GFAP promoter, or a SLC6A14 promoter. In some embodiments, the third promoter is an inner ear cell type-specific promoter listed in Table 12 (e.g., a supporting cell- or hair cell-specific promoter listed in Table 12). In some embodiments, the third polynucleotide is a transgene encoding a protein, is a polynucleotide that can be transcribed to produce an inhibitory RNA, or encodes a component of a gene editing system. In some embodiments, the third polynucleotide is a transgene encoding a protein. In some embodiments, the transgene is a wild-type version of a gene listed in Table 4. In some embodiments, the transgene is a polynucleotide listed in Table 5. In some embodiments, the third polynucleotide can be transcribed to produce an inhibitory RNA. In some embodiments, the inhibitory RNA is an siRNA, shRNA, or shRNA-mir. In some embodiments, the inhibitory RNA is an inhibitory RNA targeting Sox2 (e.g., an inhibitory RNA described herein). In some embodiments, the third polynucleotide encodes a component of a gene editing system. In some embodiments, the third polynucleotide can be transcribed to produce a guide RNA. In some embodiments, the third polynucleotide encodes a nuclease. In some embodiments, the third polynucleotide encodes Atoh1, Gfi1, Pou4f3, Ikzf2, dnSox2, or Gjb2. In some embodiments, one or more (e.g., 1, 2, 3, 4, 5, 6, 7, 8, 9, 10, or more) polynucleotides that can be transcribed to produce a miRNA target sequence are operably linked to the third promoter. In some embodiments, each polynucleotide that can be transcribed to produce a miRNA target sequence that is operably linked to the third promoter is independently targeted by a miRNA listed in Table 2. In some embodiments, each polynucleotide that can be transcribed to produce a miRNA target sequence that is operably linked to the third promoter is independently targeted by one of: miR-183, miR-96, miR-182, miR-18a, miR-100, miR-124a, miR-140, miR-194, miR-135, or miR-135b.

In some embodiments, each polynucleotide that can be transcribed to produce a miRNA target sequence that is operably linked to the second promoter is the same.

In some embodiments, each polynucleotide that can be transcribed to produce a miRNA target sequence that is operably linked to the third promoter is the same.

In some embodiments, each polynucleotide that can be transcribed to produce a miRNA target sequence that is operably linked to the first promoter is the same as each polynucleotide that can be transcribed to produce a miRNA target sequence that is operably linked to the second promoter. In some embodiments, each polynucleotide that can be transcribed to produce a miRNA target sequence that is operably linked to the first promoter is the same as each polynucleotide that can be transcribed to produce a miRNA target sequence that is operably linked to the third promoter. In some embodiments, each polynucleotide that can be transcribed to produce a miRNA target sequence that is operably linked to the second promoter is the same as each polynucleotide that can be transcribed to produce a miRNA target sequence that is operably linked to the third promoter. In some embodiments, each polynucleotide that can be transcribed to produce a miRNA target sequence that is operably linked to the first promoter is the same as each polynucleotide that can be transcribed to produce a miRNA target sequence that is operably linked to the second promoter and the same as each polynucleotide that can be transcribed to produce a miRNA target sequence that is operably linked to the third promoter.

In some embodiments, each polynucleotide that can be transcribed to produce a miRNA target sequence that is operably linked to the first promoter is different from each polynucleotide that can be transcribed to produce a miRNA target sequence that is operably linked to the second promoter. In some embodiments, each polynucleotide that can be transcribed to produce a miRNA target sequence that is operably linked to the first promoter is different from each polynucleotide that can be transcribed to produce a miRNA target sequence that is operably linked to the third promoter. In some embodiments, each polynucleotide that can be transcribed to produce a miRNA target sequence that is operably linked to the second promoter is different from each polynucleotide that can be transcribed to produce a miRNA target sequence that is operably linked to the third promoter. In some embodiments, each polynucleotide that can be transcribed to produce a miRNA target sequence that is operably linked to the first promoter is different from each polynucleotide that can be transcribed to produce a miRNA target sequence that is operably linked to the second promoter and different from each polynucleotide that can be transcribed to produce a miRNA target sequence that is operably linked to the third promoter.

In some embodiments, at least one polynucleotide that can be transcribed to produce a miRNA target sequence is independently operably linked to both the first promoter and the second promoter, to both the first promoter and the third promoter, to both the second promoter and the third promoter, or to the first, second, and third promoters (e.g., two or more of the polynucleotides that can be transcribed to produce an expression product are regulated by the same miRNA target sequence or by a set of miRNA target sequences that includes a shared miRNA target sequence).

In some embodiments, the third inner ear cell type is a cochlear supporting cell and the fourth inner ear cell type is a cochlear hair cell or a spiral ganglion neuron. In some embodiments, the fourth inner ear cell type is a cochlear hair cell. In some embodiments, the fourth inner ear cell type is a spiral ganglion neuron.

In some embodiments, the third inner ear cell type is a vestibular supporting cell and the fourth inner ear cell type is a vestibular hair cell or a vestibular ganglion neuron. In some embodiments, the fourth inner ear cell type is a vestibular hair cell. In some embodiments, the fourth inner ear cell type is a vestibular type I hair cell. In some embodiments, the fourth inner ear cell type is a vestibular ganglion neuron.

In some embodiments, the third inner ear cell type is a vestibular type II hair cell and the fourth inner ear cell type is a vestibular type I hair cell.

In some embodiments, the third inner ear cell type is a vestibular type II hair cell and the fourth inner ear cell type is a vestibular ganglion neuron.

In some embodiments, the fifth inner ear cell type is a cochlear supporting cell and the sixth inner ear cell type is a cochlear hair cell or a spiral ganglion neuron. In some embodiments, the sixth inner ear cell type is a cochlear hair cell. In some embodiments, the sixth inner ear cell type is a spiral ganglion neuron.

In some embodiments, the fifth inner ear cell type is a vestibular supporting cell and the sixth inner ear cell type is a vestibular hair cell or a vestibular ganglion neuron. In some embodiments, the sixth inner ear cell type is a vestibular hair cell. In some embodiments, the sixth inner ear cell type is a vestibular type I hair cell. In some embodiments, the sixth inner ear cell type is a vestibular ganglion neuron.

In some embodiments, the fifth inner ear cell type is a vestibular type II hair cell and the sixth inner ear cell type is a vestibular type I hair cell.

In some embodiments, the fifth inner ear cell type is a vestibular type II hair cell and the sixth inner ear cell type is a vestibular ganglion neuron.

In some embodiments, (a) the first polynucleotide encodes Atoh1, Gfi1, Pou4f3, Ikzf2, dnSox2, or Gjb2 or can be transcribed to produce an inhibitory RNA targeting Sox2; (b) the first promoter is a CMV promoter, an FGFR3 promoter, an LFNG promoter, or a SLC1A3 promoter; (c) each miRNA target sequence transcribed from a polynucleotide operably linked to the first promoter is independently targeted by one of: miR-183, miR-96, miR-182, miR-18a, miR-140, or miR-194; (d) the first inner ear cell type is a cochlear supporting cell; and (e) the second inner ear cell type is cochlear hair cell. In some embodiments, the first polynucleotide encodes Atoh1 and the second polynucleotide encodes is Ikzf2. In some embodiments, the first polynucleotide encodes Atoh1, the second polynucleotide encodes Gfi1, and the third polynucleotide encodes Pou4f3.

In some embodiments, (a) the first polynucleotide encodes GJB2; (b) the first promoter is a GJB2 promoter, a CMV promoter, an FGFR3 promoter, an LFNG promoter, or a SLC1A3 promoter; (c) each miRNA target sequence transcribed from a polynucleotide operably linked to the first promoter is independently targeted by one of: miR-183, miR-96, miR-182, miR-18a, miR-124, or miR-194; (d) the first inner ear cell type is a cochlear supporting cell; and (e) the second inner ear cell type is spiral ganglion neuron.

In some embodiments, (a) the first polynucleotide encodes Atoh1 or dnSox2 or can be transcribed to produce an inhibitory RNA targeting Sox2; (b) the first promoter is a CMV promoter, a GFAP promoter, a SLC6A14 promoter, or a SLC1A3 promoter; (c) each miRNA target sequence transcribed from a polynucleotide operably linked to the first promoter is independently targeted by one of: miR-183, miR-96, miR-182, miR-18a, miR-140, or miR-135b; (d) the first inner ear cell type is a vestibular supporting cell; and (e) the second inner ear cell type is vestibular hair cell.

In some embodiments, (a) the first polynucleotide encodes Atoh1 or dnSox2 or can be transcribed to produce an inhibitory RNA targeting Sox2; (b) the first promoter is a CMV promoter, a GFAP promoter, a SLC6A14 promoter, or a SLC1A3 promoter; (c) each miRNA target sequence transcribed from a polynucleotide operably linked to the first promoter is independently targeted by one of: miR-183, miR-96, miR-182, miR-18a, miR-124a, miR-100, or miR-135; (d) the first inner ear cell type is a vestibular supporting cell; and (e) the second inner ear cell type is vestibular ganglion neuron.

In some embodiments, (a) the first polynucleotide encodes dnSox2 or can be transcribed to produce an inhibitory RNA targeting Sox2; (b) the first promoter is a MYO15 promoter; (c) each miRNA target sequence transcribed from a polynucleotide operably linked to the first promoter is independently targeted by one of: miR-183, miR-96, miR-182, miR-18a, miR-124a, miR-100, or miR-135; (d) the first inner ear cell type is a type II hair cell; and (e) the second inner ear cell type is vestibular ganglion neuron. In some embodiments, each miRNA target sequence present is independently targeted by one of: miR-18a, miR-124a, miR-100, or miR-135.

In some embodiments, the inhibitory RNA targeting Sox2 is an siRNA. In some embodiments, the inhibitory RNA targeting Sox2 is an shRNA. In some embodiments, the siRNA or shRNA targeting Sox2 has a nucleobase sequence containing a portion of at least 8 contiguous nucleobases having at least 80% complementarity to an equal length portion of a target region of an mRNA transcript of a human or murine SOX2 gene. In some embodiments, the target region is an mRNA transcript of the human SOX2 gene. In some embodiments, the target region is at least 8 to 21 contiguous nucleobases of any one of SEQ ID NOs: 52-70, at least 8 to 22 contiguous nucleobases of SEQ ID NO: 74 or SEQ ID NO: 75, or at least 8 to 19 contiguous nucleobases of any one of SEQ ID NOs: 71-73. In some embodiments, the siRNA or shRNA has a nucleobase sequence containing a portion of at least 8 contiguous nucleobases having at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) complementarity to an equal length portion of any one of SEQ ID NOs: 52-75. In some embodiments, the siRNA or shRNA has a nucleobase sequence having at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) complementarity to any one of SEQ ID NO: 58, SEQ ID NO: 71, SEQ ID NO: 72, SEQ ID NO: 73, SEQ ID NO: 74, and SEQ ID NO: 75. In some embodiments, the shRNA comprises the sequence of nucleotides 2234-2296 of SEQ ID NO: 76 or nucleotides 2234-2296 of SEQ ID NO: 78. In some embodiments, the shRNA is embedded in a microRNA (miRNA) backbone. In some embodiments, the shRNA is embedded in a miR-30 or mir-E backbone. In some embodiments, the shRNA includes the sequence of nucleotides 2109-2426 of SEQ ID NO: 76, nucleotides 2109-2408 of SEQ ID NO: 66, nucleotides 2109-2426 of SEQ ID NO: 78, or nucleotides 2109-2408 of SEQ ID NO: 79. In some embodiments, the siRNA contains a sense strand and an antisense strand selected from the following pairs: SEQ ID NO: 80 and SEQ ID NO: 81; SEQ ID NO: 82 and SEQ ID NO: 83; SEQ ID NO: 84 and SEQ ID NO: 85; and SEQ ID NO: 86 and SEQ ID NO: 87.

In some embodiments, the polynucleotide encoding the dnSox2 protein has the sequence of SEQ ID NO: 50 or SEQ ID NO: 51. In some embodiments, the dnSox2 protein is a Sox2 protein that lacks most or all of the high mobility group domain (HMGD), a Sox2 protein in which the nuclear localization signals in the HMGD are mutated, a Sox2 protein in which the HMGD is fused to an engrailed repressor domain, or a c-terminally truncated Sox2 protein comprising only the DNA binding domain.

In some embodiments, the nucleic acid vector is a plasmid, cosmid, artificial chromosome, or viral vector. In some embodiments, the nucleic acid vector is a viral vector. In some embodiments, the viral vector is selected from the group consisting of an adeno-associated virus (AAV), an adenovirus, and a lentivirus. In some embodiments, the viral vector is an AAV vector. In some embodiments, the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.B2, PBP.B3, PHP.A, PHP.eb, or PHP.S capsid. In some embodiments, the AAV vector has an AAV1 capsid. In some embodiments, the AAV vector has an AAV2 capsid. In some embodiments, the AAV vector has an AAV8 capsid. In some embodiments, the AAV vector has an AAV9 capsid. In some embodiments, the AAV vector has an AAV2(quadY-F) capsid. In some embodiments, the AAV vector has an AAV6 capsid. In some embodiments, the AAV vector has a 7m8 capsid. In some embodiments, the AAV vector has an Anc80 capsid. In some embodiments, the AAV vector has an Anc80L65 capsid. In some embodiments, the AAV vector has a DJ/9 capsid. In some embodiments, the AAV vector has a PHP.B capsid. In some embodiments, the AAV vector has a PHP.eb capsid.

In another aspect, the invention provides a pharmaceutical composition including the nucleic acid vector of the invention and a pharmaceutically acceptable carrier, excipient, or diluent.

In another aspect, the invention provides a kit including a nucleic acid vector or pharmaceutical composition of the invention.

In another aspect, the invention provides a method of expressing a polynucleotide in a first inner ear cell type and not in a second inner ear cell type in a subject in need thereof by locally administering to the middle or inner ear of the subject an effective amount of a nucleic acid vector or pharmaceutical composition of the invention.

In another aspect, the invention provides a method of reducing off-target expression of a polynucleotide in an inner ear of a subject (e.g., reducing off target expression in a particular inner ear cell type) by locally administering to the middle or inner ear of the subject an effective amount of a nucleic acid vector or pharmaceutical composition of the invention.

In some embodiments of any of the foregoing aspects, the subject has or is at risk of developing hearing loss, vestibular dysfunction, or tinnitus.

In another aspect, the invention provides a method of treating a subject having or at risk of developing hearing loss, vestibular dysfunction, or tinnitus, comprising administering to the subject an effective amount of a nucleic acid vector or pharmaceutical composition of the invention.

In some embodiments of any of the foregoing aspects, the subject has or is at risk of developing vestibular dysfunction.

In some embodiments of any of the foregoing aspects, the vestibular dysfunction is vertigo, dizziness, imbalance, bilateral vestibulopathy, oscillopsia, or a balance disorder. In some embodiments of any of the foregoing aspects, the vestibular dysfunction is age-related vestibular dysfunction, head trauma-related vestibular dysfunction, disease or infection-related vestibular dysfunction, or ototoxic drug-induced vestibular dysfunction. In some embodiments of any of the foregoing aspects, the vestibular dysfunction is associated with a genetic mutation. In some embodiments, the genetic mutation is a mutation in a gene listed in Table 4. In some embodiments of any of the foregoing aspects, the vestibular dysfunction is idiopathic vestibular dysfunction.

In some embodiments of any of the foregoing aspects, the subject has or is at risk of developing hearing loss (e.g., sensorineural hearing loss, including auditory neuropathy and deafness). In some embodiments of any of the foregoing aspects, the hearing loss is genetic hearing loss. In some embodiments, the genetic hearing loss is autosomal dominant hearing loss, autosomal recessive hearing loss, or X-linked hearing loss. In some embodiments, the genetic hearing loss is a condition associated with a mutation in a gene listed in Table 4. In some embodiments of any of the foregoing aspects, the hearing loss is acquired hearing loss. In some embodiments, the acquired hearing loss is noise-induced hearing loss, age-related hearing loss, disease or infection-related hearing loss, head trauma-related hearing loss, or ototoxic drug-induced hearing loss.

In some embodiments of any of the foregoing aspects, the ototoxic drug is an aminoglycoside, an antineoplastic drug, ethacrynic acid, furosemide, a salicylate, or quinine.

In some embodiments of any of the foregoing aspects, the hearing loss or vestibular dysfunction is or is associated with age-related hearing loss, noise-induced hearing loss, DFNB61, DFNB1, DFNB7/11, DFNA2, DFNB77, DFNB28, DFNA41, DFNB8, DFNB37, DFNA22, DFNB3, Usher syndrome type 1, Usher syndrome type 2, or bilateral vestibulopathy.

In some embodiments of any of the foregoing aspects, the hearing loss is or is associated with age-related hearing loss, noise-induced hearing loss, DFNB61, DFNB1, DFNB7/11, DFNA2, DFNB77, DFNB28, DFNA41, DFNB8, DFNB37, DFNA22, DFNB3, Usher syndrome type 1, or Usher syndrome type 2 and the first polynucleotide encodes Atoh1. In some embodiments, the second polynucleotide encodes Ikzf2. In some embodiments, the second polynucleotide encodes Pou4f3 and the third polynucleotide encodes Gfi1.

In some embodiments of any of the foregoing aspects, the method further includes administering to the subject one or more (e.g., 1, 2, 3, 4, 5, or more) additional nucleic acid vectors. In some embodiments, the subject is additionally administered a vector comprising a polynucleotide encoding Ikzf2. In some embodiments, the subject is additionally administered a vector comprising a polynucleotide encoding Pou4f3 and a vector comprising a polynucleotide encoding Gfi1.

In some embodiments of any of the foregoing aspects, the hearing loss or vestibular dysfunction is or is associated with DFNB1, DFNB7/11, DFNA2, DFNB77, DFNB28, DFNA41, DFNB8, DFNB37, DFNA22, DFNB3, Usher syndrome type 1, Usher syndrome type 2, or bilateral vestibulopathy and the first polynucleotide encodes dnSox2. In some embodiments, the second polynucleotide encodes Atoh1. In some embodiments, the subject is additionally administered a vector comprising a polynucleotide encoding Atoh1.

In some embodiments of any of the foregoing aspects, at least one of the one or more additional nucleic acid vectors comprises a promoter operably linked to a polynucleotide that can be transcribed to produce an expression product (e.g., Ikzf2, Pou4f3, Gfi1, or Atoh1) and to a polynucleotide that can be transcribed to produce a miRNA target sequence.

In some embodiments of any of the foregoing aspects, none of the additional nucleic acid vectors comprise a polynucleotide that can be transcribed to produce a miRNA target sequence.

In another aspect, the invention provides a method of treating a condition listed in Table 4 in a subject in need thereof by locally administering to the middle or inner ear of the subject an effective amount of a nucleic acid vector or pharmaceutical composition of the invention, in which the first polynucleotide is a wild-type version of a gene associated with the condition listed in Table 4 that is mutated in the subject.

In some embodiments of any of the foregoing aspects, the method further includes evaluating the vestibular function of the subject prior to administering the nucleic acid vector or pharmaceutical composition. In some embodiments of any of the foregoing aspects, the method further includes evaluating the vestibular function of the subject after administering the nucleic acid vector or pharmaceutical composition.

In some embodiments of any of the foregoing aspects, the method further includes evaluating the hearing of the subject prior to administering the nucleic acid vector or pharmaceutical composition. In some embodiments of any of the foregoing aspects, the method further includes evaluating the hearing of the subject after administering the nucleic acid vector or pharmaceutical composition.

In some embodiments of any of the foregoing aspects, the nucleic acid vector or pharmaceutical composition is administered to the inner ear. In some embodiments of any of the foregoing aspects, the nucleic acid vector or pharmaceutical composition is administered to the middle ear. In some embodiments of any of the foregoing aspects, the nucleic acid vector or pharmaceutical composition is administered to a semicircular canal. In some embodiments of any of the foregoing aspects, the nucleic acid vector or pharmaceutical composition is administered transtympanically or intratympanically. In some embodiments of any of the foregoing aspects, the nucleic acid vector or pharmaceutical composition is administered into the perilymph. In some embodiments of any of the foregoing aspects, the nucleic acid vector or pharmaceutical composition is administered into the endolymph. In some embodiments of any of the foregoing aspects, the nucleic acid vector or pharmaceutical composition is administered to or through the oval window. In some embodiments of any of the foregoing aspects, the nucleic acid vector or pharmaceutical composition is administered to or through the round window.

In some embodiments of any of the foregoing aspects, the nucleic acid vector or pharmaceutical composition is administered in an amount sufficient to prevent or reduce vestibular dysfunction, delay the development of vestibular dysfunction, slow the progression of vestibular dysfunction, improve vestibular function, prevent or reduce hearing loss, prevent or reduce tinnitus, delay the development of hearing loss, slow the progression of hearing loss, improve hearing, increase vestibular and/or cochlear hair cell numbers, increase vestibular and/or cochlear hair cell maturation, increase vestibular and/or cochlear hair cell regeneration, treat bilateral vestibulopathy, treat oscillopsia, treat a balance disorder, improve the function of one or more inner ear cell types, improve inner ear cell survival, increase inner ear cell proliferation, increase the generation of Type I vestibular hair cells, or increase the number of Type I vestibular hair cells.

In some embodiments of any of the foregoing aspects, the subject is a human.

In another aspect, the invention provides an inner ear cell containing a nucleic acid vector or pharmaceutical composition of the invention. In some embodiments, the inner ear cell is a cochlear supporting cell. In some embodiments, the inner ear cell is a vestibular supporting cell. In some embodiments, the inner ear cell is a cochlear hair cell. In some embodiments, the inner ear cell is a vestibular hair cell. In some embodiments, the inner ear cell is a vestibular type I hair cell. In some embodiments, the inner ear cell is a vestibular type II hair cell. In some embodiments, the inner ear cell is a spiral ganglion neuron. In some embodiments, the inner ear cell is a vestibular ganglion neuron. In some embodiments, the inner ear cell is a human inner ear cell.

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, “administration” refers to providing or giving a subject a therapeutic agent (e.g., a vector for expressing a transgene in an inner ear cell), by any effective route. Exemplary routes of administration are described herein below.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the term “cochlear hair cell” refers to group of specialized cells in the inner ear that are involved in sensing sound. There are two types of cochlear hair cells: inner hair cells and outer hair cells. Damage to cochlear hair cells and genetic mutations that disrupt cochlear hair cell function are implicated in hearing loss and deafness.

As used herein, the terms “complementarity” or “complementary” of nucleic acids means that a nucleotide sequence in one strand of nucleic acid, due to orientation of its nucleobase groups, forms hydrogen bonds with another sequence on an opposing nucleic acid strand. The complementary bases in DNA are typically A with T and C with G. In RNA, they are typically C with G and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm (melting temperature) of hybridized strands, or by empirical determination of Tm by using routine methods. Tm includes the temperature at which a population of hybridization complexes formed between two nucleic acid strands are 50% denatured (i.e., a population of double-stranded nucleic acid molecules becomes half dissociated into single strands). At a temperature below the Tm, formation of a hybridization complex is favored, whereas at a temperature above the Tm, melting or separation of the strands in the hybridization complex is favored. Tm may be estimated for a nucleic acid having a known G+C content in an aqueous 1 M NaCl solution by using, e.g., Tm=81.5+0.41(% G+C), although other known Tm computations take into account nucleic acid structural characteristics.

As used herein, the terms “effective amount,” “therapeutically effective amount,” and a “sufficient amount” of a composition, vector construct, or viral vector described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating hearing loss or vestibular dysfunction, it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, vector construct, or viral vector of the present disclosure is an amount which results in a beneficial or desired result in a subject as compared to a control. As defined herein, a therapeutically effective amount of a composition, vector construct, or viral vector of the present disclosure may be readily determined by one of ordinary skill by routine methods known in the art. Dosage regimen may be adjusted to provide the optimum therapeutic response.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human vestibular supporting cell).

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. The term “expression product” refers to a protein or RNA molecule produced by any of these events.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human vestibular supporting cell). Exogenous materials include those that are provided from an external source to an organism or to cultured matter extracted there from.

As used herein, the term “heterologous” refers to a combination of elements that is not naturally occurring. For example, a heterologous transgene refers to a transgene that is not naturally expressed by the promoter to which it is operably linked.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., transgene expression) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “inner ear cell type” refers to a cell type found in the inner ear (e.g., cochlea and/or vestibular system) of a subject (e.g., a human subject). Inner ear cell types include cochlear hair cells (which can be further divided into inner hair cells and outer hair cells), Type I vestibular hair cells, Type II vestibular hair cells, vestibular dark cells, vestibular fibrocytes, Scarpa's ganglion neurons (vestibular ganglion neurons), endothelial cells of vestibular capillaries, vestibular supporting cells, cochlear supporting cells (which includes border cells, inner phalangeal cells, inner pillar cells, outer pillar cells, first row Deiters' cells, second row Deiters' cells, third row Deiters' cells, and Hensen's cells), Claudius cells, spiral prominence cells, root cells, interdental cells, basal cells of the stria vascularis, intermediate cells of the stria vascularis, marginal cells of the stria vascularis, spiral ganglion neurons, endothelial cells of cochlear capillaries, fibrocytes, cells of Reissner's membrane, and glial cells.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the middle or inner ear, and administration to a mucous membrane of the subject, wherein the administration is intended to have a local and not a systemic effect.

As used herein, the term “operably linked” refers to a first molecule joined to a second molecule, wherein the molecules are so arranged that the first molecule affects the function of the second molecule.

The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “polynucleotide” refers to a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. The term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the term “promoter” refers to a recognition site on DNA that is bound by an RNA polymerase. The polymerase drives transcription of the transgene.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response, and other problem complications commensurate with a reasonable benefit/risk ratio.

As used herein, the term “supporting cell” refers specialized epithelial cells in the cochlea and vestibular system of the inner ear that reside between hair cells. Supporting cells maintain the structural integrity of the sensory organs during sound stimulation and head movements and help to maintain an environment in the epithelium that allows hair cells to function. Supporting cells are also involved in cochlear and vestibular hair cell development, survival, death, and phagocytosis.

As used herein, the term “transcription regulatory element” refers to a nucleic acid that controls, at least in part, the transcription of a gene of interest. Transcription regulatory elements may include promoters, enhancers, and other nucleic acids (e.g., polyadenylation signals) that control or help to control gene transcription. Examples of transcription regulatory elements are described, for example, in Lorence, Recombinant Gene Expression: Reviews and Protocols (Humana Press, New York, NY, 2012).

As used herein, the term “transfection” refers to any of a wide variety of techniques commonly used for the introduction of exogenous DNA into a prokaryotic or eukaryotic host cell, e.g., electroporation, lipofection, calcium phosphate precipitation, DEAE-dextran transfection, Nucleofection, squeeze-poration, sonoporation, optical transfection, magnetofection, impalefection and the like.

As used herein, the terms “subject” and “patient” refer to an animal (e.g., a mammal, such as a human). A subject to be treated according to the methods described herein may be one who has been diagnosed with hearing loss (e.g., sensorineural hearing loss or deafness) and/or vestibular dysfunction (e.g., dizziness, vertigo, imbalance or loss of balance, bilateral vestibulopathy, oscillopsia, or a balance disorder) or one at risk of developing these conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the phrase “suitable for expression” refers to a polynucleotide that is intended for expression in an inner ear cell type, including but not limited to (i) polynucleotides that are expressed in the inner ear cell type and (ii) polynucleotides that modulate a gene or protein that is expressed in the inner ear cell type.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” includes a nucleic acid vector, e.g., a DNA vector, such as a plasmid, cosmid, or artificial chromosome, an RNA vector, a virus, or any other suitable replicon (e.g., viral vector). A variety of vectors have been developed for the delivery of polynucleotides encoding exogenous proteins into a prokaryotic or eukaryotic cell. Examples of such expression vectors are described in, e.g., Gellissen, Production of Recombinant Proteins: Novel Microbial and Eukaryotic Expression Systems (John Wiley & Sons, Marblehead, M A, 2006). Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of transgene as described herein include vectors that contain regulatory sequences, such as promoter and enhancer regions, which direct gene transcription. Other useful vectors for expression of a transgene contain polynucleotide sequences that enhance the rate of translation of the transgene or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the term “vestibular hair cell” refers to group of specialized cells in the inner ear that are involved in sensing movement and contribute to the sense of balance and spatial orientation. There are two types of vestibular hair cells: Type I and Type II hair cells. Vestibular hair cells are located in the semicircular canal end organs and otolith organs of the inner ear. Damage to vestibular hair cells and genetic mutations that disrupt vestibular hair cell function are implicated in vestibular dysfunction such as vertigo, bilateral vestibulopathy, oscillopsia, and balance disorders.

As used herein, the term “vestibular sensory epithelium” refers to any of vestibular Type I hair cells, vestibular Type II hair cells, and vestibular supporting cells.

As used herein, the term “wild-type” refers to a genotype with the highest frequency for a particular gene in a given organism.

### BRIEF DESCRIPTION OF THE DRAWINGS

FIG. 1 is a plasmid map of transgene plasmid P742.

FIG. 2 is a plasmid map of transgene plasmid P744.

FIG. 3 is a plasmid map of transgene plasmid P745.

FIG. 4 is a plasmid map of transgene plasmid P746.

FIG. 5 is a plasmid map of transgene plasmid P747.

FIG. 6 is a plasmid map of transgene plasmid P002.

FIG. 7 is a series of micrographs showing expression of GFP in HEK293-T cells transfected with different AAV vectors. Each pair of panels (e.g., A and A′; B and B′, etc.) shows the same field of cells displaying GFP expression (A, B, C, D, E, F and G) and nuclear staining with DAPI (A′, B′, C′, D′, E′, F′ and G′) for each different AAV vector.

FIG. 8 is a plasmid map of transgene plasmid P740.

FIG. 9 is a plasmid map of transgene plasmid P741.

FIG. 10 is a plasmid map of transgene plasmid P743.

FIG. 11 is a plasmid map of transgene plasmid P750.

FIG. 12 is a plasmid map of transgene plasmid P752.

FIG. 13 is a plasmid map of transgene plasmid P753.

FIG. 14 is a plasmid map of transgene plasmid P754.

FIG. 15 is a plasmid map of transgene plasmid P755.

FIG. 16 is a plasmid map of transgene plasmid P748.

FIG. 17 is a plasmid map of transgene plasmid P749.

FIG. 18 is a plasmid map of transgene plasmid P751.

FIG. 19 is a plasmid map of transgene plasmid P1137.

FIG. 20 is a plasmid map of transgene plasmid P1138.

FIG. 21 is a plasmid map of transgene plasmid P1139.

FIG. 22 is a plasmid map of transgene plasmid P1140.

FIG. 23 is a plasmid map of transgene plasmid P1141.

FIG. 24 is a plasmid map of transgene plasmid P1142.

FIG. 25 is a plasmid map of transgene plasmid P1143.

FIG. 26 is a plasmid map of transgene plasmid P1144.

FIGS. 27A-27B are a series of micrographs of cells transfected with plasmid P1137, which contains one copy of a polynucleotide that can be transcribed to produce an miR-96 target sequence (FIGS. 27A and 27B, top row), or plasmid P1142, which contains four copies of a polynucleotide that can be transcribed to produce an miR-96 target sequence (FIGS. 27A and 27B, bottom row), alone (−miR96) (FIG. 27A) or co-transfected with miR-96 (+miR-96) (FIG. 27B). The bright field and fluorescent (GFP) channels from the same field of cells are shown separately.

FIGS. 28A-28B are a series of micrographs of cells transfected with plasmid P1138, which contains one copy of a polynucleotide that can be transcribed to produce an miR-182 target sequence (FIGS. 28A and 28B, top row), or plasmid P1143, which contains four copies of a polynucleotide that can be transcribed to produce an miR-182 target sequence (FIGS. 28A and 28B, bottom row), alone (−miR-182) (FIG. 28A) or co-transfected with miR-182 (+miR-182) (FIG. 28B). The bright field and fluorescent (GFP) channels from the same field of cells are shown separately.

FIGS. 29A-29B are a series of micrographs of cells transfected with plasmid P1139, which contains one copy of a polynucleotide that can be transcribed to produce an miR-183 target sequence (FIGS. 29A and 29B, top row), or plasmid P1144, which contains four copies of a polynucleotide that can be transcribed to produce an miR-183 target sequence (FIGS. 29A and 29B, bottom row), alone (−miR-183) (FIG. 29A) or co-transfected with miR-183 (+miR-183) (FIG. 29B). The bright field and fluorescent (GFP) channels from the same field of cells are shown separately.

FIGS. 30A-30B are a series of micrographs of cells transfected with plasmid P1140, which contains one copy of each polynucleotide that can be transcribed to produce a miR-96 target sequence, a miR-182 target sequence, and a miR-183 target sequence (FIGS. 30A and 30B, top row), or plasmid P1141, which contains three copies of each polynucleotide that can be transcribed to produce a miR-96 target sequence, a miR-182 target sequence, and a miR-183 target sequence (FIGS. 30A and 30B, bottom row), alone (−miR-183/96/182) (FIG. 30A) or co-transfected with miR-96, miR-182 and miR-183 (+miR-183/96/182) (FIG. 30B). The bright field and fluorescent (GFP) channels from the same field of cells are shown separately.

FIG. 31 is a bar graph showing the percentage of cells expressing GFP after being transfected with the indicated plasmid alone or co-transfected with the appropriate miRNA(s). The number of copies of the miRNA target sequences is indicated for each plasmid.

FIGS. 32A-32B are a series of micrographs of regions of a neonatal mouse cochlear explant taken five days after infection with various AAV vectors that express eGFP under control of a CMV promoter. FIG. 32A shows explants sequentially infected with AAV807 (a control vector that expresses eGFP under control of a CMV promoter, but lacks any miRNA target sequences) (“AAV807”), with AAV 1026 (created from transgene plasmid P1142 containing four copies of a polynucleotide that can be transcribed to produce a miR-96 target sequence) (“AAV1026”), or with AAV 1027 (created from transgene plasmid P1143 containing four copies of a polynucleotide that can be transcribed to produce a miR-182 target sequence) (“AAV1027”). FIG. 32B shows explants infected with AAV807 (“AAV807”), with AAV 1028 (created from transgene plasmid P1144 containing four copies of a polynucleotide that can be transcribed to produce a miR-183 target sequence) (“AAV1028”), or with AAV1029 (created from transgene plasmid P1141 containing three copies of each polynucleotide that can be transcribed to produce a miR-96 target sequence, a miR-182 target sequence, and a miR-183 target sequence) (“AAV1029”). The sections were also stained with an antibody against Myo7a to stain hair cells and an antibody against Sox2 to stain supporting cells. Channels displaying Myo7a staining alone (top row), Sox2 staining alone (middle row) and GFP alone (bottom row) are shown for each AAV vector infection.

FIG. 33 is a plasmid map of transgene plasmid P1315.

FIG. 34 is a plasmid map of transgene plasmid P1316.

FIG. 35 is a plasmid map of transgene plasmid P1317.

FIG. 36 is a plasmid map of transgene plasmid P1318.

FIGS. 37A-37B are a series of micrographs of regions of a neonatal mouse cochlear explant taken five days after infection with various AAV vectors that express eGFP under control of a LFNG promoter. FIG. 37A shows explants infected with AAV851 (a control vector that expresses eGFP under control of a LFNG promoter, but lacks any miRNA target sequences) (“AAV851”), with AAV 1146 (created from transgene plasmid P1316 containing four copies of a polynucleotide that can be transcribed to produce a miR-96 target sequence) (“AAV1146”), or with AAV1147 (created from transgene plasmid P1317 containing four copies of a polynucleotide that can be transcribed to produce a miR-182 target sequence) (“AAV1147”). FIG. 37B shows explants infected with AAV851 (“AAV851”), with AAV1148 (created from transgene plasmid P1318 containing four copies of a polynucleotide that can be transcribed to produce a miR-183 target sequence) (“AAV1148”), or with AAV1145 (created from transgene plasmid P1315 containing three copies of each polynucleotide that can be transcribed to produce a miR-96 target sequence, a miR-182 target sequence, and a miR-183 target sequence) (“AAV1145”). The tissues were also stained with an antibody against Myo7a to stain hair cells and an antibody against Sox2 to stain supporting cells. Channels displaying Myo7a staining alone (top row), Sox2 staining alone (middle row) and GFP alone (bottom row) are shown for each AAV vector transfection.

FIGS. 38A-38B are a series of micrographs of neonatal mouse cochlear explants taken five days after infection with various AAV vectors that express eGFP under control of a CMV promoter. FIG. 38A shows explants sequentially infected with AAV807, AAV1026, or AAV1027. FIG. 38B shows explants infected with AAV807, AAV1028, or AAV1029. The sections were also stained with an antibody against Pou4f3 to stain hair cell nuclei and an antibody against Sox2 to stain supporting cell nuclei. Channels displaying Pou4f3 staining alone (top row), Sox2 staining alone (middle row) and GFP alone (bottom row) are shown for each AAV vector infection.

FIG. 39 is a bar graph showing the percentage of hair cells in mouse utricle explants that were GFP positive when infected with AAV851, AAV1145, AAVV1146, AAV1147, or AAV1148.

### DETAILED DESCRIPTION OF THE INVENTION

Described herein are compositions and methods for treating hearing loss and/or vestibular dysfunction. The invention features nucleic acid vectors (e.g., viral vectors, such as adeno-associated virus (AAV) vectors) containing at least one promoter, at least one polynucleotide that can be transcribed to produce a desired expression product (e.g., a transgene encoding a protein of interest), and at least one polynucleotide that can be transcribed to produce a microRNA (miRNA) target sequence. The nucleic acid vectors described herein can be used to express the polynucleotide that can be transcribed to produce a desired expression product (e.g., to produce a protein encoded by a transgene) in a first type of inner ear cell (e.g., an inner ear cell type that does not express an endogenous miRNA that binds to the miRNA target sequence transcribed from the vector) and to reduce or inhibit expression of the polynucleotide that can be transcribed to produce a desired expression product (e.g., production of a protein encoded by a transgene) in a second type of inner ear cell (e.g., an inner ear cell type that expresses an endogenous miRNA that recognizes the miRNA target sequence transcribed from the vector). Therefore, the compositions described herein can be used to achieve cell type-specific expression of a polynucleotide of interest in certain inner ear cell types, and, accordingly, can be administered to a subject (a mammalian subject, for example, a human) to treat disorders caused by a genetic mutation in an inner ear cell, such as genetic hearing loss (e.g., sensorineural hearing loss), deafness, or auditory neuropathy, or to treat disorders caused by loss of or damage to cochlear or vestibular inner ear cells (e.g., hair cells or ganglion neurons), such as sensorineural hearing loss, deafness, auditory neuropathy, tinnitus, dizziness, vertigo, imbalance, bilateral vestibulopathy, and oscillopsia.

### Inner Ear Cells

The inner ear has two main parts: the cochlea, which is responsible for hearing, and the vestibular system, which is dedicated to balance. Both the cochlea and the vestibular system contain specialized cell types, including hair cells, supporting cells, and ganglion neurons.

Hair cells are sensory cells of the auditory and vestibular systems that reside in the inner ear. Cochlear hair cells are the sensory cells of the auditory system and are made up of two main cell types: inner hair cells, which are responsible for sensing sound, and outer hair cells, which are thought to amplify low-level sound. Vestibular hair cells, which include Type I and Type II hair cells, are located in the semicircular canal end organs and otolith organs of the inner ear and are involved in the sensation of movement that contributes to the sense of balance and spatial orientation. Cochlear hair cells are essential for normal hearing, and damage to or loss of cochlear hair cells and genetic mutations that disrupt cochlear hair cell function are implicated in hearing loss and deafness. Damage to or loss of vestibular hair cells and genetic mutations that disrupt vestibular hair cell function are implicated in vestibular dysfunction, such as dizziness, vertigo, balance loss, bilateral vestibulopathy, oscillopsia, and balance disorders.

Supporting cells, which are non-sensory cells that reside between hair cells, perform a diverse set of functions in the cochlea and vestibular system, such as providing a structural scaffold to allow for mechanical stimulation of hair cells, maintaining the ionic composition of the endolymph and perilymph, and regulating synaptogenesis of ribbon synapses. Following trauma or toxicity, supporting cells can eject injured hair cells from the epithelium, phagocytose hair cell debris, and, in some cases, generate new hair cells. Within the cochlea, supporting cells can be subdivided into five different types: 1) Hensen's cells, 2) Deiters' cells, 3) pillar cells; 4) inner phalangeal cells; and 5) border cells, all of which have distinct morphologies and patterns of gene expression. Mutations in genes expressed in cochlear supporting cells have been associated with hearing loss (e.g., sensorineural hearing loss, auditory neuropathy, and deafness) and tinnitus, as has damage, injury, degeneration, or loss (e.g., death) of these cells. Similarly, mutations in genes expressed in vestibular supporting cells and damage, injury, degeneration, or loss (e.g., death) of these cells have been associated with vestibular dysfunction.

Ganglion neurons are bipolar neurons that form a connection between the hair cells of the inner ear and the brain. The cochlea contains spiral ganglion neurons, which form afferent synapses with inner and outer hair cells. The axons of the spiral ganglion neurons make up the cochlear nerve, which is the auditory portion of the eighth cranial nerve. Death, damage to, or degeneration of spiral ganglion neurons can cause sensorineural hearing loss, and certain types of deafness are thought to result from mutations in genes that are expressed in spiral ganglion neurons. The vestibular system includes vestibular ganglion neurons (also called Scarpa's ganglion neurons), which innervate vestibular hair cells in the vestibular system (e.g., in the utricle, saccule, and semicircular canals). Axons of vestibular ganglion neurons make up the vestibular nerve, which is the vestibular portion of the eighth cranial nerve. Death, damage to, or degeneration of vestibular ganglion neurons, whether due to a genetic mutation or to disease or infection, head trauma, ototoxic drugs, or aging, can lead to vestibular dysfunction.

### Cell Type-Specific Gene Expression in Inner Ear Cells

Gene therapy has emerged as a promising therapeutic for treating hearing loss and vestibular dysfunction. It offers the possibility of restoring hearing to subjects suffering from hearing loss, deafness, auditory neuropathy, or vestibular dysfunction due to specific genetic mutations, and may also be used to deliver genes that regulate the formation or differentiation of inner ear cells to promote hair cell regeneration in subjects whose hearing loss or vestibular dysfunction results from hair cell loss or damage. However, the development of gene therapies for the treatment of hearing loss and vestibular dysfunction is made more challenging by the variety of different cell types in the inner ear. Off-target gene expression (e.g., expression of a gene in a cell in which it is not normally expressed) may lead to toxicity, potentially damaging or killing cells. Therefore, there is a need for new approaches that can be used to promote cell type-specific gene expression in a particular cell type (e.g., in the cell type in which the gene would normally be expressed, or in the cell type that is to be genetically modified) and limit off-target expression.

The present inventors have developed a new approach for cell type-specific gene expression in the inner ear based on the use of miRNA target sequences. This approach involves nucleic acid vectors containing at least one promoter, at least one polynucleotide that can be transcribed to produce a desired expression product (e.g., 1, 2, 3, or more polynucleotides, such as a transgene encoding a protein or a polynucleotide that can be transcribed to produce an inhibitory RNA molecule), and at least one polynucleotide that can be transcribed to produce a miRNA target sequence. The polynucleotide that can be transcribed to produce a miRNA target sequence is located within the vector such that it is operably linked to the same promoter as the polynucleotide it regulates (e.g., the polynucleotide that can be transcribed to produce a desired expression product), and it is typically transcribed as part of the same RNA transcript as the desired expression product. The miRNA target sequences for use in the vectors described herein are target sequences for miRNAs that are differentially expressed by different inner ear cell types. For example, a vector may contain a polynucleotide that can be transcribed to produce a target sequence for a miRNA that is not expressed in a first inner ear cell type but that is expressed in a second inner ear cell type. If both cell types were transduced with the vector, the miRNA expressed in the second cell type could recognize (e.g., bind to) the miRNA target sequence and could, therefore, block translation of or degrade the messenger RNA (mRNA) transcribed from the vector in the second cell type. In this example, only the first cell type could produce the expression product (e.g., the protein) encoded by the polynucleotide. Further selectivity can be achieved through the use of a cell type-specific promoter or through the use of multiple, different miRNA target sequences (e.g., target sequences that are recognized by different miRNAs). A vector described herein may include a single polynucleotide that can be transcribed to produce a desired expression product or multiple, different polynucleotides that can be transcribed to produce different expression products (e.g., two, three, four, five, six, seven, eight, or more polynucleotides, each of which can be transcribed to produce a different expression product), which can be expressed using the same or different promoters and regulated by the same or different miRNA target sequences. In embodiments in which a vector contains multiple polynucleotides that can be transcribed to produce different expression products (e.g., multiple transgene sequences), the vector may be designed such that some or all of the polynucleotides are expressed in a cell type-specific manner (e.g., associated with polynucleotide that can be transcribed to produce a miRNA target sequence that regulates expression). In some embodiments in which a vector contains multiple polynucleotides that can be transcribed to produce desired expression products (e.g., multiple transgene sequences), not all of the polynucleotides are necessarily associated with a polynucleotide that can be transcribed to produce a miRNA target sequence that regulates expression. The different configurations of promoters, polynucleotides that can be transcribed to produce desired expression products, and polynucleotides that can be transcribed to produce miRNA target sequences that can be used to regulate gene expression are described in further detail herein.

The vectors described herein can be used to solve two different problems related to cell type-specific gene expression. While both problems relate to expressing a polynucleotide (e.g., a transgene encoding a protein) in a first inner ear cell type and not in a second inner ear cell type, they differ in the relationship between the first and second inner ear cell types. The first problem relates to expressing a polynucleotide that can be transcribed to produce a desired expression product in a first inner ear cell type and not in a second inner ear cell type (e.g., to increase specificity of expression). For example, a vector described herein may be used to express a polynucleotide in a cochlear hair cell and not in a spiral ganglion neuron. To achieve this, the vector would contain a polynucleotide that can be transcribed to produce a target sequence for a miRNA that is expressed by the spiral ganglion neuron but not expressed by the hair cell. The second problem relates to expressing a polynucleotide that can be transcribed to produce a desired expression product in a first inner ear cell type and not in a second inner ear cell type in which expression of the polynucleotide alters the identity of the first inner ear cell type (e.g., by inducing differentiation of the first inner ear cell type) to produce the second inner ear cell type. For example, a vector described herein may be used to express a transgene in a vestibular supporting cell that promotes differentiation of the vestibular supporting cell into a vestibular hair cell. Once the hair cell has been produced, transgene expression may no longer be needed and could potentially impair the further maturation or function of the hair cell. In such embodiments, the vector would need to include a polynucleotide that can be transcribed to produce a target sequence for a miRNA that is expressed by the second inner ear cell type (e.g., the inner ear cell type that the first inner ear cell transforms into) but that is not expressed by the first inner ear cell type. Vectors containing polynucleotides that can be transcribed to produce miRNA target sequences can be used to address both of these problems.

### Expression of a Single Polynucleotide

In some embodiments, the vector for cell type-specific expression of a polynucleotide contains a promoter operably linked to a polynucleotide that can be transcribed to produce a desired expression product (e.g., a transgene encoding a protein or a polynucleotide that can be transcribed to produce an inhibitory RNA molecule) and to one or more polynucleotides that can be transcribed to produce a miRNA target sequence. The promoter can be a cell type-specific promoter (e.g., an inner ear cell type-specific promoter, such as a promoter listed in Table 12) or a ubiquitous promoter. In some embodiments, the vector contains a polynucleotide that can be transcribed to produce a single miRNA target sequence (e.g., the target sequence for one miRNA). One or more copies of the polynucleotide that can be transcribed to produce the single miRNA target sequence (e.g., 1, 2, 3, 4, 5, 6, 7, 8, 9, 10, or more copies of the polynucleotide that can be transcribed to produce the miRNA target sequence) may be included in the vector. In other embodiments, the vector contains polynucleotides that can be transcribed to produce target sequences for at least two different miRNAs (e.g., the vector contains at least two different polynucleotides that can be transcribed to produce a miRNA target sequence, each of which can be transcribed to produce a target sequence for a different miRNA, such that the vector can be used to produce target sequences for 2, 3, 4, 5, 6, 7, 8, 9, 10, or more different miRNAs). The vector can include one or more copies (e.g., 1, 2, 3, 4, 5, 6, 7, 8, 9, 10, or more copies) of each of the different polynucleotides that can be transcribed to produce different miRNA target sequences.

### Expression of Two Polynucleotides

In some embodiments, the vector contains two polynucleotides that can be transcribed to produce desired expression products (e.g., two different polynucleotides, such as two transgenes, each of which encodes a different protein). A vector containing two such polynucleotides can be designed such that expression of both polynucleotides is regulated by at least one miRNA target sequence or such that expression of only one of the two polynucleotides is regulated by at least one miRNA target sequence. In embodiments in which the vector is designed such that expression of both polynucleotides is regulated by at least one miRNA target sequence, expression of both polynucleotides may be regulated by the same miRNA target sequence(s) or by different miRNA target sequences.

In one embodiment, a single promoter is operably linked to both polynucleotides that can be transcribed to produce desired expression products. In this embodiment, expression of both polynucleotides is regulated by the same miRNA target sequence(s). The promoter can be a cell type-specific promoter (e.g., an inner ear cell type-specific promoter, such as a promoter listed in Table 12) or a ubiquitous promoter. In some embodiments, the vector contains a polynucleotide that can be transcribed to produce a single miRNA target sequence (e.g., the target sequence for one miRNA). One or more copies of the polynucleotide that can be transcribed to produce the single miRNA target sequence (e.g., 1, 2, 3, 4, 5, 6, 7, 8, 9, 10, or more copies of the polynucleotide that can be transcribed to produce the miRNA target sequence) may be included in the vector. In other embodiments, the vector contains polynucleotides that can be transcribed to produce target sequences for at least two different miRNAs (e.g., the vector contains at least two different polynucleotides that can be transcribed to produce a miRNA target sequence, each of which can be transcribed to produce a target sequence for a different miRNA, such that the vector can be used to produce target sequences for 2, 3, 4, 5, 6, 7, 8, 9, 10, or more different miRNAs). The vector can include one or more copies (e.g., 1, 2, 3, 4, 5, 6, 7, 8, 9, 10, or more copies) of each of the different polynucleotides that can be transcribed to produce different miRNA target sequences. The vector can include the following components in 5′ to 3′ order: a promoter, a first polynucleotide that can be transcribed to produce a desired expression product (e.g., a first transgene), a second polynucleotide that can be transcribed to produce a desired expression product (e.g., a second transgene), and one or more polynucleotides that can be transcribed to produce a miRNA target sequence (e.g., one or more copies of a polynucleotide that can be transcribed to produce a single miRNA target sequence, or one or more copies of each of multiple different polynucleotides, each of which can be transcribed to produce a different miRNA target sequence). Such a vector can be used to achieve cell type-specific expression of both the first and second polynucleotides in a first inner ear cell type relative to a second inner ear cell type (e.g., to increase specificity of expression of both polynucleotides and/or to “turn off” expression of both polynucleotides when the first inner ear cell type converts into the second inner ear cell type). An element that allows for co-expression of the two polynucleotides that can be transcribed to produce desired expression products can be positioned between the first and second polynucleotides, such as an internal ribosome entry site (IRES) or a sequence encoding 2A peptide (e.g., a foot-and-mouth disease virus 2A sequence (F2A), an equine rhinitis A virus 2A sequence (E2A), a porcine teschovirus-1 2A sequence (P2A), or a Thosea asigna virus 2A sequence (T2A)).

In some embodiments, each polynucleotide that can be transcribed to produce a desired expression product is operably linked to its own promoter (e.g., the vector contains two promoters, one operably linked to each polynucleotide). Each promoter can be independently selected from a cell type-specific promoter and a ubiquitous promoter. In some embodiments, the two promoters are different. The two promoters can have different cell type specificities (e.g., one promoter is a supporting cell-specific promoter and the other promoter is a hair cell-specific promoter, or one promoter is a hair cell-specific promoter and the other promoter is a ubiquitous promoter) or the same cell type-specificity (e.g., one promoter is a supporting cell-specific promoter and the other promoter is a different supporting cell-specific promoter). In other embodiments, the first promoter and the second promoter are two copies of the same promoter (e.g., each polynucleotide that can be transcribed to produce a desired expression product is operably linked to a different copy of the same ubiquitous promoter or the same hair cell-specific promoter, which could allow one polynucleotide to be regulated by a miRNA target sequence and the other polynucleotide not to be regulated by a miRNA target sequence or to be regulated by a different miRNA target sequence).

In some embodiments in a vector containing two promoters, expression of only one polynucleotide that can be transcribed to produce a desired expression product is regulated by a miRNA target sequence. For example, the vector can include in 5′ to 3′ order: a first promoter, a first polynucleotide that can be transcribed to produce a desired expression product (e.g., a first transgene), one or more polynucleotides that can be transcribed to produce a miRNA target sequence, a second promoter, and a second polynucleotide that can be transcribed to produce a desired expression product (e.g., a second transgene); or a first promoter, a first polynucleotide that can be transcribed to produce a desired expression product (e.g., a first transgene), a second promoter, a second polynucleotide that can be transcribed to produce a desired expression product (e.g., a second transgene), and one or more polynucleotides that can be transcribed to produce a miRNA target sequence. As above, the vector can contain one or more copies (e.g., 1, 2, 3, 4, 5, 6, 7, 8, 9, 10, or more copies) of a polynucleotide that can be transcribed to produce a miRNA target sequence for only one miRNA, or it can contain one or more copies (e.g., 1, 2, 3, 4, 5, 6, 7, 8, 9, 10, or more copies) of at least two different polynucleotides (e.g., 2, 3, 4, 5, 6, 7, 8, 9, 10 or more different polynucleotides), each of which can be transcribed to produce a target sequence for a different miRNA. Such a vector can be used to express one polynucleotide that can be transcribed to produce a desired expression product (e.g., the polynucleotide associated with a polynucleotide that can be transcribed to produce a miRNA target sequence) in a specific inner ear cell type and to express the other polynucleotide that can be transcribed to produce a desired expression product more broadly or in a different cell type. Such a vector can also be used to “turn off” expression of one polynucleotide that can be transcribed to produce a desired expression product once a cell differentiates (e.g., in an embodiment in which a miRNA expressed in the “differentiated” cell type recognizes the miRNA target sequence associated with the expression product) while allowing the other polynucleotide that can be transcribed to produce a desired expression product that is not regulated by a miRNA target sequence to be expressed both before and after differentiation.

In some embodiments in a vector containing two promoters, expression of both polynucleotides is regulated by miRNA target sequences. The vector can include the following components in 5′ to 3′ order: a first promoter, a first polynucleotide that can be transcribed to produce a desired expression product (e.g., a first transgene), one or more polynucleotides that can be transcribed to produce a miRNA target sequence (e.g., one or more copies of a polynucleotide that can be transcribed to produce a single miRNA target sequence, or one or more copies of each of multiple different polynucleotides, each of which can be transcribed to produce a different miRNA target sequence), a second promoter, a second polynucleotide that can be transcribed to produce a desired expression product (e.g., a second transgene), and one or more polynucleotides that can be transcribed to produce a miRNA target sequence (e.g., one or more copies of a polynucleotide that can be transcribed to produce single miRNA target sequence, or one or more copies of each of multiple different polynucleotides, each of which can be transcribed to produce a different miRNA target sequence). The miRNA target sequences regulating expression of the first polynucleotide and the second polynucleotide may be completely different (e.g., each polynucleotide is regulated by a different miRNA target sequence or by a set of completely different miRNA target sequences), may be the same, or may be partially different (e.g., the first polynucleotide is regulated by a first set of miRNA target sequences and the second polynucleotide is regulated by a second set of miRNA target sequences, in which at least one miRNA target sequence differs between the first and second set of miRNA target sequences and at least one miRNA target sequence is included in both the first and second set of miRNA target sequences). Vectors in which the first polynucleotide and the second polynucleotide are associated with polynucleotides that can be transcribed to produce different (e.g., completely different or partially different) miRNA target sequences can be used to regulate expression (e.g., reduce or inhibit off-target expression) of the first polynucleotide and second polynucleotide in different inner ear cell types. Such vectors can also be used to “turn off” expression of a first polynucleotide when a first cell type differentiates into a second cell type (e.g., in an embodiment in which a miRNA expressed in the second cell type recognizes the miRNA target sequence associated with the first polynucleotide) and/or to “turn on” expression of a second polynucleotide in the “differentiated” second cell type (e.g., in an embodiment in which a miRNA expressed in the first cell type but not the second cell type recognizes the miRNA target sequence associated with the second polynucleotide).

### Expression of Three Polynucleotides

In some embodiments, the vector contains three polynucleotides that can be transcribed to produce desired expression products (e.g., three different polynucleotides, such as three transgenes, each of which encodes a different protein). A vector containing three polynucleotides can be designed such that expression of only one polynucleotide is regulated by at least one miRNA target sequence, such that expression of two of the three polynucleotides is regulated by at least one miRNA target sequence, or such that expression of all three polynucleotides is regulated by at least one miRNA target sequence. In embodiments in which the vector is designed such that expression of two or all three polynucleotides is regulated by at least one miRNA target sequence, expression of all three polynucleotides can be regulated using the same miRNA target sequence or set of miRNA target sequences, expression of each polynucleotide that is regulated by a miRNA target sequence (e.g., two or all three of the polynucleotides) can be independently regulated by one or more miRNA target sequences (e.g., expression of each polynucleotide is regulated by a different miRNA target sequence or set of miRNA target sequences), or expression of two polynucleotides may be regulated by the same miRNA target sequence or set of miRNA target sequences while the third polynucleotide is not regulated by a miRNA target sequence or is independently regulated by a different miRNA target sequence or set of miRNA target sequences.

In one embodiment, a single promoter is operably linked to all three polynucleotides that can be transcribed to produce desired expression products. In this embodiment, expression of all three polynucleotides is regulated by the same miRNA target sequence(s). The promoter can be a cell type-specific promoter (e.g., an inner ear cell type-specific promoter, such as a promoter listed in Table 12) or a ubiquitous promoter. In some embodiments, the vector contains a polynucleotide that can be transcribed to produce a single miRNA target sequence (e.g., the target sequence for one miRNA). One or more copies of the polynucleotide that can be transcribed to produce the single miRNA target sequence (e.g., 1, 2, 3, 4, 5, 6, 7, 8, 9, 10, or more copies of the miRNA target sequence) may be included in the vector. In other embodiments, the vector contains polynucleotides that can be transcribed to produce target sequences for at least two different miRNAs (e.g., the vector contains at least two different polynucleotides that can be transcribed to produce a miRNA target sequence, each of which can be transcribed to produce a target sequence for a different miRNA, such that the vector can be used to produce target sequences for 2, 3, 4, 5, 6, 7, 8, 9, 10, or more different miRNAs). The vector can include one or more copies (e.g., 1, 2, 3, 4, 5, 6, 7, 8, 9, 10, or more copies) of each of the different polynucleotides that can be transcribed to produce different miRNA target sequences. The vector can include the following components in 5′ to 3′ order: a promoter, a first polynucleotide that can be transcribed to produce a desired expression product (e.g., a first transgene), a second polynucleotide that can be transcribed to produce a desired expression product (e.g., a second transgene), a third polynucleotide that can be transcribed to produce a desired expression product (e.g., a third transgene), and one or more polynucleotides that can be transcribed to produce a miRNA target sequence (e.g., one or more copies of a polynucleotide that can be transcribed to produce a single miRNA target sequence, or one or more copies of each of multiple different polynucleotides, each of which can be transcribed to produce a different miRNA target sequence). Such a vector can be used to achieve cell type-specific expression of all three transgenes in a first inner ear cell type relative to a second inner ear cell type (e.g., to increase specificity of expression of all three polynucleotides and/or to “turn off” expression of all three polynucleotides when the first inner ear cell type converts into the second inner ear cell type). An element that allows for co-expression of the three polynucleotides can be positioned between the first, second, and third polynucleotides, such as an IRES or a sequence encoding a 2A peptide (e.g., an F2A, E2A, P2A, or T2A sequence).

In some embodiments, each polynucleotide that can be transcribed to produce a desired expression product is operably linked to its own promoter. Each promoter can be independently selected from a cell type-specific promoter and a ubiquitous promoter. In some embodiments, all three promoters are different. The three promoters can have different cell type specificities (e.g., one promoter is a ubiquitous promoter while the other two promoters are supporting cell-specific promoters, or the promoters include one of each of a supporting cell-specific promoter, a hair cell-specific promoter, and a ubiquitous promoter) or the same cell type-specificity (e.g., all three promoters are supporting cell-specific promoters or hair cell-specific promoters). In some embodiments, all three promoters are the same (e.g., the vector contains three copies of the same promoter, such that each polynucleotide is operably linked to a different copy of the same supporting cell-specific promoter, the same hair cell-specific promoter, or the same ubiquitous promoter, which could allow polynucleotides associated with the same promoter to be regulated differently, e.g., a first polynucleotide can be regulated by one or more miRNA target sequences, a second polynucleotide can be regulated by a different miRNA target sequence or a different set of miRNA target sequences, and a third polynucleotide can be regulated by yet another different miRNA target sequence or a different set of miRNA target sequences or may not be regulated by a miRNA target sequence). In some embodiments, two of the promoters are the same (e.g., the vector includes two copies of the same promoter, such as two copies of the same supporting cell-specific promoter or ubiquitous promoter, such that two of the polynucleotides are independently operably linked to the different copies of the same promoter) and the third promoter is different (e.g., a different supporting cell-specific promoter or a different ubiquitous promoter, or a promoter with a different cell type specificity, such as a hair cell-specific promoter). This also allows the two polynucleotides associated with the same promoter to be regulated differently (e.g., each polynucleotide can be associated with a different miRNA target sequence or set of miRNA target sequences, or one polynucleotide may be regulated by a miRNA target sequence while the other is not regulated by a miRNA target sequence), while the third polynucleotide associated with a different promoter can be regulated by the same miRNA target sequence or set of miRNA target sequences, regulated by a different miRNA target sequence or a different set of miRNA target sequences, or not regulated by a miRNA target sequence.

In some embodiments, the vector containing three polynucleotides that can be transcribed to produce desired expression products (e.g., three transgenes) may contain two promoters, such that one promoter is operably linked to one polynucleotide and the other promoter is operably linked to two polynucleotides. Each promoter can be independently selected from a cell type-specific promoter and a ubiquitous promoter. In some embodiments, the two promoters are different. The promoters can have different cell type specificities (e.g., one promoter is a ubiquitous promoter while the other promoter is a supporting cell-specific promoter, or one promoter is a supporting cell-specific promoter and the other promoter is a hair cell-specific promoter) or the same cell type-specificity (e.g., both promoters are supporting cell-specific promoters or hair cell-specific promoters). In other embodiments, the two promoters are the same (e.g., the vector includes two copies of the same promoter, such as the same ubiquitous promoter or the same supporting cell- or hair cell-specific promoter, such that one copy of the promoter is operably linked to the one polynucleotide and the other copy of the promoter is operably linked to the two polynucleotides, which could allow polynucleotides associated with the same promoter to be regulated differently, e.g., the one polynucleotide is regulated by one or more miRNA target sequences while the two polynucleotides are not regulated by a miRNA target sequence or are regulated by one or more different miRNA target sequences). An element that allows for co-expression of the two polynucleotides that can be transcribed to produce desired expression products can be positioned between the two polynucleotides that are operably linked to a single promoter, such as an IRES or a sequence encoding a 2A peptide (e.g., an F2A, E2A, P2A, or T2A sequence).

In some embodiments in a vector containing two or three promoters, expression of only one polynucleotide that can be transcribed to produce a desired expression product is regulated by a miRNA target sequence. An example of a vector containing two promoters can include in 5′ to 3′ order: a first promoter, a first polynucleotide that can be transcribed to produce a desired expression product (e.g., a first transgene), one or more polynucleotides that can be transcribed to produce a miRNA target sequence, a second promoter, a second polynucleotide that can be transcribed to produce a desired expression product (e.g., a second transgene), and a third polynucleotide that can be transcribed to produce a desired expression product (e.g., a third transgene). In another example, the vector can include in 5′ to 3′ order: a first promoter, a first polynucleotide that can be transcribed to produce a desired expression product (e.g., a first transgene), a second polynucleotide that can be transcribed to produce a desired expression product (e.g., a second transgene), a second promoter, a third polynucleotide that can be transcribed to produce a desired expression product (e.g., a third transgene), and one or more polynucleotides that can be transcribed to produce a miRNA target sequence. An IRES or a sequence encoding a 2A peptide (e.g., an F2A, E2A, P2A, or T2A sequence) can be positioned between the two polynucleotides that can be transcribed to produce a desired expression product that are operably linked to the same promoter in both of these vectors. An example of a vector containing three promoters in which only one gene is regulated by a miRNA target sequence can include in 5′ to 3′ order: a first promoter, a first polynucleotide that can be transcribed to produce a desired expression product (e.g., a first transgene), one or more polynucleotides that can be transcribed to produce a miRNA target sequence, a second promoter, a second polynucleotide that can be transcribed to produce a desired expression product (e.g., a second transgene), a third promoter, and a third polynucleotide that can be transcribed to produce a desired expression product (e.g., a third transgene). In other examples, the one or more polynucleotides that can be transcribed to produce a miRNA target sequence may be positioned 3′ of the second polynucleotide and 5′ of the third promoter, or 3′ of the third polynucleotide. Such a vector can be used to express one polynucleotide (e.g., the polynucleotide associated with one or more polynucleotides that can be transcribed to produce a miRNA target sequence) in a specific cell type and to express the other transgenes more broadly or in one or more different cell types. Such a vector can also be used to “turn off” expression of one polynucleotide once a cell differentiates (e.g., in an embodiment in which a miRNA expressed in the “differentiated” cell type recognizes the miRNA target sequence associated with the polynucleotide) while allowing the other polynucleotides to be expressed both before and after differentiation.

In some embodiments in a vector containing two or three promoters, two polynucleotides that can be transcribed to produce a desired expression product are regulated by a miRNA target sequence. An example of a vector containing two promoters can include in 5′ to 3′ order: a first promoter, a first polynucleotide that can be transcribed to produce a desired expression product (e.g., a first transgene), a second polynucleotide that can be transcribed to produce a desired expression product (e.g., a second transgene), one or more polynucleotides that can be transcribed to produce a miRNA target sequence, a second promoter, and a third polynucleotide that can be transcribed to produce a desired expression product (e.g., a third transgene). In another example, the first polynucleotide may be expressed by a first promoter and not regulated by a miRNA target sequence and a second promoter may be operably linked to the second and third polynucleotides and to one or more polynucleotides that can be transcribed to produce a miRNA target sequence (the vector can include in 5′ to 3′ order: a first promoter, a first polynucleotide that can be transcribed to produce a desired expression product (e.g., a first transgene), a second promoter, a second polynucleotide that can be transcribed to produce a desired expression product (e.g., a second transgene), a third polynucleotide that can be transcribed to produce a desired expression product (e.g., a third transgene), and one or more polynucleotides that can be transcribed to produce a miRNA target sequence). An IRES or a sequence encoding a 2A peptide (e.g., an F2A, E2A, P2A, or T2A sequence) can be positioned between the two polynucleotides that can be transcribed to produce a desired expression product and that are operably linked to the same promoter in both of these vectors. An example of a vector containing three promoters can include in 5′ to 3′ order: a first promoter, a first polynucleotide that can be transcribed to produce a desired expression product (e.g., a first transgene), one or more polynucleotides that can be transcribed to produce a miRNA target sequence, a second promoter, a second polynucleotide that can be transcribed to produce a desired expression product (e.g., a second transgene), one or more polynucleotides that can be transcribed to produce a miRNA target sequence, a third promoter, and a third polynucleotide that can be transcribed to produce a desired expression product (e.g., a third transgene). In such a vector, the first and second, the first and third, or the second and third polynucleotides can be regulated by one or more miRNA target sequences. The one or more miRNA target sequences used to regulate the two polynucleotides in the vector containing three promoters can be the same (e.g., the same miRNA target sequence or set of miRNA target sequences) or different (e.g., completely different miRNA target sequences or partially different sets of miRNA target sequences).

In some embodiments in a vector containing two or three promoters, all three polynucleotides are regulated by a miRNA target sequence. An example of a vector containing two promoters can include in 5′ to 3′ order: a first promoter, a first polynucleotide that can be transcribed to produce a desired expression product (e.g., a first transgene), one or more polynucleotides that can be transcribed to produce a miRNA target sequence, a second promoter, a second polynucleotide that can be transcribed to produce a desired expression product (e.g., a second transgene), a third polynucleotide that can be transcribed to produce a desired expression product (e.g., a third transgene), and one or more polynucleotides that can be transcribed to produce a miRNA target sequence. In a vector containing two promoters, either the first and second polynucleotides or the second and third polynucleotides are operably linked to a single promoter and regulated by the same miRNA target sequence or set of miRNA target sequences. The one or more miRNA target sequences used to regulate the one polynucleotide and the two remaining polynucleotides in such a vector can be the same (e.g., the same miRNA target sequence or set of miRNA target sequences) or different (e.g., completely different miRNA target sequences or partially different sets of miRNA target sequences). An example of a vector containing three promoters can include in 5′ to 3′ order: a first promoter, a first polynucleotide that can be transcribed to produce a desired expression product (e.g., a first transgene), one or more polynucleotides that can be transcribed to produce a miRNA target sequence, a second promoter, a second polynucleotide that can be transcribed to produce a desired expression product (e.g., a second transgene), one or more polynucleotides that can be transcribed to produce a miRNA target sequence, a third promoter, a third polynucleotide that can be transcribed to produce a desired expression product (e.g., a third transgene), and one or more polynucleotides that can be transcribed to produce a miRNA target sequence. In such a vector the one or more miRNA target sequences used to regulate the three polynucleotides can be completely different (e.g., each polynucleotide is regulated by a different miRNA target sequence or set of miRNA target sequences), the same (e.g., all three polynucleotides are regulated by the same miRNA target sequence or set of miRNA target sequences), or partially different (e.g., each polynucleotide is regulated by a set of miRNA target sequences, and each set includes at least one miRNA target sequence that is shared by all three sets and at least one miRNA target sequence that is unique to each set). In some embodiments, two of the three nucleic acids may be regulated by the same miRNA target sequence or set of miRNA target sequences while the third nucleic acid is regulated by a different miRNA target sequence or a completely or partially different set of miRNA target sequences. In some embodiments, two of the three polynucleotides are each regulated by a set of partially different miRNA target sequences and the third nucleic acid is regulated by a completely different miRNA target sequence or set of completely different miRNA target sequences.

Any of the vectors containing three polynucleotides that can be transcribed to produce a desired expression product can include a polynucleotide that can be transcribed to produce a miRNA target sequence for only one miRNA, or can include at least two or more (e.g., 2, 3, 4, 5, 6, 7, 8, 9, 10 or more) different polynucleotides, each of which can be transcribed to produce a target sequence for a different miRNA, and each polynucleotide that can be transcribed to produce a miRNA target sequence may be present in the vector in one or more copies (e.g., 1, 2, 3, 4, 5, 6, 7, 8, 9, 10, or more copies). In vectors containing two promoters in which all three polynucleotides are regulated by miRNA target sequences and in vectors containing three promoters in which two or all three polynucleotides are regulated by miRNA target sequences, the miRNA target sequences regulating expression of each polynucleotide (or pair of polynucleotides, as in the case of the vector containing two promoters) may be completely different, may be the same, or may be partially different (e.g., the first polynucleotide is associated with a first set of miRNA target sequences and each of the second and third polynucleotides, or the pair of polynucleotides, is associated with a second (and/or third, in the case of a vector containing three independently regulated polynucleotides) set of miRNA target sequences, in which at least one miRNA target sequence differs between the first and second (and/or third) set of miRNA target sequences, and at least one miRNA target sequence is included in both the first and second (and/or third) set of miRNA target sequences). Vectors in which two or all three polynucleotides are associated with different (e.g., completely different or partially different) miRNA target sequences can be used to regulate expression (e.g., reduce or inhibit off-target expression) of the first polynucleotide, second polynucleotide, and/or third polynucleotide in different inner ear cell types. Such vectors can also be used to “turn off” expression of one or two polynucleotides when a first inner ear cell type differentiates into a second inner ear cell type (e.g., in an embodiment in which a miRNA expressed in the second inner ear cell type recognizes the miRNA target sequence associated with the one or two polynucleotides) and/or to “turn on” expression of the remaining polynucleotide(s) in the “differentiated” second cell type (e.g., in an embodiment in which a miRNA expressed in the first cell type but not the second cell type recognizes the miRNA target sequence associated with the remaining polynucleotide(s)).

Expression of More than Three Polynucleotides

In some embodiments, the vector contains more than three polynucleotides that can be transcribed to produce desired expression products (e.g., 4, 5, 6, 7, 8, 9, 10, or more different polynucleotides). Such a vector can be designed such that expression of only one of the polynucleotides contained in the vector is regulated by at least one miRNA target sequence, such that expression of a subset (fewer than all) of the polynucleotides contained in the vector is regulated by at least one miRNA target sequence, or such that expression of all of the polynucleotides contained in the vector is regulated by at least one miRNA target sequence. Vectors containing more than three polynucleotides can be constructed by extending the principles described hereinabove for three polynucleotides to encompass four more polynucleotides. For example, polynucleotides that are to be expressed in the same cell types can be operably linked to the same promoter and/or associated with polynucleotides that can be transcribed to produce the same miRNA target sequence(s). Polynucleotides that are to be expressed in different cell types can be operably linked to different promoters (e.g., promoters with different cell type-specificities) and associated with polynucleotides that can be transcribed to produce different miRNA target sequences (e.g., completely different miRNA target sequences or sets of partially different miRNA target sequences) or with polynucleotides that can be transcribed to produce an the same miRNA target sequences (e.g., to prevent off-target expression of the polynucleotides in the same cell type). Polynucleotides that are not intended for regulation using a miRNA target sequence can be operably linked to a promoter that is not operably linked to a polynucleotide that can be transcribed to produce a miRNA target sequence. The promoter(s) used to express the polynucleotides that can be transcribed to produce a desired expression product can be cell type-specific promoters (e.g., an inner ear cell type-specific promoter, such as a promoter listed in Table 12) or ubiquitous promoters. Each polynucleotide to be regulated by a miRNA target sequence can be associated with at least one polynucleotide that can be transcribed to produce a miRNA target sequence (e.g., 1, 2, 3, 4, 5, 6, 7, 8, 9, 10 or more polynucleotides that can be transcribed to produce a miRNA target sequence). If a polynucleotide that can be transcribed to produce a desired expression product is associated with multiple polynucleotides that can be transcribed to produce miRNA target sequences, the polynucleotides that can be transcribed to produce miRNA target sequences can be the same (e.g., a polynucleotide that can be transcribed to produce a target sequence for a single miRNA can be present in multiple copies) or different (e.g., at least two different polynucleotides, each of which can be transcribed to produce a target sequence for a different miRNA, in which case each polynucleotide that can be transcribed to produce a different miRNA target sequence can be present in one or more copies). If more than one polynucleotide that can be transcribed to produce a desired expression product is operably linked to a single promoter, an element that allows for co-expression of the polynucleotides can be positioned between each of the polynucleotides operably linked to the promoter, such as an IRES or a sequence encoding a 2A peptide (e.g., an F2A, E2A, P2A, or T2A sequence).

### Delivery of Multiple Vectors

A vector described herein (e.g., a vector containing a promoter operably linked to a polynucleotide that can be transcribed to produce a desired expression product and to one or more polynucleotides that can be transcribed to produce a miRNA target sequence) can be administered in combination with one or more additional vectors (e.g., 1, 2, 3, 4, 5, or more additional vectors). In some embodiments, a vector described herein is administered in combination with one additional vector. In some embodiments, the one or more additional vectors are also vectors of the invention (e.g., vectors containing a promoter operably linked to a polynucleotide that can be transcribed to produce a desired expression product and to one or more polynucleotides that can be transcribed to produce a miRNA target sequence). For example, two or more vectors described herein (e.g., 2, 3, 4, 5, 6, or more vectors described herein) can be administered in combination. In some embodiments, the one or more additional vectors do not contain a polynucleotide that can be transcribed to produce a miRNA target sequence.

In some embodiments, the vector described herein and the one or more additional vectors are administered simultaneously (e.g., administration of all vectors occurs within 15 minutes, 10 minutes, 5 minutes, 2 minutes or less). The vectors can also be administered simultaneously via co-formulation. The vector described herein and the one or more additional vectors can also be administered sequentially. Sequential or substantially simultaneous administration of each of the vectors can be performed by any appropriate route including local administration to the middle or inner ear (e.g., administration to or through the round window, the oval window, or a semicircular canal). The vectors can be administered by the same route or by different routes. For example, both vectors can be administered locally to the inner ear. The vector described herein may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the one or more additional vectors.

miRNA Target Sequences

The vectors described herein contain one or more polynucleotides that can be transcribed to produce a miRNA target sequence, each of which is recognized by a miRNA that is differentially expressed between different inner ear cell types (e.g., expressed in a first type of inner ear cell and not in a second type of inner ear cell). Each vector can contain one or more copies of a polynucleotide that can be transcribed to produce a single miRNA target sequence (e.g., 1, 2, 3, 4, 5, 6, 7, 8, 9, 10, or more copies of a polynucleotide that can be transcribed to produce a single miRNA target sequence) and/or one or more different polynucleotides, each of which can be transcribed to produce a miRNA target sequence recognized by a different miRNA (e.g., 2, 3, 4, 5, 6, 7, 8, 9, 10, or more different polynucleotides, each of which can be transcribed to produce a target sequence for a different miRNA), each of which may be included in the vector in one or more copies (e.g., 1, 2, 3, 4, 5, 6, 7, 8, 9, 10, or more copies).

The polynucleotide that can be transcribed to produce a miRNA target sequence is positioned within the vector such that it is operably linked to the same promoter as the polynucleotide to be regulated by the miRNA target sequence (e.g., the polynucleotide that can be transcribed to produce a desired expression product). For example, if the polynucleotide to be regulated by a miRNA target sequence is a transgene (a polynucleotide encoding a protein), the polynucleotide that can be transcribed to produce a miRNA target sequence can be located in the 3′ untranslated region (UTR) of the transgene (e.g., between the stop codon of the transgene and the end of the polyA sequence). The polynucleotide that can be transcribed to produce a miRNA target sequence can also be located in the 5′ UTR of the transgene or within the transgene coding sequence as long as the position of the polynucleotide that can be transcribed to produce a miRNA target sequence does not disrupt expression of the transgene in cells that do not express a miRNA that binds to the miRNA target sequence. If the polynucleotide that can be transcribed to produce a miRNA target sequence is located in a transgene coding sequence, it may be flanked by cleavage sites so that, if translation is not inhibited by a miRNA that recognizes the miRNA target sequence, the resulting polypeptide can be cleaved to excise the miRNA target sequence and form a full-length protein by joining the 5′ and 3′ portions of the protein encoded by the transgene coding sequence. To regulate the expression of multiple polynucleotides (e.g., in an embodiment in which a single promoter is operably linked to two, three, or more polynucleotides that can be transcribed to produce desired expression products), the polynucleotide that can be transcribed to produce a miRNA target sequence can be operably linked to the promoter that drives expression of the polynucleotides and positioned 3′ of the final polynucleotide operably linked to the promoter (e.g., in the 3′ UTR of the final polynucleotide) or positioned 5′ of the first polynucleotide operably linked to the promoter (e.g., in the 5′ UTR of the first polynucleotide).

Table 2 below provides a list of miRNAs expressed in one or more inner ear cell types along with the target sequence for each miRNA.

TABLE 2
miRNAs expressed in inner ear cell types

| miRNA | Target Sequence | Inner Ear Cell Types |
|---|---|---|
| miR-183 | UAUGGCACUGGUAGAAUUCACU | cochlear hair cells, spiral ganglion neurons, |
| | (SEQ ID NO: 25) | spiral limbus, inner sulcus, vestibular hair |
| | | cells, vestibular ganglion neurons |
| miR-96 | UUUGGCACUAGCACAUUUUUGCU | cochlear hair cells, spiral ganglion neurons, |
| | (SEQ ID NO: 26) | spiral limbus, inner sulcus, vestibular hair |
| | | cells, vestibular ganglion neurons |
| miR-182 | UUUGGCAAUGGUAGAACUCACACCG | cochlear hair cells, spiral ganglion neurons, |
| | (SEQ ID NO: 27) | spiral limbus, inner sulcus, vestibular hair |
| | | cells, vestibular ganglion neurons |
| miR-18a | UAAGGUGCAUCUAGUGCAGAUAG | cochlear hair cells, spiral ganglion neurons, |
| | (SEQ ID NO: 28) | vestibular hair cells, vestibular ganglion |
| | | neurons |
| miR-140 | CAGUGGUUUUACCCUAUGGUAG | cochlear hair cells, vestibular hair cells |
| | (SEQ ID NO: 29) | |
| miR-194 | UGUAACAGCAACUCCAUGUGGA | cochlear hair cells, spiral ganglion neurons |
| | (SEQ ID NO: 30) | |
| miR-15a | UAGCAGCACAUAAUGGUUUGUG | cochlear hair cells, cochlear supporting cells, |
| | (SEQ ID NO: 31) | spiral ganglion neurons, basilar membrane, |
| | | vestibular hair cells |
| miR-30b | UGUAAACAUCCUACACUCAGCU | cochlear hair cells, cochlear supporting cells, |
| | (SEQ ID NO: 32) | spiral ganglion neurons, basilar membrane, |
| | | vestibular hair cells |
| miR-99a | AACCCGUAGAUCCGAUCUUGUG | cochlear hair cells, cochlear supporting cells, |
| | (SEQ ID NO: 33) | spiral ganglion neurons, basilar membrane, |
| | | vestibular hair cells |
| miR-124a | UAAGGCACGCGGUGAAUGCC | spiral ganglion neurons, vestibular ganglion |
| | (SEQ ID NO: 34) | neurons |
| miR-205 | UCCUUCAUUCCACCGGAGUCUG | Reissner′s membrane, spiral limbus, basilar |
| | (SEQ ID NO: 35) | membrane, spiral ligament |
| miR-376a | AUCGUAGAGGAAAAUCCACGU | marginal cells |
| | (SEQ ID NO: 36) | |
| miR-376b | AUCAUAGAGGAACAUCCACUU | marginal cells |
| | (SEQ ID NO: 37) | |
| miR-135b | UAUGGCUUUUCAUUCCUAUGUGA | vestibular hair cells |
| | (SEQ ID NO: 38) | |
| miR-100 | AACCCGUAGAUCCGAACUUGUG | vestibular ganglion neurons |
| | (SEQ ID NO: 39) | |
| miR-135 | UAUGGCUUUUUAUUCCUAUGUGA | vestibular ganglion neurons |
| | (SEQ ID NO: 40) | |
| miR-376b-3p | AUCAUAGAGGAACAUCCACUU | vestibular sensory epithelium, vestibular |
| | (SEQ ID NO: 41) | ganglion neurons |
| miR-376a-3p | AUCGUAGAGGAAAAUCCACGU | vestibular sensory epithelium |
| | (SEQ ID NO: 42) | |

Inclusion of one or more polynucleotides that can be transcribed to produce a miRNA target sequence from Table 2 in a vector described herein can prevent or reduce off-target expression of a polynucleotide included in the vector (e.g., a polynucleotide operably linked to the same promoter as the polynucleotide that can be transcribed to produce the miRNA target sequence) to improve or achieve cell type-specific expression of the polynucleotide in a particular cell type of interest. For example, for cell type-specific expression of a polynucleotide in a cochlear supporting cell, the vector can include a ubiquitous promoter (e.g., CMV) or a supporting cell-specific promoter (e.g., an FGFR3 promoter, an LFNG promoter, a GJB2 promoter, or a SLC1A3 promoter) operably linked to a polynucleotide that can be transcribed to produce a desired expression product (e.g., a transgene encoding Atoh1, Gfi1, Pou4f3, Ikzf2, dnSox2, and/or Gjb2) and to one or more polynucleotides that can be transcribed to produce a target sequence for a miRNA expressed in cell types other than cochlear supporting cells (e.g., a miRNA target sequence for a miRNA expressed in cochlear hair cells and not cochlear supporting cells, such as miR-183, miR-96, miR-182, miR-18a, miR-140, and/or miR-194, and/or a miRNA target sequence for a miRNA expressed in spiral ganglion neurons and not cochlear supporting cells, such as miR-183, miR-96, miR-182, miR-18a, miR-124a, and/or miR-194). For cell type-specific expression of a polynucleotide in a vestibular supporting cell, the vector can include a ubiquitous promoter (e.g., CMV) or a supporting cell-specific promoter (e.g., a GFAP promoter, a SLC6A14 promoter, or a SLC1A3 promoter) operably linked to a polynucleotide that can be transcribed to produce a desired expression product (e.g., a transgene encoding Atoh1, Gfi1, Pou4f3, Ikzf2, dnSox2, and/or Gjb2) and to one or more polynucleotides that can be transcribed to produce a target sequence for a miRNA expressed in cell types other than vestibular supporting cells (e.g., a miRNA target sequence for a miRNA expressed in vestibular ganglion neurons and not vestibular supporting cells, such as miR-183, miR-96, miR-182, miR-18a, miR-124a, miR-100, and/or miR-135). To specifically express a polynucleotide in a Type II vestibular hair cell, the vector can include a hair cell-specific promoter (e.g., a MYO15 promoter) operably linked to a polynucleotide that can be transcribed to produce a desired expression product (e.g., a transgene encoding a dominant negative Sox2 protein (dnSox2) or a polynucleotide that can be transcribed to produce an inhibitory RNA, such as an shRNA, directed to Sox2) and to one or more polynucleotides that can be transcribed to produce a target sequence for a miRNA expressed in cell types other than vestibular hair cells (e.g., a miRNA target sequence for a miRNA expressed in vestibular ganglion neurons and not vestibular hair cells, such as miR-18a, miR-124a, miR-100, and/or miR-135). Sequences for exemplary plasmids containing a promoter operably linked to a transgene and to one or more polynucleotides that can be transcribed to produce a miRNA target sequence are provided in Table 3, below.

TABLE 3
Sequences for transgene plasmids containing a polynucleotide that can be transcribed to
produce a miRNA target sequence

| SEQ ID NO: and | |
|---|---|
| annotation | Sequence |
| SEQ ID NO: 1 | CCTTAATTAGGCTGCGCGCTCGCTCGCTCACTGAGGCCGCCCGGGCAAAG |
| P742 sequence | CCCGGGCGTCGGGCGACCTTTGGTCGCCCGGCCTCAGTGAGCGAGCGAGC |
| 5′ ITR-12-141 | GCGCAGAGAGGGAGTGGCCAACTCCATCACTAGGGGTTCCTTGTAGTTAAT |
| CMV Enhancer at | GATTAACCCGCCATGCTACTTATCTACGTAGCCATGCTCTAGGAAGATCGGA |
| positions 244-547 | ATTCGCCCTTAAGCTAGCGGCGCGCCACCGGTGCGATCGCCGTTACATAAC |
| CMV promoter at | TTACGGTAAATGGCCCGCCTGGCTGACCGCCCAACGACCCCCGCCCATTGA |
| positions 548-751 | CGTCAATAATGACGTATGTTCCCATAGTAACGCCAATAGGGACTTTCCATTG |
| AcGFP1 at | ACGTCAATGGGTGGAGTATTTACGGTAAACTGCCCACTTGGCAGTACATCAA |
| positions 801-1517 | GTGTATCATATGCCAAGTACGCCCCCTATTGACGTCAATGACGGTAAATGGC |
| miR-183 target | CCGCCTGGCATTATGCCCAGTACATGACCTTATGGGACTTTCCTACTTGGCA |
| sequence at | GTACATCTACGTATTAGTCATCGCTATTACCATGGTGATGCGGTTTTGGCAG |
| positions 1531-1552 | TACATCAATGGGCGTGGATAGCGGTTTGACTCACGGGGATTTCCAAGTCTC |
| miR-96 target | CACCCCATTGACGTCAATGGGAGTTTGTTTTGGCACCAAAATCAACGGGACT |
| sequence at | TTCCAAAATGTCGTAACAACTCCGCCCCATTGACGCAAATGGGGGGTAGGC |
| positions 1553-1575 | GTGTACGGTGGGAGGTCTATATAAGCAGAGCTGGTTTAGTGAACCGTCAGA |
| miR-182 target | TCCTGCAGGGCCGGCCGCGGCCGCACGCGTATGGTGAGCAAGGGCGCCG |
| sequence at | AGCTGTTCACCGGCATCGTGCCCATCCTGATCGAGCTGAATGGCGATGTGA |
| positions 1576-1600 | ATGGCCACAAGTTCAGCGTGAGCGGCGAGGGCGAGGGCGATGCCACCTAC |
| WPRE at | GGCAAGCTGACCCTGAAGTTCATCTGCACCACCGGCAAGCTGCCTGTGCCC |
| positions 1602-2149 | TGGCCCACCCTGGTGACCACCCTGAGCTACGGCGTGCAGTGCTTCTCACGC |
| bGH polyA at | TACCCCGATCACATGAAGCAGCACGACTTCTTCAAGAGCGCCATGCCTGAG |
| positions 2162-2369 | GGCTACATCCAGGAGCGCACCATCTTCTTCGAGGATGACGGCAACTACAAG |
| 3′ ITR at positions | TCGCGCGCCGAGGTGAAGTTCGAGGGCGATACCCTGGTGAATCGCATCGA |
| 2457-2586 | GCTGACCGGCACCGATTTCAAGGAGGATGGCAACATCCTGGGCAATAAGAT |
| | GGAGTACAACTACAACGCCCACAATGTGTACATCATGACCGACAAGGCCAA |
| | GAATGGCATCAAGGTGAACTTCAAGATCCGCCACAACATCGAGGATGGCAG |
| | CGTGCAGCTGGCCGACCACTACCAGCAGAATACCCCCATCGGCGATGGCC |
| | CTGTGCTGCTGCCCGATAACCACTACCTGTCCACCCAGAGCGCCCTGTCCA |
| | AGGACCCCAACGAGAAGCGCGATCACATGATCTACTTCGGCTTCGTGACCG |
| | CCGCCGCCATCACCCACGGCATGGATGAGCTGTACAAGTAATAATAAGCTTA |
| | GTGAATTCTACCAGTGCCATAAGCAAAAATGTGCTAGTGCCAAACGGTGTGA |
| | GTTCTACCATTGCCAAAGGATCCAATCAACCTCTGGATTACAAAATTTGTGAA |
| | AGATTGACTGGTATTCTTAACTATGTTGCTCCTTTTACGCTATGTGGATACGC |
| | TGCTTTAATGCCTTTGTATCATGCTATTGCTTCCCGTATGGCTTTCATTTTCTC |
| | CTCCTTGTATAAATCCTGGTTGCTGTCTCTTTATGAGGAGTTGTGGCCCGTT |
| | GTCAGGCAACGTGGCGTGGTGTGCACTGTGTTTGCTGACGCAACCCCCACT |
| | GGTTGGGGCATTGCCACCACCTGTCAGCTCCTTTCCGGGACTTTCGCTTTC |
| | CCCCTCCCTATTGCCACGGCGGAACTCATCGCCGCCTGCCTTGCCCGCTGC |
| | TGGACAGGGGCTCGGCTGTTGGGCACTGACAATTCCGTGGTGTTGTCGGG |
| | GAAATCATCGTCCTTTCCTTGGCTGCTCGCCTGTGTTGCCACCTGGATTCTG |
| | CGCGGGACGTCCTTCTGCTACGTCCCTTCGGCCCTCAATCCAGCGGACCTT |
| | CCTTCCCGCGGCCTGCTGCCGGCTCTGCGGCCTCTTCCGCGTCTTCGAGAT |
| | CTGCCTCGACTGTGCCTTCTAGTTGCCAGCCATCTGTTGTTTGCCCCTCCCC |
| | CGTGCCTTCCTTGACCCTGGAAGGTGCCACTCCCACTGTCCTTTCCTAATAA |
| | AATGAGGAAATTGCATCGCATTGTCTGAGTAGGTGTCATTCTATTCTGGGGG |
| | GTGGGGTGGGGCAGGACAGCAAGGGGGAGGATTGGGAAGACAATAGCAG |
| | GCATGCTGGGGACTCGAGTTAAGGGCGAATTCCCGATAAGGATCTTCCTAG |
| | AGCATGGCTACGTAGATAAGTAGCATGGGGGGTTAATCATTAACTACAAGGA |
| | ACCCCTAGTGATGGAGTTGGCCACTCCCTCTCTGCGCGCTCGCTCGCTCAC |
| | TGAGGCCGGGCGACCAAAGGTCGCCCGACGCCCGGGCTTTGCCCGGGCG |
| | GCCTCAGTGAGCGAGCGAGCGCGCAGCCTTAATTAACCTAATTCACTGGCC |
| | GTCGTTTTACAACGTCGTGACTGGGAAAACCCTGGCGTTACCCAACTTAATC |
| | GCCTTGCAGCACATCCCCCTTTCGCCAGCTGGCGTAATAGCGAAGAGGCCC |
| | GCACCGATCGCCCTTCCCAACAGTTGCGCAGCCTGAATGGCGAATGGGACG |
| | CGCCCTGTAGCGGCGCATTAAGCGCGGCGGGTGTGGTGGTTACGCGCAGC |
| | GTGACCGCTACACTTGCCAGCGCCCTAGCGCCCGCTCCTTTCGCTTTCTTC |
| | CCTTCCTTTCTCGCCACGTTCGCCGGCTTTCCCCGTCAAGCTCTAAATCGGG |
| | GGCTCCCTTTAGGGTTCCGATTTAGTGCTTTACGGCACCTCGACCCCAAAAA |
| | ACTTGATTAGGGTGATGGTTCACGTAGTGGGCCATCGCCCTGATAGACGGT |
| | TTTTCGCCCTTTGACGTTGGAGTCCACGTTCTTTAATAGTGGACTCTTGTTCC |
| | AAACTGGAACAACACTCAACCCTATCTCGGTCTATTCTTTTGATTTATAAGGG |
| | ATTTTGCCGATTTCGGCCTATTGGTTAAAAAATGAGCTGATTTAACAAAAATT |
| | TAACGCGAATTTTAACAAAATATTAACGCTTACAATTTAGGTGGCACTTTTCG |
| | GGGAAATGTGCGCGGAACCCCTATTTGTTTATTTTTCTAAATACATTCAAATA |
| | TGTATCCGCTCATGAGACAATAACCCTGATAAATGCTTCAATAATATTGAAAA |
| | AGGAAGAGTATGAGTATTCAACATTTCCGTGTCGCCCTTATTCCCTTTTTTGC |
| | GGCATTTTGCCTTCCTGTTTTTGCTCACCCAGAAACGCTGGTGAAAGTAAAA |
| | GATGCTGAAGATCAGTTGGGTGCACGAGTGGGTTACATCGAACTGGATCTC |
| | AACAGCGGTAAGATCCTTGAGAGTTTTCGCCCCGAAGAACGTTTTCCAATGA |
| | TGAGCACTTTTAAAGTTCTGCTATGTGGCGCGGTATTATCCCGTATTGACGC |
| | CGGGCAAGAGCAACTCGGTCGCCGCATACACTATTCTCAGAATGACTTGGT |
| | TGAGTACTCACCAGTCACAGAAAAGCATCTTACGGATGGCATGACAGTAAGA |
| | GAATTATGCAGTGCTGCCATAACCATGAGTGATAACACTGCGGCCAACTTAC |
| | TTCTGACAACGATCGGAGGACCGAAGGAGCTAACCGCTTTTTTGCACAACAT |
| | GGGGGATCATGTAACTCGCCTTGATCGTTGGGAACCGGAGCTGAATGAAGC |
| | CATACCAAACGACGAGCGTGACACCACGATGCCTGTAGCAATGGCAACAAC |
| | GTTGCGCAAACTATTAACTGGCGAACTACTTACTCTAGCTTCCCGGCAACAA |
| | TTAATAGACTGGATGGAGGCGGATAAAGTTGCAGGACCACTTCTGCGCTCG |
| | GCCCTTCCGGCTGGCTGGTTTATTGCTGATAAATCTGGAGCCGGTGAGCGT |
| | GGGTCTCGCGGTATCATTGCAGCACTGGGGCCAGATGGTAAGCCCTCCCGT |
| | ATCGTAGTTATCTACACGACGGGGAGTCAGGCAACTATGGATGAACGAAATA |
| | GACAGATCGCTGAGATAGGTGCCTCACTGATTAAGCATTGGTAACTGTCAGA |
| | CCAAGTTTACTCATATATACTTTAGATTGATTTAAAACTTCATTTTTAATTTAAA |
| | AGGATCTAGGTGAAGATCCTTTTTGATAATCTCATGACCAAAATCCCTTAACG |
| | TGAGTTTTCGTTCCACTGAGCGTCAGACCCCGTAGAAAAGATCAAAGGATCT |
| | TCTTGAGATCCTTTTTTTCTGCGCGTAATCTGCTGCTTGCAAACAAAAAAACC |
| | ACCGCTACCAGCGGTGGTTTGTTTGCCGGATCAAGAGCTACCAACTCTTTTT |
| | CCGAAGGTAACTGGCTTCAGCAGAGCGCAGATACCAAATACTGTTCTTCTAG |
| | TGTAGCCGTAGTTAGGCCACCACTTCAAGAACTCTGTAGCACCGCCTACATA |
| | CCTCGCTCTGCTAATCCTGTTACCAGTGGCTGCTGCCAGTGGCGATAAGTC |
| | GTGTCTTACCGGGTTGGACTCAAGACGATAGTTACCGGATAAGGCGCAGCG |
| | GTCGGGCTGAACGGGGGGTTCGTGCACACAGCCCAGCTTGGAGCGAACGA |
| | CCTACACCGAACTGAGATACCTACAGCGTGAGCTATGAGAAAGCGCCACGC |
| | TTCCCGAAGGGAGAAAGGCGGACAGGTATCCGGTAAGCGGCAGGGTCGGA |
| | ACAGGAGAGCGCACGAGGGAGCTTCCAGGGGGAAACGCCTGGTATCTTTAT |
| | AGTCCTGTCGGGTTTCGCCACCTCTGACTTGAGCGTCGATTTTTGTGATGCT |
| | CGTCAGGGGGGCGGAGCCTATGGAAAAACGCCAGCAACGCGGCCTTTTTA |
| | CGGTTCCTGGCCTTTTGCTGGCCTTTTGCTCACATGTTCTTTCCTGCGTTATC |
| | CCCTGATTCTGTGGATAACCGTATTACCGCCTTTGAGTGAGCTGATACCGCT |
| | CGCCGCAGCCGAACGACCGAGCGCAGCGAGTCAGTGAGCGAGGAAGCGG |
| | AAGAGCGCCCAATACGCAAACCGCCTCTCCCCGCGCGTTGGCCGATTCATT |
| | AATGCAGCTGGCACGACAGGTTTCCCGACTGGAAAGCGGGCAGTGAGCGC |
| | AACGCAATTAATGTGAGTTAGCTCACTCATTAGGCACCCCAGGCTTTACACT |
| | TTATGCTTCCGGCTCGTATGTTGTGTGGAATTGTGAGCGGATAACAATTTCA |
| | CACAGGAAACAGCTATGACCATGATTACGCCAGATTTAATTAAGG |
| SEQ ID NO: 2 | CCTTAATTAGGCTGCGCGCTCGCTCGCTCACTGAGGCCGCCCGGGCAAAG |
| P744 Sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATCGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTOCCATAGTAACGCCAATAGGGACTTTCCATTG |
| positions 548-751 | ACGTCAATGGGTGGAGTATTTACGGTAAACTGCCCACTTGGCAGTACATCAA |
| AcGFP1 at | GTGTATCATATGCCAAGTACGCCCCCTATTGACGTCAATGACGGTAAATGGC |
| positions 801-1517 | CCGCCTGGCATTATGCCCAGTACATGACCTTATGGGACTTTCCTACTTGGCA |
| miR-183 target | GTACATCTACGTATTAGTCATCGCTATTACCATGGTGATGCGGTTTTGGCAG |
| sequences (3) at | TACATCAATGGGCGTGGATAGCGGTTTGACTCACGGGGATTTCCAAGTCTC |
| positions 1531- | CACCCCATTGACGTCAATGGGAGTTTGTTTTGGCACCAAAATCAACGGGACT |
| 1552, 1553-1574, | TTCCAAAATGTCGTAACAACTCCGCCCCATTGACGCAAATGGGGGGTAGGC |
| and 1575-1596 | GTGTACGGTGGGAGGTCTATATAAGCAGAGCTGGTTTAGTGAACCGTCAGA |
| miR-96 target | TCCTGCAGGGCCGGCCGCGGCCGCACGCGTATGGTGAGCAAGGGCGCCG |
| sequences (3) at | AGCTGTTCACCGGCATCGTGCCCATCCTGATCGAGCTGAATGGCGATGTGA |
| positions 1597- | ATGGCCACAAGTTCAGCGTGAGCGGCGAGGGCGAGGGCGATGCCACCTAC |
| 1619, 1620-1642, | GGCAAGCTGACCCTGAAGTTCATCTGCACCACCGGCAAGCTGCCTGTGCCC |
| and 1643-1665 | TGGCCCACCCTGGTGACCACCCTGAGCTACGGCGTGCAGTGCTTCTCACGC |
| miR-182 target | TACCCCGATCACATGAAGCAGCACGACTTCTTCAAGAGCGCCATGCCTGAG |
| sequences (3) at | GGCTACATCCAGGAGCGCACCATCTTCTTCGAGGATGACGGCAACTACAAG |
| positions 1666- | TCGCGCGCCGAGGTGAAGTTCGAGGGCGATACCCTGGTGAATCGCATCGA |
| 1690, 1691-1715, | GCTGACCGGCACCGATTTCAAGGAGGATGGCAACATCCTGGGCAATAAGAT |
| and 1716-1740 | GGAGTACAACTACAACGCCCACAATGTGTACATCATGACCGACAAGGCCAA |
| WPRE at | GAATGGCATCAAGGTGAACTTCAAGATCCGCCACAACATCGAGGATGGCAG |
| positions 1742-2289 | CGTGCAGCTGGCCGACCACTACCAGCAGAATACCCCCATCGGCGATGGCC |
| bGH polyA at | CTGTGCTGCTGCCCGATAACCACTACCTGTCCACCCAGAGCGCCCTGTCCA |
| positions 2302-2509 | AGGACCCCAACGAGAAGCGCGATCACATGATCTACTTCGGCTTCGTGACCG |
| 3′ ITR at positions | CCGCCGCCATCACCCACGGCATGGATGAGCTGTACAAGTAATAATAAGCTTA |
| 2597-2726 | GTGAATTCTACCAGTGCCATAAGTGAATTCTACCAGTGCCATAAGTGAATTCT |
| | ACCAGTGCCATAAGCAAAAATGTGCTAGTGCCAAAAGCAAAAATGTGCTAGT |
| | GCCAAAAGCAAAAATGTGCTAGTGCCAAACGGTGTGAGTTCTACCATTGCCA |
| | AACGGTGTGAGTTCTACCATTGCCAAACGGTGTGAGTTCTACCATTGCCAAA |
| | GGATCCAATCAACCTCTGGATTACAAAATTTGTGAAAGATTGACTGGTATTCT |
| | TAACTATGTTGCTCCTTTTACGCTATGTGGATACGCTGCTTTAATGCCTTTGT |
| | ATCATGCTATTGCTTCCCGTATGGCTTTCATTTTCTCCTCCTTGTATAAATCCT |
| | GGTTGCTGTCTCTTTATGAGGAGTTGTGGCCCGTTGTCAGGCAACGTGGCG |
| | TGGTGTGCACTGTGTTTGCTGACGCAACCCCCACTGGTTGGGGCATTGCCA |
| | CCACCTGTCAGCTCCTTTCCGGGACTTTCGOTTTCCCCCTCCCTATTGCCAC |
| | GGCGGAACTCATCGCCGCCTGCCTTGCCCGCTGCTGGACAGGGGCTCGGC |
| | TGTTGGGCACTGACAATTCCGTGGTGTTGTCGGGGAAATCATCGTCCTTTCC |
| | TTGGCTGCTCGCCTGTGTTGCCACCTGGATTCTGCGCGGGACGTCCTTCTG |
| | CTACGTCCCTTCGGCCCTCAATCCAGCGGACCTTCCTTCCCGCGGCCTGCT |
| | GCCGGCTCTGCGGCCTCTTCCGCGTCTTCGAGATCTGCCTCGACTGTGCCT |
| | TCTAGTTGCCAGCCATCTGTTGTTTGCCCCTCCCCCGTGCCTTCCTTGACCC |
| | TGGAAGGTGCCACTCCCACTGTCCTTTCCTAATAAAATGAGGAAATTGCATC |
| | GCATTGTCTGAGTAGGTGTCATTCTATTCTGGGGGGTGGGGTGGGGCAGGA |
| | CAGCAAGGGGGAGGATTGGGAAGACAATAGCAGGCATGCTGGGGACTCGA |
| | GTTAAGGGCGAATTCCCGATAAGGATCTTCCTAGAGCATGGCTACGTAGATA |
| | AGTAGCATGGCGGGTTAATCATTAACTACAAGGAACCCCTAGTGATGGAGTT |
| | GGCCACTCCCTCTCTGCGCGCTCGCTCGCTCACTGAGGCCGGGCGACCAA |
| | AGGTCGCCCGACGCCCGGGCTTTGCCCGGGGGGCCTCAGTGAGCGAGCG |
| | AGCGCGCAGCCTTAATTAACCTAATTCACTGGCCGTCGTTTTACAACGTCGT |
| | GACTGGGAAAACCCTGGCGTTACCCAACTTAATCGCCTTGCAGCACATCCC |
| | CCTTTCGCCAGCTGGCGTAATAGCGAAGAGGCCCGCACCGATCGCCCTTCC |
| | CAACAGTTGCGCAGCCTGAATGGCGAATGGGACGCGCCCTGTAGCGGCGC |
| | ATTAAGCGCGGCGGGTGTGGTGGTTACGCGCAGCGTGACCGCTACACTTG |
| | CCAGCGCCCTAGCGCCCGCTCCTTTCGCTTTCTTCCCTTCCTTTCTCGCCAC |
| | GTTCGCCGGCTTTCCCCGTCAAGCTCTAAATCGGGGGCTCCCTTTAGGGTT |
| | CCGATTTAGTGCTTTACGGCACCTCGACCCCAAAAAACTTGATTAGGGTGAT |
| | GGTTCACGTAGTGGGCCATCGCCCTGATAGACGGTTTTTCGCCCTTTGACG |
| | TTGGAGTCCACGTTCTTTAATAGTGGACTCTTGTTCCAAACTGGAACAACACT |
| | CAACCCTATCTCGGTCTATTCTTTTGATTTATAAGGGATTTTGCCGATTTCGG |
| | CCTATTGGTTAAAAAATGAGCTGATTTAACAAAAATTTAACGCGAATTTTAAC |
| | AAAATATTAACGCTTACAATTTAGGTGGCACTTTTCGGGGAAATGTGCGCGG |
| | AACCCCTATTTGTTTATTTTTCTAAATACATTCAAATATGTATCCGCTCATGAG |
| | ACAATAACCCTGATAAATGCTTCAATAATATTGAAAAAGGAAGAGTATGAGTA |
| | TTCAACATTTCCGTGTCGCCCTTATTCCCTTTTTTGCGGCATTTTGCCTTCCT |
| | GTTTTTGCTCACCCAGAAACGCTGGTGAAAGTAAAAGATGCTGAAGATCAGT |
| | TGGGTGCACGAGTGGGTTACATCGAACTGGATCTCAACAGCGGTAAGATCC |
| | TTGAGAGTTTTCGCCCCGAAGAACGTTTTCCAATGATGAGCACTTTTAAAGTT |
| | CTGCTATGTGGCGCGGTATTATCCCGTATTGACGCCGGGCAAGAGCAACTC |
| | GGTCGCCGCATACACTATTCTCAGAATGACTTGGTTGAGTACTCACCAGTCA |
| | CAGAAAAGCATCTTACGGATGGCATGACAGTAAGAGAATTATGCAGTGCTGC |
| | CATAACCATGAGTGATAACACTGCGGCCAACTTACTTCTGACAACGATCGGA |
| | GGACCGAAGGAGCTAACCGCTTTTTTGCACAACATGGGGGATCATGTAACT |
| | CGCCTTGATCGTTGGGAACCGGAGCTGAATGAAGCCATACCAAACGACGAG |
| | CGTGACACCACGATGCCTGTAGCAATGGCAACAACGTTGCGCAAACTATTAA |
| | CTGGCGAACTACTTACTCTAGCTTCCCGGCAACAATTAATAGACTGGATGGA |
| | GGCGGATAAAGTTGCAGGACCACTTCTGCGCTCGGCCCTTCCGGCTGGCTG |
| | GTTTATTGCTGATAAATCTGGAGCCGGTGAGCGTGGGTCTCGCGGTATCATT |
| | GCAGCACTGGGGCCAGATGGTAAGCCCTCCCGTATCGTAGTTATCTACACG |
| | ACGGGGAGTCAGGCAACTATGGATGAACGAAATAGACAGATCGCTGAGATA |
| | GGTGCCTCACTGATTAAGCATTGGTAACTGTCAGACCAAGTTTACTCATATAT |
| | ACTTTAGATTGATTTAAAACTTCATTTTTAATTTAAAAGGATCTAGGTGAAGAT |
| | CCTTTTTGATAATCTCATGACCAAAATCCCTTAACGTGAGTTTTCGTTCCACT |
| | GAGCGTCAGACCCCGTAGAAAAGATCAAAGGATCTTCTTGAGATCCTTTTTT |
| | TCTGCGCGTAATCTGCTGCTTGCAAACAAAAAAACCACCGCTACCAGCGGT |
| | GGTTTGTTTGCCGGATCAAGAGCTACCAACTCTTTTTCCGAAGGTAACTGGC |
| | TTCAGCAGAGCGCAGATACCAAATACTGTTCTTCTAGTGTAGCCGTAGTTAG |
| | GCCACCACTTCAAGAACTCTGTAGCACCGCCTACATACCTCGCTCTGCTAAT |
| | CCTGTTACCAGTGGCTGCTGCCAGTGGCGATAAGTCGTGTCTTACCGGGTT |
| | GGACTCAAGACGATAGTTACCGGATAAGGCGCAGCGGTCGGGCTGAACGG |
| | GGGGTTCGTGCACACAGCCCAGCTTGGAGCGAACGACCTACACCGAACTGA |
| | GATACCTACAGCGTGAGCTATGAGAAAGCGCCACGCTTCCCGAAGGGAGAA |
| | AGGCGGACAGGTATCCGGTAAGCGGCAGGGTCGGAACAGGAGAGCGCACG |
| | AGGGAGCTTCCAGGGGGAAACGCCTGGTATCTTTATAGTCCTGTCGGGTTT |
| | CGCCACCTCTGACTTGAGCGTCGATTTTTGTGATGCTCGTCAGGGGGGCGG |
| | AGCCTATGGAAAAACGCCAGCAACGCGGCCTTTTTACGGTTCCTGGCCTTTT |
| | GCTGGCCTTTTGCTCACATGTTCTTTCCTGCGTTATCCCCTGATTCTGTGGAT |
| | AACCGTATTACCGCCTTTGAGTGAGCTGATACCGCTCGCCGCAGCCGAACG |
| | ACCGAGCGCAGCGAGTCAGTGAGCGAGGAAGCGGAAGAGCGCCCAATACG |
| | CAAACCGCCTCTCCCCGCGCGTTGGCCGATTCATTAATGCAGCTGGCACGA |
| | CAGGTTTCCCGACTGGAAAGCGGGCAGTGAGCGCAACGCAATTAATGTGAG |
| | TTAGCTCACTCATTAGGCACCCCAGGCTTTACACTTTATGCTTCCGGCTCGT |
| | ATGTTGTGTGGAATTGTGAGCGGATAACAATTTCACACAGGAAACAGCTATG |
| | ACCATGATTACGCCAGATTTAATTAAGG |
| SEQ ID NO: 3 | CCTTAATTAGGCTGCGCGCTCGCTCGCTCACTGAGGCCGCCCGGGCAAAG |
| P745 Sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATCGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTCCCATAGTAACGCCAATAGGGACTTTCCATTG |
| positions 548-751 | ACGTCAATGGGTGGAGTATTTACGGTAAACTGCCCACTTGGCAGTACATCAA |
| AcGFP1 at | GTGTATCATATGCCAAGTACGCCCCCTATTGACGTCAATGACGGTAAATGGC |
| positions 801-1517 | CCGCCTGGCATTATGCCCAGTACATGACCTTATGGGACTTTCCTACTTGGCA |
| miR-96 target | GTACATCTACGTATTAGTCATCGCTATTACCATGGTGATGCGGTTTTGGCAG |
| sequences (4) at | TACATCAATGGGCGTGGATAGCGGTTTGACTCACGGGGATTTCCAAGTCTC |
| positions 1531- | CACCCCATTGACGTCAATGGGAGTTTGTTTTGGCACCAAAATCAACGGGACT |
| 1553, 1554-1576, | TTCCAAAATGTCGTAACAACTCCGCCCCATTGACGCAAATGGGGGGTAGGC |
| 1577-1599, and | GTGTACGGTGGGAGGTCTATATAAGCAGAGCTGGTTTAGTGAACCGTCAGA |
| 1600-1622 | TCCTGCAGGGCCGGCCGCGGCCGCACGCGTATGGTGAGCAAGGGCGCCG |
| WPRE at | AGCTGTTCACCGGCATCGTGCCCATCCTGATCGAGCTGAATGGCGATGTGA |
| positions 1624-2171 | ATGGCCACAAGTTCAGCGTGAGCGGCGAGGGCGAGGGCGATGCCACCTAC |
| bGH polyA at | GGCAAGCTGACCCTGAAGTTCATCTGCACCACCGGCAAGCTGCCTGTGCCC |
| positions 2184-2391 | TGGCCCACCCTGGTGACCACCCTGAGCTACGGCGTGCAGTGCTTCTCACGC |
| 3′ ITR at positions | TACCCCGATCACATGAAGCAGCACGACTTCTTCAAGAGCGCCATGCCTGAG |
| 2479-2608 | GGCTACATCCAGGAGCGCACCATCTTCTTCGAGGATGACGGCAACTACAAG |
| | TCGCGCGCCGAGGTGAAGTTCGAGGGCGATACCCTGGTGAATCGCATCGA |
| | GCTGACCGGCACCGATTTCAAGGAGGATGGCAACATCCTGGGCAATAAGAT |
| | GGAGTACAACTACAACGCCCACAATGTGTACATCATGACCGACAAGGCCAA |
| | GAATGGCATCAAGGTGAACTTCAAGATCCGCCACAACATCGAGGATGGCAG |
| | CGTGCAGCTGGCCGACCACTACCAGCAGAATACCCCCATCGGCGATGGCC |
| | CTGTGCTGCTGCCCGATAACCACTACCTGTCCACCCAGAGCGCCCTGTCCA |
| | AGGACCCCAACGAGAAGCGCGATCACATGATCTACTTCGGCTTCGTGACCG |
| | CCGCCGCCATCACCCACGGCATGGATGAGCTGTACAAGTAATAATAAGCTTA |
| | GCAAAAATGTGCTAGTGCCAAAAGCAAAAATGTGCTAGTGCCAAAAGCAAAA |
| | ATGTGCTAGTGCCAAAAGCAAAAATGTGCTAGTGCCAAAGGATCCAATCAAC |
| | CTCTGGATTACAAAATTTGTGAAAGATTGACTGGTATTCTTAACTATGTTGCT |
| | CCTTTTACGCTATGTGGATACGCTGCTTTAATGCCTTTGTATCATGCTATTGC |
| | TTCCCGTATGGCTTTCATTTTCTCCTCCTTGTATAAATCCTGGTTGCTGTCTC |
| | TTTATGAGGAGTTGTGGCCCGTTGTCAGGCAACGTGGCGTGGTGTGCACTG |
| | TGTTTGCTGACGCAACCCCCACTGGTTGGGGCATTGCCACCACCTGTCAGC |
| | TCCTTTCCGGGACTTTCGCTTTCCCCCTCCCTATTGCCACGGCGGAACTCAT |
| | CGCCGCCTGCCTTGCCCGCTGCTGGACAGGGGCTCGGCTGTTGGGCACTG |
| | ACAATTCCGTGGTGTTGTCGGGGAAATCATCGTCCTTTCCTTGGCTGCTCGC |
| | CTGTGTTGCCACCTGGATTCTGCGCGGGACGTCCTTCTGCTACGTCCCTTC |
| | GGCCCTCAATCCAGCGGACCTTCCTTCCCGCGGCCTGCTGCCGGCTCTGC |
| | GGCCTCTTCCGCGTCTTCGAGATCTGCCTCGACTGTGCCTTCTAGTTGCCA |
| | GCCATCTGTTGTTTGCCCCTCCCCCGTGCCTTCCTTGACCCTGGAAGGTGC |
| | CACTCCCACTGTCCTTTCCTAATAAAATGAGGAAATTGCATCGCATTGTCTGA |
| | GTAGGTGTCATTCTATTCTGGGGGGTGGGGTGGGGCAGGACAGCAAGGGG |
| | GAGGATTGGGAAGACAATAGCAGGCATGCTGGGGACTCGAGTTAAGGGCG |
| | AATTCCCGATAAGGATCTTCCTAGAGCATGGCTACGTAGATAAGTAGCATGG |
| | CGGGTTAATCATTAACTACAAGGAACCCCTAGTGATGGAGTTGGCCACTCCC |
| | TCTCTGCGCGCTCGCTCGCTCACTGAGGCCGGGCGACCAAAGGTCGCCCG |
| | ACGCCCGGGCTTTGCCCGGGGGGCCTCAGTGAGCGAGCGAGCGCGCAGC |
| | CTTAATTAACCTAATTCACTGGCCGTCGTTTTACAACGTCGTGACTGGGAAA |
| | ACCCTGGCGTTACCCAACTTAATCGCCTTGCAGCACATCCCCCTTTCGCCAG |
| | CTGGCGTAATAGCGAAGAGGCCCGCACCGATCGCCCTTCCCAACAGTTGCG |
| | CAGCCTGAATGGCGAATGGGACGCGCCCTGTAGCGGCGCATTAAGCGCGG |
| | CGGGTGTGGTGGTTACGCGCAGCGTGACCGCTACACTTGCCAGCGCCCTA |
| | GCGCCCGCTCCTTTCGCTTTCTTCCCTTCCTTTCTCGCCACGTTCGCCGGCT |
| | TTCCCCGTCAAGCTCTAAATCGGGGGCTCCCTTTAGGGTTCCGATTTAGTGC |
| | TTTACGGCACCTCGACCCCAAAAAACTTGATTAGGGTGATGGTTCACGTAGT |
| | GGGCCATCGCCCTGATAGACGGTTTTTCGCCCTTTGACGTTGGAGTCCACG |
| | TTCTTTAATAGTGGACTCTTGTTCCAAACTGGAACAACACTCAACCCTATCTC |
| | GGTCTATTCTTTTGATTTATAAGGGATTTTGCCGATTTCGGCCTATTGGTTAA |
| | AAAATGAGCTGATTTAACAAAAATTTAACGCGAATTTTAACAAAATATTAACG |
| | CTTACAATTTAGGTGGCACTTTTCGGGGAAATGTGCGCGGAACCCCTATTTG |
| | TTTATTTTTCTAAATACATTCAAATATGTATCCGCTCATGAGACAATAACCCTG |
| | ATAAATGCTTCAATAATATTGAAAAAGGAAGAGTATGAGTATTCAACATTTCC |
| | GTGTCGCCCTTATTCCCTTTTTTGCGGCATTTTGCCTTCCTGTTTTTGCTCAC |
| | CCAGAAACGCTGGTGAAAGTAAAAGATGCTGAAGATCAGTTGGGTGCACGA |
| | GTGGGTTACATCGAACTGGATCTCAACAGCGGTAAGATCCTTGAGAGTTTTC |
| | GCCCCGAAGAACGTTTTCCAATGATGAGCACTTTTAAAGTTCTGCTATGTGG |
| | CGCGGTATTATCCCGTATTGACGCCGGGCAAGAGCAACTCGGTCGCCGCAT |
| | ACACTATTCTCAGAATGACTTGGTTGAGTACTCACCAGTCACAGAAAAGCAT |
| | CTTACGGATGGCATGACAGTAAGAGAATTATGCAGTGCTGCCATAACCATGA |
| | GTGATAACACTGCGGCCAACTTACTTCTGACAACGATCGGAGGACCGAAGG |
| | AGCTAACCGCTTTTTTGCACAACATGGGGGATCATGTAACTCGCCTTGATCG |
| | TTGGGAACCGGAGCTGAATGAAGCCATACCAAACGACGAGCGTGACACCAC |
| | GATGCCTGTAGCAATGGCAACAACGTTGCGCAAACTATTAACTGGCGAACTA |
| | CTTACTCTAGCTTCCCGGCAACAATTAATAGACTGGATGGAGGCGGATAAAG |
| | TTGCAGGACCACTTCTGCGCTCGGCCCTTCCGGCTGGCTGGTTTATTGCTG |
| | ATAAATCTGGAGCCGGTGAGCGTGGGTCTCGCGGTATCATTGCAGCACTGG |
| | GGCCAGATGGTAAGCCCTCCCGTATCGTAGTTATCTACACGACGGGGAGTC |
| | AGGCAACTATGGATGAACGAAATAGACAGATCGCTGAGATAGGTGCCTCAC |
| | TGATTAAGCATTGGTAACTGTCAGACCAAGTTTACTCATATATACTTTAGATT |
| | GATTTAAAACTTCATTTTTAATTTAAAAGGATCTAGGTGAAGATCCTTTTTGAT |
| | AATCTCATGACCAAAATCCCTTAACGTGAGTTTTCGTTCCACTGAGCGTCAG |
| | ACCCCGTAGAAAAGATCAAAGGATCTTCTTGAGATCCTTTTTTTCTGCGCGTA |
| | ATCTGCTGCTTGCAAACAAAAAAACCACCGCTACCAGCGGTGGTTTGTTTGC |
| | CGGATCAAGAGCTACCAACTCTTTTTCCGAAGGTAACTGGCTTCAGCAGAGC |
| | GCAGATACCAAATACTGTTCTTCTAGTGTAGCCGTAGTTAGGCCACCACTTC |
| | AAGAACTCTGTAGCACCGCCTACATACCTCGCTCTGCTAATCCTGTTACCAG |
| | TGGCTGCTGCCAGTGGCGATAAGTCGTGTCTTACCGGGTTGGACTCAAGAC |
| | GATAGTTACCGGATAAGGCGCAGCGGTCGGGCTGAACGGGGGGTTCGTGC |
| | ACACAGCCCAGCTTGGAGCGAACGACCTACACCGAACTGAGATACCTACAG |
| | CGTGAGCTATGAGAAAGCGCCACGCTTCCCGAAGGGAGAAAGGCGGACAG |
| | GTATCCGGTAAGCGGCAGGGTCGGAACAGGAGAGCGCACGAGGGAGCTTC |
| | CAGGGGGAAACGCCTGGTATCTTTATAGTCCTGTCGGGTTTCGCCACCTCT |
| | GACTTGAGCGTCGATTTTTGTGATGCTCGTCAGGGGGGGGAGCCTATGGA |
| | AAAACGCCAGCAACGCGGCCTTTTTACGGTTCCTGGCCTTTTGCTGGCCTTT |
| | TGCTCACATGTTCTTTCCTGCGTTATCCCCTGATTCTGTGGATAACCGTATTA |
| | CCGCCTTTGAGTGAGCTGATACCGCTCGCCGCAGCCGAACGACCGAGCGC |
| | AGCGAGTCAGTGAGCGAGGAAGCGGAAGAGCGCCCAATACGCAAACCGCC |
| | TCTCCCCGCGCGTTGGCCGATTCATTAATGCAGCTGGCACGACAGGTTTCC |
| | CGACTGGAAAGCGGGCAGTGAGCGCAACGCAATTAATGTGAGTTAGCTCAC |
| | TCATTAGGCACCCCAGGCTTTACACTTTATGCTTCCGGCTCGTATGTTGTGT |
| | GGAATTGTGAGCGGATAACAATTTCACACAGGAAACAGCTATGACCATGATT |
| | ACGCCAGATTTAATTAAGG |
| SEQ ID NO: 4 | CCTTAATTAGGCTGCGCGCTCGCTCGCTCACTGAGGCCGCCCGGGCAAAG |
| P746 Sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATCGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTCCCATAGTAACGCCAATAGGGACTTTCCATTG |
| positions 548-751 | ACGTCAATGGGTGGAGTATTTACGGTAAACTGCCCACTTGGCAGTACATCAA |
| AcGFP1 at | GTGTATCATATGCCAAGTACGCCCCCTATTGACGTCAATGACGGTAAATGGC |
| positions 801-1517 | CCGCCTGGCATTATGCCCAGTACATGACCTTATGGGACTTTCCTACTTGGCA |
| miR-182 target | GTACATCTACGTATTAGTCATCGCTATTACCATGGTGATGCGGTTTTGGCAG |
| sequences (4) at | TACATCAATGGGCGTGGATAGCGGTTTGACTCACGGGGATTTCCAAGTCTC |
| positions 1531- | CACCCCATTGACGTCAATGGGAGTTTGTTTTGGCACCAAAATCAACGGGACT |
| 1555, 1556-1580, | TTCCAAAATGTCGTAACAACTCCGCCCCATTGACGCAAATGGGGGGTAGGC |
| 1581-1605, and | GTGTACGGTGGGAGGTCTATATAAGCAGAGCTGGTTTAGTGAACCGTCAGA |
| 1606-1630 | TCCTGCAGGGCCGGCCGCGGCCGCACGCGTATGGTGAGCAAGGGCGCCG |
| WPRE at | AGCTGTTCACCGGCATCGTGCCCATCCTGATCGAGCTGAATGGCGATGTGA |
| positions 1632-2179 | ATGGCCACAAGTTCAGCGTGAGCGGCGAGGGCGAGGGCGATGCCACCTAC |
| bGH polyA at | GGCAAGCTGACCCTGAAGTTCATCTGCACCACCGGCAAGCTGCCTGTGCCC |
| positions 2192-2399 | TGGCCCACCCTGGTGACCACCCTGAGCTACGGCGTGCAGTGCTTCTCACGC |
| 3′ ITR at positions | TACCCCGATCACATGAAGCAGCACGACTTCTTCAAGAGCGCCATGCCTGAG |
| 2487-2616 | GGCTACATCCAGGAGCGCACCATCTTCTTCGAGGATGACGGCAACTACAAG |
| | TCGCGCGCCGAGGTGAAGTTCGAGGGCGATACCCTGGTGAATCGCATCGA |
| | GCTGACCGGCACCGATTTCAAGGAGGATGGCAACATCCTGGGCAATAAGAT |
| | GGAGTACAACTACAACGCCCACAATGTGTACATCATGACCGACAAGGCCAA |
| | GAATGGCATCAAGGTGAACTTCAAGATCCGCCACAACATCGAGGATGGCAG |
| | CGTGCAGCTGGCCGACCACTACCAGCAGAATACCCCCATCGGCGATGGCC |
| | CTGTGCTGCTGCCCGATAACCACTACCTGTCCACCCAGAGCGCCCTGTCCA |
| | AGGACCCCAACGAGAAGCGCGATCACATGATCTACTTCGGCTTCGTGACCG |
| | CCGCCGCCATCACCCACGGCATGGATGAGCTGTACAAGTAATAATAAGCTT |
| | CGGTGTGAGTTCTACCATTGCCAAACGGTGTGAGTTCTACCATTGCCAAACG |
| | GTGTGAGTTCTACCATTGCCAAACGGTGTGAGTTCTACCATTGCCAAAGGAT |
| | CCAATCAACCTCTGGATTACAAAATTTGTGAAAGATTGACTGGTATTCTTAAC |
| | TATGTTGCTCCTTTTACGCTATGTGGATACGCTGCTTTAATGCCTTTGTATCA |
| | TGCTATTGCTTCCCGTATGGCTTTCATTTTCTCCTCCTTGTATAAATCCTGGT |
| | TGCTGTCTCTTTATGAGGAGTTGTGGCCCGTTGTCAGGCAACGTGGCGTGG |
| | TGTGCACTGTGTTTGCTGACGCAACCCCCACTGGTTGGGGCATTGCCACCA |
| | CCTGTCAGCTCCTTTCCGGGACTTTCGOTTTCCCCCTCCCTATTGCCACGGC |
| | GGAACTCATCGCCGCCTGCCTTGCCCGCTGCTGGACAGGGGCTCGGCTGT |
| | TGGGCACTGACAATTCCGTGGTGTTGTCGGGGAAATCATCGTCCTTTCCTTG |
| | GCTGCTCGCCTGTGTTGCCACCTGGATTCTGCGCGGGACGTCCTTCTGCTA |
| | CGTCCCTTCGGCCCTCAATCCAGCGGACCTTCCTTCCCGCGGCCTGCTGCC |
| | GGCTCTGCGGCCTCTTCCGCGTCTTCGAGATCTGCCTCGACTGTGCCTTCT |
| | AGTTGCCAGCCATCTGTTGTTTGCCCCTCCCCCGTGCCTTCCTTGACCCTGG |
| | AAGGTGCCACTCCCACTGTCCTTTCCTAATAAAATGAGGAAATTGCATCGCA |
| | TTGTCTGAGTAGGTGTCATTCTATTCTGGGGGGTGGGGTGGGGCAGGACAG |
| | CAAGGGGGAGGATTGGGAAGACAATAGCAGGCATGCTGGGGACTCGAGTT |
| | AAGGGCGAATTCCCGATAAGGATOTTCCTAGAGCATGGCTACGTAGATAAGT |
| | AGCATGGCGGGTTAATCATTAACTACAAGGAACCCCTAGTGATGGAGTTGG |
| | CCACTCCCTCTCTGCGCGCTCGCTCGCTCACTGAGGCCGGGCGACCAAAG |
| | GTCGCCCGACGCCCGGGCTTTGCCCGGGGGGCCTCAGTGAGCGAGCGAG |
| | CGCGCAGCCTTAATTAACCTAATTCACTGGCCGTCGTTTTACAACGTCGTGA |
| | CTGGGAAAACCCTGGCGTTACCCAACTTAATCGCCTTGCAGCACATCCCCCT |
| | TTCGCCAGCTGGCGTAATAGCGAAGAGGCCCGCACCGATCGCCCTTCCCAA |
| | CAGTTGCGCAGCCTGAATGGCGAATGGGACGCGCCCTGTAGCGGCGCATT |
| | AAGCGCGGCGGGTGTGGTGGTTACGCGCAGCGTGACCGCTACACTTGCCA |
| | GCGCCCTAGCGCCCGCTCCTTTCGCTTTCTTCCCTTCCTTTCTCGCCACGTT |
| | CGCCGGCTTTCCCCGTCAAGCTCTAAATCGGGGGCTCCCTTTAGGGTTCCG |
| | ATTTAGTGCTTTACGGCACCTCGACCCCAAAAAACTTGATTAGGGTGATGGT |
| | TCACGTAGTGGGCCATCGCCCTGATAGACGGTTTTTCGCCCTTTGACGTTG |
| | GAGTCCACGTTCTTTAATAGTGGACTCTTGTTCCAAACTGGAACAACACTCA |
| | ACCCTATCTCGGTCTATTCTTTTGATTTATAAGGGATTTTGCCGATTTCGGCC |
| | TATTGGTTAAAAAATGAGCTGATTTAACAAAAATTTAACGCGAATTTTAACAAA |
| | ATATTAACGCTTACAATTTAGGTGGCACTTTTCGGGGAAATGTGCGCGGAAC |
| | CCCTATTTGTTTATTTTTCTAAATACATTCAAATATGTATCCGCTCATGAGACA |
| | ATAACCCTGATAAATGCTTCAATAATATTGAAAAAGGAAGAGTATGAGTATTC |
| | AACATTTCCGTGTCGCCCTTATTCCCTTTTTTGCGGCATTTTGCCTTCCTGTT |
| | TTTGCTCACCCAGAAACGCTGGTGAAAGTAAAAGATGCTGAAGATCAGTTGG |
| | GTGCACGAGTGGGTTACATCGAACTGGATCTCAACAGCGGTAAGATCCTTG |
| | AGAGTTTTCGCCCCGAAGAACGTTTTCCAATGATGAGCACTTTTAAAGTTCT |
| | GCTATGTGGCGCGGTATTATCCCGTATTGACGCCGGGCAAGAGCAACTCGG |
| | TCGCCGCATACACTATTCTCAGAATGACTTGGTTGAGTACTCACCAGTCACA |
| | GAAAAGCATCTTACGGATGGCATGACAGTAAGAGAATTATGCAGTGCTGCCA |
| | TAACCATGAGTGATAACACTGCGGCCAACTTACTTCTGACAACGATCGGAGG |
| | ACCGAAGGAGCTAACCGCTTTTTTGCACAACATGGGGGATCATGTAACTCGC |
| | CTTGATCGTTGGGAACCGGAGCTGAATGAAGCCATACCAAACGACGAGCGT |
| | GACACCACGATGCCTGTAGCAATGGCAACAACGTTGCGCAAACTATTAACTG |
| | GCGAACTACTTACTCTAGCTTCCCGGCAACAATTAATAGACTGGATGGAGGC |
| | GGATAAAGTTGCAGGACCACTTCTGCGCTCGGCCCTTCCGGCTGGCTGGTT |
| | TATTGCTGATAAATCTGGAGCCGGTGAGCGTGGGTCTCGCGGTATCATTGC |
| | AGCACTGGGGCCAGATGGTAAGCCCTCCCGTATCGTAGTTATCTACACGAC |
| | GGGGAGTCAGGCAACTATGGATGAACGAAATAGACAGATCGCTGAGATAGG |
| | TGCCTCACTGATTAAGCATTGGTAACTGTCAGACCAAGTTTACTCATATATAC |
| | TTTAGATTGATTTAAAACTTCATTTTTAATTTAAAAGGATCTAGGTGAAGATCC |
| | TTTTTGATAATCTCATGACCAAAATCCCTTAACGTGAGTTTTCGTTCCACTGA |
| | GCGTCAGACCCCGTAGAAAAGATCAAAGGATCTTCTTGAGATCCTTTTTTTCT |
| | GCGCGTAATCTGCTGCTTGCAAACAAAAAAACCACCGCTACCAGCGGTGGT |
| | TTGTTTGCCGGATCAAGAGCTACCAACTCTTTTTCCGAAGGTAACTGGCTTC |
| | AGCAGAGCGCAGATACCAAATACTGTTCTTCTAGTGTAGCCGTAGTTAGGCC |
| | ACCACTTCAAGAACTCTGTAGCACCGCCTACATACCTCGCTCTGCTAATCCT |
| | GTTACCAGTGGCTGCTGCCAGTGGCGATAAGTCGTGTCTTACCGGGTTGGA |
| | CTCAAGACGATAGTTACCGGATAAGGCGCAGCGGTCGGGCTGAACGGGGG |
| | GTTCGTGCACACAGCCCAGCTTGGAGCGAACGACCTACACCGAACTGAGAT |
| | ACCTACAGCGTGAGCTATGAGAAAGCGCCACGCTTCCCGAAGGGAGAAAGG |
| | CGGACAGGTATCCGGTAAGCGGCAGGGTCGGAACAGGAGAGCGCACGAGG |
| | GAGCTTCCAGGGGGAAACGCCTGGTATCTTTATAGTCCTGTCGGGTTTCGC |
| | CACCTCTGACTTGAGCGTCGATTTTTGTGATGCTCGTCAGGGGGGCGGAGC |
| | CTATGGAAAAACGCCAGCAACGCGGCCTTTTTACGGTTCCTGGCCTTTTGCT |
| | GGCCTTTTGCTCACATGTTCTTTCCTGCGTTATCCCCTGATTCTGTGGATAAC |
| | CGTATTACCGCCTTTGAGTGAGCTGATACCGCTCGCCGCAGCCGAACGACC |
| | GAGCGCAGCGAGTCAGTGAGCGAGGAAGCGGAAGAGCGCCCAATACGCAA |
| | ACCGCCTCTCCCCGCGCGTTGGCCGATTCATTAATGCAGCTGGCACGACAG |
| | GTTTCCCGACTGGAAAGCGGGCAGTGAGCGCAACGCAATTAATGTGAGTTA |
| | GCTCACTCATTAGGCACCCCAGGCTTTACACTTTATGCTTCCGGCTCGTATG |
| | TTGTGTGGAATTGTGAGCGGATAACAATTTCACACAGGAAACAGCTATGACC |
| | ATGATTACGCCAGATTTAATTAAGG |
| SEQ ID NO: 5 | CCTTAATTAGGCTGCGCGCTCGCTCGCTCACTGAGGCCGCCCGGGCAAAG |
| P747 Sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATCGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTCCCATAGTAACGCCAATAGGGACTTTCCATTG |
| positions 548-751 | ACGTCAATGGGTGGAGTATTTACGGTAAACTGCCCACTTGGCAGTACATCAA |
| AcGFP1 at | GTGTATCATATGCCAAGTACGCCCCCTATTGACGTCAATGACGGTAAATGGC |
| positions 801-1517 | CCGCCTGGCATTATGCCCAGTACATGACCTTATGGGACTTTCCTACTTGGCA |
| miR-183 target | GTACATCTACGTATTAGTCATCGCTATTACCATGGTGATGCGGTTTTGGCAG |
| sequences (4) at | TACATCAATGGGCGTGGATAGCGGTTTGACTCACGGGGATTTCCAAGTCTC |
| positions 1531- | CACCCCATTGACGTCAATGGGAGTTTGTTTTGGCACCAAAATCAACGGGACT |
| 1552, 1553-1574, | TTCCAAAATGTCGTAACAACTCCGCCCCATTGACGCAAATGGGGGGTAGGC |
| 1575-1596, and | GTGTACGGTGGGAGGTCTATATAAGCAGAGCTGGTTTAGTGAACCGTCAGA |
| 1597-1618 | TCCTGCAGGGCCGGCCGCGGCCGCACGCGTATGGTGAGCAAGGGCGCCG |
| WPRE at | AGCTGTTCACCGGCATCGTGCCCATCCTGATCGAGCTGAATGGCGATGTGA |
| positions 1620-2167 | ATGGCCACAAGTTCAGCGTGAGCGGCGAGGGCGAGGGCGATGCCACCTAC |
| bGH polyA at | GGCAAGCTGACCCTGAAGTTCATCTGCACCACCGGCAAGCTGCCTGTGCCC |
| positions 2180-2387 | TGGCCCACCCTGGTGACCACCCTGAGCTACGGCGTGCAGTGCTTCTCACGC |
| 3′ ITR at positions | TACCCCGATCACATGAAGCAGCACGACTTCTTCAAGAGCGCCATGCCTGAG |
| 2475-2604 | GGCTACATCCAGGAGCGCACCATCTTCTTCGAGGATGACGGCAACTACAAG |
| | TCGCGCGCCGAGGTGAAGTTCGAGGGCGATACCCTGGTGAATCGCATCGA |
| | GCTGACCGGCACCGATTTCAAGGAGGATGGCAACATCCTGGGCAATAAGAT |
| | GGAGTACAACTACAACGCCCACAATGTGTACATCATGACCGACAAGGCCAA |
| | GAATGGCATCAAGGTGAACTTCAAGATCCGCCACAACATCGAGGATGGCAG |
| | CGTGCAGCTGGCCGACCACTACCAGCAGAATACCCCCATCGGCGATGGCC |
| | CTGTGCTGCTGCCCGATAACCACTACCTGTCCACCCAGAGCGCCCTGTCCA |
| | AGGACCCCAACGAGAAGCGCGATCACATGATCTACTTCGGCTTCGTGACCG |
| | CCGCCGCCATCACCCACGGCATGGATGAGCTGTACAAGTAATAATAAGCTTA |
| | GTGAATTCTACCAGTGCCATAAGTGAATTCTACCAGTGCCATAAGTGAATTCT |
| | ACCAGTGCCATAAGTGAATTCTACCAGTGCCATAGGATCCAATCAACCTCTG |
| | GATTACAAAATTTGTGAAAGATTGACTGGTATTCTTAACTATGTTGCTCCTTTT |
| | ACGCTATGTGGATACGCTGCTTTAATGCCTTTGTATCATGCTATTGOTTCCCG |
| | TATGGCTTTCATTTTCTCCTCCTTGTATAAATCCTGGTTGCTGTCTCTTTATGA |
| | GGAGTTGTGGCCCGTTGTCAGGCAACGTGGCGTGGTGTGCACTGTGTTTGC |
| | TGACGCAACCCCCACTGGTTGGGGCATTGCCACCACCTGTCAGCTCCTTTC |
| | CGGGACTTTCGCTTTCCCCCTCCCTATTGCCACGGCGGAACTCATCGCCGC |
| | CTGCCTTGCCCGCTGCTGGACAGGGGCTCGGCTGTTGGGCACTGACAATTC |
| | CGTGGTGTTGTCGGGGAAATCATCGTCCTTTCCTTGGCTGCTCGCCTGTGTT |
| | GCCACCTGGATTCTGCGCGGGACGTCCTTCTGCTACGTCCCTTCGGCCCTC |
| | AATCCAGCGGACCTTCCTTCCCGCGGCCTGCTGCCGGCTCTGCGGCCTCTT |
| | CCGCGTCTTCGAGATCTGCCTCGACTGTGCCTTCTAGTTGCCAGCCATCTGT |
| | TGTTTGCCCCTCCCCCGTGCCTTCCTTGACCCTGGAAGGTGCCACTCCCAC |
| | TGTCCTTTCCTAATAAAATGAGGAAATTGCATCGCATTGTCTGAGTAGGTGTC |
| | ATTCTATTCTGGGGGGTGGGGTGGGGCAGGACAGCAAGGGGGAGGATTGG |
| | GAAGACAATAGCAGGCATGCTGGGGACTCGAGTTAAGGGCGAATTCCCGAT |
| | AAGGATCTTCCTAGAGCATGGCTACGTAGATAAGTAGCATGGGGGGTTAATC |
| | ATTAACTACAAGGAACCCCTAGTGATGGAGTTGGCCACTCCCTCTCTGCGC |
| | GCTCGCTCGCTCACTGAGGCCGGGCGACCAAAGGTCGCCCGACGCCCGGG |
| | CTTTGCCCGGGGGGCCTCAGTGAGCGAGCGAGCGCGCAGCCTTAATTAAC |
| | CTAATTCACTGGCCGTCGTTTTACAACGTCGTGACTGGGAAAACCCTGGCGT |
| | TACCCAACTTAATCGCCTTGCAGCACATCCCCCTTTCGCCAGCTGGCGTAAT |
| | AGCGAAGAGGCCCGCACCGATCGCCCTTCCCAACAGTTGCGCAGCCTGAAT |
| | GGCGAATGGGACGCGCCCTGTAGCGGCGCATTAAGCGCGGCGGGTGTGGT |
| | GGTTACGCGCAGCGTGACCGCTACACTTGCCAGCGCCCTAGCGCCCGCTC |
| | CTTTCGCTTTCTTCCCTTCCTTTCTCGCCACGTTCGCCGGCTTTCCCCGTCA |
| | AGCTCTAAATCGGGGGCTCCCTTTAGGGTTCCGATTTAGTGCTTTACGGCAC |
| | CTCGACCCCAAAAAACTTGATTAGGGTGATGGTTCACGTAGTGGGCCATCG |
| | CCCTGATAGACGGTTTTTCGCCCTTTGACGTTGGAGTCCACGTTCTTTAATA |
| | GTGGACTCTTGTTCCAAACTGGAACAACACTCAACCCTATCTCGGTCTATTC |
| | TTTTGATTTATAAGGGATTTTGCCGATTTCGGCCTATTGGTTAAAAAATGAGC |
| | TGATTTAACAAAAATTTAACGCGAATTTTAACAAAATATTAACGCTTACAATTT |
| | AGGTGGCACTTTTCGGGGAAATGTGCGCGGAACCCCTATTTGTTTATTTTTC |
| | TAAATACATTCAAATATGTATCCGCTCATGAGACAATAACCCTGATAAATGCT |
| | TCAATAATATTGAAAAAGGAAGAGTATGAGTATTCAACATTTCCGTGTCGCCC |
| | TTATTCCCTTTTTTGCGGCATTTTGCCTTCCTGTTTTTGCTCACCCAGAAACG |
| | CTGGTGAAAGTAAAAGATGCTGAAGATCAGTTGGGTGCACGAGTGGGTTAC |
| | ATCGAACTGGATCTCAACAGCGGTAAGATCCTTGAGAGTTTTCGCCCCGAAG |
| | AACGTTTTCCAATGATGAGCACTTTTAAAGTTCTGCTATGTGGCGCGGTATTA |
| | TCCCGTATTGACGCCGGGCAAGAGCAACTCGGTCGCCGCATACACTATTCT |
| | CAGAATGACTTGGTTGAGTACTCACCAGTCACAGAAAAGCATCTTACGGATG |
| | GCATGACAGTAAGAGAATTATGCAGTGCTGCCATAACCATGAGTGATAACAC |
| | TGCGGCCAACTTACTTCTGACAACGATCGGAGGACCGAAGGAGCTAACCGC |
| | TTTTTTGCACAACATGGGGGATCATGTAACTCGCCTTGATCGTTGGGAACCG |
| | GAGCTGAATGAAGCCATACCAAACGACGAGCGTGACACCACGATGCCTGTA |
| | GCAATGGCAACAACGTTGCGCAAACTATTAACTGGCGAACTACTTACTCTAG |
| | CTTCCCGGCAACAATTAATAGACTGGATGGAGGCGGATAAAGTTGCAGGAC |
| | CACTTCTGCGCTCGGCCCTTCCGGCTGGCTGGTTTATTGCTGATAAATCTGG |
| | AGCCGGTGAGCGTGGGTCTCGCGGTATCATTGCAGCACTGGGGCCAGATG |
| | GTAAGCCCTCCCGTATCGTAGTTATCTACACGACGGGGAGTCAGGCAACTA |
| | TGGATGAACGAAATAGACAGATCGCTGAGATAGGTGCCTCACTGATTAAGCA |
| | TTGGTAACTGTCAGACCAAGTTTACTCATATATACTTTAGATTGATTTAAAACT |
| | TCATTTTTAATTTAAAAGGATCTAGGTGAAGATCCTTTTTGATAATCTCATGAC |
| | CAAAATCCCTTAACGTGAGTTTTCGTTCCACTGAGCGTCAGACCCCGTAGAA |
| | AAGATCAAAGGATCTTCTTGAGATCCTTTTTTTCTGCGCGTAATCTGCTGCTT |
| | GCAAACAAAAAAACCACCGCTACCAGCGGTGGTTTGTTTGCCGGATCAAGA |
| | GCTACCAACTCTTTTTCCGAAGGTAACTGGCTTCAGCAGAGCGCAGATACCA |
| | AATACTGTTCTTCTAGTGTAGCCGTAGTTAGGCCACCACTTCAAGAACTCTG |
| | TAGCACCGCCTACATACCTCGCTCTGCTAATCCTGTTACCAGTGGCTGCTGC |
| | CAGTGGCGATAAGTCGTGTCTTACCGGGTTGGACTCAAGACGATAGTTACC |
| | GGATAAGGCGCAGCGGTCGGGCTGAACGGGGGGTTCGTGCACACAGCCCA |
| | GCTTGGAGCGAACGACCTACACCGAACTGAGATACCTACAGCGTGAGCTAT |
| | GAGAAAGCGCCACGCTTCCCGAAGGGAGAAAGGCGGACAGGTATCCGGTA |
| | AGCGGCAGGGTCGGAACAGGAGAGCGCACGAGGGAGCTTCCAGGGGGAA |
| | ACGCCTGGTATCTTTATAGTCCTGTCGGGTTTCGCCACCTCTGACTTGAGCG |
| | TCGATTTTTGTGATGCTCGTCAGGGGGGCGGAGCCTATGGAAAAACGCCAG |
| | CAACGCGGCCTTTTTACGGTTCCTGGCCTTTTGCTGGCCTTTTGCTCACATG |
| | TTCTTTCCTGCGTTATCCCCTGATTCTGTGGATAACCGTATTACCGCCTTTGA |
| | GTGAGCTGATACCGCTCGCCGCAGCCGAACGACCGAGCGCAGCGAGTCAG |
| | TGAGCGAGGAAGCGGAAGAGCGCCCAATACGCAAACCGCCTCTCCCCGCG |
| | CGTTGGCCGATTCATTAATGCAGCTGGCACGACAGGTTTCCCGACTGGAAA |
| | GCGGGCAGTGAGCGCAACGCAATTAATGTGAGTTAGCTCACTCATTAGGCA |
| | CCCCAGGCTTTACACTTTATGCTTCCGGCTCGTATGTTGTGTGGAATTGTGA |
| | GCGGATAACAATTTCACACAGGAAACAGCTATGACCATGATTACGCCAGATT |
| | TAATTAAGG |
| SEQ ID NO: 6 | CCTTAATTAGGCTGCGCGCTCGCTCGCTCACTGAGGCCGCCCGGGCAAAG |
| P740 Sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATCGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTCCCATAGTAACGCCAATAGGGACTTTCCATTG |
| positions 548-751 | ACGTCAATGGGTGGAGTATTTACGGTAAACTGCCCACTTGGCAGTACATCAA |
| AcGFP1 at | GTGTATCATATGCCAAGTACGCCCCCTATTGACGTCAATGACGGTAAATGGC |
| positions 801-1517 | CCGCCTGGCATTATGCCCAGTACATGACCTTATGGGACTTTCCTACTTGGCA |
| miR-96 target | GTACATCTACGTATTAGTCATCGCTATTACCATGGTGATGCGGTTTTGGCAG |
| sequence at | TACATCAATGGGCGTGGATAGCGGTTTGACTCACGGGGATTTCCAAGTCTC |
| positions 1531-1553 | CACCCCATTGACGTCAATGGGAGTTTGTTTTGGCACCAAAATCAACGGGACT |
| WPRE at | TTCCAAAATGTCGTAACAACTCCGCCCCATTGACGCAAATGGGGGGTAGGC |
| positions 1555-2102 | GTGTACGGTGGGAGGTCTATATAAGCAGAGCTGGTTTAGTGAACCGTCAGA |
| bGH polyA at | TCCTGCAGGGCCGGCCGCGGCCGCACGCGTATGGTGAGCAAGGGCGCCG |
| positions 2115-2322 | AGCTGTTCACCGGCATCGTGCCCATCCTGATCGAGCTGAATGGCGATGTGA |
| 3′ ITR at positions | ATGGCCACAAGTTCAGCGTGAGCGGCGAGGGCGAGGGCGATGCCACCTAC |
| 2410-2539 | GGCAAGCTGACCCTGAAGTTCATCTGCACCACCGGCAAGCTGCCTGTGCCC |
| | TGGCCCACCCTGGTGACCACCCTGAGCTACGGCGTGCAGTGCTTCTCACGC |
| | TACCCCGATCACATGAAGCAGCACGACTTCTTCAAGAGCGCCATGCCTGAG |
| | GGCTACATCCAGGAGCGCACCATCTTCTTCGAGGATGACGGCAACTACAAG |
| | TCGCGCGCCGAGGTGAAGTTCGAGGGCGATACCCTGGTGAATCGCATCGA |
| | GCTGACCGGCACCGATTTCAAGGAGGATGGCAACATCCTGGGCAATAAGAT |
| | GGAGTACAACTACAACGCCCACAATGTGTACATCATGACCGACAAGGCCAA |
| | GAATGGCATCAAGGTGAACTTCAAGATCCGCCACAACATCGAGGATGGCAG |
| | CGTGCAGCTGGCCGACCACTACCAGCAGAATACCCCCATCGGCGATGGCC |
| | CTGTGCTGCTGCCCGATAACCACTACCTGTCCACCCAGAGCGCCCTGTCCA |
| | AGGACCCCAACGAGAAGCGCGATCACATGATCTACTTCGGCTTCGTGACCG |
| | CCGCCGCCATCACCCACGGCATGGATGAGCTGTACAAGTAATAATAAGCTTA |
| | GCAAAAATGTGCTAGTGCCAAAGGATCCAATCAACCTCTGGATTACAAAATT |
| | TGTGAAAGATTGACTGGTATTCTTAACTATGTTGCTCCTTTTACGCTATGTGG |
| | ATACGCTGCTTTAATGCCTTTGTATCATGCTATTGOTTCCCGTATGGCTTTCA |
| | TTTTCTCCTCCTTGTATAAATCCTGGTTGCTGTCTCTTTATGAGGAGTTGTGG |
| | CCCGTTGTCAGGCAACGTGGCGTGGTGTGCACTGTGTTTGCTGACGCAACC |
| | CCCACTGGTTGGGGCATTGCCACCACCTGTCAGCTCCTTTCCGGGACTTTC |
| | GCTTTCCCCCTCCCTATTGCCACGGCGGAACTCATCGCCGCCTGCCTTGCC |
| | CGCTGCTGGACAGGGGCTCGGCTGTTGGGCACTGACAATTCCGTGGTGTTG |
| | TCGGGGAAATCATCGTCCTTTCCTTGGCTGCTCGCCTGTGTTGCCACCTGG |
| | ATTCTGCGCGGGACGTCCTTCTGCTACGTCCCTTCGGCCCTCAATCCAGCG |
| | GACCTTCCTTCCCGCGGCCTGCTGCCGGCTCTGCGGCCTCTTCCGCGTCTT |
| | CGAGATCTGCCTCGACTGTGCCTTCTAGTTGCCAGCCATCTGTTGTTTGCCC |
| | CTCCCCCGTGCCTTCCTTGACCCTGGAAGGTGCCACTCCCACTGTCCTTTC |
| | CTAATAAAATGAGGAAATTGCATCGCATTGTCTGAGTAGGTGTCATTCTATTC |
| | TGGGGGGTGGGGTGGGGCAGGACAGCAAGGGGGAGGATTGGGAAGACAA |
| | TAGCAGGCATGCTGGGGACTCGAGTTAAGGGCGAATTCCCGATAAGGATCT |
| | TCCTAGAGCATGGCTACGTAGATAAGTAGCATGGCGGGTTAATCATTAACTA |
| | CAAGGAACCCCTAGTGATGGAGTTGGCCACTCCCTCTCTGCGCGCTCGCTC |
| | GCTCACTGAGGCCGGGCGACCAAAGGTCGCCCGACGCCCGGGCTTTGCCC |
| | GGGCGGCCTCAGTGAGCGAGCGAGCGCGCAGCCTTAATTAACCTAATTCAC |
| | TGGCCGTCGTTTTACAACGTCGTGACTGGGAAAACCCTGGCGTTACCCAAC |
| | TTAATCGCCTTGCAGCACATCCCCCTTTCGCCAGCTGGCGTAATAGCGAAGA |
| | GGCCCGCACCGATCGCCCTTCCCAACAGTTGCGCAGCCTGAATGGCGAATG |
| | GGACGCGCCCTGTAGCGGCGCATTAAGCGCGGCGGGTGTGGTGGTTACGC |
| | GCAGCGTGACCGCTACACTTGCCAGCGCCCTAGCGCCCGCTCCTTTCGCTT |
| | TCTTCCCTTCCTTTCTCGCCACGTTCGCCGGCTTTCCCCGTCAAGCTCTAAA |
| | TCGGGGGCTCCCTTTAGGGTTCCGATTTAGTGCTTTACGGCACCTCGACCC |
| | CAAAAAACTTGATTAGGGTGATGGTTCACGTAGTGGGCCATCGCCCTGATAG |
| | ACGGTTTTTCGCCCTTTGACGTTGGAGTCCACGTTCTTTAATAGTGGACTCTT |
| | GTTCCAAACTGGAACAACACTCAACCCTATCTCGGTCTATTCTTTTGATTTAT |
| | AAGGGATTTTGCCGATTTCGGCCTATTGGTTAAAAAATGAGCTGATTTAACAA |
| | AAATTTAACGCGAATTTTAACAAAATATTAACGCTTACAATTTAGGTGGCACT |
| | TTTCGGGGAAATGTGCGCGGAACCCCTATTTGTTTATTTTTCTAAATACATTC |
| | AAATATGTATCCGCTCATGAGACAATAACCCTGATAAATGCTTCAATAATATT |
| | GAAAAAGGAAGAGTATGAGTATTCAACATTTCCGTGTCGCCCTTATTCCCTTT |
| | TTTGCGGCATTTTGCCTTCCTGTTTTTGCTCACCCAGAAACGCTGGTGAAAG |
| | TAAAAGATGCTGAAGATCAGTTGGGTGCACGAGTGGGTTACATCGAACTGG |
| | ATCTCAACAGCGGTAAGATCCTTGAGAGTTTTCGCCCCGAAGAACGTTTTCC |
| | AATGATGAGCACTTTTAAAGTTCTGCTATGTGGCGCGGTATTATCCCGTATT |
| | GACGCCGGGCAAGAGCAACTCGGTCGCCGCATACACTATTCTCAGAATGAC |
| | TTGGTTGAGTACTCACCAGTCACAGAAAAGCATCTTACGGATGGCATGACAG |
| | TAAGAGAATTATGCAGTGCTGCCATAACCATGAGTGATAACACTGCGGCCAA |
| | CTTACTTCTGACAACGATCGGAGGACCGAAGGAGCTAACCGCTTTTTTGCAC |
| | AACATGGGGGATCATGTAACTCGCCTTGATCGTTGGGAACCGGAGCTGAAT |
| | GAAGCCATACCAAACGACGAGCGTGACACCACGATGCCTGTAGCAATGGCA |
| | ACAACGTTGCGCAAACTATTAACTGGCGAACTACTTACTCTAGCTTCCCGGC |
| | AACAATTAATAGACTGGATGGAGGCGGATAAAGTTGCAGGACCACTTCTGC |
| | GCTCGGCCCTTCCGGCTGGCTGGTTTATTGCTGATAAATCTGGAGCCGGTG |
| | AGCGTGGGTCTCGCGGTATCATTGCAGCACTGGGGCCAGATGGTAAGCCCT |
| | CCCGTATCGTAGTTATCTACACGACGGGGAGTCAGGCAACTATGGATGAAC |
| | GAAATAGACAGATCGCTGAGATAGGTGCCTCACTGATTAAGCATTGGTAACT |
| | GTCAGACCAAGTTTACTCATATATACTTTAGATTGATTTAAAACTTCATTTTTA |
| | ATTTAAAAGGATCTAGGTGAAGATCCTTTTTGATAATCTCATGACCAAAATCC |
| | CTTAACGTGAGTTTTCGTTCCACTGAGCGTCAGACCCCGTAGAAAAGATCAA |
| | AGGATCTTCTTGAGATCCTTTTTTTCTGCGCGTAATCTGCTGCTTGCAAACAA |
| | AAAAACCACCGCTACCAGCGGTGGTTTGTTTGCCGGATCAAGAGCTACCAA |
| | CTCTTTTTCCGAAGGTAACTGGCTTCAGCAGAGCGCAGATACCAAATACTGT |
| | TCTTCTAGTGTAGCCGTAGTTAGGCCACCACTTCAAGAACTCTGTAGCACCG |
| | CCTACATACCTCGCTCTGCTAATCCTGTTACCAGTGGCTGCTGCCAGTGGC |
| | GATAAGTCGTGTCTTACCGGGTTGGACTCAAGACGATAGTTACCGGATAAG |
| | GCGCAGCGGTCGGGCTGAACGGGGGGTTCGTGCACACAGCCCAGCTTGGA |
| | GCGAACGACCTACACCGAACTGAGATACCTACAGCGTGAGCTATGAGAAAG |
| | CGCCACGCTTCCCGAAGGGAGAAAGGCGGACAGGTATCCGGTAAGCGGCA |
| | GGGTCGGAACAGGAGAGCGCACGAGGGAGCTTCCAGGGGGAAACGCCTG |
| | GTATCTTTATAGTCCTGTCGGGTTTCGCCACCTCTGACTTGAGCGTCGATTT |
| | TTGTGATGCTCGTCAGGGGGGCGGAGCCTATGGAAAAACGCCAGCAACGC |
| | GGCCTTTTTACGGTTCCTGGCCTTTTGCTGGCCTTTTGCTCACATGTTCTTTC |
| | CTGCGTTATCCCCTGATTCTGTGGATAACCGTATTACCGCCTTTGAGTGAGC |
| | TGATACCGCTCGCCGCAGCCGAACGACCGAGCGCAGCGAGTCAGTGAGCG |
| | AGGAAGCGGAAGAGCGCCCAATACGCAAACCGCCTCTCCCCGCGCGTTGG |
| | CCGATTCATTAATGCAGCTGGCACGACAGGTTTCCCGACTGGAAAGCGGGC |
| | AGTGAGCGCAACGCAATTAATGTGAGTTAGCTCACTCATTAGGCACCCCAG |
| | GCTTTACACTTTATGCTTCCGGCTCGTATGTTGTGTGGAATTGTGAGCGGAT |
| | AACAATTTCACACAGGAAACAGCTATGACCATGATTACGCCAGATTTAATTAA |
| | GG |
| SEQ ID NO: 7 | CCTTAATTAGGCTGCGCGCTCGCTCGCTCACTGAGGCCGCCCGGGCAAAG |
| P741 Sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATCGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTOCCATAGTAACGCCAATAGGGACTTTCCATTG |
| positions 548-751 | ACGTCAATGGGTGGAGTATTTACGGTAAACTGCCCACTTGGCAGTACATCAA |
| AcGFP1 at | GTGTATCATATGCCAAGTACGCCCCCTATTGACGTCAATGACGGTAAATGGC |
| positions 801-1517 | CCGCCTGGCATTATGCCCAGTACATGACCTTATGGGACTTTCCTACTTGGCA |
| miR-182 target | GTACATCTACGTATTAGTCATCGCTATTACCATGGTGATGCGGTTTTGGCAG |
| sequence at | TACATCAATGGGCGTGGATAGCGGTTTGACTCACGGGGATTTCCAAGTCTC |
| positions 1531-1555 | CACCCCATTGACGTCAATGGGAGTTTGTTTTGGCACCAAAATCAACGGGACT |
| WPRE at | TTCCAAAATGTCGTAACAACTCCGCCCCATTGACGCAAATGGGGGGTAGGC |
| positions 1557-2104 | GTGTACGGTGGGAGGTCTATATAAGCAGAGCTGGTTTAGTGAACCGTCAGA |
| bGH polyA at | TCCTGCAGGGCCGGCCGCGGCCGCACGCGTATGGTGAGCAAGGGCGCCG |
| positions 2117-2324 | AGCTGTTCACCGGCATCGTGCCCATCCTGATCGAGCTGAATGGCGATGTGA |
| 3′ ITR at positions | ATGGCCACAAGTTCAGCGTGAGCGGCGAGGGCGAGGGCGATGCCACCTAC |
| 2412-2541 | GGCAAGCTGACCCTGAAGTTCATCTGCACCACCGGCAAGCTGCCTGTGCCC |
| | TGGCCCACCCTGGTGACCACCCTGAGCTACGGCGTGCAGTGCTTCTCACGC |
| | TACCCCGATCACATGAAGCAGCACGACTTCTTCAAGAGCGCCATGCCTGAG |
| | GGCTACATCCAGGAGCGCACCATCTTCTTCGAGGATGACGGCAACTACAAG |
| | TCGCGCGCCGAGGTGAAGTTCGAGGGCGATACCCTGGTGAATCGCATCGA |
| | GCTGACCGGCACCGATTTCAAGGAGGATGGCAACATCCTGGGCAATAAGAT |
| | GGAGTACAACTACAACGCCCACAATGTGTACATCATGACCGACAAGGCCAA |
| | GAATGGCATCAAGGTGAACTTCAAGATCCGCCACAACATCGAGGATGGCAG |
| | CGTGCAGCTGGCCGACCACTACCAGCAGAATACCCCCATCGGCGATGGCC |
| | CTGTGCTGCTGCCCGATAACCACTACCTGTCCACCCAGAGCGCCCTGTCCA |
| | AGGACCCCAACGAGAAGCGCGATCACATGATCTACTTCGGCTTCGTGACCG |
| | CCGCCGCCATCACCCACGGCATGGATGAGCTGTACAAGTAATAATAAGCTT |
| | CGGTGTGAGTTCTACCATTGCCAAAGGATCCAATCAACCTCTGGATTACAAA |
| | ATTTGTGAAAGATTGACTGGTATTCTTAACTATGTTGCTCCTTTTACGCTATG |
| | TGGATACGCTGCTTTAATGCCTTTGTATCATGCTATTGCTTCCCGTATGGCTT |
| | TCATTTTCTCCTCCTTGTATAAATCCTGGTTGCTGTCTCTTTATGAGGAGTTG |
| | TGGCCCGTTGTCAGGCAACGTGGCGTGGTGTGCACTGTGTTTGCTGACGCA |
| | ACCCCCACTGGTTGGGGCATTGCCACCACCTGTCAGCTCCTTTCCGGGACT |
| | TTCGCTTTCCCCCTCCCTATTGCCACGGCGGAACTCATCGCCGCCTGCCTT |
| | GCCCGCTGCTGGACAGGGGCTCGGCTGTTGGGCACTGACAATTCCGTGGT |
| | GTTGTCGGGGAAATCATCGTCCTTTCCTTGGCTGCTCGCCTGTGTTGCCACC |
| | TGGATTCTGCGCGGGACGTCCTTCTGCTACGTCCCTTCGGCCCTCAATCCA |
| | GCGGACCTTCCTTCCCGCGGCCTGCTGCCGGCTCTGCGGCCTCTTCCGCG |
| | TCTTCGAGATCTGCCTCGACTGTGCCTTCTAGTTGCCAGCCATCTGTTGTTT |
| | GCCCCTCCCCCGTGCCTTCCTTGACCCTGGAAGGTGCCACTCCCACTGTCC |
| | TTTCCTAATAAAATGAGGAAATTGCATCGCATTGTCTGAGTAGGTGTCATTCT |
| | ATTCTGGGGGGTGGGGTGGGGCAGGACAGCAAGGGGGAGGATTGGGAAG |
| | ACAATAGCAGGCATGCTGGGGACTCGAGTTAAGGGCGAATTCCCGATAAGG |
| | ATCTTCCTAGAGCATGGCTACGTAGATAAGTAGCATGGCGGGTTAATCATTA |
| | ACTACAAGGAACCCCTAGTGATGGAGTTGGCCACTCCCTCTCTGCGCGCTC |
| | GCTCGCTCACTGAGGCCGGGCGACCAAAGGTCGCCCGACGCCCGGGCTTT |
| | GCCCGGGGGGCCTCAGTGAGCGAGCGAGCGCGCAGCCTTAATTAACCTAA |
| | TTCACTGGCCGTCGTTTTACAACGTCGTGACTGGGAAAACCCTGGCGTTACC |
| | CAACTTAATCGCCTTGCAGCACATCCCCCTTTCGCCAGCTGGCGTAATAGCG |
| | AAGAGGCCCGCACCGATCGCCCTTCCCAACAGTTGCGCAGCCTGAATGGC |
| | GAATGGGACGCGCCCTGTAGCGGCGCATTAAGCGCGGCGGGTGTGGTGGT |
| | TACGCGCAGCGTGACCGCTACACTTGCCAGCGCCCTAGCGCCCGCTCCTTT |
| | CGCTTTCTTCCCTTCCTTTCTCGCCACGTTCGCCGGCTTTCCCCGTCAAGCT |
| | CTAAATCGGGGGCTCCCTTTAGGGTTCCGATTTAGTGCTTTACGGCACCTCG |
| | ACCCCAAAAAACTTGATTAGGGTGATGGTTCACGTAGTGGGCCATCGCCCT |
| | GATAGACGGTTTTTCGCCCTTTGACGTTGGAGTCCACGTTCTTTAATAGTGG |
| | ACTCTTGTTCCAAACTGGAACAACACTCAACCCTATCTCGGTCTATTCTTTTG |
| | ATTTATAAGGGATTTTGCCGATTTCGGCCTATTGGTTAAAAAATGAGCTGATT |
| | TAACAAAAATTTAACGCGAATTTTAACAAAATATTAACGCTTACAATTTAGGTG |
| | GCACTTTTCGGGGAAATGTGCGCGGAACCCCTATTTGTTTATTTTTCTAAATA |
| | CATTCAAATATGTATCCGCTCATGAGACAATAACCCTGATAAATGCTTCAATA |
| | ATATTGAAAAAGGAAGAGTATGAGTATTCAACATTTCCGTGTCGCCCTTATTC |
| | CCTTTTTTGCGGCATTTTGCCTTCCTGTTTTTGCTCACCCAGAAACGCTGGT |
| | GAAAGTAAAAGATGCTGAAGATCAGTTGGGTGCACGAGTGGGTTACATCGA |
| | ACTGGATCTCAACAGCGGTAAGATCCTTGAGAGTTTTCGCCCCGAAGAACGT |
| | TTTCCAATGATGAGCACTTTTAAAGTTCTGCTATGTGGCGCGGTATTATCCC |
| | GTATTGACGCCGGGCAAGAGCAACTCGGTCGCCGCATACACTATTCTCAGA |
| | ATGACTTGGTTGAGTACTCACCAGTCACAGAAAAGCATCTTACGGATGGCAT |
| | GACAGTAAGAGAATTATGCAGTGCTGCCATAACCATGAGTGATAACACTGCG |
| | GCCAACTTACTTCTGACAACGATCGGAGGACCGAAGGAGCTAACCGCTTTTT |
| | TGCACAACATGGGGGATCATGTAACTCGCCTTGATCGTTGGGAACCGGAGC |
| | TGAATGAAGCCATACCAAACGACGAGCGTGACACCACGATGCCTGTAGCAA |
| | TGGCAACAACGTTGCGCAAACTATTAACTGGCGAACTACTTACTCTAGCTTC |
| | CCGGCAACAATTAATAGACTGGATGGAGGCGGATAAAGTTGCAGGACCACT |
| | TCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTA |
| | AGCCCTCCCGTATCGTAGTTATCTACACGACGGGGAGTCAGGCAACTATGG |
| | ATGAACGAAATAGACAGATCGCTGAGATAGGTGCCTCACTGATTAAGCATTG |
| | GTAACTGTCAGACCAAGTTTACTCATATATACTTTAGATTGATTTAAAACTTCA |
| | TTTTTAATTTAAAAGGATCTAGGTGAAGATCCTTTTTGATAATCTCATGACCAA |
| | AATCCCTTAACGTGAGTTTTCGTTCCACTGAGCGTCAGACCCCGTAGAAAAG |
| | ATCAAAGGATCTTCTTGAGATCCTTTTTTTCTGCGCGTAATCTGCTGCTTGCA |
| | AACAAAAAAACCACCGCTACCAGCGGTGGTTTGTTTGCCGGATCAAGAGCTA |
| | CCAACTCTTTTTCCGAAGGTAACTGGCTTCAGCAGAGCGCAGATACCAAATA |
| | CTGTTCTTCTAGTGTAGCCGTAGTTAGGCCACCACTTCAAGAACTCTGTAGC |
| | ACCGCCTACATACCTCGCTCTGCTAATCCTGTTACCAGTGGCTGCTGCCAGT |
| | GGCGATAAGTCGTGTCTTACCGGGTTGGACTCAAGACGATAGTTACCGGAT |
| | AAGGCGCAGCGGTCGGGCTGAACGGGGGGTTCGTGCACACAGCCCAGCTT |
| | GGAGCGAACGACCTACACCGAACTGAGATACCTACAGCGTGAGCTATGAGA |
| | AAGCGCCACGCTTCCCGAAGGGAGAAAGGCGGACAGGTATCCGGTAAGCG |
| | GCAGGGTCGGAACAGGAGAGCGCACGAGGGAGCTTCCAGGGGGAAACGC |
| | CTGGTATCTTTATAGTCCTGTCGGGTTTCGCCACCTCTGACTTGAGCGTCGA |
| | TTTTTGTGATGCTCGTCAGGGGGGCGGAGCCTATGGAAAAACGCCAGCAAC |
| | GCGGCCTTTTTACGGTTCCTGGCCTTTTGCTGGCCTTTTGCTCACATGTTCT |
| | TTCCTGCGTTATCCCCTGATTCTGTGGATAACCGTATTACCGCCTTTGAGTG |
| | AGCTGATACCGCTCGCCGCAGCCGAACGACCGAGCGCAGCGAGTCAGTGA |
| | GCGAGGAAGCGGAAGAGCGCCCAATACGCAAACCGCCTCTCCCCGCGCGT |
| | TGGCCGATTCATTAATGCAGCTGGCACGACAGGTTTCCCGACTGGAAAGCG |
| | GGCAGTGAGCGCAACGCAATTAATGTGAGTTAGCTCACTCATTAGGCACCC |
| | CAGGCTTTACACTTTATGCTTCCGGCTCGTATGTTGTGTGGAATTGTGAGCG |
| | GATAACAATTTCACACAGGAAACAGCTATGACCATGATTACGCCAGATTTAAT |
| | TAAGG |
| SEQ ID NO: 8 | CCTTAATTAGGCTGCGCGCTCGCTCGCTCACTGAGGCCGCCCGGGCAAAG |
| P743 Sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATCGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTCCCATAGTAACGCCAATAGGGACTTTCCATTG |
| positions 548-751 | ACGTCAATGGGTGGAGTATTTACGGTAAACTGCCCACTTGGCAGTACATCAA |
| AcGFP1 at | GTGTATCATATGCCAAGTACGCCCCCTATTGACGTCAATGACGGTAAATGGC |
| positions 801-1517 | CCGCCTGGCATTATGCCCAGTACATGACCTTATGGGACTTTCCTACTTGGCA |
| miR-183 target | GTACATCTACGTATTAGTCATCGCTATTACCATGGTGATGCGGTTTTGGCAG |
| sequence at | TACATCAATGGGCGTGGATAGCGGTTTGACTCACGGGGATTTCCAAGTCTC |
| positions 1531-1552 | CACCCCATTGACGTCAATGGGAGTTTGTTTTGGCACCAAAATCAACGGGACT |
| WPRE at | TTCCAAAATGTCGTAACAACTCCGCCCCATTGACGCAAATGGGGGGTAGGC |
| positions 1554-2101 | GTGTACGGTGGGAGGTCTATATAAGCAGAGCTGGTTTAGTGAACCGTCAGA |
| bGH polyA at | TCCTGCAGGGCCGGCCGCGGCCGCACGCGTATGGTGAGCAAGGGCGCCG |
| positions 2114-2321 | AGCTGTTCACCGGCATCGTGCCCATCCTGATCGAGCTGAATGGCGATGTGA |
| 3′ ITR at positions | ATGGCCACAAGTTCAGCGTGAGCGGCGAGGGCGAGGGCGATGCCACCTAC |
| 2409-2538 | GGCAAGCTGACCCTGAAGTTCATCTGCACCACCGGCAAGCTGCCTGTGCCC |
| | TGGCCCACCCTGGTGACCACCCTGAGCTACGGCGTGCAGTGCTTCTCACGC |
| | TACCCCGATCACATGAAGCAGCACGACTTCTTCAAGAGCGCCATGCCTGAG |
| | GGCTACATCCAGGAGCGCACCATCTTCTTCGAGGATGACGGCAACTACAAG |
| | TCGCGCGCCGAGGTGAAGTTCGAGGGCGATACCCTGGTGAATCGCATCGA |
| | GCTGACCGGCACCGATTTCAAGGAGGATGGCAACATCCTGGGCAATAAGAT |
| | GGAGTACAACTACAACGCCCACAATGTGTACATCATGACCGACAAGGCCAA |
| | GAATGGCATCAAGGTGAACTTCAAGATCCGCCACAACATCGAGGATGGCAG |
| | CGTGCAGCTGGCCGACCACTACCAGCAGAATACCCCCATCGGCGATGGCC |
| | CTGTGCTGCTGCCCGATAACCACTACCTGTCCACCCAGAGCGCCCTGTCCA |
| | AGGACCCCAACGAGAAGCGCGATCACATGATCTACTTCGGCTTCGTGACCG |
| | CCGCCGCCATCACCCACGGCATGGATGAGCTGTACAAGTAATAATAAGOTTA |
| | GTGAATTCTACCAGTGCCATAGGATCCAATCAACCTCTGGATTACAAAATTTG |
| | TGAAAGATTGACTGGTATTCTTAACTATGTTGCTCCTTTTACGCTATGTGGAT |
| | ACGCTGCTTTAATGCCTTTGTATCATGCTATTGCTTCCCGTATGGCTTTCATT |
| | TTCTCCTCCTTGTATAAATCCTGGTTGCTGTCTCTTTATGAGGAGTTGTGGCC |
| | CGTTGTCAGGCAACGTGGCGTGGTGTGCACTGTGTTTGCTGACGCAACCCC |
| | CACTGGTTGGGGCATTGCCACCACCTGTCAGCTCCTTTCCGGGACTTTCGC |
| | TTTCCCCCTCCCTATTGCCACGGCGGAACTCATCGCCGCCTGCCTTGCCCG |
| | CTGCTGGACAGGGGCTCGGCTGTTGGGCACTGACAATTCCGTGGTGTTGTC |
| | GGGGAAATCATCGTCCTTTCCTTGGCTGCTCGCCTGTGTTGCCACCTGGATT |
| | CTGCGCGGGACGTCCTTCTGCTACGTCCCTTCGGCCCTCAATCCAGCGGAC |
| | CTTCCTTCCCGCGGCCTGCTGCCGGCTCTGCGGCCTCTTCCGCGTCTTCGA |
| | GATCTGCCTCGACTGTGCCTTCTAGTTGCCAGCCATCTGTTGTTTGCCCCTC |
| | CCCCGTGCCTTCCTTGACCCTGGAAGGTGCCACTCCCACTGTCCTTTCCTAA |
| | TAAAATGAGGAAATTGCATCGCATTGTCTGAGTAGGTGTCATTCTATTCTGG |
| | GGGGTGGGGGGGGCAGGACAGCAAGGGGGAGGATTGGGAAGACAATAG |
| | CAGGCATGCTGGGGACTCGAGTTAAGGGCGAATTCCCGATAAGGATOTTCC |
| | TAGAGCATGGCTACGTAGATAAGTAGCATGGCGGGTTAATCATTAACTACAA |
| | GGAACCCCTAGTGATGGAGTTGGCCACTCCCTCTCTGCGCGCTCGCTCGCT |
| | CACTGAGGCCGGGCGACCAAAGGTCGCCCGACGCCCGGGCTTTGCCCGG |
| | GCGGCCTCAGTGAGCGAGCGAGCGCGCAGCCTTAATTAACCTAATTCACTG |
| | GCCGTCGTTTTACAACGTCGTGACTGGGAAAACCCTGGCGTTACCCAACTTA |
| | ATCGCCTTGCAGCACATCCCCCTTTCGCCAGCTGGCGTAATAGCGAAGAGG |
| | CCCGCACCGATCGCCCTTCCCAACAGTTGCGCAGCCTGAATGGCGAATGGG |
| | ACGCGCCCTGTAGCGGCGCATTAAGCGCGGCGGGTGTGGTGGTTACGCGC |
| | AGCGTGACCGCTACACTTGCCAGCGCCCTAGCGCCCGCTCCTTTCGCTTTC |
| | TTCCCTTCCTTTCTCGCCACGTTCGCCGGCTTTCCCCGTCAAGCTCTAAATC |
| | GGGGGCTCCCTTTAGGGTTCCGATTTAGTGCTTTACGGCACCTCGACCCCA |
| | AAAAACTTGATTAGGGTGATGGTTCACGTAGTGGGCCATCGCCCTGATAGAC |
| | GGTTTTTCGCCCTTTGACGTTGGAGTCCACGTTCTTTAATAGTGGACTCTTGT |
| | TCCAAACTGGAACAACACTCAACCCTATCTCGGTCTATTCTTTTGATTTATAA |
| | GGGATTTTGCCGATTTCGGCCTATTGGTTAAAAAATGAGCTGATTTAACAAAA |
| | ATTTAACGCGAATTTTAACAAAATATTAACGCTTACAATTTAGGTGGCACTTTT |
| | CGGGGAAATGTGCGCGGAACCCCTATTTGTTTATTTTTCTAAATACATTCAAA |
| | TATGTATCCGCTCATGAGACAATAACCCTGATAAATGCTTCAATAATATTGAA |
| | AAAGGAAGAGTATGAGTATTCAACATTTCCGTGTCGCCCTTATTCCCTTTTTT |
| | GCGGCATTTTGCCTTCCTGTTTTTGCTCACCCAGAAACGCTGGTGAAAGTAA |
| | AAGATGCTGAAGATCAGTTGGGTGCACGAGTGGGTTACATCGAACTGGATC |
| | TCAACAGCGGTAAGATCCTTGAGAGTTTTCGCCCCGAAGAACGTTTTCCAAT |
| | GATGAGCACTTTTAAAGTTCTGCTATGTGGCGCGGTATTATCCCGTATTGAC |
| | GCCGGGCAAGAGCAACTCGGTCGCCGCATACACTATTCTCAGAATGACTTG |
| | GTTGAGTACTCACCAGTCACAGAAAAGCATCTTACGGATGGCATGACAGTAA |
| | GAGAATTATGCAGTGCTGCCATAACCATGAGTGATAACACTGCGGCCAACTT |
| | ACTTCTGACAACGATCGGAGGACCGAAGGAGCTAACCGCTTTTTTGCACAAC |
| | ATGGGGGATCATGTAACTCGCCTTGATCGTTGGGAACCGGAGCTGAATGAA |
| | GCCATACCAAACGACGAGCGTGACACCACGATGCCTGTAGCAATGGCAACA |
| | ACGTTGCGCAAACTATTAACTGGCGAACTACTTACTCTAGCTTCCCGGCAAC |
| | AATTAATAGACTGGATGGAGGCGGATAAAGTTGCAGGACCACTTCTGCGCT |
| | CGGCCCTTCCGGCTGGCTGGTTTATTGCTGATAAATCTGGAGCCGGTGAGC |
| | GTGGGTCTCGCGGTATCATTGCAGCACTGGGGCCAGATGGTAAGCCCTCCC |
| | GTATCGTAGTTATCTACACGACGGGGAGTCAGGCAACTATGGATGAACGAA |
| | ATAGACAGATCGCTGAGATAGGTGCCTCACTGATTAAGCATTGGTAACTGTC |
| | AGACCAAGTTTACTCATATATACTTTAGATTGATTTAAAACTTCATTTTTAATTT |
| | AAAAGGATCTAGGTGAAGATCCTTTTTGATAATCTCATGACCAAAATCCCTTA |
| | ACGTGAGTTTTCGTTCCACTGAGCGTCAGACCCCGTAGAAAAGATCAAAGGA |
| | TCTTCTTGAGATCCTTTTTTTCTGCGCGTAATCTGCTGCTTGCAAACAAAAAA |
| | ACCACCGCTACCAGCGGTGGTTTGTTTGCCGGATCAAGAGCTACCAACTCTT |
| | TTTCCGAAGGTAACTGGCTTCAGCAGAGCGCAGATACCAAATACTGTTCTTC |
| | TAGTGTAGCCGTAGTTAGGCCACCACTTCAAGAACTCTGTAGCACCGCCTAC |
| | ATACCTCGCTCTGCTAATCCTGTTACCAGTGGCTGCTGCCAGTGGCGATAAG |
| | TCGTGTCTTACCGGGTTGGACTCAAGACGATAGTTACCGGATAAGGCGCAG |
| | CGGTCGGGCTGAACGGGGGGTTCGTGCACACAGCCCAGCTTGGAGCGAAC |
| | GACCTACACCGAACTGAGATACCTACAGCGTGAGCTATGAGAAAGCGCCAC |
| | GCTTCCCGAAGGGAGAAAGGCGGACAGGTATCCGGTAAGCGGCAGGGTCG |
| | GAACAGGAGAGCGCACGAGGGAGCTTCCAGGGGGAAACGCCTGGTATCTT |
| | TATAGTCCTGTCGGGTTTCGCCACCTCTGACTTGAGCGTCGATTTTTGTGAT |
| | GCTCGTCAGGGGGGCGGAGCCTATGGAAAAACGCCAGCAACGCGGCCTTT |
| | TTACGGTTCCTGGCCTTTTGCTGGCCTTTTGCTCACATGTTCTTTCCTGCGTT |
| | ATCCCCTGATTCTGTGGATAACCGTATTACCGCCTTTGAGTGAGCTGATACC |
| | GCTCGCCGCAGCCGAACGACCGAGCGCAGCGAGTCAGTGAGCGAGGAAGC |
| | GGAAGAGCGCCCAATACGCAAACCGCCTCTCCCCGCGCGTTGGCCGATTCA |
| | TTAATGCAGCTGGCACGACAGGTTTCCCGACTGGAAAGCGGGCAGTGAGCG |
| | CAACGCAATTAATGTGAGTTAGCTCACTCATTAGGCACCCCAGGCTTTACAC |
| | TTTATGCTTCCGGCTCGTATGTTGTGTGGAATTGTGAGCGGATAACAATTTC |
| | ACACAGGAAACAGCTATGACCATGATTACGCCAGATTTAATTAAGG |
| SEQ ID NO: 9 | CCTTAATTAGGCTGCGCGCTCGCTCGCTCACTGAGGCCGCCCGGGCAAAG |
| P750 sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATOGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTCCCATAGTAACGCCAATAGGGACTTTCCATTG |
| positions 548-751 | ACGTCAATGGGTGGAGTATTTACGGTAAACTGCCCACTTGGCAGTACATCAA |
| mGjb2 at | GTGTATCATATGCCAAGTACGCCCCCTATTGACGTCAATGACGGTAAATGGC |
| positions 801-1481 | CCGCCTGGCATTATGCCCAGTACATGACCTTATGGGACTTTCCTACTTGGCA |
| miR-183 target | GTACATCTACGTATTAGTCATCGCTATTACCATGGTGATGCGGTTTTGGCAG |
| sequence at | TACATCAATGGGCGTGGATAGCGGTTTGACTCACGGGGATTTCCAAGTCTC |
| positions 1498-1519 | CACCCCATTGACGTCAATGGGAGTTTGTTTTGGCACCAAAATCAACGGGACT |
| miR-96 target | TTCCAAAATGTCGTAACAACTCCGCCCCATTGACGCAAATGGGGGGTAGGC |
| sequence at | GTGTACGGTGGGAGGTCTATATAAGCAGAGCTGGTTTAGTGAACCGTCAGA |
| positions 1520-1542 | TCCTGCAGGGCCGGCCGCGGCCGCGCCGCCATGGATTGGGGCACACTCCA |
| miR-182 target | GAGCATCCTCGGGGGTGTCAACAAACACTCCACCAGCATTGGAAAGATCTG |
| sequence at | GCTCACGGTCCTCTTCATCTTCCGCATCATGATCCTCGTGGTGGCTGCAAAG |
| positions 1543-1567 | GAGGTGTGGGGAGATGAGCAAGCCGATTTTGTCTGCAACACGCTCCAGCCT |
| WPRE at | GGCTGCAAGAATGTATGCTACGACCACCACTTCCCCATCTCTCACATCCGGC |
| positions 1569-2116 | TCTGGGCTCTGCAGCTGATCATGGTGTCCACGCCAGCCCTCCTGGTAGCTA |
| bGH polyA at | TGCATGTGGCCTACCGGAGACATGAAAAGAAACGGAAGTTCATGAAGGGAG |
| positions 2129-2336 | AGATAAAGAACGAGTTTAAGGACATCGAAGAGATCAAAACCCAGAAGGTCC |
| 3′ ITR at positions | GTATCGAAGGGTCCCTGTGGTGGACCTACACCACCAGCATCTTCTTCCGGG |
| 2424-2553 | TCATCTTTGAAGCCGTCTTCATGTACGTCTTTTACATCATGTACAATGGCTTC |
| | TTCATGCAACGTCTGGTGAAATGCAACGCTTGGCCCTGCCCCAATACAGTG |
| | GACTGCTTCATTTCCAGGCCCACAGAAAAGACTGTCTTCACCGTGTTTATGA |
| | TTTCTGTGTCTGGAATTTGCATTCTGCTAAATATCACAGAGCTGTGCTATTTG |
| | TTCGTTAGGTATTGCTCAGGAAAGTCCAAAAGACCAGTCTAAACGCGTTAAT |
| | AAGCTTAGTGAATTCTACCAGTGCCATAAGCAAAAATGTGCTAGTGCCAAAC |
| | GGTGTGAGTTCTACCATTGCCAAAGGATCCAATCAACCTCTGGATTACAAAA |
| | TTTGTGAAAGATTGACTGGTATTCTTAACTATGTTGCTCCTTTTACGCTATGT |
| | GGATACGCTGCTTTAATGCCTTTGTATCATGCTATTGCTTCCCGTATGGCTTT |
| | CATTTTCTCCTCCTTGTATAAATCCTGGTTGCTGTCTCTTTATGAGGAGTTGT |
| | GGCCCGTTGTCAGGCAACGTGGCGTGGTGTGCACTGTGTTTGCTGACGCAA |
| | CCCCCACTGGTTGGGGCATTGCCACCACCTGTCAGCTCCTTTCCGGGACTT |
| | TCGCTTTCCCCCTCCCTATTGCCACGGCGGAACTCATCGCCGCCTGCCTTG |
| | CCCGCTGCTGGACAGGGGCTCGGCTGTTGGGCACTGACAATTCCGTGGTG |
| | TTGTCGGGGAAATCATCGTCCTTTCCTTGGCTGCTCGCCTGTGTTGCCACCT |
| | GGATTCTGCGCGGGACGTCCTTCTGCTACGTCCCTTCGGCCCTCAATCCAG |
| | CGGACCTTCCTTCCCGCGGCCTGCTGCCGGCTCTGCGGCCTCTTCCGCGT |
| | CTTCGAGATCTGCCTCGACTGTGCCTTCTAGTTGCCAGCCATCTGTTGTTTG |
| | CCCCTCCCCCGTGCCTTCCTTGACCCTGGAAGGTGCCACTCCCACTGTCCT |
| | TTCCTAATAAAATGAGGAAATTGCATCGCATTGTCTGAGTAGGTGTCATTCTA |
| | TTCTGGGGGGGGGGTGGGGCAGGACAGCAAGGGGGAGGATTGGGAAGA |
| | CAATAGCAGGCATGCTGGGGACTCGAGTTAAGGGCGAATTCCCGATAAGGA |
| | TCTTCCTAGAGCATGGCTACGTAGATAAGTAGCATGGCGGGTTAATCATTAA |
| | CTACAAGGAACCCCTAGTGATGGAGTTGGCCACTCCCTCTCTGCGCGCTCG |
| | CTCGCTCACTGAGGCCGGGCGACCAAAGGTCGCCCGACGCCCGGGCTTTG |
| | CCCGGGCGGCCTCAGTGAGCGAGCGAGCGCGCAGCCTTAATTAACCTAATT |
| | CACTGGCCGTCGTTTTACAACGTCGTGACTGGGAAAACCCTGGCGTTACCC |
| | AACTTAATCGCCTTGCAGCACATCCCCCTTTCGCCAGCTGGCGTAATAGCGA |
| | AGAGGCCCGCACCGATCGCCCTTCCCAACAGTTGCGCAGCCTGAATGGCG |
| | AATGGGACGCGCCCTGTAGCGGCGCATTAAGCGCGGCGGGTGTGGTGGTT |
| | ACGCGCAGCGTGACCGCTACACTTGCCAGCGCCCTAGCGCCCGCTCCTTTC |
| | GCTTTCTTCCCTTCCTTTCTCGCCACGTTCGCCGGCTTTCCCCGTCAAGCTC |
| | TAAATCGGGGGCTCCCTTTAGGGTTCCGATTTAGTGCTTTACGGCACCTCGA |
| | CCCCAAAAAACTTGATTAGGGTGATGGTTCACGTAGTGGGCCATCGCCCTG |
| | ATAGACGGTTTTTCGCCCTTTGACGTTGGAGTCCACGTTCTTTAATAGTGGA |
| | CTCTTGTTCCAAACTGGAACAACACTCAACCCTATCTCGGTCTATTCTTTTGA |
| | TTTATAAGGGATTTTGCCGATTTCGGCCTATTGGTTAAAAAATGAGCTGATTT |
| | AACAAAAATTTAACGCGAATTTTAACAAAATATTAACGCTTACAATTTAGGTG |
| | GCACTTTTCGGGGAAATGTGCGCGGAACCCCTATTTGTTTATTTTTCTAAATA |
| | CATTCAAATATGTATCCGCTCATGAGACAATAACCCTGATAAATGCTTCAATA |
| | ATATTGAAAAAGGAAGAGTATGAGTATTCAACATTTCCGTGTCGCCCTTATTC |
| | CCTTTTTTGCGGCATTTTGCCTTCCTGTTTTTGCTCACCCAGAAACGCTGGT |
| | GAAAGTAAAAGATGCTGAAGATCAGTTGGGTGCACGAGTGGGTTACATCGA |
| | ACTGGATCTCAACAGCGGTAAGATCCTTGAGAGTTTTCGCCCCGAAGAACGT |
| | TTTCCAATGATGAGCACTTTTAAAGTTCTGCTATGTGGCGCGGTATTATCCC |
| | GTATTGACGCCGGGCAAGAGCAACTCGGTCGCCGCATACACTATTCTCAGA |
| | ATGACTTGGTTGAGTACTCACCAGTCACAGAAAAGCATCTTACGGATGGCAT |
| | GACAGTAAGAGAATTATGCAGTGCTGCCATAACCATGAGTGATAACACTGCG |
| | GCCAACTTACTTCTGACAACGATCGGAGGACCGAAGGAGCTAACCGCTTTTT |
| | TGCACAACATGGGGGATCATGTAACTCGCCTTGATCGTTGGGAACCGGAGC |
| | TGAATGAAGCCATACCAAACGACGAGCGTGACACCACGATGCCTGTAGCAA |
| | TGGCAACAACGTTGCGCAAACTATTAACTGGCGAACTACTTACTCTAGCTTC |
| | CCGGCAACAATTAATAGACTGGATGGAGGCGGATAAAGTTGCAGGACCACT |
| | TCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTA |
| | AGCCCTCCCGTATCGTAGTTATCTACACGACGGGGAGTCAGGCAACTATGG |
| | ATGAACGAAATAGACAGATCGCTGAGATAGGTGCCTCACTGATTAAGCATTG |
| | GTAACTGTCAGACCAAGTTTACTCATATATACTTTAGATTGATTTAAAACTTCA |
| | TTTTTAATTTAAAAGGATCTAGGTGAAGATCCTTTTTGATAATCTCATGACCAA |
| | AATCCCTTAACGTGAGTTTTCGTTCCACTGAGCGTCAGACCCCGTAGAAAAG |
| | ATCAAAGGATCTTCTTGAGATCCTTTTTTTCTGCGCGTAATCTGCTGCTTGCA |
| | AACAAAAAAACCACCGCTACCAGCGGTGGTTTGTTTGCCGGATCAAGAGCTA |
| | CCAACTCTTTTTCCGAAGGTAACTGGCTTCAGCAGAGCGCAGATACCAAATA |
| | CTGTTCTTCTAGTGTAGCCGTAGTTAGGCCACCACTTCAAGAACTCTGTAGC |
| | ACCGCCTACATACCTCGCTCTGCTAATCCTGTTACCAGTGGCTGCTGCCAGT |
| | GGCGATAAGTCGTGTCTTACCGGGTTGGACTCAAGACGATAGTTACCGGAT |
| | AAGGCGCAGCGGTCGGGCTGAACGGGGGGTTCGTGCACACAGCCCAGCTT |
| | GGAGCGAACGACCTACACCGAACTGAGATACCTACAGCGTGAGCTATGAGA |
| | AAGCGCCACGCTTCCCGAAGGGAGAAAGGCGGACAGGTATCCGGTAAGCG |
| | GCAGGGTCGGAACAGGAGAGCGCACGAGGGAGCTTCCAGGGGGAAACGC |
| | CTGGTATCTTTATAGTCCTGTCGGGTTTCGCCACCTCTGACTTGAGCGTCGA |
| | TTTTTGTGATGCTCGTCAGGGGGGCGGAGCCTATGGAAAAACGCCAGCAAC |
| | GCGGCCTTTTTACGGTTCCTGGCCTTTTGCTGGCCTTTTGCTCACATGTTCT |
| | TTCCTGCGTTATCCCCTGATTCTGTGGATAACCGTATTACCGCCTTTGAGTG |
| | AGCTGATACCGCTCGCCGCAGCCGAACGACCGAGCGCAGCGAGTCAGTGA |
| | GCGAGGAAGCGGAAGAGCGCCCAATACGCAAACCGCCTCTCCCCGCGCGT |
| | TGGCCGATTCATTAATGCAGCTGGCACGACAGGTTTCCCGACTGGAAAGCG |
| | GGCAGTGAGCGCAACGCAATTAATGTGAGTTAGCTCACTCATTAGGCACCC |
| | CAGGCTTTACACTTTATGCTTCCGGCTCGTATGTTGTGTGGAATTGTGAGCG |
| | GATAACAATTTCACACAGGAAACAGCTATGACCATGATTACGCCAGATTTAAT |
| | TAAGG |
| SEQ ID NO: 10 | CCTTAATTAGGCTGCGCGCTCGCTCGCTCACTGAGGCCGCCCGGGCAAAG |
| P752 Sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATCGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTCCCATAGTAACGCCAATAGGGACTTTCCATTG |
| positions 548-751 | ACGTCAATGGGTGGAGTATTTACGGTAAACTGCCCACTTGGCAGTACATCAA |
| mGjb2 at | GTGTATCATATGCCAAGTACGCCCCCTATTGACGTCAATGACGGTAAATGGC |
| positions 801-1481 | CCGCCTGGCATTATGCCCAGTACATGACCTTATGGGACTTTCCTACTTGGCA |
| miR-183 target | GTACATCTACGTATTAGTCATCGCTATTACCATGGTGATGCGGTTTTGGCAG |
| sequences (3) at | TACATCAATGGGCGTGGATAGCGGTTTGACTCACGGGGATTTCCAAGTCTC |
| positions 1498- | CACCCCATTGACGTCAATGGGAGTTTGTTTTGGCACCAAAATCAACGGGACT |
| 1519, 1520-1541, | TTCCAAAATGTCGTAACAACTCCGCCCCATTGACGCAAATGGGGGGTAGGC |
| and 1542-1563 | GTGTACGGTGGGAGGTCTATATAAGCAGAGCTGGTTTAGTGAACCGTCAGA |
| miR-96 target | TCCTGCAGGGCCGGCCGCGGCCGCGCCGCCATGGATTGGGGCACACTCCA |
| sequences (3) at | GAGCATCCTCGGGGGTGTCAACAAACACTCCACCAGCATTGGAAAGATCTG |
| positions 1564- | GCTCACGGTCCTCTTCATCTTCCGCATCATGATCCTCGTGGTGGCTGCAAAG |
| 1586, 1587-1609, | GAGGTGTGGGGAGATGAGCAAGCCGATTTTGTCTGCAACACGCTCCAGCCT |
| and 1610-1632 | GGCTGCAAGAATGTATGCTACGACCACCACTTCCCCATCTCTCACATCCGGC |
| miR-182 target | TCTGGGCTCTGCAGCTGATCATGGTGTCCACGCCAGCCCTCCTGGTAGCTA |
| sequences (3) at | TGCATGTGGCCTACCGGAGACATGAAAAGAAACGGAAGTTCATGAAGGGAG |
| positions 1633- | AGATAAAGAACGAGTTTAAGGACATCGAAGAGATCAAAACCCAGAAGGTCC |
| 1657, 1658-1682, | GTATCGAAGGGTCCCTGTGGTGGACCTACACCACCAGCATCTTCTTCCGGG |
| and 1683-1707 | TCATCTTTGAAGCCGTCTTCATGTACGTCTTTTACATCATGTACAATGGCTTC |
| WPRE at | TTCATGCAACGTCTGGTGAAATGCAACGCTTGGCCCTGCCCCAATACAGTG |
| positions 1709-2256 | GACTGCTTCATTTCCAGGCCCACAGAAAAGACTGTCTTCACCGTGTTTATGA |
| bGH polyA at | TTTCTGTGTCTGGAATTTGCATTCTGCTAAATATCACAGAGCTGTGCTATTTG |
| positions 2269-2476 | TTCGTTAGGTATTGCTCAGGAAAGTCCAAAAGACCAGTCTAAACGCGTTAAT |
| 3′ ITR at positions | AAGCTTAGTGAATTCTACCAGTGCCATAAGTGAATTCTACCAGTGCCATAAG |
| 2564-2693 | TGAATTCTACCAGTGCCATAAGCAAAAATGTGCTAGTGCCAAAAGCAAAAAT |
| | GTGCTAGTGCCAAAAGCAAAAATGTGCTAGTGCCAAACGGTGTGAGTTCTAC |
| | CATTGCCAAACGGTGTGAGTTCTACCATTGCCAAACGGTGTGAGTTCTACCA |
| | TTGCCAAAGGATCCAATCAACCTCTGGATTACAAAATTTGTGAAAGATTGACT |
| | GGTATTCTTAACTATGTTGCTCCTTTTACGCTATGTGGATACGCTGCTTTAAT |
| | GCCTTTGTATCATGCTATTGCTTCCCGTATGGCTTTCATTTTCTCCTCCTTGT |
| | ATAAATCCTGGTTGCTGTCTCTTTATGAGGAGTTGTGGCCCGTTGTCAGGCA |
| | ACGTGGCGTGGTGTGCACTGTGTTTGCTGACGCAACCCCCACTGGTTGGGG |
| | CATTGCCACCACCTGTCAGCTCCTTTCCGGGACTTTCGCTTTCCCCCTCCCT |
| | ATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAATCATC |
| | GTCCTTTCCTTGGCTGCTCGCCTGTGTTGCCACCTGGATTCTGCGCGGGAC |
| | GTCCTTCTGCTACGTCCCTTCGGCCCTCAATCCAGCGGACCTTCCTTCCCG |
| | CGGCCTGCTGCCGGCTCTGCGGCCTCTTCCGCGTCTTCGAGATCTGCCTCG |
| | ACTGTGCCTTCTAGTTGCCAGCCATCTGTTGTTTGCCCCTCCCCCGTGCCTT |
| | CCTTGACCCTGGAAGGTGCCACTCCCACTGTCCTTTCCTAATAAAATGAGGA |
| | AATTGCATCGCATTGTCTGAGTAGGTGTCATTCTATTCTGGGGGGTGGGGTG |
| | GGGCAGGACAGCAAGGGGGAGGATTGGGAAGACAATAGCAGGCATGCTGG |
| | GGACTCGAGTTAAGGGCGAATTCCCGATAAGGATCTTCCTAGAGCATGGCT |
| | ACGTAGATAAGTAGCATGGGGGGTTAATCATTAACTACAAGGAACCCCTAGT |
| | GATGGAGTTGGCCACTCCCTCTCTGCGCGCTCGCTCGCTCACTGAGGCCG |
| | GGCGACCAAAGGTCGCCCGACGCCCGGGCTTTGCCCGGGGGGCCTCAGTG |
| | AGCGAGCGAGCGCGCAGCCTTAATTAACCTAATTCACTGGCCGTCGTTTTAC |
| | AACGTCGTGACTGGGAAAACCCTGGCGTTACCCAACTTAATCGCCTTGCAG |
| | CACATCCCCCTTTCGCCAGCTGGCGTAATAGCGAAGAGGCCCGCACCGATC |
| | GCCCTTCCCAACAGTTGCGCAGCCTGAATGGCGAATGGGACGCGCCCTGTA |
| | GCGGCGCATTAAGCGCGGCGGGTGTGGTGGTTACGCGCAGCGTGACCGCT |
| | ACACTTGCCAGCGCCCTAGCGCCCGCTCCTTTCGCTTTCTTCCCTTCCTTTC |
| | TCGCCACGTTCGCCGGCTTTCCCCGTCAAGCTCTAAATCGGGGGCTCCCTT |
| | TAGGGTTCCGATTTAGTGCTTTACGGCACCTCGACCCCAAAAAACTTGATTA |
| | GGGTGATGGTTCACGTAGTGGGCCATCGCCCTGATAGACGGTTTTTCGCCC |
| | TTTGACGTTGGAGTCCACGTTCTTTAATAGTGGACTCTTGTTCCAAACTGGAA |
| | CAACACTCAACCCTATCTCGGTCTATTCTTTTGATTTATAAGGGATTTTGCCG |
| | ATTTCGGCCTATTGGTTAAAAAATGAGCTGATTTAACAAAAATTTAACGCGAA |
| | TTTTAACAAAATATTAACGCTTACAATTTAGGTGGCACTTTTCGGGGAAATGT |
| | GCGCGGAACCCCTATTTGTTTATTTTTCTAAATACATTCAAATATGTATCCGC |
| | TCATGAGACAATAACCCTGATAAATGCTTCAATAATATTGAAAAAGGAAGAGT |
| | ATGAGTATTCAACATTTCCGTGTCGCCCTTATTCCCTTTTTTGCGGCATTTTG |
| | CCTTCCTGTTTTTGCTCACCCAGAAACGCTGGTGAAAGTAAAAGATGCTGAA |
| | GATCAGTTGGGTGCACGAGTGGGTTACATCGAACTGGATCTCAACAGCGGT |
| | AAGATCCTTGAGAGTTTTCGCCCCGAAGAACGTTTTCCAATGATGAGCACTT |
| | TTAAAGTTCTGCTATGTGGCGCGGTATTATCCCGTATTGACGCCGGGCAAGA |
| | GCAACTCGGTCGCCGCATACACTATTCTCAGAATGACTTGGTTGAGTACTCA |
| | CCAGTCACAGAAAAGCATCTTACGGATGGCATGACAGTAAGAGAATTATGCA |
| | GTGCTGCCATAACCATGAGTGATAACACTGCGGCCAACTTACTTCTGACAAC |
| | GATCGGAGGACCGAAGGAGCTAACCGCTTTTTTGCACAACATGGGGGATCA |
| | TGTAACTCGCCTTGATCGTTGGGAACCGGAGCTGAATGAAGCCATACCAAA |
| | CGACGAGCGTGACACCACGATGCCTGTAGCAATGGCAACAACGTTGCGCAA |
| | ACTATTAACTGGCGAACTACTTACTCTAGCTTCCCGGCAACAATTAATAGACT |
| | GGATGGAGGCGGATAAAGTTGCAGGACCACTTCTGCGCTCGGCCCTTCCG |
| | GCTGGCTGGTTTATTGCTGATAAATCTGGAGCCGGTGAGCGTGGGTCTCGC |
| | GGTATCATTGCAGCACTGGGGCCAGATGGTAAGCCCTCCCGTATCGTAGTT |
| | ATCTACACGACGGGGAGTCAGGCAACTATGGATGAACGAAATAGACAGATC |
| | GCTGAGATAGGTGCCTCACTGATTAAGCATTGGTAACTGTCAGACCAAGTTT |
| | ACTCATATATACTTTAGATTGATTTAAAACTTCATTTTTAATTTAAAAGGATCTA |
| | GGTGAAGATCCTTTTTGATAATCTCATGACCAAAATCCCTTAACGTGAGTTTT |
| | CGTTCCACTGAGCGTCAGACCCCGTAGAAAAGATCAAAGGATOTTCTTGAGA |
| | TCCTTTTTTTCTGCGCGTAATCTGCTGCTTGCAAACAAAAAAACCACCGCTAC |
| | CAGCGGTGGTTTGTTTGCCGGATCAAGAGCTACCAACTCTTTTTCCGAAGGT |
| | AACTGGCTTCAGCAGAGCGCAGATACCAAATACTGTTCTTCTAGTGTAGCCG |
| | TAGTTAGGCCACCACTTCAAGAACTCTGTAGCACCGCCTACATACCTCGCTC |
| | TGCTAATCCTGTTACCAGTGGCTGCTGCCAGTGGCGATAAGTCGTGTCTTAC |
| | CGGGTTGGACTCAAGACGATAGTTACCGGATAAGGCGCAGCGGTCGGGCT |
| | GAACGGGGGGTTCGTGCACACAGCCCAGCTTGGAGCGAACGACCTACACC |
| | GAACTGAGATACCTACAGCGTGAGCTATGAGAAAGCGCCACGCTTCCCGAA |
| | GGGAGAAAGGCGGACAGGTATCCGGTAAGCGGCAGGGTCGGAACAGGAGA |
| | GCGCACGAGGGAGCTTCCAGGGGGAAACGCCTGGTATCTTTATAGTCCTGT |
| | CGGGTTTCGCCACCTCTGACTTGAGCGTCGATTTTTGTGATGCTCGTCAGG |
| | GGGGCGGAGCCTATGGAAAAACGCCAGCAACGCGGCCTTTTTACGGTTCCT |
| | GGCCTTTTGCTGGCCTTTTGCTCACATGTTCTTTCCTGCGTTATCCCCTGATT |
| | CTGTGGATAACCGTATTACCGCCTTTGAGTGAGCTGATACCGCTCGCCGCA |
| | GCCGAACGACCGAGCGCAGCGAGTCAGTGAGCGAGGAAGCGGAAGAGCG |
| | CCCAATACGCAAACCGCCTCTCCCCGCGCGTTGGCCGATTCATTAATGCAG |
| | CTGGCACGACAGGTTTCCCGACTGGAAAGCGGGCAGTGAGCGCAACGCAA |
| | TTAATGTGAGTTAGCTCACTCATTAGGCACCCCAGGCTTTACACTTTATGOTT |
| | CCGGCTCGTATGTTGTGTGGAATTGTGAGCGGATAACAATTTCACACAGGAA |
| | ACAGCTATGACCATGATTACGCCAGATTTAATTAAGG |
| SEQ ID NO: 11 | CCTTAATTAGGCTGCGCGCTCGCTCGCTCACTGAGGCCGCCCGGGCAAAG |
| P753 Sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATCGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTCCCATAGTAACGCCAATAGGGACTTTCCATTG |
| positions 548-751 | ACGTCAATGGGTGGAGTATTTACGGTAAACTGCCCACTTGGCAGTACATCAA |
| mGjb2 at | GTGTATCATATGCCAAGTACGCCCCCTATTGACGTCAATGACGGTAAATGGC |
| positions 801-1481 | CCGCCTGGCATTATGCCCAGTACATGACCTTATGGGACTTTCCTACTTGGCA |
| miR-96 target | GTACATCTACGTATTAGTCATCGCTATTACCATGGTGATGCGGTTTTGGCAG |
| sequences (4) at | TACATCAATGGGCGTGGATAGCGGTTTGACTCACGGGGATTTCCAAGTCTC |
| positions 1498- | CACCCCATTGACGTCAATGGGAGTTTGTTTTGGCACCAAAATCAACGGGACT |
| 1520, 1521-1543, | TTCCAAAATGTCGTAACAACTCCGCCCCATTGACGCAAATGGGGGGTAGGC |
| 1544-1566, and | GTGTACGGTGGGAGGTCTATATAAGCAGAGCTGGTTTAGTGAACCGTCAGA |
| 1567-1589 | TCCTGCAGGGCCGGCCGCGGCCGCGCCGCCATGGATTGGGGCACACTCCA |
| WPRE at | GAGCATCCTCGGGGGTGTCAACAAACACTCCACCAGCATTGGAAAGATCTG |
| positions 1591-2138 | GCTCACGGTCCTCTTCATCTTCCGCATCATGATCCTCGTGGTGGCTGCAAAG |
| bGH polyA at | GAGGTGTGGGGAGATGAGCAAGCCGATTTTGTCTGCAACACGCTCCAGCCT |
| positions 2151-2358 | GGCTGCAAGAATGTATGCTACGACCACCACTTCCCCATCTCTCACATCCGGC |
| 3′ ITR at positions | TCTGGGCTCTGCAGCTGATCATGGTGTCCACGCCAGCCCTCCTGGTAGCTA |
| 2446-2575 | TGCATGTGGCCTACCGGAGACATGAAAAGAAACGGAAGTTCATGAAGGGAG |
| | AGATAAAGAACGAGTTTAAGGACATCGAAGAGATCAAAACCCAGAAGGTCC |
| | GTATCGAAGGGTCCCTGTGGTGGACCTACACCACCAGCATCTTCTTCCGGG |
| | TCATCTTTGAAGCCGTCTTCATGTACGTCTTTTACATCATGTACAATGGCTTC |
| | TTCATGCAACGTCTGGTGAAATGCAACGCTTGGCCCTGCCCCAATACAGTG |
| | GACTGCTTCATTTCCAGGCCCACAGAAAAGACTGTCTTCACCGTGTTTATGA |
| | TTTCTGTGTCTGGAATTTGCATTCTGCTAAATATCACAGAGCTGTGCTATTTG |
| | TTCGTTAGGTATTGCTCAGGAAAGTCCAAAAGACCAGTCTAAACGCGTTAAT |
| | AAGCTTAGCAAAAATGTGCTAGTGCCAAAAGCAAAAATGTGCTAGTGCCAAA |
| | AGCAAAAATGTGCTAGTGCCAAAAGCAAAAATGTGCTAGTGCCAAAGGATCC |
| | AATCAACCTCTGGATTACAAAATTTGTGAAAGATTGACTGGTATTCTTAACTA |
| | TGTTGCTCCTTTTACGCTATGTGGATACGCTGCTTTAATGCCTTTGTATCATG |
| | CTATTGCTTCCCGTATGGCTTTCATTTTCTCCTCCTTGTATAAATCCTGGTTG |
| | CTGTCTCTTTATGAGGAGTTGTGGCCCGTTGTCAGGCAACGTGGCGTGGTG |
| | TGCACTGTGTTTGCTGACGCAACCCCCACTGGTTGGGGCATTGCCACCACC |
| | TGTCAGCTCCTTTCCGGGACTTTCGCTTTCCCCCTCCCTATTGCCACGGCGG |
| | AACTCATCGCCGCCTGCCTTGCCCGCTGCTGGACAGGGGCTCGGCTGTTG |
| | GGCACTGACAATTCCGTGGTGTTGTCGGGGAAATCATCGTCCTTTCCTTGGC |
| | TGCTCGCCTGTGTTGCCACCTGGATTCTGCGCGGGACGTCCTTCTGCTACG |
| | TCCCTTCGGCCCTCAATCCAGCGGACCTTCCTTCCCGCGGCCTGCTGCCGG |
| | CTCTGCGGCCTCTTCCGCGTCTTCGAGATCTGCCTCGACTGTGCCTTCTAGT |
| | TGCCAGCCATCTGTTGTTTGCCCCTCCCCCGTGCCTTCCTTGACCCTGGAA |
| | GGTGCCACTCCCACTGTCCTTTCCTAATAAAATGAGGAAATTGCATCGCATT |
| | GTCTGAGTAGGTGTCATTCTATTCTGGGGGGTGGGGTGGGGCAGGACAGC |
| | AAGGGGGAGGATTGGGAAGACAATAGCAGGCATGCTGGGGACTCGAGTTA |
| | AGGGCGAATTCCCGATAAGGATCTTCCTAGAGCATGGCTACGTAGATAAGTA |
| | GCATGGCGGGTTAATCATTAACTACAAGGAACCCCTAGTGATGGAGTTGGC |
| | CACTCCCTCTCTGCGCGCTCGCTCGCTCACTGAGGCCGGGCGACCAAAGG |
| | TCGCCCGACGCCCGGGCTTTGCCCGGGGGGCCTCAGTGAGCGAGCGAGC |
| | GCGCAGCCTTAATTAACCTAATTCACTGGCCGTCGTTTTACAACGTCGTGAC |
| | TGGGAAAACCCTGGCGTTACCCAACTTAATCGCCTTGCAGCACATCCCCCTT |
| | TCGCCAGCTGGCGTAATAGCGAAGAGGCCCGCACCGATCGCCCTTCCCAAC |
| | AGTTGCGCAGCCTGAATGGCGAATGGGACGCGCCCTGTAGCGGCGCATTA |
| | AGCGCGGCGGGTGTGGTGGTTACGCGCAGCGTGACCGCTACACTTGCCAG |
| | CGCCCTAGCGCCCGCTCCTTTCGCTTTCTTCCCTTCCTTTCTCGCCACGTTC |
| | GCCGGCTTTCCCCGTCAAGCTCTAAATCGGGGGCTCCCTTTAGGGTTCCGA |
| | TTTAGTGCTTTACGGCACCTCGACCCCAAAAAACTTGATTAGGGTGATGGTT |
| | CACGTAGTGGGCCATCGCCCTGATAGACGGTTTTTCGCCCTTTGACGTTGG |
| | AGTCCACGTTCTTTAATAGTGGACTCTTGTTCCAAACTGGAACAACACTCAAC |
| | CCTATCTCGGTCTATTCTTTTGATTTATAAGGGATTTTGCCGATTTCGGCCTA |
| | TTGGTTAAAAAATGAGCTGATTTAACAAAAATTTAACGCGAATTTTAACAAAAT |
| | ATTAACGCTTACAATTTAGGTGGCACTTTTCGGGGAAATGTGCGCGGAACCC |
| | CTATTTGTTTATTTTTCTAAATACATTCAAATATGTATCCGCTCATGAGACAAT |
| | AACCCTGATAAATGCTTCAATAATATTGAAAAAGGAAGAGTATGAGTATTCAA |
| | CATTTCCGTGTCGCCCTTATTCCCTTTTTTGCGGCATTTTGCCTTCCTGTTTT |
| | TGCTCACCCAGAAACGCTGGTGAAAGTAAAAGATGCTGAAGATCAGTTGGG |
| | TGCACGAGTGGGTTACATCGAACTGGATCTCAACAGCGGTAAGATCCTTGA |
| | GAGTTTTCGCCCCGAAGAACGTTTTCCAATGATGAGCACTTTTAAAGTTCTG |
| | CTATGTGGCGCGGTATTATCCCGTATTGACGCCGGGCAAGAGCAACTCGGT |
| | CGCCGCATACACTATTCTCAGAATGACTTGGTTGAGTACTCACCAGTCACAG |
| | AAAAGCATCTTACGGATGGCATGACAGTAAGAGAATTATGCAGTGCTGCCAT |
| | AACCATGAGTGATAACACTGCGGCCAACTTACTTCTGACAACGATCGGAGGA |
| | CCGAAGGAGCTAACCGCTTTTTTGCACAACATGGGGGATCATGTAACTCGC |
| | CTTGATCGTTGGGAACCGGAGCTGAATGAAGCCATACCAAACGACGAGCGT |
| | GACACCACGATGCCTGTAGCAATGGCAACAACGTTGCGCAAACTATTAACTG |
| | GCGAACTACTTACTCTAGCTTCCCGGCAACAATTAATAGACTGGATGGAGGC |
| | GGATAAAGTTGCAGGACCACTTCTGCGCTCGGCCCTTCCGGCTGGCTGGTT |
| | TATTGCTGATAAATCTGGAGCCGGTGAGCGTGGGTCTCGCGGTATCATTGC |
| | AGCACTGGGGCCAGATGGTAAGCCCTCCCGTATCGTAGTTATCTACACGAC |
| | GGGGAGTCAGGCAACTATGGATGAACGAAATAGACAGATCGCTGAGATAGG |
| | TGCCTCACTGATTAAGCATTGGTAACTGTCAGACCAAGTTTACTCATATATAC |
| | TTTAGATTGATTTAAAACTTCATTTTTAATTTAAAAGGATCTAGGTGAAGATCC |
| | TTTTTGATAATCTCATGACCAAAATCCCTTAACGTGAGTTTTCGTTCCACTGA |
| | GCGTCAGACCCCGTAGAAAAGATCAAAGGATCTTCTTGAGATCCTTTTTTTCT |
| | GCGCGTAATCTGCTGCTTGCAAACAAAAAAACCACCGCTACCAGCGGTGGT |
| | TTGTTTGCCGGATCAAGAGCTACCAACTCTTTTTCCGAAGGTAACTGGCTTC |
| | AGCAGAGCGCAGATACCAAATACTGTTCTTCTAGTGTAGCCGTAGTTAGGCC |
| | ACCACTTCAAGAACTCTGTAGCACCGCCTACATACCTCGCTCTGCTAATCCT |
| | GTTACCAGTGGCTGCTGCCAGTGGCGATAAGTCGTGTCTTACCGGGTTGGA |
| | CTCAAGACGATAGTTACCGGATAAGGCGCAGCGGTCGGGCTGAACGGGGG |
| | GTTCGTGCACACAGCCCAGCTTGGAGCGAACGACCTACACCGAACTGAGAT |
| | ACCTACAGCGTGAGCTATGAGAAAGCGCCACGCTTCCCGAAGGGAGAAAGG |
| | CGGACAGGTATCCGGTAAGCGGCAGGGTCGGAACAGGAGAGCGCACGAGG |
| | GAGCTTCCAGGGGGAAACGCCTGGTATCTTTATAGTCCTGTCGGGTTTCGC |
| | CACCTCTGACTTGAGCGTCGATTTTTGTGATGCTCGTCAGGGGGGGGGAGC |
| | CTATGGAAAAACGCCAGCAACGCGGCCTTTTTACGGTTCCTGGCCTTTTGCT |
| | GGCCTTTTGCTCACATGTTCTTTCCTGCGTTATCCCCTGATTCTGTGGATAAC |
| | CGTATTACCGCCTTTGAGTGAGCTGATACCGCTCGCCGCAGCCGAACGACC |
| | GAGCGCAGCGAGTCAGTGAGCGAGGAAGCGGAAGAGCGCCCAATACGCAA |
| | ACCGCCTCTCCCCGCGCGTTGGCCGATTCATTAATGCAGCTGGCACGACAG |
| | GTTTCCCGACTGGAAAGCGGGCAGTGAGCGCAACGCAATTAATGTGAGTTA |
| | GCTCACTCATTAGGCACCCCAGGCTTTACACTTTATGCTTCCGGCTCGTATG |
| | TTGTGTGGAATTGTGAGCGGATAACAATTTCACACAGGAAACAGCTATGACC |
| | ATGATTACGCCAGATTTAATTAAGG |
| SEQ ID NO: 12 | CCTTAATTAGGCTGCGCGCTCGCTCGCTCACTGAGGCCGCCCGGGCAAAG |
| P754 Sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATCGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTCCCATAGTAACGCCAATAGGGACTTTCCATTG |
| positions 548-751 | ACGTCAATGGGTGGAGTATTTACGGTAAACTGCCCACTTGGCAGTACATCAA |
| mGjb2 at | GTGTATCATATGCCAAGTACGCCCCCTATTGACGTCAATGACGGTAAATGGC |
| positions 801-1481 | CCGCCTGGCATTATGCCCAGTACATGACCTTATGGGACTTTCCTACTTGGCA |
| miR-182 target | GTACATCTACGTATTAGTCATCGCTATTACCATGGTGATGCGGTTTTGGCAG |
| sequences (4) at | TACATCAATGGGCGTGGATAGCGGTTTGACTCACGGGGATTTCCAAGTCTC |
| positions 1498- | CACCCCATTGACGTCAATGGGAGTTTGTTTTGGCACCAAAATCAACGGGACT |
| 1522, 1523-1547, | TTCCAAAATGTCGTAACAACTCCGCCCCATTGACGCAAATGGGGGGTAGGC |
| 1548-1572, and | GTGTACGGTGGGAGGTCTATATAAGCAGAGCTGGTTTAGTGAACCGTCAGA |
| 1573-1597 | TCCTGCAGGGCCGGCCGCGGCCGCGCCGCCATGGATTGGGGCACACTCCA |
| WPRE at | GAGCATCCTCGGGGGTGTCAACAAACACTCCACCAGCATTGGAAAGATCTG |
| positions 1599-2146 | GCTCACGGTCCTCTTCATCTTCCGCATCATGATCCTCGTGGTGGCTGCAAAG |
| bGH polyA at | GAGGTGTGGGGAGATGAGCAAGCCGATTTTGTCTGCAACACGCTCCAGCCT |
| positions 2159-2366 | GGCTGCAAGAATGTATGCTACGACCACCACTTCCCCATCTCTCACATCCGGC |
| 3′ ITR at positions | TCTGGGCTCTGCAGCTGATCATGGTGTCCACGCCAGCCCTCCTGGTAGCTA |
| 2454-2583 | TGCATGTGGCCTACCGGAGACATGAAAAGAAACGGAAGTTCATGAAGGGAG |
| | AGATAAAGAACGAGTTTAAGGACATCGAAGAGATCAAAACCCAGAAGGTCC |
| | GTATCGAAGGGTCCCTGTGGTGGACCTACACCACCAGCATCTTCTTCCGGG |
| | TCATCTTTGAAGCCGTCTTCATGTACGTCTTTTACATCATGTACAATGGCTTC |
| | TTCATGCAACGTCTGGTGAAATGCAACGCTTGGCCCTGCCCCAATACAGTG |
| | GACTGCTTCATTTCCAGGCCCACAGAAAAGACTGTCTTCACCGTGTTTATGA |
| | TTTCTGTGTCTGGAATTTGCATTCTGCTAAATATCACAGAGCTGTGCTATTTG |
| | TTCGTTAGGTATTGCTCAGGAAAGTCCAAAAGACCAGTCTAAACGCGTTAAT |
| | AAGCTTCGGTGTGAGTTCTACCATTGCCAAACGGTGTGAGTTCTACCATTGC |
| | CAAACGGTGTGAGTTCTACCATTGCCAAACGGTGTGAGTTCTACCATTGCCA |
| | AAGGATCCAATCAACCTCTGGATTACAAAATTTGTGAAAGATTGACTGGTATT |
| | CTTAACTATGTTGCTCCTTTTACGCTATGTGGATACGCTGCTTTAATGCCTTT |
| | GTATCATGCTATTGCTTCCCGTATGGCTTTCATTTTCTCCTCCTTGTATAAAT |
| | CCTGGTTGCTGTCTCTTTATGAGGAGTTGTGGCCCGTTGTCAGGCAACGTG |
| | GCGTGGTGTGCACTGTGTTTGCTGACGCAACCCCCACTGGTTGGGGCATTG |
| | CCACCACCTGTCAGCTCCTTTCCGGGACTTTCGCTTTCCCCCTCCCTATTGC |
| | CACGGCGGAACTCATCGCCGCCTGCCTTGCCCGCTGCTGGACAGGGGCTC |
| | GGCTGTTGGGCACTGACAATTCCGTGGTGTTGTCGGGGAAATCATCGTCCT |
| | TTCCTTGGCTGCTCGCCTGTGTTGCCACCTGGATTCTGCGCGGGACGTCCT |
| | TCTGCTACGTCCCTTCGGCCCTCAATCCAGCGGACCTTCCTTCCCGCGGCC |
| | TGCTGCCGGCTCTGCGGCCTCTTCCGCGTCTTCGAGATCTGCCTCGACTGT |
| | GCCTTCTAGTTGCCAGCCATCTGTTGTTTGCCCCTCCCCCGTGCCTTCCTTG |
| | ACCCTGGAAGGTGCCACTCCCACTGTCCTTTCCTAATAAAATGAGGAAATTG |
| | CATCGCATTGTCTGAGTAGGTGTCATTCTATTCTGGGGGGTGGGGTGGGGC |
| | AGGACAGCAAGGGGGAGGATTGGGAAGACAATAGCAGGCATGCTGGGGAC |
| | TCGAGTTAAGGGCGAATTCCCGATAAGGATCTTCCTAGAGCATGGCTACGTA |
| | GATAAGTAGCATGGCGGGTTAATCATTAACTACAAGGAACCCCTAGTGATGG |
| | AGTTGGCCACTCCCTCTCTGCGCGCTCGCTCGCTCACTGAGGCCGGGCGA |
| | CCAAAGGTCGCCCGACGCCCGGGCTTTGCCCGGGGGGCCTCAGTGAGCGA |
| | GCGAGCGCGCAGCCTTAATTAACCTAATTCACTGGCCGTCGTTTTACAACGT |
| | CGTGACTGGGAAAACCCTGGCGTTACCCAACTTAATCGCCTTGCAGCACAT |
| | CCCCCTTTCGCCAGCTGGCGTAATAGCGAAGAGGCCCGCACCGATCGCCCT |
| | TCCCAACAGTTGCGCAGCCTGAATGGCGAATGGGACGCGCCCTGTAGCGG |
| | CGCATTAAGCGCGGCGGGTGTGGTGGTTACGCGCAGCGTGACCGCTACAC |
| | TTGCCAGCGCCCTAGCGCCCGCTCCTTTCGCTTTCTTCCCTTCCTTTCTCGC |
| | CACGTTCGCCGGCTTTCCCCGTCAAGCTCTAAATCGGGGGCTCCCTTTAGG |
| | GTTCCGATTTAGTGCTTTACGGCACCTCGACCCCAAAAAACTTGATTAGGGT |
| | GATGGTTCACGTAGTGGGCCATCGCCCTGATAGACGGTTTTTCGCCCTTTGA |
| | CGTTGGAGTCCACGTTCTTTAATAGTGGACTCTTGTTCCAAACTGGAACAAC |
| | ACTCAACCCTATCTCGGTCTATTCTTTTGATTTATAAGGGATTTTGCCGATTT |
| | CGGCCTATTGGTTAAAAAATGAGCTGATTTAACAAAAATTTAACGCGAATTTT |
| | AACAAAATATTAACGCTTACAATTTAGGTGGCACTTTTCGGGGAAATGTGCG |
| | CGGAACCCCTATTTGTTTATTTTTCTAAATACATTCAAATATGTATCCGCTCAT |
| | GAGACAATAACCCTGATAAATGCTTCAATAATATTGAAAAAGGAAGAGTATGA |
| | GTATTCAACATTTCCGTGTCGCCCTTATTCCCTTTTTTGCGGCATTTTGCCTT |
| | CCTGTTTTTGCTCACCCAGAAACGCTGGTGAAAGTAAAAGATGCTGAAGATC |
| | AGTTGGGTGCACGAGTGGGTTACATCGAACTGGATCTCAACAGCGGTAAGA |
| | TCCTTGAGAGTTTTCGCCCCGAAGAACGTTTTCCAATGATGAGCACTTTTAAA |
| | GTTCTGCTATGTGGCGCGGTATTATCCCGTATTGACGCCGGGCAAGAGCAA |
| | CTCGGTCGCCGCATACACTATTCTCAGAATGACTTGGTTGAGTACTCACCAG |
| | TCACAGAAAAGCATCTTACGGATGGCATGACAGTAAGAGAATTATGCAGTGC |
| | TGCCATAACCATGAGTGATAACACTGCGGCCAACTTACTTCTGACAACGATC |
| | GGAGGACCGAAGGAGCTAACCGCTTTTTTGCACAACATGGGGGATCATGTA |
| | ACTCGCCTTGATCGTTGGGAACCGGAGCTGAATGAAGCCATACCAAACGAC |
| | GAGCGTGACACCACGATGCCTGTAGCAATGGCAACAACGTTGCGCAAACTA |
| | TTAACTGGCGAACTACTTACTCTAGCTTCCCGGCAACAATTAATAGACTGGA |
| | TGGAGGCGGATAAAGTTGCAGGACCACTTCTGCGCTCGGCCCTTCCGGCTG |
| | GCTGGTTTATTGCTGATAAATCTGGAGCCGGTGAGCGTGGGTCTCGCGGTA |
| | TCATTGCAGCACTGGGGCCAGATGGTAAGCCCTCCCGTATCGTAGTTATCTA |
| | CACGACGGGGAGTCAGGCAACTATGGATGAACGAAATAGACAGATCGCTGA |
| | GATAGGTGCCTCACTGATTAAGCATTGGTAACTGTCAGACCAAGTTTACTCA |
| | TATATACTTTAGATTGATTTAAAACTTCATTTTTAATTTAAAAGGATCTAGGTG |
| | AAGATCCTTTTTGATAATCTCATGACCAAAATCCCTTAACGTGAGTTTTOGTT |
| | CCACTGAGCGTCAGACCCCGTAGAAAAGATCAAAGGATCTTCTTGAGATCCT |
| | TTTTTTCTGCGCGTAATCTGCTGCTTGCAAACAAAAAAACCACCGCTACCAG |
| | CGGTGGTTTGTTTGCCGGATCAAGAGCTACCAACTCTTTTTCCGAAGGTAAC |
| | TGGCTTCAGCAGAGCGCAGATACCAAATACTGTTCTTCTAGTGTAGCCGTAG |
| | TTAGGCCACCACTTCAAGAACTCTGTAGCACCGCCTACATACCTCGCTCTGC |
| | TAATCCTGTTACCAGTGGCTGCTGCCAGTGGCGATAAGTCGTGTCTTACCG |
| | GGTTGGACTCAAGACGATAGTTACCGGATAAGGCGCAGCGGTCGGGCTGAA |
| | CGGGGGGTTCGTGCACACAGCCCAGCTTGGAGCGAACGACCTACACCGAA |
| | CTGAGATACCTACAGCGTGAGCTATGAGAAAGCGCCACGCTTCCCGAAGGG |
| | AGAAAGGCGGACAGGTATCCGGTAAGCGGCAGGGTCGGAACAGGAGAGCG |
| | CACGAGGGAGCTTCCAGGGGGAAACGCCTGGTATCTTTATAGTCCTGTCGG |
| | GTTTCGCCACCTCTGACTTGAGCGTCGATTTTTGTGATGCTCGTCAGGGGG |
| | GCGGAGCCTATGGAAAAACGCCAGCAACGCGGCCTTTTTACGGTTCCTGGC |
| | CTTTTGCTGGCCTTTTGCTCACATGTTCTTTCCTGCGTTATCCCCTGATTCTG |
| | TGGATAACCGTATTACCGCCTTTGAGTGAGCTGATACCGCTCGCCGCAGCC |
| | GAACGACCGAGCGCAGCGAGTCAGTGAGCGAGGAAGCGGAAGAGCGCCCA |
| | ATACGCAAACCGCCTCTCCCCGCGCGTTGGCCGATTCATTAATGCAGCTGG |
| | CACGACAGGTTTCCCGACTGGAAAGCGGGCAGTGAGCGCAACGCAATTAAT |
| | GTGAGTTAGCTCACTCATTAGGCACCCCAGGCTTTACACTTTATGCTTCCGG |
| | CTCGTATGTTGTGTGGAATTGTGAGCGGATAACAATTTCACACAGGAAACAG |
| | CTATGACCATGATTACGCCAGATTTAATTAAGG |
| SEQ ID NO: 13 | CCTTAATTAGGCTGCGCGCTCGCTCGCTCACTGAGGCCGCCCGGGCAAAG |
| P755 Sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATCGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTCCCATAGTAACGCCAATAGGGACTTTCCATTG |
| positions 548-751 | ACGTCAATGGGTGGAGTATTTACGGTAAACTGCCCACTTGGCAGTACATCAA |
| mGjb2 at | GTGTATCATATGCCAAGTACGCCCCCTATTGACGTCAATGACGGTAAATGGC |
| positions 801-1481 | CCGCCTGGCATTATGCCCAGTACATGACCTTATGGGACTTTCCTACTTGGCA |
| miR-183 target | GTACATCTACGTATTAGTCATCGCTATTACCATGGTGATGCGGTTTTGGCAG |
| sequences (4) at | TACATCAATGGGCGTGGATAGCGGTTTGACTCACGGGGATTTCCAAGTCTC |
| positions 1498- | CACCCCATTGACGTCAATGGGAGTTTGTTTTGGCACCAAAATCAACGGGACT |
| 1519, 1520-1541, | TTCCAAAATGTCGTAACAACTCCGCCCCATTGACGCAAATGGGGGGTAGGC |
| 1542-1563, and | GTGTACGGTGGGAGGTCTATATAAGCAGAGCTGGTTTAGTGAACCGTCAGA |
| 1564-1585 | TCCTGCAGGGCCGGCCGCGGCCGCGCCGCCATGGATTGGGGCACACTCCA |
| WPRE at | GAGCATCCTCGGGGGTGTCAACAAACACTCCACCAGCATTGGAAAGATCTG |
| positions 1587-2134 | GCTCACGGTCCTCTTCATCTTCCGCATCATGATCCTCGTGGTGGCTGCAAAG |
| bGH polyA at | GAGGTGTGGGGAGATGAGCAAGCCGATTTTGTCTGCAACACGCTCCAGCCT |
| positions 2147-2354 | GGCTGCAAGAATGTATGCTACGACCACCACTTCCCCATCTCTCACATCCGGC |
| 3′ ITR at positions | TCTGGGCTCTGCAGCTGATCATGGTGTCCACGCCAGCCCTCCTGGTAGCTA |
| 2442-2571 | TGCATGTGGCCTACCGGAGACATGAAAAGAAACGGAAGTTCATGAAGGGAG |
| | AGATAAAGAACGAGTTTAAGGACATCGAAGAGATCAAAACCCAGAAGGTCC |
| | GTATCGAAGGGTCCCTGTGGTGGACCTACACCACCAGCATCTTCTTCCGGG |
| | TCATCTTTGAAGCCGTCTTCATGTACGTCTTTTACATCATGTACAATGGCTTC |
| | TTCATGCAACGTCTGGTGAAATGCAACGCTTGGCCCTGCCCCAATACAGTG |
| | GACTGCTTCATTTCCAGGCCCACAGAAAAGACTGTCTTCACCGTGTTTATGA |
| | TTTCTGTGTCTGGAATTTGCATTCTGCTAAATATCACAGAGCTGTGCTATTTG |
| | TTCGTTAGGTATTGCTCAGGAAAGTCCAAAAGACCAGTCTAAACGCGTTAAT |
| | AAGCTTAGTGAATTCTACCAGTGCCATAAGTGAATTCTACCAGTGCCATAAG |
| | TGAATTCTACCAGTGCCATAAGTGAATTCTACCAGTGCCATAGGATCCAATC |
| | AACCTCTGGATTACAAAATTTGTGAAAGATTGACTGGTATTCTTAACTATGTT |
| | GCTCCTTTTACGCTATGTGGATACGCTGCTTTAATGCCTTTGTATCATGCTAT |
| | TGCTTCCCGTATGGCTTTCATTTTCTCCTCCTTGTATAAATCCTGGTTGCTGT |
| | CTCTTTATGAGGAGTTGTGGCCCGTTGTCAGGCAACGTGGCGTGGTGTGCA |
| | CTGTGTTTGCTGACGCAACCCCCACTGGTTGGGGCATTGCCACCACCTGTC |
| | AGCTCCTTTCCGGGACTTTCGCTTTCCCCCTCCCTATTGCCACGGCGGAACT |
| | CATCGCCGCCTGCCTTGCCCGCTGCTGGACAGGGGCTCGGCTGTTGGGCA |
| | CTGACAATTCCGTGGTGTTGTCGGGGAAATCATCGTCCTTTCCTTGGCTGCT |
| | CGCCTGTGTTGCCACCTGGATTCTGCGCGGGACGTCCTTCTGCTACGTCCC |
| | TTCGGCCCTCAATCCAGCGGACCTTCCTTCCCGCGGCCTGCTGCCGGCTCT |
| | GCGGCCTCTTCCGCGTCTTCGAGATCTGCCTCGACTGTGCCTTCTAGTTGC |
| | CAGCCATCTGTTGTTTGCCCCTCCCCCGTGCCTTCCTTGACCCTGGAAGGT |
| | GCCACTCCCACTGTCCTTTCCTAATAAAATGAGGAAATTGCATCGCATTGTCT |
| | GAGTAGGTGTCATTCTATTCTGGGGGGTGGGGTGGGGCAGGACAGCAAGG |
| | GGGAGGATTGGGAAGACAATAGCAGGCATGCTGGGGACTCGAGTTAAGGG |
| | CGAATTCCCGATAAGGATCTTCCTAGAGCATGGCTACGTAGATAAGTAGCAT |
| | GGCGGGTTAATCATTAACTACAAGGAACCCCTAGTGATGGAGTTGGCCACT |
| | CCCTCTCTGCGCGCTCGCTCGCTCACTGAGGCCGGGCGACCAAAGGTCGC |
| | CCGACGCCCGGGCTTTGCCCGGGCGGCCTCAGTGAGCGAGCGAGCGCGC |
| | AGCCTTAATTAACCTAATTCACTGGCCGTCGTTTTACAACGTCGTGACTGGG |
| | AAAACCCTGGCGTTACCCAACTTAATCGCCTTGCAGCACATCCCCCTTTCGC |
| | CAGCTGGCGTAATAGCGAAGAGGCCCGCACCGATCGCCCTTCCCAACAGTT |
| | GCGCAGCCTGAATGGCGAATGGGACGCGCCCTGTAGCGGCGCATTAAGCG |
| | CGGCGGGTGTGGTGGTTACGCGCAGCGTGACCGCTACACTTGCCAGCGCC |
| | CTAGCGCCCGCTCCTTTCGCTTTCTTCCCTTCCTTTCTCGCCACGTTCGCCG |
| | GCTTTCCCCGTCAAGCTCTAAATCGGGGGCTCCCTTTAGGGTTCCGATTTAG |
| | TGCTTTACGGCACCTCGACCCCAAAAAACTTGATTAGGGTGATGGTTCACGT |
| | AGTGGGCCATCGCCCTGATAGACGGTTTTTCGCCCTTTGACGTTGGAGTCC |
| | ACGTTCTTTAATAGTGGACTCTTGTTCCAAACTGGAACAACACTCAACCCTAT |
| | CTCGGTCTATTCTTTTGATTTATAAGGGATTTTGCCGATTTCGGCCTATTGGT |
| | TAAAAAATGAGCTGATTTAACAAAAATTTAACGCGAATTTTAACAAAATATTAA |
| | CGCTTACAATTTAGGTGGCACTTTTCGGGGAAATGTGCGCGGAACCCCTATT |
| | TGTTTATTTTTCTAAATACATTCAAATATGTATCCGCTCATGAGACAATAACCC |
| | TGATAAATGCTTCAATAATATTGAAAAAGGAAGAGTATGAGTATTCAACATTT |
| | CCGTGTCGCCCTTATTCCCTTTTTTGCGGCATTTTGCCTTCCTGTTTTTGCTC |
| | ACCCAGAAACGCTGGTGAAAGTAAAAGATGCTGAAGATCAGTTGGGTGCAC |
| | GAGTGGGTTACATCGAACTGGATCTCAACAGCGGTAAGATCCTTGAGAGTTT |
| | TCGCCCCGAAGAACGTTTTCCAATGATGAGCACTTTTAAAGTTCTGCTATGT |
| | GGCGCGGTATTATCCCGTATTGACGCCGGGCAAGAGCAACTCGGTCGCCG |
| | CATACACTATTCTCAGAATGACTTGGTTGAGTACTCACCAGTCACAGAAAAG |
| | CATCTTACGGATGGCATGACAGTAAGAGAATTATGCAGTGCTGCCATAACCA |
| | TGAGTGATAACACTGCGGCCAACTTACTTCTGACAACGATCGGAGGACCGA |
| | AGGAGCTAACCGCTTTTTTGCACAACATGGGGGATCATGTAACTCGCCTTGA |
| | TCGTTGGGAACCGGAGCTGAATGAAGCCATACCAAACGACGAGCGTGACAC |
| | CACGATGCCTGTAGCAATGGCAACAACGTTGCGCAAACTATTAACTGGCGAA |
| | CTACTTACTCTAGCTTCCCGGCAACAATTAATAGACTGGATGGAGGCGGATA |
| | AAGTTGCAGGACCACTTCTGCGCTCGGCCCTTCCGGCTGGCTGGTTTATTG |
| | CTGATAAATCTGGAGCCGGTGAGCGTGGGTCTCGCGGTATCATTGCAGCAC |
| | TGGGGCCAGATGGTAAGCCCTCCCGTATCGTAGTTATCTACACGACGGGGA |
| | GTCAGGCAACTATGGATGAACGAAATAGACAGATCGCTGAGATAGGTGCCT |
| | CACTGATTAAGCATTGGTAACTGTCAGACCAAGTTTACTCATATATACTTTAG |
| | ATTGATTTAAAACTTCATTTTTAATTTAAAAGGATCTAGGTGAAGATCCTTTTT |
| | GATAATCTCATGACCAAAATCCCTTAACGTGAGTTTTCGTTCCACTGAGCGT |
| | CAGACCCCGTAGAAAAGATCAAAGGATCTTCTTGAGATCCTTTTTTTCTGCG |
| | CGTAATCTGCTGCTTGCAAACAAAAAAACCACCGCTACCAGCGGTGGTTTGT |
| | TTGCCGGATCAAGAGCTACCAACTCTTTTTCCGAAGGTAACTGGCTTCAGCA |
| | GAGCGCAGATACCAAATACTGTTCTTCTAGTGTAGCCGTAGTTAGGCCACCA |
| | CTTCAAGAACTCTGTAGCACCGCCTACATACCTCGCTCTGCTAATCCTGTTA |
| | CCAGTGGCTGCTGCCAGTGGCGATAAGTCGTGTCTTACCGGGTTGGACTCA |
| | AGACGATAGTTACCGGATAAGGCGCAGCGGTCGGGCTGAACGGGGGGTTC |
| | GTGCACACAGCCCAGCTTGGAGCGAACGACCTACACCGAACTGAGATACCT |
| | ACAGCGTGAGCTATGAGAAAGCGCCACGCTTCCCGAAGGGAGAAAGGCGG |
| | ACAGGTATCCGGTAAGCGGCAGGGTCGGAACAGGAGAGCGCACGAGGGAG |
| | CTTCCAGGGGGAAACGCCTGGTATCTTTATAGTCCTGTCGGGTTTCGCCAC |
| | CTCTGACTTGAGCGTCGATTTTTGTGATGCTCGTCAGGGGGGCGGAGCCTA |
| | TGGAAAAACGCCAGCAACGCGGCCTTTTTACGGTTCCTGGCCTTTTGCTGG |
| | CCTTTTGCTCACATGTTCTTTCCTGCGTTATCCCCTGATTCTGTGGATAACCG |
| | TATTACCGCCTTTGAGTGAGCTGATACCGCTCGCCGCAGCCGAACGACCGA |
| | GCGCAGCGAGTCAGTGAGCGAGGAAGCGGAAGAGCGCCCAATACGCAAAC |
| | CGCCTCTCCCCGCGCGTTGGCCGATTCATTAATGCAGCTGGCACGACAGGT |
| | TTCCCGACTGGAAAGCGGGCAGTGAGCGCAACGCAATTAATGTGAGTTAGC |
| | TCACTCATTAGGCACCCCAGGCTTTACACTTTATGCTTCCGGCTCGTATGTT |
| | GTGTGGAATTGTGAGCGGATAACAATTTCACACAGGAAACAGCTATGACCAT |
| | GATTACGCCAGATTTAATTAAGG |
| SEQ ID NO: 14 | CCTTAATTAGGCTGCGCGCTCGCTCGCTCACTGAGGCCGCCCGGGCAAAG |
| P748 Sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATCGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTCCCATAGTAACGCCAATAGGGACTTTCCATTG |
| positions 548-751 | ACGTCAATGGGTGGAGTATTTACGGTAAACTGCCCACTTGGCAGTACATCAA |
| mGjb2 at | GTGTATCATATGCCAAGTACGCCCCCTATTGACGTCAATGACGGTAAATGGC |
| positions 801-1481 | CCGCCTGGCATTATGCCCAGTACATGACCTTATGGGACTTTCCTACTTGGCA |
| miR-96 target | GTACATCTACGTATTAGTCATCGCTATTACCATGGTGATGCGGTTTTGGCAG |
| sequence at | TACATCAATGGGCGTGGATAGCGGTTTGACTCACGGGGATTTCCAAGTCTC |
| positions 1498-1520 | CACCCCATTGACGTCAATGGGAGTTTGTTTTGGCACCAAAATCAACGGGACT |
| WPRE at | TTCCAAAATGTCGTAACAACTCCGCCCCATTGACGCAAATGGGGGGTAGGC |
| positions 1522-2069 | GTGTACGGTGGGAGGTCTATATAAGCAGAGCTGGTTTAGTGAACCGTCAGA |
| bGH polyA at | TCCTGCAGGGCCGGCCGCGGCCGCGCCGCCATGGATTGGGGCACACTCCA |
| positions 2082-2289 | GAGCATCCTCGGGGGTGTCAACAAACACTCCACCAGCATTGGAAAGATCTG |
| 3′ ITR at positions | GCTCACGGTCCTCTTCATCTTCCGCATCATGATCCTCGTGGTGGCTGCAAAG |
| 2377-2506 | GAGGTGTGGGGAGATGAGCAAGCCGATTTTGTCTGCAACACGCTCCAGCCT |
| | GGCTGCAAGAATGTATGCTACGACCACCACTTCCCCATCTCTCACATCCGGC |
| | TCTGGGCTCTGCAGCTGATCATGGTGTCCACGCCAGCCCTCCTGGTAGCTA |
| | TGCATGTGGCCTACCGGAGACATGAAAAGAAACGGAAGTTCATGAAGGGAG |
| | AGATAAAGAACGAGTTTAAGGACATCGAAGAGATCAAAACCCAGAAGGTCC |
| | GTATCGAAGGGTCCCTGTGGTGGACCTACACCACCAGCATCTTCTTCCGGG |
| | TCATCTTTGAAGCCGTCTTCATGTACGTCTTTTACATCATGTACAATGGCTTC |
| | TTCATGCAACGTCTGGTGAAATGCAACGCTTGGCCCTGCCCCAATACAGTG |
| | GACTGCTTCATTTCCAGGCCCACAGAAAAGACTGTCTTCACCGTGTTTATGA |
| | TTTCTGTGTCTGGAATTTGCATTCTGCTAAATATCACAGAGCTGTGCTATTTG |
| | TTCGTTAGGTATTGCTCAGGAAAGTCCAAAAGACCAGTCTAAACGCGTTAAT |
| | AAGCTTAGCAAAAATGTGCTAGTGCCAAAGGATCCAATCAACCTCTGGATTA |
| | CAAAATTTGTGAAAGATTGACTGGTATTCTTAACTATGTTGCTCCTTTTACGC |
| | TATGTGGATACGCTGCTTTAATGCCTTTGTATCATGCTATTGCTTCCCGTATG |
| | GCTTTCATTTTCTCCTCCTTGTATAAATCCTGGTTGCTGTCTCTTTATGAGGA |
| | GTTGTGGCCCGTTGTCAGGCAACGTGGCGTGGTGTGCACTGTGTTTGCTGA |
| | CGCAACCCCCACTGGTTGGGGCATTGCCACCACCTGTCAGCTCCTTTCCGG |
| | GACTTTCGCTTTCCCCCTCCCTATTGCCACGGCGGAACTCATCGCCGCCTG |
| | CCTTGCCCGCTGCTGGACAGGGGCTCGGCTGTTGGGCACTGACAATTCCGT |
| | GGTGTTGTCGGGGAAATCATCGTCCTTTCCTTGGCTGCTCGCCTGTGTTGC |
| | CACCTGGATTCTGCGCGGGACGTCCTTCTGCTACGTCCCTTCGGCCCTCAA |
| | TCCAGCGGACCTTCCTTCCCGCGGCCTGCTGCCGGCTCTGCGGCCTCTTCC |
| | GCGTCTTCGAGATCTGCCTCGACTGTGCCTTCTAGTTGCCAGCCATCTGTTG |
| | TTTGCCCCTCCCCCGTGCCTTCCTTGACCCTGGAAGGTGCCACTCCCACTG |
| | TCCTTTCCTAATAAAATGAGGAAATTGCATCGCATTGTCTGAGTAGGTGTCAT |
| | TCTATTCTGGGGGGTGGGGTGGGGCAGGACAGCAAGGGGGAGGATTGGGA |
| | AGACAATAGCAGGCATGCTGGGGACTCGAGTTAAGGGCGAATTCCCGATAA |
| | GGATCTTCCTAGAGCATGGCTACGTAGATAAGTAGCATGGCGGGTTAATCAT |
| | TAACTACAAGGAACCCCTAGTGATGGAGTTGGCCACTCCCTCTCTGCGCGC |
| | TCGCTCGCTCACTGAGGCCGGGCGACCAAAGGTCGCCCGACGCCCGGGCT |
| | TTGCCCGGGGGGCCTCAGTGAGCGAGCGAGCGCGCAGCCTTAATTAACCTA |
| | ATTCACTGGCCGTCGTTTTACAACGTCGTGACTGGGAAAACCCTGGCGTTAC |
| | CCAACTTAATCGCCTTGCAGCACATCCCCCTTTCGCCAGCTGGCGTAATAGC |
| | GAAGAGGCCCGCACCGATCGCCCTTCCCAACAGTTGCGCAGCCTGAATGG |
| | CGAATGGGACGCGCCCTGTAGCGGCGCATTAAGCGCGGCGGGTGTGGTGG |
| | TTACGCGCAGCGTGACCGCTACACTTGCCAGCGCCCTAGCGCCCGCTCCTT |
| | TCGCTTTCTTCCCTTCCTTTCTCGCCACGTTCGCCGGCTTTCCCCGTCAAGC |
| | TCTAAATCGGGGGCTCCCTTTAGGGTTCCGATTTAGTGCTTTACGGCACCTC |
| | GACCCCAAAAAACTTGATTAGGGTGATGGTTCACGTAGTGGGCCATCGCCC |
| | TGATAGACGGTTTTTCGCCCTTTGACGTTGGAGTCCACGTTCTTTAATAGTG |
| | GACTCTTGTTCCAAACTGGAACAACACTCAACCCTATCTCGGTCTATTCTTTT |
| | GATTTATAAGGGATTTTGCCGATTTCGGCCTATTGGTTAAAAAATGAGCTGAT |
| | TTAACAAAAATTTAACGCGAATTTTAACAAAATATTAACGCTTACAATTTAGGT |
| | GGCACTTTTCGGGGAAATGTGCGCGGAACCCCTATTTGTTTATTTTTCTAAAT |
| | ACATTCAAATATGTATCCGCTCATGAGACAATAACCCTGATAAATGCTTCAAT |
| | AATATTGAAAAAGGAAGAGTATGAGTATTCAACATTTCCGTGTCGCCCTTATT |
| | CCCTTTTTTGCGGCATTTTGCCTTCCTGTTTTTGCTCACCCAGAAACGCTGGT |
| | GAAAGTAAAAGATGCTGAAGATCAGTTGGGTGCACGAGTGGGTTACATCGA |
| | ACTGGATCTCAACAGCGGTAAGATCCTTGAGAGTTTTCGCCCCGAAGAACGT |
| | TTTCCAATGATGAGCACTTTTAAAGTTCTGCTATGTGGCGCGGTATTATCCC |
| | GTATTGACGCCGGGCAAGAGCAACTCGGTCGCCGCATACACTATTCTCAGA |
| | ATGACTTGGTTGAGTACTCACCAGTCACAGAAAAGCATCTTACGGATGGCAT |
| | GACAGTAAGAGAATTATGCAGTGCTGCCATAACCATGAGTGATAACACTGCG |
| | GCCAACTTACTTCTGACAACGATCGGAGGACCGAAGGAGCTAACCGCTTTTT |
| | TGCACAACATGGGGGATCATGTAACTCGCCTTGATCGTTGGGAACCGGAGC |
| | TGAATGAAGCCATACCAAACGACGAGCGTGACACCACGATGCCTGTAGCAA |
| | TGGCAACAACGTTGCGCAAACTATTAACTGGCGAACTACTTACTCTAGCTTC |
| | CCGGCAACAATTAATAGACTGGATGGAGGCGGATAAAGTTGCAGGACCACT |
| | TCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTA |
| | AGCCCTCCCGTATCGTAGTTATCTACACGACGGGGAGTCAGGCAACTATGG |
| | ATGAACGAAATAGACAGATCGCTGAGATAGGTGCCTCACTGATTAAGCATTG |
| | GTAACTGTCAGACCAAGTTTACTCATATATACTTTAGATTGATTTAAAACTTCA |
| | TTTTTAATTTAAAAGGATCTAGGTGAAGATCCTTTTTGATAATCTCATGACCAA |
| | AATCCCTTAACGTGAGTTTTCGTTCCACTGAGCGTCAGACCCCGTAGAAAAG |
| | ATCAAAGGATCTTCTTGAGATCCTTTTTTTCTGCGCGTAATCTGCTGCTTGCA |
| | AACAAAAAAACCACCGCTACCAGCGGTGGTTTGTTTGCCGGATCAAGAGCTA |
| | CCAACTCTTTTTCCGAAGGTAACTGGCTTCAGCAGAGCGCAGATACCAAATA |
| | CTGTTCTTCTAGTGTAGCCGTAGTTAGGCCACCACTTCAAGAACTCTGTAGC |
| | ACCGCCTACATACCTCGCTCTGCTAATCCTGTTACCAGTGGCTGCTGCCAGT |
| | GGCGATAAGTCGTGTCTTACCGGGTTGGACTCAAGACGATAGTTACCGGAT |
| | AAGGCGCAGCGGTCGGGCTGAACGGGGGGTTCGTGCACACAGCCCAGCTT |
| | GGAGCGAACGACCTACACCGAACTGAGATACCTACAGCGTGAGCTATGAGA |
| | AAGCGCCACGCTTCCCGAAGGGAGAAAGGCGGACAGGTATCCGGTAAGCG |
| | GCAGGGTCGGAACAGGAGAGCGCACGAGGGAGCTTCCAGGGGGAAACGC |
| | CTGGTATCTTTATAGTCCTGTCGGGTTTCGCCACCTCTGACTTGAGCGTCGA |
| | TTTTTGTGATGCTCGTCAGGGGGGCGGAGCCTATGGAAAAACGCCAGCAAC |
| | GCGGCCTTTTTACGGTTCCTGGCCTTTTGCTGGCCTTTTGCTCACATGTTCT |
| | TTCCTGCGTTATCCCCTGATTCTGTGGATAACCGTATTACCGCCTTTGAGTG |
| | AGCTGATACCGCTCGCCGCAGCCGAACGACCGAGCGCAGCGAGTCAGTGA |
| | GCGAGGAAGCGGAAGAGCGCCCAATACGCAAACCGCCTCTCCCCGCGCGT |
| | TGGCCGATTCATTAATGCAGCTGGCACGACAGGTTTCCCGACTGGAAAGCG |
| | GGCAGTGAGCGCAACGCAATTAATGTGAGTTAGCTCACTCATTAGGCACCC |
| | CAGGCTTTACACTTTATGCTTCCGGCTCGTATGTTGTGTGGAATTGTGAGCG |
| | GATAACAATTTCACACAGGAAACAGCTATGACCATGATTACGCCAGATTTAAT |
| | TAAGG |
| SEQ ID NO: 15 | CCTTAATTAGGCTGCGCGCTCGCTCGCTCACTGAGGCCGCCCGGGCAAAG |
| P749 Sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATCGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTCCCATAGTAACGCCAATAGGGACTTTCCATTG |
| positions 548-751 | ACGTCAATGGGTGGAGTATTTACGGTAAACTGCCCACTTGGCAGTACATCAA |
| mGjb2 at | GTGTATCATATGCCAAGTACGCCCCCTATTGACGTCAATGACGGTAAATGGC |
| positions 801-1481 | CCGCCTGGCATTATGCCCAGTACATGACCTTATGGGACTTTCCTACTTGGCA |
| miR-182 target | GTACATCTACGTATTAGTCATCGCTATTACCATGGTGATGCGGTTTTGGCAG |
| sequence at | TACATCAATGGGCGTGGATAGCGGTTTGACTCACGGGGATTTCCAAGTCTC |
| positions 1498-1522 | CACCCCATTGACGTCAATGGGAGTTTGTTTTGGCACCAAAATCAACGGGACT |
| WPRE at | TTCCAAAATGTCGTAACAACTCCGCCCCATTGACGCAAATGGGGGGTAGGC |
| positions 1524-2071 | GTGTACGGTGGGAGGTCTATATAAGCAGAGCTGGTTTAGTGAACCGTCAGA |
| bGH polyA at | TCCTGCAGGGCCGGCCGCGGCCGCGCCGCCATGGATTGGGGCACACTCCA |
| positions 2084-2291 | GAGCATCCTCGGGGGTGTCAACAAACACTCCACCAGCATTGGAAAGATCTG |
| 3′ ITR at positions | GCTCACGGTCCTCTTCATCTTCCGCATCATGATCCTCGTGGTGGCTGCAAAG |
| 2379-2508 | GAGGTGTGGGGAGATGAGCAAGCCGATTTTGTCTGCAACACGCTCCAGCCT |
| | GGCTGCAAGAATGTATGCTACGACCACCACTTCCCCATCTCTCACATCCGGC |
| | TCTGGGCTCTGCAGCTGATCATGGTGTCCACGCCAGCCCTCCTGGTAGCTA |
| | TGCATGTGGCCTACCGGAGACATGAAAAGAAACGGAAGTTCATGAAGGGAG |
| | AGATAAAGAACGAGTTTAAGGACATCGAAGAGATCAAAACCCAGAAGGTCC |
| | GTATCGAAGGGTCCCTGTGGTGGACCTACACCACCAGCATCTTCTTCCGGG |
| | TCATCTTTGAAGCCGTCTTCATGTACGTCTTTTACATCATGTACAATGGCTTC |
| | TTCATGCAACGTCTGGTGAAATGCAACGCTTGGCCCTGCCCCAATACAGTG |
| | GACTGCTTCATTTCCAGGCCCACAGAAAAGACTGTCTTCACCGTGTTTATGA |
| | TTTCTGTGTCTGGAATTTGCATTCTGCTAAATATCACAGAGCTGTGCTATTTG |
| | TTCGTTAGGTATTGCTCAGGAAAGTCCAAAAGACCAGTCTAAACGCGTTAAT |
| | AAGCTTCGGTGTGAGTTCTACCATTGCCAAAGGATCCAATCAACCTCTGGAT |
| | TACAAAATTTGTGAAAGATTGACTGGTATTCTTAACTATGTTGCTCCTTTTAC |
| | GCTATGTGGATACGCTGCTTTAATGCCTTTGTATCATGCTATTGOTTCCCGTA |
| | TGGCTTTCATTTTCTCCTCCTTGTATAAATCCTGGTTGCTGTCTCTTTATGAG |
| | GAGTTGTGGCCCGTTGTCAGGCAACGTGGCGTGGTGTGCACTGTGTTTGCT |
| | GACGCAACCCCCACTGGTTGGGGCATTGCCACCACCTGTCAGCTCCTTTCC |
| | GGGACTTTCGCTTTCCCCCTCCCTATTGCCACGGCGGAACTCATCGCCGCC |
| | TGCCTTGCCCGCTGCTGGACAGGGGCTCGGCTGTTGGGCACTGACAATTCC |
| | GTGGTGTTGTCGGGGAAATCATCGTCCTTTCCTTGGCTGCTCGCCTGTGTTG |
| | CCACCTGGATTCTGCGCGGGACGTCCTTCTGCTACGTCCCTTCGGCCCTCA |
| | ATCCAGCGGACCTTCCTTCCCGCGGCCTGCTGCCGGCTCTGCGGCCTCTTC |
| | CGCGTCTTCGAGATCTGCCTCGACTGTGCCTTCTAGTTGCCAGCCATCTGTT |
| | GTTTGCCCCTCCCCCGTGCCTTCCTTGACCCTGGAAGGTGCCACTCCCACT |
| | GTCCTTTCCTAATAAAATGAGGAAATTGCATCGCATTGTCTGAGTAGGTGTC |
| | ATTCTATTCTGGGGGGGGGGTGGGGCAGGACAGCAAGGGGGAGGATTGG |
| | GAAGACAATAGCAGGCATGCTGGGGACTCGAGTTAAGGGCGAATTCCCGAT |
| | AAGGATCTTCCTAGAGCATGGCTACGTAGATAAGTAGCATGGGGGTTAATC |
| | ATTAACTACAAGGAACCCCTAGTGATGGAGTTGGCCACTCCCTCTCTGCGC |
| | GCTCGCTCGCTCACTGAGGCCGGGCGACCAAAGGTCGCCCGACGCCCGGG |
| | CTTTGCCCGGGGGGCCTCAGTGAGCGAGCGAGCGCGCAGCCTTAATTAAC |
| | CTAATTCACTGGCCGTCGTTTTACAACGTCGTGACTGGGAAAACCCTGGCGT |
| | TACCCAACTTAATCGCCTTGCAGCACATCCCCCTTTCGCCAGCTGGCGTAAT |
| | AGCGAAGAGGCCCGCACCGATCGCCCTTCCCAACAGTTGCGCAGCCTGAAT |
| | GGCGAATGGGACGCGCCCTGTAGCGGCGCATTAAGCGCGGCGGGTGTGGT |
| | GGTTACGCGCAGCGTGACCGCTACACTTGCCAGCGCCCTAGCGCCCGCTC |
| | CTTTCGCTTTCTTCCCTTCCTTTCTCGCCACGTTCGCCGGCTTTCCCCGTCA |
| | AGCTCTAAATCGGGGGCTCCCTTTAGGGTTCCGATTTAGTGCTTTACGGCAC |
| | CTCGACCCCAAAAAACTTGATTAGGGTGATGGTTCACGTAGTGGGCCATCG |
| | CCCTGATAGACGGTTTTTCGCCCTTTGACGTTGGAGTCCACGTTCTTTAATA |
| | GTGGACTCTTGTTCCAAACTGGAACAACACTCAACCCTATCTCGGTCTATTC |
| | TTTTGATTTATAAGGGATTTTGCCGATTTCGGCCTATTGGTTAAAAAATGAGC |
| | TGATTTAACAAAAATTTAACGCGAATTTTAACAAAATATTAACGCTTACAATTT |
| | AGGTGGCACTTTTCGGGGAAATGTGCGCGGAACCCCTATTTGTTTATTTTTC |
| | TAAATACATTCAAATATGTATCCGCTCATGAGACAATAACCCTGATAAATGCT |
| | TCAATAATATTGAAAAAGGAAGAGTATGAGTATTCAACATTTCCGTGTCGCCC |
| | TTATTCCCTTTTTTGCGGCATTTTGCCTTCCTGTTTTTGCTCACCCAGAAACG |
| | CTGGTGAAAGTAAAAGATGCTGAAGATCAGTTGGGTGCACGAGTGGGTTAC |
| | ATCGAACTGGATCTCAACAGCGGTAAGATCCTTGAGAGTTTTCGCCCCGAAG |
| | AACGTTTTCCAATGATGAGCACTTTTAAAGTTCTGCTATGTGGCGCGGTATTA |
| | TCCCGTATTGACGCCGGGCAAGAGCAACTCGGTCGCCGCATACACTATTCT |
| | CAGAATGACTTGGTTGAGTACTCACCAGTCACAGAAAAGCATCTTACGGATG |
| | GCATGACAGTAAGAGAATTATGCAGTGCTGCCATAACCATGAGTGATAACAC |
| | TGCGGCCAACTTACTTCTGACAACGATCGGAGGACCGAAGGAGCTAACCGC |
| | TTTTTTGCACAACATGGGGGATCATGTAACTCGCCTTGATCGTTGGGAACCG |
| | GAGCTGAATGAAGCCATACCAAACGACGAGCGTGACACCACGATGCCTGTA |
| | GCAATGGCAACAACGTTGCGCAAACTATTAACTGGCGAACTACTTACTCTAG |
| | CTTCCCGGCAACAATTAATAGACTGGATGGAGGCGGATAAAGTTGCAGGAC |
| | CACTTCTGCGCTCGGCCCTTCCGGCTGGCTGGTTTATTGCTGATAAATCTGG |
| | AGCCGGTGAGCGTGGGTCTCGCGGTATCATTGCAGCACTGGGGCCAGATG |
| | GTAAGCCCTCCCGTATCGTAGTTATCTACACGACGGGGAGTCAGGCAACTA |
| | TGGATGAACGAAATAGACAGATCGCTGAGATAGGTGCCTCACTGATTAAGCA |
| | TTGGTAACTGTCAGACCAAGTTTACTCATATATACTTTAGATTGATTTAAAACT |
| | TCATTTTTAATTTAAAAGGATCTAGGTGAAGATCCTTTTTGATAATCTCATGAC |
| | CAAAATCCCTTAACGTGAGTTTTCGTTCCACTGAGCGTCAGACCCCGTAGAA |
| | AAGATCAAAGGATCTTCTTGAGATCCTTTTTTTCTGCGCGTAATCTGCTGCTT |
| | GCAAACAAAAAAACCACCGCTACCAGCGGTGGTTTGTTTGCCGGATCAAGA |
| | GCTACCAACTCTTTTTCCGAAGGTAACTGGCTTCAGCAGAGCGCAGATACCA |
| | AATACTGTTCTTCTAGTGTAGCCGTAGTTAGGCCACCACTTCAAGAACTCTG |
| | TAGCACCGCCTACATACCTCGCTCTGCTAATCCTGTTACCAGTGGCTGCTGC |
| | CAGTGGCGATAAGTCGTGTCTTACCGGGTTGGACTCAAGACGATAGTTACC |
| | GGATAAGGCGCAGCGGTCGGGCTGAACGGGGGGTTCGTGCACACAGCCCA |
| | GCTTGGAGCGAACGACCTACACCGAACTGAGATACCTACAGCGTGAGCTAT |
| | GAGAAAGCGCCACGCTTCCCGAAGGGAGAAAGGCGGACAGGTATCCGGTA |
| | AGCGGCAGGGTCGGAACAGGAGAGCGCACGAGGGAGCTTCCAGGGGGAA |
| | ACGCCTGGTATCTTTATAGTCCTGTCGGGTTTCGCCACCTCTGACTTGAGCG |
| | TCGATTTTTGTGATGCTCGTCAGGGGGGCGGAGCCTATGGAAAAACGCCAG |
| | CAACGCGGCCTTTTTACGGTTCCTGGCCTTTTGCTGGCCTTTTGCTCACATG |
| | TTCTTTCCTGCGTTATCCCCTGATTCTGTGGATAACCGTATTACCGCCTTTGA |
| | GTGAGCTGATACCGCTCGCCGCAGCCGAACGACCGAGCGCAGCGAGTCAG |
| | TGAGCGAGGAAGCGGAAGAGCGCCCAATACGCAAACCGCCTCTCCCCGCG |
| | CGTTGGCCGATTCATTAATGCAGCTGGCACGACAGGTTTCCCGACTGGAAA |
| | GCGGGCAGTGAGCGCAACGCAATTAATGTGAGTTAGCTCACTCATTAGGCA |
| | CCCCAGGCTTTACACTTTATGCTTCCGGCTCGTATGTTGTGTGGAATTGTGA |
| | GCGGATAACAATTTCACACAGGAAACAGCTATGACCATGATTACGCCAGATT |
| | TAATTAAGG |
| SEQ ID NO: 16 | CCTTAATTAGGCTGCGCGCTCGCTCGCTCACTGAGGCCGCCCGGGCAAAG |
| P751 Sequence | CCCGGGCGTCGGGCGACCTTTGGTCGCCCGGCCTCAGTGAGCGAGCGAGC |
| 5′ ITR at positions | GCGCAGAGAGGGAGTGGCCAACTCCATCACTAGGGGTTCCTTGTAGTTAAT |
| 12-141 | GATTAACCCGCCATGCTACTTATCTACGTAGCCATGCTCTAGGAAGATCGGA |
| CMV Enhancer at | ATTCGCCCTTAAGCTAGCGGCGCGCCACCGGTGCGATCGCCGTTACATAAC |
| positions 244-547 | TTACGGTAAATGGCCCGCCTGGCTGACCGCCCAACGACCCCCGCCCATTGA |
| CMV promoter at | CGTCAATAATGACGTATGTTCCCATAGTAACGCCAATAGGGACTTTCCATTG |
| positions 548-751 | ACGTCAATGGGTGGAGTATTTACGGTAAACTGCCCACTTGGCAGTACATCAA |
| mGjb2 at | GTGTATCATATGCCAAGTACGCCCCCTATTGACGTCAATGACGGTAAATGGC |
| positions 801-1481 | CCGCCTGGCATTATGCCCAGTACATGACCTTATGGGACTTTCCTACTTGGCA |
| miR-183 target | GTACATCTACGTATTAGTCATCGCTATTACCATGGTGATGCGGTTTTGGCAG |
| sequence at | TACATCAATGGGCGTGGATAGCGGTTTGACTCACGGGGATTTCCAAGTCTC |
| positions 1498-1519 | CACCCCATTGACGTCAATGGGAGTTTGTTTTGGCACCAAAATCAACGGGACT |
| WPRE at | TTCCAAAATGTCGTAACAACTCCGCCCCATTGACGCAAATGGGGGGTAGGC |
| positions 1521-2068 | GTGTACGGTGGGAGGTCTATATAAGCAGAGCTGGTTTAGTGAACCGTCAGA |
| bGH polyA at | TCCTGCAGGGCCGGCCGCGGCCGCGCCGCCATGGATTGGGGCACACTCCA |
| positions 2081-2288 | GAGCATCCTCGGGGGTGTCAACAAACACTCCACCAGCATTGGAAAGATCTG |
| 3′ ITR at positions | GCTCACGGTCCTCTTCATCTTCCGCATCATGATCCTCGTGGTGGCTGCAAAG |
| 2376-2505 | GAGGTGTGGGGAGATGAGCAAGCCGATTTTGTCTGCAACACGCTCCAGCCT |
| | GGCTGCAAGAATGTATGCTACGACCACCACTTCCCCATCTCTCACATCCGGC |
| | TCTGGGCTCTGCAGCTGATCATGGTGTCCACGCCAGCCCTCCTGGTAGCTA |
| | TGCATGTGGCCTACCGGAGACATGAAAAGAAACGGAAGTTCATGAAGGGAG |
| | AGATAAAGAACGAGTTTAAGGACATCGAAGAGATCAAAACCCAGAAGGTCC |
| | GTATCGAAGGGTCCCTGTGGTGGACCTACACCACCAGCATCTTCTTCCGGG |
| | TCATCTTTGAAGCCGTCTTCATGTACGTCTTTTACATCATGTACAATGGCTTC |
| | TTCATGCAACGTCTGGTGAAATGCAACGCTTGGCCCTGCCCCAATACAGTG |
| | GACTGCTTCATTTCCAGGCCCACAGAAAAGACTGTCTTCACCGTGTTTATGA |
| | TTTCTGTGTCTGGAATTTGCATTCTGCTAAATATCACAGAGCTGTGCTATTTG |
| | TTCGTTAGGTATTGCTCAGGAAAGTCCAAAAGACCAGTCTAAACGCGTTAAT |
| | AAGCTTAGTGAATTCTACCAGTGCCATAGGATCCAATCAACCTCTGGATTAC |
| | AAAATTTGTGAAAGATTGACTGGTATTCTTAACTATGTTGCTCCTTTTACGCT |
| | ATGTGGATACGCTGCTTTAATGCCTTTGTATCATGCTATTGOTTCCCGTATGG |
| | CTTTCATTTTCTCCTCCTTGTATAAATCCTGGTTGCTGTCTCTTTATGAGGAG |
| | TTGTGGCCCGTTGTCAGGCAACGTGGCGTGGTGTGCACTGTGTTTGCTGAC |
| | GCAACCCCCACTGGTTGGGGCATTGCCACCACCTGTCAGCTCCTTTCCGGG |
| | ACTTTCGCTTTCCCCCTCCCTATTGCCACGGCGGAACTCATCGCCGCCTGC |
| | CTTGCCCGCTGCTGGACAGGGGCTCGGCTGTTGGGCACTGACAATTCCGTG |
| | GTGTTGTCGGGGAAATCATCGTCCTTTCCTTGGCTGCTCGCCTGTGTTGCCA |
| | CCTGGATTCTGCGCGGGACGTCCTTCTGCTACGTCCCTTCGGCCCTCAATC |
| | CAGCGGACCTTCCTTCCCGCGGCCTGCTGCCGGCTCTGCGGCCTCTTCCG |
| | CGTCTTCGAGATCTGCCTCGACTGTGCCTTCTAGTTGCCAGCCATCTGTTGT |
| | TTGCCCCTCCCCCGTGCCTTCCTTGACCCTGGAAGGTGCCACTCCCACTGT |
| | CCTTTCCTAATAAAATGAGGAAATTGCATCGCATTGTCTGAGTAGGTGTCATT |
| | CTATTCTGGGGGGTGGGGTGGGGCAGGACAGCAAGGGGGAGGATTGGGAA |
| | GACAATAGCAGGCATGCTGGGGACTCGAGTTAAGGGCGAATTCCCGATAAG |
| | GATCTTCCTAGAGCATGGCTACGTAGATAAGTAGCATGGGGGTTAATCATT |
| | AACTACAAGGAACCCCTAGTGATGGAGTTGGCCACTCCCTCTCTGCGCGCT |
| | CGCTCGCTCACTGAGGCCGGGCGACCAAAGGTCGCCCGACGCCCGGGCTT |
| | TGCCCGGGCGGCCTCAGTGAGCGAGCGAGCGCGCAGCCTTAATTAACCTA |
| | ATTCACTGGCCGTCGTTTTACAACGTCGTGACTGGGAAAACCCTGGCGTTAC |
| | CCAACTTAATCGCCTTGCAGCACATCCCCCTTTCGCCAGCTGGCGTAATAGC |
| | GAAGAGGCCCGCACCGATCGCCCTTCCCAACAGTTGCGCAGCCTGAATGG |
| | CGAATGGGACGCGCCCTGTAGCGGCGCATTAAGCGCGGCGGGTGTGGTGG |
| | TTACGCGCAGCGTGACCGCTACACTTGCCAGCGCCCTAGCGCCCGCTCCTT |
| | TCGCTTTCTTCCCTTCCTTTCTCGCCACGTTCGCCGGCTTTCCCCGTCAAGC |
| | TCTAAATCGGGGGCTCCCTTTAGGGTTCCGATTTAGTGCTTTACGGCACCTC |
| | GACCCCAAAAAACTTGATTAGGGTGATGGTTCACGTAGTGGGCCATCGCCC |
| | TGATAGACGGTTTTTCGCCCTTTGACGTTGGAGTCCACGTTCTTTAATAGTG |
| | GACTCTTGTTCCAAACTGGAACAACACTCAACCCTATCTCGGTCTATTCTTTT |
| | GATTTATAAGGGATTTTGCCGATTTCGGCCTATTGGTTAAAAAATGAGCTGAT |
| | TTAACAAAAATTTAACGCGAATTTTAACAAAATATTAACGCTTACAATTTAGGT |
| | GGCACTTTTCGGGGAAATGTGCGCGGAACCCCTATTTGTTTATTTTTCTAAAT |
| | ACATTCAAATATGTATCCGCTCATGAGACAATAACCCTGATAAATGCTTCAAT |
| | AATATTGAAAAAGGAAGAGTATGAGTATTCAACATTTCCGTGTCGCCCTTATT |
| | CCCTTTTTTGCGGCATTTTGCCTTCCTGTTTTTGCTCACCCAGAAACGCTGGT |
| | GAAAGTAAAAGATGCTGAAGATCAGTTGGGTGCACGAGTGGGTTACATCGA |
| | ACTGGATCTCAACAGCGGTAAGATCCTTGAGAGTTTTCGCCCCGAAGAACGT |
| | TTTCCAATGATGAGCACTTTTAAAGTTCTGCTATGTGGCGCGGTATTATCCC |
| | GTATTGACGCCGGGCAAGAGCAACTCGGTCGCCGCATACACTATTCTCAGA |
| | ATGACTTGGTTGAGTACTCACCAGTCACAGAAAAGCATCTTACGGATGGCAT |
| | GACAGTAAGAGAATTATGCAGTGCTGCCATAACCATGAGTGATAACACTGCG |
| | GCCAACTTACTTCTGACAACGATCGGAGGACCGAAGGAGCTAACCGCTTTTT |
| | TGCACAACATGGGGGATCATGTAACTCGCCTTGATCGTTGGGAACCGGAGC |
| | TGAATGAAGCCATACCAAACGACGAGCGTGACACCACGATGCCTGTAGCAA |
| | TGGCAACAACGTTGCGCAAACTATTAACTGGCGAACTACTTACTCTAGCTTC |
| | CCGGCAACAATTAATAGACTGGATGGAGGCGGATAAAGTTGCAGGACCACT |
| | TCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTA |
| | AGCCCTCCCGTATCGTAGTTATCTACACGACGGGGAGTCAGGCAACTATGG |
| | ATGAACGAAATAGACAGATCGCTGAGATAGGTGCCTCACTGATTAAGCATTG |
| | GTAACTGTCAGACCAAGTTTACTCATATATACTTTAGATTGATTTAAAACTTCA |
| | TTTTTAATTTAAAAGGATCTAGGTGAAGATCCTTTTTGATAATCTCATGACCAA |
| | AATCCCTTAACGTGAGTTTTCGTTCCACTGAGCGTCAGACCCCGTAGAAAAG |
| | ATCAAAGGATCTTCTTGAGATCCTTTTTTTCTGCGCGTAATCTGCTGCTTGCA |
| | AACAAAAAAACCACCGCTACCAGCGGTGGTTTGTTTGCCGGATCAAGAGCTA |
| | CCAACTCTTTTTCCGAAGGTAACTGGCTTCAGCAGAGCGCAGATACCAAATA |
| | CTGTTCTTCTAGTGTAGCCGTAGTTAGGCCACCACTTCAAGAACTCTGTAGC |
| | ACCGCCTACATACCTCGCTCTGCTAATCCTGTTACCAGTGGCTGCTGCCAGT |
| | GGCGATAAGTCGTGTCTTACCGGGTTGGACTCAAGACGATAGTTACCGGAT |
| | AAGGCGCAGCGGTCGGGCTGAACGGGGGGTTCGTGCACACAGCCCAGCTT |
| | GGAGCGAACGACCTACACCGAACTGAGATACCTACAGCGTGAGCTATGAGA |
| | AAGCGCCACGCTTCCCGAAGGGAGAAAGGCGGACAGGTATCCGGTAAGCG |
| | GCAGGGTCGGAACAGGAGAGCGCACGAGGGAGCTTCCAGGGGGAAACGC |
| | CTGGTATCTTTATAGTCCTGTCGGGTTTCGCCACCTCTGACTTGAGCGTCGA |
| | TTTTTGTGATGCTCGTCAGGGGGGCGGAGCCTATGGAAAAACGCCAGCAAC |
| | GCGGCCTTTTTACGGTTCCTGGCCTTTTGCTGGCCTTTTGCTCACATGTTCT |
| | TTCCTGCGTTATCCCCTGATTCTGTGGATAACCGTATTACCGCCTTTGAGTG |
| | AGCTGATACCGCTCGCCGCAGCCGAACGACCGAGCGCAGCGAGTCAGTGA |
| | GCGAGGAAGCGGAAGAGCGCCCAATACGCAAACCGCCTCTCCCCGCGCGT |
| | TGGCCGATTCATTAATGCAGCTGGCACGACAGGTTTCCCGACTGGAAAGCG |
| | GGCAGTGAGCGCAACGCAATTAATGTGAGTTAGCTCACTCATTAGGCACCC |
| | CAGGCTTTACACTTTATGCTTCCGGCTCGTATGTTGTGTGGAATTGTGAGCG |
| | GATAACAATTTCACACAGGAAACAGCTATGACCATGATTACGCCAGATTTAAT |
| | TAAGG |
| SEQ ID NO: 17 | CTGCGCGCTCGCTCGCTCACTGAGGCCGCCCGGGCAAAGCCCGGGCGTCG |
| P1137 Sequence | GGCGACCTTTGGTCGCCCGGCCTCAGTGAGCGAGCGAGCGCGCAGAGAGG |
| 5′ ITR at positions | GAGTGGCCAACTCCATCACTAGGGGTTCCTTGTAGTTAATGATTAACCCGCC |
| 1-130 | ATGCTACTTATCTACGTAGCCATGCTCTAGGAAGATCGGAATTCGCCCTTAA |
| CMV enhancer at | GCTAGCGGCGCGCCACCGGTGCGATCGCCGTTACATAACTTACGGTAAATG |
| positions 233-536 | GCCCGCCTGGCTGACCGCCCAACGACCCCCGCCCATTGACGTCAATAATGA |
| CMV promoter at | CGTATGTTOCCATAGTAACGCCAATAGGGACTTTCCATTGACGTCAATGGGT |
| positions 537-740 | GGAGTATTTACGGTAAACTGCCCACTTGGCAGTACATCAAGTGTATCATATG |
| Chimeric intron at | CCAAGTACGCCCCCTATTGACGTCAATGACGGTAAATGGCCCGCCTGGCAT |
| positions 793-925 | TATGCCCAGTACATGACCTTATGGGACTTTCCTACTTGGCAGTACATCTACG |
| H2B at positions | TATTAGTCATCGCTATTACCATGGTGATGCGGTTTTGGCAGTACATCAATGG |
| 948-1325 | GCGTGGATAGCGGTTTGACTCACGGGGATTTCCAAGTCTCCACCCCATTGA |
| EGFP at positions | CGTCAATGGGAGTTTGTTTTGGCACCAAAATCAACGGGACTTTCCAAAATGT |
| 1344-2063 | CGTAACAACTCCGCCCCATTGACGCAAATGGGGGGTAGGCGTGTACGGTGG |
| miR-96 target | GAGGTCTATATAAGCAGAGCTGGTTTAGTGAACCGTCAGATCCTGCAGAAGT |
| sequence at | TGGTCGTGAGGCACTGGGCAGGTAAGTATCAAGGTTACAAGACAGGTTTAA |
| positions 2071-2093 | GGAGACCAATAGAAACTGGGCTTGTCGAGACAGAGAAGACTCTTGCGTTTC |
| bGH polyA signal | TGATAGGCACCTATTGGTCTTACTGACATCCACTTTGCCTTTCTCTCCACAG |
| at positions 2101-2308 | GTGTCCAGGCGGCCGCGCCACCATGCCAGAGCCAGCGAAGTCTGCTCCCG |
| 3′ ITR at positions | CCCCGAAAAAGGGCTCCAAGAAGGCGGTGACTAAGGCGCAGAAGAAAGGC |
| 2396-2525 | GGCAAGAAGCGCAAGCGCAGCCGCAAGGAGAGCTATTCCATCTATGTGTAC |
| | AAGGTTCTGAAGCAGGTCCACCCTGACACCGGCATTTCGTCCAAGGCCATG |
| | GGCATCATGAATTCGTTTGTGAACGACATTTTCGAGCGCATCGCAGGTGAG |
| | GCTTCCCGCCTGGCGCATTACAACAAGCGCTCGACCATCACCTCCAGGGAG |
| | ATCCAGACGGCCGTGCGCCTGCTGCTGCCTGGGGAGTTGGCCAAGCACGC |
| | CGTGTCCGAGGGTACTAAGGCCATCACCAAGTACACCAGCGCTAAGGATCC |
| | ACCGGTCGCCACCATGGTGAGCAAGGGCGAGGAGCTGTTCACCGGGGTGG |
| | TGCCCATCCTGGTCGAGCTGGACGGCGACGTAAACGGCCACAAGTTCAGC |
| | GTGTCCGGCGAGGGCGAGGGCGATGCCACCTACGGCAAGCTGACCCTGAA |
| | GTTCATCTGCACCACCGGCAAGCTGCCCGTGCCCTGGCCCACCCTCGTGAC |
| | CACCCTGACCTACGGCGTGCAGTGCTTCAGCCGCTACCCCGACCACATGAA |
| | GCAGCACGACTTCTTCAAGTCCGCCATGCCCGAAGGCTACGTCCAGGAGCG |
| | CACCATCTTCTTCAAGGACGACGGCAACTACAAGACCCGCGCCGAGGTGAA |
| | GTTCGAGGGCGACACCCTGGTGAACCGCATCGAGCTGAAGGGCATCGACTT |
| | CAAGGAGGACGGCAACATCCTGGGGCACAAGCTGGAGTACAACTACAACAG |
| | CCACAACGTCTATATCATGGCCGACAAGCAGAAGAACGGCATCAAGGTGAA |
| | CTTCAAGATCCGCCACAACATCGAGGACGGCAGCGTGCAGCTCGCCGACCA |
| | CTACCAGCAGAACACCCCCATCGGCGACGGCCCCGTGCTGCTGCCCGACA |
| | ACCACTACCTGAGCACCCAGTCCGCCCTGAGCAAAGACCCCAACGAGAAGC |
| | GCGATCACATGGTCCTGCTGGAGTTCGTGACCGCCGCCGGGATCACTCTCG |
| | GCATGGACGAGCTGTACAAGTAATAAGCTTAGCAAAAATGTGCTAGTGCCAA |
| | AGCCTCGACTGTGCCTTCTAGTTGCCAGCCATCTGTTGTTTGCCCCTCCCCC |
| | GTGCCTTCCTTGACCCTGGAAGGTGCCACTCCCACTGTCCTTTCCTAATAAA |
| | ATGAGGAAATTGCATCGCATTGTCTGAGTAGGTGTCATTCTATTCTGGGGGG |
| | TGGGGGGGGCAGGACAGCAAGGGGGAGGATTGGGAAGACAATAGCAGGC |
| | ATGCTGGGGACTCGAGTTAAGGGCGAATTCCCGATAAGGATOTTCCTAGAG |
| | CATGGCTACGTAGATAAGTAGCATGGCGGGTTAATCATTAACTACAAGGAAC |
| | CCCTAGTGATGGAGTTGGCCACTCCCTCTCTGCGCGCTCGCTCGCTCACTG |
| | AGGCCGGGCGACCAAAGGTCGCCCGACGCCCGGGCTTTGCCCGGGCGGC |
| | CTCAGTGAGCGAGCGAGCGCGCAGCCTTAATTAACCTAATTCACTGGCCGT |
| | CGTTTTACAACGTCGTGACTGGGAAAACCCTGGCGTTACCCAACTTAATCGC |
| | CTTGCAGCACATCCCCCTTTCGCCAGCTGGCGTAATAGCGAAGAGGCCCGC |
| | ACCGATCGCCCTTCCCAACAGTTGCGCAGCCTGAATGGCGAATGGGACGCG |
| | CCCTGTAGCGGCGCATTAAGCGCGGGGGGTGTGGTGGTTACGCGCAGCGT |
| | GACCGCTACACTTGCCAGCGCCCTAGCGCCCGCTCCTTTCGCTTTCTTCCC |
| | TTCCTTTCTCGCCACGTTCGCCGGCTTTCCCCGTCAAGCTCTAAATCGGGG |
| | GCTCCCTTTAGGGTTCCGATTTAGTGCTTTACGGCACCTCGACCCCAAAAAA |
| | CTTGATTAGGGTGATGGTTCACGTAGTGGGCCATCGCCCTGATAGACGGTT |
| | TTTCGCCCTTTGACGTTGGAGTCCACGTTCTTTAATAGTGGACTCTTGTTCCA |
| | AACTGGAACAACACTCAACCCTATCTCGGTCTATTCTTTTGATTTATAAGGGA |
| | TTTTGCCGATTTCGGCCTATTGGTTAAAAAATGAGCTGATTTAACAAAAATTT |
| | AACGCGAATTTTAACAAAATATTAACGCTTACAATTTAGGTGGCACTTTTCGG |
| | GGAAATGTGCGCGGAACCCCTATTTGTTTATTTTTCTAAATACATTCAAATAT |
| | GTATCCGCTCATGAGACAATAACCCTGATAAATGCTTCAATAATATTGAAAAA |
| | GGAAGAGTATGAGCCATATTCAACGGGAAACGTCGAGGCCGCGATTAAATT |
| | CCAACATGGATGCTGATTTATATGGGTATAAATGGGCTCGCGATAATGTCGG |
| | GCAATCAGGTGCGACAATCTATCGCTTGTATGGGAAGCCCGATGCGCCAGA |
| | GTTGTTTCTGAAACATGGCAAAGGTAGCGTTGCCAATGATGTTACAGATGAG |
| | ATGGTCAGACTAAACTGGCTGACGGAATTTATGCCTCTTCCGACCATCAAGC |
| | ATTTTATCCGTACTCCTGATGATGCATGGTTACTCACCACTGCGATCCCCGG |
| | AAAAACAGCATTCCAGGTATTAGAAGAATATCCTGATTCAGGTGAAAATATTG |
| | TTGATGCGCTGGCAGTGTTCCTGCGCCGGTTGCATTCGATTCCTGTTTGTAA |
| | TTGTCCTTTTAACAGCGATCGCGTATTTCGTCTTGCTCAGGCGCAATCACGA |
| | ATGAATAACGGTTTGGTTGATGCGAGTGATTTTGATGACGAGCGTAATGGCT |
| | GGCCTGTTGAACAAGTCTGGAAAGAAATGCATAAACTTTTGCCATTCTCACC |
| | GGATTCAGTCGTCACTCATGGTGATTTCTCACTTGATAACCTTATTTTTGACG |
| | AGGGGAAATTAATAGGTTGTATTGATGTTGGACGAGTCGGAATCGCAGACC |
| | GATACCAGGATCTTGCCATCCTATGGAACTGCCTCGGTGAGTTTTCTCCTTC |
| | ATTACAGAAACGGCTTTTTCAAAAATATGGTATTGATAATCCTGATATGAATA |
| | AATTGCAGTTTCATTTGATGCTCGATGAGTTTTTCTAACTGTCAGACCAAGTT |
| | TACTCATATATACTTTAGATTGATTTAAAACTTCATTTTTAATTTAAAAGGATCT |
| | AGGTGAAGATCCTTTTTGATAATCTCATGACCAAAATCCCTTAACGTGAGTTT |
| | TCGTTCCACTGAGCGTCAGACCCCGTAGAAAAGATCAAAGGATCTTCTTGAG |
| | ATCCTTTTTTTCTGCGCGTAATCTGCTGCTTGCAAACAAAAAAACCACCGCTA |
| | CCAGCGGTGGTTTGTTTGCCGGATCAAGAGCTACCAACTCTTTTTCCGAAGG |
| | TAACTGGCTTCAGCAGAGCGCAGATACCAAATACTGTTCTTCTAGTGTAGCC |
| | GTAGTTAGGCCACCACTTCAAGAACTCTGTAGCACCGCCTACATACCTCGCT |
| | CTGCTAATCCTGTTACCAGTGGCTGCTGCCAGTGGCGATAAGTCGTGTCTTA |
| | CCGGGTTGGACTCAAGACGATAGTTACCGGATAAGGCGCAGCGGTCGGGC |
| | TGAACGGGGGGTTCGTGCACACAGCCCAGCTTGGAGCGAACGACCTACAC |
| | CGAACTGAGATACCTACAGCGTGAGCTATGAGAAAGCGCCACGCTTCCCGA |
| | AGGGAGAAAGGCGGACAGGTATCCGGTAAGCGGCAGGGTCGGAACAGGAG |
| | AGCGCACGAGGGAGCTTCCAGGGGGAAACGCCTGGTATCTTTATAGTCCTG |
| | TCGGGTTTCGCCACCTCTGACTTGAGCGTCGATTTTTGTGATGCTCGTCAGG |
| | GGGGCGGAGCCTATGGAAAAACGCCAGCAACGCGGCCTTTTTACGGTTCCT |
| | GGCCTTTTGCTGGCCTTTTGCTCACATGTTCTTTCCTGCGTTATCCCCTGATT |
| | CTGTGGATAACCGTATTACCGCCTTTGAGTGAGCTGATACCGCTCGCCGCA |
| | GCCGAACGACCGAGCGCAGCGAGTCAGTGAGCGAGGAAGCGGAAGAGCG |
| | CCCAATACGCAAACCGCCTCTCCCCGCGCGTTGGCCGATTCATTAATGCAG |
| | CTGGCACGACAGGTTTCCCGACTGGAAAGCGGGCAGTGAGCGCAACGCAA |
| | TTAATGTGAGTTAGCTCACTCATTAGGCACCCCAGGCTTTACACTTTATGCTT |
| | CCGGCTCGTATGTTGTGTGGAATTGTGAGCGGATAACAATTTCACACAGGAA |
| | ACAGCTATGACCATGATTACGCCAGATTTAATTAAGGCCTTAATTAGG |
| SEQ ID NO: 18 | CTGCGCGCTCGCTCGCTCACTGAGGCCGCCCGGGCAAAGCCCGGGCGTCG |
| P1138 Sequence | GGCGACCTTTGGTCGCCCGGCCTCAGTGAGCGAGCGAGCGCGCAGAGAGG |
| 5′ ITR at positions | GAGTGGCCAACTCCATCACTAGGGGTTCCTTGTAGTTAATGATTAACCCGCC |
| 1-130 | ATGCTACTTATCTACGTAGCCATGCTCTAGGAAGATCGGAATTCGCCCTTAA |
| CMV enhancer at | GCTAGCGGCGCGCCACCGGTGCGATCGCCGTTACATAACTTACGGTAAATG |
| positions 233-536 | GCCCGCCTGGCTGACCGCCCAACGACCCCCGCCCATTGACGTCAATAATGA |
| CMV promoter at | CGTATGTTOCCATAGTAACGCCAATAGGGACTTTCCATTGACGTCAATGGGT |
| positions 537-740 | GGAGTATTTACGGTAAACTGCCCACTTGGCAGTACATCAAGTGTATCATATG |
| Chimeric intron at | CCAAGTACGCCCCCTATTGACGTCAATGACGGTAAATGGCCCGCCTGGCAT |
| positions 793-925 | TATGCCCAGTACATGACCTTATGGGACTTTCCTACTTGGCAGTACATCTACG |
| H2B at positions | TATTAGTCATCGCTATTACCATGGTGATGCGGTTTTGGCAGTACATCAATGG |
| 948-1325 | GCGTGGATAGCGGTTTGACTCACGGGGATTTCCAAGTCTCCACCCCATTGA |
| EGFP at positions | CGTCAATGGGAGTTTGTTTTGGCACCAAAATCAACGGGACTTTCCAAAATGT |
| 1344-2063 | CGTAACAACTCCGCCCCATTGACGCAAATGGGGGGTAGGCGTGTACGGTGG |
| miR-182 target | GAGGTCTATATAAGCAGAGCTGGTTTAGTGAACCGTCAGATCCTGCAGAAGT |
| sequence at | TGGTCGTGAGGCACTGGGCAGGTAAGTATCAAGGTTACAAGACAGGTTTAA |
| positions 2071-2095 | GGAGACCAATAGAAACTGGGCTTGTCGAGACAGAGAAGACTCTTGCGTTTC |
| bGH polyA signal | TGATAGGCACCTATTGGTCTTACTGACATCCACTTTGCCTTTCTCTCCACAG |
| at positions 2103-2310 | GTGTCCAGGCGGCCGCGCCACCATGCCAGAGCCAGCGAAGTCTGCTCCCG |
| 3′ ITR at positions | CCCCGAAAAAGGGCTCCAAGAAGGCGGTGACTAAGGCGCAGAAGAAAGGC |
| 2398-2527 | GGCAAGAAGCGCAAGCGCAGCCGCAAGGAGAGCTATTCCATCTATGTGTAC |
| | AAGGTTCTGAAGCAGGTCCACCCTGACACCGGCATTTCGTCCAAGGCCATG |
| | GGCATCATGAATTCGTTTGTGAACGACATTTTCGAGCGCATCGCAGGTGAG |
| | GCTTCCCGCCTGGCGCATTACAACAAGCGCTCGACCATCACCTCCAGGGAG |
| | ATCCAGACGGCCGTGCGCCTGCTGCTGCCTGGGGAGTTGGCCAAGCACGC |
| | CGTGTCCGAGGGTACTAAGGCCATCACCAAGTACACCAGCGCTAAGGATCC |
| | ACCGGTCGCCACCATGGTGAGCAAGGGCGAGGAGCTGTTCACCGGGGTGG |
| | TGCCCATCCTGGTCGAGCTGGACGGCGACGTAAACGGCCACAAGTTCAGC |
| | GTGTCCGGCGAGGGCGAGGGCGATGCCACCTACGGCAAGCTGACCCTGAA |
| | GTTCATCTGCACCACCGGCAAGCTGCCCGTGCCCTGGCCCACCCTCGTGAC |
| | CACCCTGACCTACGGCGTGCAGTGCTTCAGCCGCTACCCCGACCACATGAA |
| | GCAGCACGACTTCTTCAAGTCCGCCATGCCCGAAGGCTACGTCCAGGAGCG |
| | CACCATCTTCTTCAAGGACGACGGCAACTACAAGACCCGCGCCGAGGTGAA |
| | GTTCGAGGGCGACACCCTGGTGAACCGCATCGAGCTGAAGGGCATCGACTT |
| | CAAGGAGGACGGCAACATCCTGGGGCACAAGCTGGAGTACAACTACAACAG |
| | CCACAACGTCTATATCATGGCCGACAAGCAGAAGAACGGCATCAAGGTGAA |
| | CTTCAAGATCCGCCACAACATCGAGGACGGCAGCGTGCAGCTCGCCGACCA |
| | CTACCAGCAGAACACCCCCATCGGCGACGGCCCCGTGCTGCTGCCCGACA |
| | ACCACTACCTGAGCACCCAGTCCGCCCTGAGCAAAGACCCCAACGAGAAGC |
| | GCGATCACATGGTCCTGCTGGAGTTCGTGACCGCCGCCGGGATCACTCTCG |
| | GCATGGACGAGCTGTACAAGTAATAAGCTTCGGTGTGAGTTCTACCATTGCC |
| | AAAGCCTCGACTGTGCCTTCTAGTTGCCAGCCATCTGTTGTTTGCCCCTCCC |
| | CCGTGCCTTCCTTGACCCTGGAAGGTGCCACTCCCACTGTCCTTTCCTAATA |
| | AAATGAGGAAATTGCATCGCATTGTCTGAGTAGGTGTCATTCTATTCTGGGG |
| | GGTGGGGTGGGGCAGGACAGCAAGGGGGAGGATTGGGAAGACAATAGCA |
| | GGCATGCTGGGGACTCGAGTTAAGGGCGAATTCCCGATAAGGATOTTCCTA |
| | GAGCATGGCTACGTAGATAAGTAGCATGGGGGGTTAATCATTAACTACAAGG |
| | AACCCCTAGTGATGGAGTTGGCCACTCCCTCTCTGCGCGCTCGCTCGCTCA |
| | CTGAGGCCGGGCGACCAAAGGTCGCCCGACGCCCGGGCTTTGCCCGGGC |
| | GGCCTCAGTGAGCGAGCGAGCGCGCAGCCTTAATTAACCTAATTCACTGGC |
| | CGTCGTTTTACAACGTCGTGACTGGGAAAACCCTGGCGTTACCCAACTTAAT |
| | CGCCTTGCAGCACATCCCCCTTTCGCCAGCTGGCGTAATAGCGAAGAGGCC |
| | CGCACCGATCGCCCTTCCCAACAGTTGCGCAGCCTGAATGGCGAATGGGAC |
| | GCGCCCTGTAGCGGCGCATTAAGCGCGGCGGGTGTGGTGGTTACGCGCAG |
| | CGTGACCGCTACACTTGCCAGCGCCCTAGCGCCCGCTCCTTTCGCTTTCTT |
| | CCCTTCCTTTCTCGCCACGTTCGCCGGCTTTCCCCGTCAAGCTCTAAATCGG |
| | GGGCTCCCTTTAGGGTTCCGATTTAGTGCTTTACGGCACCTCGACCCCAAAA |
| | AACTTGATTAGGGTGATGGTTCACGTAGTGGGCCATCGCCCTGATAGACGG |
| | TTTTTCGCCCTTTGACGTTGGAGTCCACGTTCTTTAATAGTGGACTCTTGTTC |
| | CAAACTGGAACAACACTCAACCCTATCTCGGTCTATTCTTTTGATTTATAAGG |
| | GATTTTGCCGATTTCGGCCTATTGGTTAAAAAATGAGCTGATTTAACAAAAAT |
| | TTAACGCGAATTTTAACAAAATATTAACGCTTACAATTTAGGTGGCACTTTTC |
| | GGGGAAATGTGCGCGGAACCCCTATTTGTTTATTTTTCTAAATACATTCAAAT |
| | ATGTATCCGCTCATGAGACAATAACCCTGATAAATGCTTCAATAATATTGAAA |
| | AAGGAAGAGTATGAGCCATATTCAACGGGAAACGTCGAGGCCGCGATTAAA |
| | TTCCAACATGGATGCTGATTTATATGGGTATAAATGGGCTCGCGATAATGTC |
| | GGGCAATCAGGTGCGACAATCTATCGCTTGTATGGGAAGCCCGATGCGCCA |
| | GAGTTGTTTCTGAAACATGGCAAAGGTAGCGTTGCCAATGATGTTACAGATG |
| | AGATGGTCAGACTAAACTGGCTGACGGAATTTATGCCTCTTCCGACCATCAA |
| | GCATTTTATCCGTACTCCTGATGATGCATGGTTACTCACCACTGCGATCCCC |
| | GGAAAAACAGCATTCCAGGTATTAGAAGAATATCCTGATTCAGGTGAAAATA |
| | TTGTTGATGCGCTGGCAGTGTTCCTGCGCCGGTTGCATTCGATTCCTGTTTG |
| | TAATTGTCCTTTTAACAGCGATCGCGTATTTCGTCTTGCTCAGGCGCAATCA |
| | CGAATGAATAACGGTTTGGTTGATGCGAGTGATTTTGATGACGAGCGTAATG |
| | GCTGGCCTGTTGAACAAGTCTGGAAAGAAATGCATAAACTTTTGCCATTCTC |
| | ACCGGATTCAGTCGTCACTCATGGTGATTTCTCACTTGATAACCTTATTTTTG |
| | ACGAGGGGAAATTAATAGGTTGTATTGATGTTGGACGAGTCGGAATCGCAG |
| | ACCGATACCAGGATCTTGCCATCCTATGGAACTGCCTCGGTGAGTTTTCTCC |
| | TTCATTACAGAAACGGCTTTTTCAAAAATATGGTATTGATAATCCTGATATGA |
| | ATAAATTGCAGTTTCATTTGATGCTCGATGAGTTTTTCTAACTGTCAGACCAA |
| | GTTTACTCATATATACTTTAGATTGATTTAAAACTTCATTTTTAATTTAAAAGGA |
| | TCTAGGTGAAGATCCTTTTTGATAATCTCATGACCAAAATCCCTTAACGTGAG |
| | TTTTCGTTCCACTGAGCGTCAGACCCCGTAGAAAAGATCAAAGGATCTTCTT |
| | GAGATCCTTTTTTTCTGCGCGTAATCTGCTGCTTGCAAACAAAAAAACCACC |
| | GCTACCAGCGGTGGTTTGTTTGCCGGATCAAGAGCTACCAACTCTTTTTCCG |
| | AAGGTAACTGGCTTCAGCAGAGCGCAGATACCAAATACTGTTCTTCTAGTGT |
| | AGCCGTAGTTAGGCCACCACTTCAAGAACTCTGTAGCACCGCCTACATACCT |
| | CGCTCTGCTAATCCTGTTACCAGTGGCTGCTGCCAGTGGCGATAAGTCGTG |
| | TCTTACCGGGTTGGACTCAAGACGATAGTTACCGGATAAGGCGCAGCGGTC |
| | GGGCTGAACGGGGGGTTCGTGCACACAGCCCAGCTTGGAGCGAACGACCT |
| | ACACCGAACTGAGATACCTACAGCGTGAGCTATGAGAAAGCGCCACGCTTC |
| | CCGAAGGGAGAAAGGCGGACAGGTATCCGGTAAGCGGCAGGGTCGGAACA |
| | GGAGAGCGCACGAGGGAGCTTCCAGGGGGAAACGCCTGGTATCTTTATAGT |
| | CCTGTCGGGTTTCGCCACCTCTGACTTGAGCGTCGATTTTTGTGATGCTCGT |
| | CAGGGGGGCGGAGCCTATGGAAAAACGCCAGCAACGCGGCCTTTTTACGG |
| | TTCCTGGCCTTTTGCTGGCCTTTTGCTCACATGTTCTTTCCTGCGTTATCCCC |
| | TGATTCTGTGGATAACCGTATTACCGCCTTTGAGTGAGCTGATACCGCTCGC |
| | CGCAGCCGAACGACCGAGCGCAGCGAGTCAGTGAGCGAGGAAGCGGAAGA |
| | GCGCCCAATACGCAAACCGCCTCTCCCCGCGCGTTGGCCGATTCATTAATG |
| | CAGCTGGCACGACAGGTTTCCCGACTGGAAAGCGGGCAGTGAGCGCAACG |
| | CAATTAATGTGAGTTAGCTCACTCATTAGGCACCCCAGGCTTTACACTTTATG |
| | CTTCCGGCTCGTATGTTGTGTGGAATTGTGAGCGGATAACAATTTCACACAG |
| | GAAACAGCTATGACCATGATTACGCCAGATTTAATTAAGGCCTTAATTAGG |
| SEQ ID NO: 19 | CTGCGCGCTCGCTCGCTCACTGAGGCCGCCCGGGCAAAGCCCGGGCGTCG |
| P1139 Sequence | GGCGACCTTTGGTCGCCCGGCCTCAGTGAGCGAGCGAGCGCGCAGAGAGG |
| 5′ ITR at positions | GAGTGGCCAACTCCATCACTAGGGGTTCCTTGTAGTTAATGATTAACCCGCC |
| 1-130 | ATGCTACTTATCTACGTAGCCATGCTCTAGGAAGATCGGAATTCGCCCTTAA |
| CMV enhancer at | GCTAGCGGCGCGCCACCGGTGCGATCGCCGTTACATAACTTACGGTAAATG |
| positions 233-536 | GCCCGCCTGGCTGACCGCCCAACGACCCCCGCCCATTGACGTCAATAATGA |
| CMV promoter at | CGTATGTTCCCATAGTAACGCCAATAGGGACTTTCCATTGACGTCAATGGGT |
| positions 537-740 | GGAGTATTTACGGTAAACTGCCCACTTGGCAGTACATCAAGTGTATCATATG |
| Chimeric intron at | CCAAGTACGCCCCCTATTGACGTCAATGACGGTAAATGGCCCGCCTGGCAT |
| positions 793-925 | TATGCCCAGTACATGACCTTATGGGACTTTCCTACTTGGCAGTACATCTACG |
| H2B at positions | TATTAGTCATCGCTATTACCATGGTGATGCGGTTTTGGCAGTACATCAATGG |
| 948-1325 | GCGTGGATAGCGGTTTGACTCACGGGGATTTCCAAGTCTCCACCCCATTGA |
| EGFP at positions | CGTCAATGGGAGTTTGTTTTGGCACCAAAATCAACGGGACTTTCCAAAATGT |
| 1344-2063 | CGTAACAACTCCGCCCCATTGACGCAAATGGGGGGTAGGCGTGTACGGTGG |
| miR-183 target | GAGGTCTATATAAGCAGAGCTGGTTTAGTGAACCGTCAGATOCTGCAGAAGT |
| sequence at | TGGTCGTGAGGCACTGGGCAGGTAAGTATCAAGGTTACAAGACAGGTTTAA |
| positions 2071-2092 | GGAGACCAATAGAAACTGGGCTTGTCGAGACAGAGAAGACTCTTGCGTTTC |
| bGH polyA signal | TGATAGGCACCTATTGGTCTTACTGACATCCACTTTGCCTTTCTCTCCACAG |
| at positions 2100-2307 | GTGTCCAGGCGGCCGCGCCACCATGCCAGAGCCAGCGAAGTCTGCTCCCG |
| 3′ ITR at positions | CCCCGAAAAAGGGCTCCAAGAAGGCGGTGACTAAGGCGCAGAAGAAAGGC |
| 2395-2524 | GGCAAGAAGCGCAAGCGCAGCCGCAAGGAGAGCTATTCCATCTATGTGTAC |
| | AAGGTTCTGAAGCAGGTCCACCCTGACACCGGCATTTCGTCCAAGGCCATG |
| | GGCATCATGAATTCGTTTGTGAACGACATTTTCGAGCGCATCGCAGGTGAG |
| | GCTTCCCGCCTGGCGCATTACAACAAGCGCTCGACCATCACCTCCAGGGAG |
| | ATCCAGACGGCCGTGCGCCTGCTGCTGCCTGGGGAGTTGGCCAAGCACGC |
| | CGTGTCCGAGGGTACTAAGGCCATCACCAAGTACACCAGCGCTAAGGATCC |
| | ACCGGTCGCCACCATGGTGAGCAAGGGCGAGGAGCTGTTCACCGGGGTGG |
| | TGCCCATCCTGGTCGAGCTGGACGGCGACGTAAACGGCCACAAGTTCAGC |
| | GTGTCCGGCGAGGGCGAGGGCGATGCCACCTACGGCAAGCTGACCCTGAA |
| | GTTCATCTGCACCACCGGCAAGCTGCCCGTGCCCTGGCCCACCCTCGTGAC |
| | CACCCTGACCTACGGCGTGCAGTGCTTCAGCCGCTACCCCGACCACATGAA |
| | GCAGCACGACTTCTTCAAGTCCGCCATGCCCGAAGGCTACGTCCAGGAGCG |
| | CACCATCTTCTTCAAGGACGACGGCAACTACAAGACCCGCGCCGAGGTGAA |
| | GTTCGAGGGCGACACCCTGGTGAACCGCATCGAGCTGAAGGGCATCGACTT |
| | CAAGGAGGACGGCAACATCCTGGGGCACAAGCTGGAGTACAACTACAACAG |
| | CCACAACGTCTATATCATGGCCGACAAGCAGAAGAACGGCATCAAGGTGAA |
| | CTTCAAGATCCGCCACAACATCGAGGACGGCAGCGTGCAGCTCGCCGACCA |
| | CTACCAGCAGAACACCCCCATCGGCGACGGCCCCGTGCTGCTGCCCGACA |
| | ACCACTACCTGAGCACCCAGTCCGCCCTGAGCAAAGACCCCAACGAGAAGC |
| | GCGATCACATGGTCCTGCTGGAGTTCGTGACCGCCGCCGGGATCACTCTCG |
| | GCATGGACGAGCTGTACAAGTAATAAGCTTAGTGAATTCTACCAGTGCCATA |
| | GCCTCGACTGTGCCTTCTAGTTGCCAGCCATCTGTTGTTTGCCCCTCCCCCG |
| | TGCCTTCCTTGACCCTGGAAGGTGCCACTCCCACTGTCCTTTCCTAATAAAA |
| | TGAGGAAATTGCATCGCATTGTCTGAGTAGGTGTCATTCTATTCTGGGGGGT |
| | GGGGGGGGCAGGACAGCAAGGGGGAGGATTGGGAAGACAATAGCAGGC |
| | ATGCTGGGGACTCGAGTTAAGGGCGAATTCCCGATAAGGATCTTCCTAGAG |
| | CATGGCTACGTAGATAAGTAGCATGGGGGGTTAATCATTAACTACAAGGAAC |
| | CCCTAGTGATGGAGTTGGCCACTCCCTCTCTGCGCGCTCGCTCGCTCACTG |
| | AGGCCGGGCGACCAAAGGTCGCCCGACGCCCGGGCTTTGCCCGGGGGGC |
| | CTCAGTGAGCGAGCGAGCGCGCAGCCTTAATTAACCTAATTCACTGGCCGT |
| | CGTTTTACAACGTCGTGACTGGGAAAACCCTGGCGTTACCCAACTTAATCGC |
| | CTTGCAGCACATCCCCCTTTCGCCAGCTGGCGTAATAGCGAAGAGGCCCGC |
| | ACCGATCGCCCTTCCCAACAGTTGCGCAGCCTGAATGGCGAATGGGACGCG |
| | CCCTGTAGCGGCGCATTAAGCGCGGGGGGTGTGGTGGTTACGCGCAGCGT |
| | GACCGCTACACTTGCCAGCGCCCTAGCGCCCGCTCCTTTCGCTTTCTTCCC |
| | TTCCTTTCTCGCCACGTTCGCCGGCTTTCCCCGTCAAGCTCTAAATCGGGG |
| | GCTCCCTTTAGGGTTCCGATTTAGTGCTTTACGGCACCTCGACCCCAAAAAA |
| | CTTGATTAGGGTGATGGTTCACGTAGTGGGCCATCGCCCTGATAGACGGTT |
| | TTTCGCCCTTTGACGTTGGAGTCCACGTTCTTTAATAGTGGACTCTTGTTCCA |
| | AACTGGAACAACACTCAACCCTATCTCGGTCTATTCTTTTGATTTATAAGGGA |
| | TTTTGCCGATTTCGGCCTATTGGTTAAAAAATGAGCTGATTTAACAAAAATTT |
| | AACGCGAATTTTAACAAAATATTAACGCTTACAATTTAGGTGGCACTTTTCGG |
| | GGAAATGTGCGCGGAACCCCTATTTGTTTATTTTTCTAAATACATTCAAATAT |
| | GTATCCGCTCATGAGACAATAACCCTGATAAATGCTTCAATAATATTGAAAAA |
| | GGAAGAGTATGAGCCATATTCAACGGGAAACGTCGAGGCCGCGATTAAATT |
| | CCAACATGGATGCTGATTTATATGGGTATAAATGGGCTCGCGATAATGTCGG |
| | GCAATCAGGTGCGACAATCTATCGCTTGTATGGGAAGCCCGATGCGCCAGA |
| | GTTGTTTCTGAAACATGGCAAAGGTAGCGTTGCCAATGATGTTACAGATGAG |
| | ATGGTCAGACTAAACTGGCTGACGGAATTTATGCCTCTTCCGACCATCAAGC |
| | ATTTTATCCGTACTCCTGATGATGCATGGTTACTCACCACTGCGATCCCCGG |
| | AAAAACAGCATTCCAGGTATTAGAAGAATATCCTGATTCAGGTGAAAATATTG |
| | TTGATGCGCTGGCAGTGTTCCTGCGCCGGTTGCATTCGATTCCTGTTTGTAA |
| | TTGTCCTTTTAACAGCGATCGCGTATTTCGTCTTGCTCAGGCGCAATCACGA |
| | ATGAATAACGGTTTGGTTGATGCGAGTGATTTTGATGACGAGCGTAATGGCT |
| | GGCCTGTTGAACAAGTCTGGAAAGAAATGCATAAACTTTTGCCATTCTCACC |
| | GGATTCAGTCGTCACTCATGGTGATTTCTCACTTGATAACCTTATTTTTGACG |
| | AGGGGAAATTAATAGGTTGTATTGATGTTGGACGAGTCGGAATCGCAGACC |
| | GATACCAGGATCTTGCCATCCTATGGAACTGCCTCGGTGAGTTTTCTCCTTC |
| | ATTACAGAAACGGCTTTTTCAAAAATATGGTATTGATAATCCTGATATGAATA |
| | AATTGCAGTTTCATTTGATGCTCGATGAGTTTTTCTAACTGTCAGACCAAGTT |
| | TACTCATATATACTTTAGATTGATTTAAAACTTCATTTTTAATTTAAAAGGATCT |
| | AGGTGAAGATCCTTTTTGATAATCTCATGACCAAAATCCCTTAACGTGAGTTT |
| | TCGTTCCACTGAGCGTCAGACCCCGTAGAAAAGATCAAAGGATOTTCTTGAG |
| | ATCCTTTTTTTCTGCGCGTAATCTGCTGCTTGCAAACAAAAAAACCACCGCTA |
| | CCAGCGGTGGTTTGTTTGCCGGATCAAGAGCTACCAACTCTTTTTCCGAAGG |
| | TAACTGGCTTCAGCAGAGCGCAGATACCAAATACTGTTCTTCTAGTGTAGCC |
| | GTAGTTAGGCCACCACTTCAAGAACTCTGTAGCACCGCCTACATACCTCGCT |
| | CTGCTAATCCTGTTACCAGTGGCTGCTGCCAGTGGCGATAAGTCGTGTCTTA |
| | CCGGGTTGGACTCAAGACGATAGTTACCGGATAAGGCGCAGCGGTCGGGC |
| | TGAACGGGGGGTTCGTGCACACAGCCCAGCTTGGAGCGAACGACCTACAC |
| | CGAACTGAGATACCTACAGCGTGAGCTATGAGAAAGCGCCACGCTTCCCGA |
| | AGGGAGAAAGGCGGACAGGTATCCGGTAAGCGGCAGGGTCGGAACAGGAG |
| | AGCGCACGAGGGAGCTTCCAGGGGGAAACGCCTGGTATCTTTATAGTCCTG |
| | TCGGGTTTCGCCACCTCTGACTTGAGCGTCGATTTTTGTGATGCTCGTCAGG |
| | GGGGCGGAGCCTATGGAAAAACGCCAGCAACGCGGCCTTTTTACGGTTCCT |
| | GGCCTTTTGCTGGCCTTTTGCTCACATGTTCTTTCCTGCGTTATCCCCTGATT |
| | CTGTGGATAACCGTATTACCGCCTTTGAGTGAGCTGATACCGCTCGCCGCA |
| | GCCGAACGACCGAGCGCAGCGAGTCAGTGAGCGAGGAAGCGGAAGAGCG |
| | CCCAATACGCAAACCGCCTCTCCCCGCGCGTTGGCCGATTCATTAATGCAG |
| | CTGGCACGACAGGTTTCCCGACTGGAAAGCGGGCAGTGAGCGCAACGCAA |
| | TTAATGTGAGTTAGCTCACTCATTAGGCACCCCAGGCTTTACACTTTATGCTT |
| | CCGGCTCGTATGTTGTGTGGAATTGTGAGCGGATAACAATTTCACACAGGAA |
| | ACAGCTATGACCATGATTACGCCAGATTTAATTAAGGCCTTAATTAGG |
| SEQ ID NO: 20 | CTGCGCGCTCGCTCGCTCACTGAGGCCGCCCGGGCAAAGCCCGGGCGTCG |
| P1140 Sequence | GGCGACCTTTGGTCGCCCGGCCTCAGTGAGCGAGCGAGCGCGCAGAGAGG |
| 5′ ITR at positions | GAGTGGCCAACTCCATCACTAGGGGTTCCTTGTAGTTAATGATTAACCCGCC |
| 1-130 | ATGCTACTTATCTACGTAGCCATGCTCTAGGAAGATCGGAATTCGCCCTTAA |
| CMV enhancer at | GCTAGCGGCGCGCCACCGGTGCGATCGCCGTTACATAACTTACGGTAAATG |
| positions 233-536 | GCCCGCCTGGCTGACCGCCCAACGACCCCCGCCCATTGACGTCAATAATGA |
| CMV promoter at | CGTATGTTCCCATAGTAACGCCAATAGGGACTTTCCATTGACGTCAATGGGT |
| positions 537-740 | GGAGTATTTACGGTAAACTGCCCACTTGGCAGTACATCAAGTGTATCATATG |
| Chimeric intron at | CCAAGTACGCCCCCTATTGACGTCAATGACGGTAAATGGCCCGCCTGGCAT |
| positions 793-925 | TATGCCCAGTACATGACCTTATGGGACTTTCCTACTTGGCAGTACATCTACG |
| H2B at positions | TATTAGTCATCGCTATTACCATGGTGATGCGGTTTTGGCAGTACATCAATGG |
| 948-1325 | GCGTGGATAGCGGTTTGACTCACGGGGATTTCCAAGTCTCCACCCCATTGA |
| EGFP at positions | CGTCAATGGGAGTTTGTTTTGGCACCAAAATCAACGGGACTTTCCAAAATGT |
| 1344-2063 | CGTAACAACTCCGCCCCATTGACGCAAATGGGGGGTAGGCGTGTACGGTGG |
| miR-183 target | GAGGTCTATATAAGCAGAGCTGGTTTAGTGAACCGTCAGATCCTGCAGAAGT |
| sequence at | TGGTCGTGAGGCACTGGGCAGGTAAGTATCAAGGTTACAAGACAGGTTTAA |
| positions 2071-2092 | GGAGACCAATAGAAACTGGGCTTGTCGAGACAGAGAAGACTCTTGCGTTTC |
| miR-96 target | TGATAGGCACCTATTGGTCTTACTGACATCCACTTTGCCTTTCTCTCCACAG |
| sequence at | GTGTCCAGGCGGCCGCGCCACCATGCCAGAGCCAGCGAAGTCTGCTCCCG |
| positions 2097-2119 | CCCCGAAAAAGGGCTCCAAGAAGGGGTGACTAAGGCGCAGAAGAAAGGC |
| miR-182 target | GGCAAGAAGCGCAAGCGCAGCCGCAAGGAGAGCTATTCCATCTATGTGTAC |
| sequence at | AAGGTTCTGAAGCAGGTCCACCCTGACACCGGCATTTCGTCCAAGGCCATG |
| positions 2124-2148 | GGCATCATGAATTCGTTTGTGAACGACATTTTCGAGCGCATCGCAGGTGAG |
| bGH polyA signal | GCTTCCCGCCTGGCGCATTACAACAAGCGCTCGACCATCACCTCCAGGGAG |
| at positions 2156-2363 | ATCCAGACGGCCGTGCGCCTGCTGCTGCCTGGGGAGTTGGCCAAGCACGC |
| 3′ ITR at positions | CGTGTCCGAGGGTACTAAGGCCATCACCAAGTACACCAGCGCTAAGGATCC |
| 2451-2580 | ACCGGTCGCCACCATGGTGAGCAAGGGCGAGGAGCTGTTCACCGGGGTGG |
| | TGCCCATCCTGGTCGAGCTGGACGGCGACGTAAACGGCCACAAGTTCAGC |
| | GTGTCCGGCGAGGGCGAGGGCGATGCCACCTACGGCAAGCTGACCCTGAA |
| | GTTCATCTGCACCACCGGCAAGCTGCCCGTGCCCTGGCCCACCCTCGTGAC |
| | CACCCTGACCTACGGCGTGCAGTGCTTCAGCCGCTACCCCGACCACATGAA |
| | GCAGCACGACTTCTTCAAGTCCGCCATGCCCGAAGGCTACGTCCAGGAGCG |
| | CACCATCTTCTTCAAGGACGACGGCAACTACAAGACCCGCGCCGAGGTGAA |
| | GTTCGAGGGCGACACCCTGGTGAACCGCATCGAGCTGAAGGGCATCGACTT |
| | CAAGGAGGACGGCAACATCCTGGGGCACAAGCTGGAGTACAACTACAACAG |
| | CCACAACGTCTATATCATGGCCGACAAGCAGAAGAACGGCATCAAGGTGAA |
| | CTTCAAGATCCGCCACAACATCGAGGACGGCAGCGTGCAGCTCGCCGACCA |
| | CTACCAGCAGAACACCCCCATCGGCGACGGCCCCGTGCTGCTGCCCGACA |
| | ACCACTACCTGAGCACCCAGTCCGCCCTGAGCAAAGACCCCAACGAGAAGC |
| | GCGATCACATGGTCCTGCTGGAGTTCGTGACCGCCGCCGGGATCACTCTCG |
| | GCATGGACGAGCTGTACAAGTAATAAGCTTAGTGAATTCTACCAGTGCCATA |
| | CGATAGCAAAAATGTGCTAGTGCCAAACGATCGGTGTGAGTTCTACCATTGC |
| | CAAAGCCTCGACTGTGCCTTCTAGTTGCCAGCCATCTGTTGTTTGCCCCTCC |
| | CCCGTGCCTTCCTTGACCCTGGAAGGTGCCACTCCCACTGTCCTTTCCTAAT |
| | AAAATGAGGAAATTGCATCGCATTGTCTGAGTAGGTGTCATTCTATTCTGGG |
| | GGGTGGGGTGGGGCAGGACAGCAAGGGGGAGGATTGGGAAGACAATAGC |
| | AGGCATGCTGGGGACTCGAGTTAAGGGCGAATTCCCGATAAGGATCTTCCT |
| | AGAGCATGGCTACGTAGATAAGTAGCATGGCGGGTTAATCATTAACTACAAG |
| | GAACCCCTAGTGATGGAGTTGGCCACTCCCTCTCTGCGCGCTCGCTCGCTC |
| | ACTGAGGCCGGGCGACCAAAGGTCGCCCGACGCCCGGGCTTTGCCCGGG |
| | CGGCCTCAGTGAGCGAGCGAGCGCGCAGCCTTAATTAACCTAATTCACTGG |
| | CCGTCGTTTTACAACGTCGTGACTGGGAAAACCCTGGCGTTACCCAACTTAA |
| | TCGCCTTGCAGCACATCCCCCTTTCGCCAGCTGGCGTAATAGCGAAGAGGC |
| | CCGCACCGATCGCCCTTCCCAACAGTTGCGCAGCCTGAATGGCGAATGGGA |
| | CGCGCCCTGTAGCGGCGCATTAAGCGCGGCGGGTGTGGTGGTTACGCGCA |
| | GCGTGACCGCTACACTTGCCAGCGCCCTAGCGCCCGCTCCTTTCGCTTTCT |
| | TCCCTTCCTTTCTCGCCACGTTCGCCGGCTTTCCCCGTCAAGCTCTAAATCG |
| | GGGGCTCCCTTTAGGGTTCCGATTTAGTGCTTTACGGCACCTCGACCCCAA |
| | AAAACTTGATTAGGGTGATGGTTCACGTAGTGGGCCATCGCCCTGATAGAC |
| | GGTTTTTCGCCCTTTGACGTTGGAGTCCACGTTCTTTAATAGTGGACTCTTGT |
| | TCCAAACTGGAACAACACTCAACCCTATCTCGGTCTATTCTTTTGATTTATAA |
| | GGGATTTTGCCGATTTCGGCCTATTGGTTAAAAAATGAGCTGATTTAACAAAA |
| | ATTTAACGCGAATTTTAACAAAATATTAACGCTTACAATTTAGGTGGCACTTTT |
| | CGGGGAAATGTGCGCGGAACCCCTATTTGTTTATTTTTCTAAATACATTCAAA |
| | TATGTATCCGCTCATGAGACAATAACCCTGATAAATGCTTCAATAATATTGAA |
| | AAAGGAAGAGTATGAGCCATATTCAACGGGAAACGTCGAGGCCGCGATTAA |
| | ATTCCAACATGGATGCTGATTTATATGGGTATAAATGGGCTCGCGATAATGT |
| | CGGGCAATCAGGTGCGACAATCTATCGCTTGTATGGGAAGCCCGATGCGCC |
| | AGAGTTGTTTCTGAAACATGGCAAAGGTAGCGTTGCCAATGATGTTACAGAT |
| | GAGATGGTCAGACTAAACTGGCTGACGGAATTTATGCCTCTTCCGACCATCA |
| | AGCATTTTATCCGTACTCCTGATGATGCATGGTTACTCACCACTGCGATCCC |
| | CGGAAAAACAGCATTCCAGGTATTAGAAGAATATCCTGATTCAGGTGAAAAT |
| | ATTGTTGATGCGCTGGCAGTGTTCCTGCGCCGGTTGCATTCGATTCCTGTTT |
| | GTAATTGTCCTTTTAACAGCGATCGCGTATTTCGTCTTGCTCAGGCGCAATC |
| | ACGAATGAATAACGGTTTGGTTGATGCGAGTGATTTTGATGACGAGCGTAAT |
| | GGCTGGCCTGTTGAACAAGTCTGGAAAGAAATGCATAAACTTTTGCCATTCT |
| | CACCGGATTCAGTCGTCACTCATGGTGATTTCTCACTTGATAACCTTATTTTT |
| | GACGAGGGGAAATTAATAGGTTGTATTGATGTTGGACGAGTCGGAATCGCA |
| | GACCGATACCAGGATCTTGCCATCCTATGGAACTGCCTCGGTGAGTTTTCTC |
| | CTTCATTACAGAAACGGCTTTTTCAAAAATATGGTATTGATAATCCTGATATG |
| | AATAAATTGCAGTTTCATTTGATGCTCGATGAGTTTTTCTAACTGTCAGACCA |
| | AGTTTACTCATATATACTTTAGATTGATTTAAAACTTCATTTTTAATTTAAAAGG |
| | ATCTAGGTGAAGATCCTTTTTGATAATCTCATGACCAAAATCCCTTAACGTGA |
| | GTTTTCGTTCCACTGAGCGTCAGACCCCGTAGAAAAGATCAAAGGATCTTCT |
| | TGAGATCCTTTTTTTCTGCGCGTAATCTGCTGCTTGCAAACAAAAAAACCACC |
| | GCTACCAGCGGTGGTTTGTTTGCCGGATCAAGAGCTACCAACTCTTTTTCCG |
| | AAGGTAACTGGCTTCAGCAGAGCGCAGATACCAAATACTGTTCTTCTAGTGT |
| | AGCCGTAGTTAGGCCACCACTTCAAGAACTCTGTAGCACCGCCTACATACCT |
| | CGCTCTGCTAATCCTGTTACCAGTGGCTGCTGCCAGTGGCGATAAGTCGTG |
| | TCTTACCGGGTTGGACTCAAGACGATAGTTACCGGATAAGGCGCAGCGGTC |
| | GGGCTGAACGGGGGGTTCGTGCACACAGCCCAGCTTGGAGCGAACGACCT |
| | ACACCGAACTGAGATACCTACAGCGTGAGCTATGAGAAAGCGCCACGCTTC |
| | CCGAAGGGAGAAAGGCGGACAGGTATCCGGTAAGCGGCAGGGTCGGAACA |
| | GGAGAGCGCACGAGGGAGCTTCCAGGGGGAAACGCCTGGTATCTTTATAGT |
| | CCTGTCGGGTTTCGCCACCTCTGACTTGAGCGTCGATTTTTGTGATGCTCGT |
| | CAGGGGGGCGGAGCCTATGGAAAAACGCCAGCAACGCGGCCTTTTTACGG |
| | TTCCTGGCCTTTTGCTGGCCTTTTGCTCACATGTTCTTTCCTGCGTTATCCCC |
| | TGATTCTGTGGATAACCGTATTACCGCCTTTGAGTGAGCTGATACCGCTCGC |
| | CGCAGCCGAACGACCGAGCGCAGCGAGTCAGTGAGCGAGGAAGCGGAAGA |
| | GCGCCCAATACGCAAACCGCCTCTCCCCGCGCGTTGGCCGATTCATTAATG |
| | CAGCTGGCACGACAGGTTTCCCGACTGGAAAGCGGGCAGTGAGCGCAACG |
| | CAATTAATGTGAGTTAGCTCACTCATTAGGCACCCCAGGCTTTACACTTTATG |
| | CTTCCGGCTCGTATGTTGTGTGGAATTGTGAGCGGATAACAATTTCACACAG |
| | GAAACAGCTATGACCATGATTACGCCAGATTTAATTAAGGCCTTAATTAGG |
| SEQ ID NO: 21 | CTGCGCGCTCGCTCGCTCACTGAGGCCGCCCGGGCAAAGCCCGGGCGTCG |
| P1141 Sequence | GGCGACCTTTGGTCGCCCGGCCTCAGTGAGCGAGCGAGCGCGCAGAGAGG |
| 5′ ITR at positions | GAGTGGCCAACTCCATCACTAGGGGTTCCTTGTAGTTAATGATTAACCCGCC |
| 1-130 | ATGCTACTTATCTACGTAGCCATGCTCTAGGAAGATCGGAATTCGCCCTTAA |
| CMV enhancer at | GCTAGCGGCGCGCCACCGGTGCGATCGCCGTTACATAACTTACGGTAAATG |
| positions 233-536 | GCCCGCCTGGCTGACCGCCCAACGACCCCCGCCCATTGACGTCAATAATGA |
| CMV promoter at | CGTATGTTOCCATAGTAACGCCAATAGGGACTTTCCATTGACGTCAATGGGT |
| positions 537-740 | GGAGTATTTACGGTAAACTGCCCACTTGGCAGTACATCAAGTGTATCATATG |
| Chimeric intron at | CCAAGTACGCCCCCTATTGACGTCAATGACGGTAAATGGCCCGCCTGGCAT |
| positions 793-925 | TATGCCCAGTACATGACCTTATGGGACTTTCCTACTTGGCAGTACATCTACG |
| H2B at positions | TATTAGTCATCGCTATTACCATGGTGATGCGGTTTTGGCAGTACATCAATGG |
| 948-1325 | GCGTGGATAGCGGTTTGACTCACGGGGATTTCCAAGTCTCCACCCCATTGA |
| EGFP at positions | CGTCAATGGGAGTTTGTTTTGGCACCAAAATCAACGGGACTTTCCAAAATGT |
| 1344-2063 | CGTAACAACTCCGCCCCATTGACGCAAATGGGGGGTAGGCGTGTACGGTGG |
| miR-183 target | GAGGTCTATATAAGCAGAGCTGGTTTAGTGAACCGTCAGATCCTGCAGAAGT |
| sequences (3) at | TGGTCGTGAGGCACTGGGCAGGTAAGTATCAAGGTTACAAGACAGGTTTAA |
| positions 2071- | GGAGACCAATAGAAACTGGGCTTGTCGAGACAGAGAAGACTCTTGCGTTTC |
| 2092, 2097-2118, | TGATAGGCACCTATTGGTCTTACTGACATCCACTTTGCCTTTCTCTCCACAG |
| and 2123-2144 | GTGTCCAGGCGGCCGCGCCACCATGCCAGAGCCAGCGAAGTCTGCTCCCG |
| miR-96 target | CCCCGAAAAAGGGCTCCAAGAAGGCGGTGACTAAGGCGCAGAAGAAAGGC |
| sequences (3) at | GGCAAGAAGCGCAAGCGCAGCCGCAAGGAGAGCTATTCCATCTATGTGTAC |
| positions 2149- | AAGGTTCTGAAGCAGGTCCACCCTGACACCGGCATTTCGTCCAAGGCCATG |
| 2171, 2176-2198, | GGCATCATGAATTCGTTTGTGAACGACATTTTCGAGCGCATCGCAGGTGAG |
| and 2203-2225 | GCTTCCCGCCTGGCGCATTACAACAAGCGCTCGACCATCACCTCCAGGGAG |
| miR-182 target | ATCCAGACGGCCGTGCGCCTGCTGCTGCCTGGGGAGTTGGCCAAGCACGC |
| sequences (3) at | CGTGTCCGAGGGTACTAAGGCCATCACCAAGTACACCAGCGCTAAGGATCC |
| positions 2230- | ACCGGTCGCCACCATGGTGAGCAAGGGCGAGGAGCTGTTCACCGGGGTGG |
| 2254, 2259-2283, | TGCCCATCCTGGTCGAGCTGGACGGCGACGTAAACGGCCACAAGTTCAGC |
| and 2288-2312 | GTGTCCGGCGAGGGCGAGGGCGATGCCACCTACGGCAAGCTGACCCTGAA |
| bGH polyA signal | GTTCATCTGCACCACCGGCAAGCTGCCCGTGCCCTGGCCCACCCTCGTGAC |
| at positions 2320- | CACCCTGACCTACGGCGTGCAGTGCTTCAGCCGCTACCCCGACCACATGAA |
| 2527 | GCAGCACGACTTCTTCAAGTCCGCCATGCCCGAAGGCTACGTCCAGGAGCG |
| 3′ ITR at positions | CACCATCTTCTTCAAGGACGACGGCAACTACAAGACCCGCGCCGAGGTGAA |
| 2615-2744 | GTTCGAGGGCGACACCCTGGTGAACCGCATCGAGCTGAAGGGCATCGACTT |
| | CAAGGAGGACGGCAACATCCTGGGGCACAAGCTGGAGTACAACTACAACAG |
| | CCACAACGTCTATATCATGGCCGACAAGCAGAAGAACGGCATCAAGGTGAA |
| | CTTCAAGATCCGCCACAACATCGAGGACGGCAGCGTGCAGCTCGCCGACCA |
| | CTACCAGCAGAACACCCCCATCGGCGACGGCCCCGTGCTGCTGCCCGACA |
| | ACCACTACCTGAGCACCCAGTCCGCCCTGAGCAAAGACCCCAACGAGAAGC |
| | GCGATCACATGGTCCTGCTGGAGTTCGTGACCGCCGCCGGGATCACTCTCG |
| | GCATGGACGAGCTGTACAAGTAATAAGCTTAGTGAATTCTACCAGTGCCATA |
| | CGATAGTGAATTCTACCAGTGCCATACGATAGTGAATTCTACCAGTGCCATA |
| | CGATAGCAAAAATGTGCTAGTGCCAAACGATAGCAAAAATGTGCTAGTGCCA |
| | AACGATAGCAAAAATGTGCTAGTGCCAAACGATCGGTGTGAGTTCTACCATT |
| | GCCAAACGATCGGTGTGAGTTCTACCATTGCCAAACGATCGGTGTGAGTTCT |
| | ACCATTGCCAAAGCCTCGACTGTGCCTTCTAGTTGCCAGCCATCTGTTGTTT |
| | GCCCCTCCCCCGTGCCTTCCTTGACCCTGGAAGGTGCCACTCCCACTGTCC |
| | TTTCCTAATAAAATGAGGAAATTGCATCGCATTGTCTGAGTAGGTGTCATTCT |
| | ATTCTGGGGGGTGGGGTGGGGCAGGACAGCAAGGGGGAGGATTGGGAAG |
| | ACAATAGCAGGCATGCTGGGGACTCGAGTTAAGGGCGAATTCCCGATAAGG |
| | ATCTTCCTAGAGCATGGCTACGTAGATAAGTAGCATGGCGGGTTAATCATTA |
| | ACTACAAGGAACCCCTAGTGATGGAGTTGGCCACTCCCTCTCTGCGCGCTC |
| | GCTCGCTCACTGAGGCCGGGCGACCAAAGGTCGCCCGACGCCCGGGCTTT |
| | GCCCGGGCGGCCTCAGTGAGCGAGCGAGCGCGCAGCCTTAATTAACCTAA |
| | TTCACTGGCCGTCGTTTTACAACGTCGTGACTGGGAAAACCCTGGCGTTACC |
| | CAACTTAATCGCCTTGCAGCACATCCCCCTTTCGCCAGCTGGCGTAATAGCG |
| | AAGAGGCCCGCACCGATCGCCCTTCCCAACAGTTGCGCAGCCTGAATGGC |
| | GAATGGGACGCGCCCTGTAGCGGCGCATTAAGCGCGGCGGGTGTGGTGGT |
| | TACGCGCAGCGTGACCGCTACACTTGCCAGCGCCCTAGCGCCCGCTCCTTT |
| | CGCTTTCTTCCCTTCCTTTCTCGCCACGTTCGCCGGCTTTCCCCGTCAAGCT |
| | CTAAATCGGGGGCTCCCTTTAGGGTTCCGATTTAGTGCTTTACGGCACCTCG |
| | ACCCCAAAAAACTTGATTAGGGTGATGGTTCACGTAGTGGGCCATCGCCCT |
| | GATAGACGGTTTTTCGCCCTTTGACGTTGGAGTCCACGTTCTTTAATAGTGG |
| | ACTCTTGTTCCAAACTGGAACAACACTCAACCCTATCTCGGTCTATTCTTTTG |
| | ATTTATAAGGGATTTTGCCGATTTCGGCCTATTGGTTAAAAAATGAGCTGATT |
| | TAACAAAAATTTAACGCGAATTTTAACAAAATATTAACGCTTACAATTTAGGTG |
| | GCACTTTTCGGGGAAATGTGCGCGGAACCCCTATTTGTTTATTTTTCTAAATA |
| | CATTCAAATATGTATCCGCTCATGAGACAATAACCCTGATAAATGCTTCAATA |
| | ATATTGAAAAAGGAAGAGTATGAGCCATATTCAACGGGAAACGTCGAGGCC |
| | GCGATTAAATTCCAACATGGATGCTGATTTATATGGGTATAAATGGGCTCGC |
| | GATAATGTCGGGCAATCAGGTGCGACAATCTATCGCTTGTATGGGAAGCCC |
| | GATGCGCCAGAGTTGTTTCTGAAACATGGCAAAGGTAGCGTTGCCAATGAT |
| | GTTACAGATGAGATGGTCAGACTAAACTGGCTGACGGAATTTATGCCTCTTC |
| | CGACCATCAAGCATTTTATCCGTACTCCTGATGATGCATGGTTACTCACCAC |
| | TGCGATCCCCGGAAAAACAGCATTCCAGGTATTAGAAGAATATCCTGATTCA |
| | GGTGAAAATATTGTTGATGCGCTGGCAGTGTTCCTGCGCCGGTTGCATTCG |
| | ATTCCTGTTTGTAATTGTCCTTTTAACAGCGATCGCGTATTTCGTCTTGCTCA |
| | GGCGCAATCACGAATGAATAACGGTTTGGTTGATGCGAGTGATTTTGATGAC |
| | GAGCGTAATGGCTGGCCTGTTGAACAAGTCTGGAAAGAAATGCATAAACTTT |
| | TGCCATTCTCACCGGATTCAGTCGTCACTCATGGTGATTTCTCACTTGATAAC |
| | CTTATTTTTGACGAGGGGAAATTAATAGGTTGTATTGATGTTGGACGAGTCG |
| | GAATCGCAGACCGATACCAGGATCTTGCCATCCTATGGAACTGCCTCGGTG |
| | AGTTTTCTCCTTCATTACAGAAACGGCTTTTTCAAAAATATGGTATTGATAATC |
| | CTGATATGAATAAATTGCAGTTTCATTTGATGCTCGATGAGTTTTTCTAACTG |
| | TCAGACCAAGTTTACTCATATATACTTTAGATTGATTTAAAACTTCATTTTTAA |
| | TTTAAAAGGATCTAGGTGAAGATCCTTTTTGATAATCTCATGACCAAAATCCC |
| | TTAACGTGAGTTTTCGTTCCACTGAGCGTCAGACCCCGTAGAAAAGATCAAA |
| | GGATCTTCTTGAGATCCTTTTTTTCTGCGCGTAATCTGCTGCTTGCAAACAAA |
| | AAAACCACCGCTACCAGCGGTGGTTTGTTTGCCGGATCAAGAGCTACCAAC |
| | TCTTTTTCCGAAGGTAACTGGCTTCAGCAGAGCGCAGATACCAAATACTGTT |
| | CTTCTAGTGTAGCCGTAGTTAGGCCACCACTTCAAGAACTCTGTAGCACCGC |
| | CTACATACCTCGCTCTGCTAATCCTGTTACCAGTGGCTGCTGCCAGTGGCGA |
| | TAAGTCGTGTCTTACCGGGTTGGACTCAAGACGATAGTTACCGGATAAGGC |
| | GCAGCGGTCGGGCTGAACGGGGGGTTCGTGCACACAGCCCAGCTTGGAGC |
| | GAACGACCTACACCGAACTGAGATACCTACAGCGTGAGCTATGAGAAAGCG |
| | CCACGCTTCCCGAAGGGAGAAAGGCGGACAGGTATCCGGTAAGCGGCAGG |
| | GTCGGAACAGGAGAGCGCACGAGGGAGCTTCCAGGGGGAAACGCCTGGTA |
| | TCTTTATAGTCCTGTCGGGTTTCGCCACCTCTGACTTGAGCGTCGATTTTTGT |
| | GATGCTCGTCAGGGGGGCGGAGCCTATGGAAAAACGCCAGCAACGCGGCC |
| | TTTTTACGGTTCCTGGCCTTTTGCTGGCCTTTTGCTCACATGTTCTTTCCTGC |
| | GTTATCCCCTGATTCTGTGGATAACCGTATTACCGCCTTTGAGTGAGCTGAT |
| | ACCGCTCGCCGCAGCCGAACGACCGAGCGCAGCGAGTCAGTGAGCGAGGA |
| | AGCGGAAGAGCGCCCAATACGCAAACCGCCTCTCCCCGCGCGTTGGCCGA |
| | TTCATTAATGCAGCTGGCACGACAGGTTTCCCGACTGGAAAGCGGGCAGTG |
| | AGCGCAACGCAATTAATGTGAGTTAGCTCACTCATTAGGCACCCCAGGCTTT |
| | ACACTTTATGCTTCCGGCTCGTATGTTGTGTGGAATTGTGAGCGGATAACAA |
| | TTTCACACAGGAAACAGCTATGACCATGATTACGCCAGATTTAATTAAGGCC |
| | TTAATTAGG |
| SEQ ID NO: 22 | CTGCGCGCTCGCTCGCTCACTGAGGCCGCCCGGGCAAAGCCCGGGCGTCG |
| P1142 Sequence | GGCGACCTTTGGTCGCCCGGCCTCAGTGAGCGAGCGAGCGCGCAGAGAGG |
| 5′ ITR at positions | GAGTGGCCAACTCCATCACTAGGGGTTCCTTGTAGTTAATGATTAACCCGCC |
| 1-130 | ATGCTACTTATCTACGTAGCCATGCTCTAGGAAGATCGGAATTCGCCCTTAA |
| CMV enhancer at | GCTAGCGGCGCGCCACCGGTGCGATCGCCGTTACATAACTTACGGTAAATG |
| positions 233-536 | GCCCGCCTGGCTGACCGCCCAACGACCCCCGCCCATTGACGTCAATAATGA |
| CMV promoter at | CGTATGTTCCCATAGTAACGCCAATAGGGACTTTCCATTGACGTCAATGGGT |
| positions 537-740 | GGAGTATTTACGGTAAACTGCCCACTTGGCAGTACATCAAGTGTATCATATG |
| Chimeric intron at | CCAAGTACGCCCCCTATTGACGTCAATGACGGTAAATGGCCCGCCTGGCAT |
| positions 793-925 | TATGCCCAGTACATGACCTTATGGGACTTTCCTACTTGGCAGTACATCTACG |
| H2B at positions | TATTAGTCATCGCTATTACCATGGTGATGCGGTTTTGGCAGTACATCAATGG |
| 948-1325 | GCGTGGATAGCGGTTTGACTCACGGGGATTTCCAAGTCTCCACCCCATTGA |
| EGFP at positions | CGTCAATGGGAGTTTGTTTTGGCACCAAAATCAACGGGACTTTCCAAAATGT |
| 1344-2063 | CGTAACAACTCCGCCCCATTGACGCAAATGGGGGGTAGGCGTGTACGGTGG |
| miR-96 target | GAGGTCTATATAAGCAGAGCTGGTTTAGTGAACCGTCAGATCCTGCAGAAGT |
| sequences (4) at | TGGTCGTGAGGCACTGGGCAGGTAAGTATCAAGGTTACAAGACAGGTTTAA |
| positions 2071- | GGAGACCAATAGAAACTGGGCTTGTCGAGACAGAGAAGACTCTTGCGTTTC |
| 2093, 2098-2120, | TGATAGGCACCTATTGGTCTTACTGACATCCACTTTGCCTTTCTCTCCACAG |
| 2125-2147, and | GTGTCCAGGCGGCCGCGCCACCATGCCAGAGCCAGCGAAGTCTGCTCCCG |
| 2152-2174 | CCCCGAAAAAGGGCTCCAAGAAGGCGGTGACTAAGGCGCAGAAGAAAGGC |
| bGH polyA signal | GGCAAGAAGCGCAAGCGCAGCCGCAAGGAGAGCTATTCCATCTATGTGTAC |
| at positions 2182-2389 | AAGGTTCTGAAGCAGGTCCACCCTGACACCGGCATTTCGTCCAAGGCCATG |
| 3′ ITR at positions | GGCATCATGAATTCGTTTGTGAACGACATTTTCGAGCGCATCGCAGGTGAG |
| 2477-2606 | GCTTCCCGCCTGGCGCATTACAACAAGCGCTCGACCATCACCTCCAGGGAG |
| | ATCCAGACGGCCGTGCGCCTGCTGCTGCCTGGGGAGTTGGCCAAGCACGC |
| | CGTGTCCGAGGGTACTAAGGCCATCACCAAGTACACCAGCGCTAAGGATCC |
| | ACCGGTCGCCACCATGGTGAGCAAGGGCGAGGAGCTGTTCACCGGGGTGG |
| | TGCCCATCCTGGTCGAGCTGGACGGCGACGTAAACGGCCACAAGTTCAGC |
| | GTGTCCGGCGAGGGCGAGGGCGATGCCACCTACGGCAAGCTGACCCTGAA |
| | GTTCATCTGCACCACCGGCAAGCTGCCCGTGCCCTGGCCCACCCTCGTGAC |
| | CACCCTGACCTACGGCGTGCAGTGCTTCAGCCGCTACCCCGACCACATGAA |
| | GCAGCACGACTTCTTCAAGTCCGCCATGCCCGAAGGCTACGTCCAGGAGCG |
| | CACCATCTTCTTCAAGGACGACGGCAACTACAAGACCCGCGCCGAGGTGAA |
| | GTTCGAGGGCGACACCCTGGTGAACCGCATCGAGCTGAAGGGCATCGACTT |
| | CAAGGAGGACGGCAACATCCTGGGGCACAAGCTGGAGTACAACTACAACAG |
| | CCACAACGTCTATATCATGGCCGACAAGCAGAAGAACGGCATCAAGGTGAA |
| | CTTCAAGATCCGCCACAACATCGAGGACGGCAGCGTGCAGCTCGCCGACCA |
| | CTACCAGCAGAACACCCCCATCGGCGACGGCCCCGTGCTGCTGCCCGACA |
| | ACCACTACCTGAGCACCCAGTCCGCCCTGAGCAAAGACCCCAACGAGAAGC |
| | GCGATCACATGGTCCTGCTGGAGTTCGTGACCGCCGCCGGGATCACTCTCG |
| | GCATGGACGAGCTGTACAAGTAATAAGOTTAGCAAAAATGTGCTAGTGCCAA |
| | ACGATAGCAAAAATGTGCTAGTGCCAAACGATAGCAAAAATGTGCTAGTGCC |
| | AAACGATAGCAAAAATGTGCTAGTGCCAAAGCCTCGACTGTGCCTTCTAGTT |
| | GCCAGCCATCTGTTGTTTGCCCCTCCCCCGTGCCTTCCTTGACCCTGGAAG |
| | GTGCCACTCCCACTGTCCTTTCCTAATAAAATGAGGAAATTGCATCGCATTG |
| | TCTGAGTAGGTGTCATTCTATTCTGGGGGGTGGGGTGGGGCAGGACAGCAA |
| | GGGGGAGGATTGGGAAGACAATAGCAGGCATGCTGGGGACTCGAGTTAAG |
| | GGCGAATTCCCGATAAGGATCTTCCTAGAGCATGGCTACGTAGATAAGTAGC |
| | ATGGCGGGTTAATCATTAACTACAAGGAACCCCTAGTGATGGAGTTGGCCAC |
| | TCCCTCTCTGCGCGCTCGCTCGCTCACTGAGGCCGGGCGACCAAAGGTCG |
| | CCCGACGCCCGGGCTTTGCCCGGGGGGCCTCAGTGAGCGAGCGAGCGCG |
| | CAGCCTTAATTAACCTAATTCACTGGCCGTCGTTTTACAACGTCGTGACTGG |
| | GAAAACCCTGGCGTTACCCAACTTAATCGCCTTGCAGCACATCCCCCTTTCG |
| | CCAGCTGGCGTAATAGCGAAGAGGCCCGCACCGATCGCCCTTCCCAACAGT |
| | TGCGCAGCCTGAATGGCGAATGGGACGCGCCCTGTAGCGGCGCATTAAGC |
| | GCGGCGGGTGTGGTGGTTACGCGCAGCGTGACCGCTACACTTGCCAGCGC |
| | CCTAGCGCCCGCTCCTTTCGCTTTCTTCCCTTCCTTTCTCGCCACGTTCGCC |
| | GGCTTTCCCCGTCAAGCTCTAAATCGGGGGCTCCCTTTAGGGTTCCGATTTA |
| | GTGCTTTACGGCACCTCGACCCCAAAAAACTTGATTAGGGTGATGGTTCACG |
| | TAGTGGGCCATCGCCCTGATAGACGGTTTTTCGCCCTTTGACGTTGGAGTC |
| | CACGTTCTTTAATAGTGGACTCTTGTTCCAAACTGGAACAACACTCAACCCTA |
| | TCTCGGTCTATTCTTTTGATTTATAAGGGATTTTGCCGATTTCGGCCTATTGG |
| | TTAAAAAATGAGCTGATTTAACAAAAATTTAACGCGAATTTTAACAAAATATTA |
| | ACGCTTACAATTTAGGTGGCACTTTTCGGGGAAATGTGCGCGGAACCCCTAT |
| | TTGTTTATTTTTCTAAATACATTCAAATATGTATCCGCTCATGAGACAATAACC |
| | CTGATAAATGCTTCAATAATATTGAAAAAGGAAGAGTATGAGCCATATTCAAC |
| | GGGAAACGTCGAGGCCGCGATTAAATTCCAACATGGATGCTGATTTATATGG |
| | GTATAAATGGGCTCGCGATAATGTCGGGCAATCAGGTGCGACAATCTATCG |
| | CTTGTATGGGAAGCCCGATGCGCCAGAGTTGTTTCTGAAACATGGCAAAGG |
| | TAGCGTTGCCAATGATGTTACAGATGAGATGGTCAGACTAAACTGGCTGACG |
| | GAATTTATGCCTCTTCCGACCATCAAGCATTTTATCCGTACTCCTGATGATGC |
| | ATGGTTACTCACCACTGCGATCCCCGGAAAAACAGCATTCCAGGTATTAGAA |
| | GAATATCCTGATTCAGGTGAAAATATTGTTGATGCGCTGGCAGTGTTCCTGC |
| | GCCGGTTGCATTCGATTCCTGTTTGTAATTGTCCTTTTAACAGCGATCGCGT |
| | ATTTCGTCTTGCTCAGGCGCAATCACGAATGAATAACGGTTTGGTTGATGCG |
| | AGTGATTTTGATGACGAGCGTAATGGCTGGCCTGTTGAACAAGTCTGGAAAG |
| | AAATGCATAAACTTTTGCCATTCTCACCGGATTCAGTCGTCACTCATGGTGAT |
| | TTCTCACTTGATAACCTTATTTTTGACGAGGGGAAATTAATAGGTTGTATTGA |
| | TGTTGGACGAGTCGGAATCGCAGACCGATACCAGGATCTTGCCATCCTATG |
| | GAACTGCCTCGGTGAGTTTTCTCCTTCATTACAGAAACGGCTTTTTCAAAAAT |
| | ATGGTATTGATAATCCTGATATGAATAAATTGCAGTTTCATTTGATGCTCGAT |
| | GAGTTTTTCTAACTGTCAGACCAAGTTTACTCATATATACTTTAGATTGATTTA |
| | AAACTTCATTTTTAATTTAAAAGGATCTAGGTGAAGATCCTTTTTGATAATCTC |
| | ATGACCAAAATCCCTTAACGTGAGTTTTCGTTCCACTGAGCGTCAGACCCCG |
| | TAGAAAAGATCAAAGGATCTTCTTGAGATCCTTTTTTTCTGCGCGTAATCTGC |
| | TGCTTGCAAACAAAAAAACCACCGCTACCAGCGGTGGTTTGTTTGCCGGATC |
| | AAGAGCTACCAACTCTTTTTCCGAAGGTAACTGGCTTCAGCAGAGCGCAGAT |
| | ACCAAATACTGTTCTTCTAGTGTAGCCGTAGTTAGGCCACCACTTCAAGAAC |
| | TCTGTAGCACCGCCTACATACCTCGCTCTGCTAATCCTGTTACCAGTGGCTG |
| | CTGCCAGTGGCGATAAGTCGTGTCTTACCGGGTTGGACTCAAGACGATAGT |
| | TACCGGATAAGGCGCAGCGGTCGGGCTGAACGGGGGGTTCGTGCACACAG |
| | CCCAGCTTGGAGCGAACGACCTACACCGAACTGAGATACCTACAGCGTGAG |
| | CTATGAGAAAGCGCCACGCTTCCCGAAGGGAGAAAGGCGGACAGGTATCC |
| | GGTAAGCGGCAGGGTCGGAACAGGAGAGCGCACGAGGGAGCTTCCAGGG |
| | GGAAACGCCTGGTATCTTTATAGTCCTGTCGGGTTTCGCCACCTCTGACTTG |
| | AGCGTCGATTTTTGTGATGCTCGTCAGGGGGGCGGAGCCTATGGAAAAACG |
| | CCAGCAACGCGGCCTTTTTACGGTTCCTGGCCTTTTGCTGGCCTTTTGCTCA |
| | CATGTTCTTTCCTGCGTTATCCCCTGATTCTGTGGATAACCGTATTACCGCCT |
| | TTGAGTGAGCTGATACCGCTCGCCGCAGCCGAACGACCGAGCGCAGCGAG |
| | TCAGTGAGCGAGGAAGCGGAAGAGCGCCCAATACGCAAACCGCCTCTCCC |
| | CGCGCGTTGGCCGATTCATTAATGCAGCTGGCACGACAGGTTTCCCGACTG |
| | GAAAGCGGGCAGTGAGCGCAACGCAATTAATGTGAGTTAGCTCACTCATTA |
| | GGCACCCCAGGCTTTACACTTTATGCTTCCGGCTCGTATGTTGTGTGGAATT |
| | GTGAGCGGATAACAATTTCACACAGGAAACAGCTATGACCATGATTACGCCA |
| | GATTTAATTAAGGCCTTAATTAGG |
| SEQ ID NO: 23 | CTGCGCGCTCGCTCGCTCACTGAGGCCGCCCGGGCAAAGCCCGGGCGTCG |
| P1143 Sequence | GGCGACCTTTGGTCGCCCGGCCTCAGTGAGCGAGCGAGCGCGCAGAGAGG |
| 5′ ITR at positions | GAGTGGCCAACTCCATCACTAGGGGTTCCTTGTAGTTAATGATTAACCCGCC |
| 1-130 | ATGCTACTTATCTACGTAGCCATGCTCTAGGAAGATCGGAATTCGCCCTTAA |
| CMV enhancer at | GCTAGCGGCGCGCCACCGGTGCGATCGCCGTTACATAACTTACGGTAAATG |
| positions 233-536 | GCCCGCCTGGCTGACCGCCCAACGACCCCCGCCCATTGACGTCAATAATGA |
| CMV promoter at | CGTATGTTCCCATAGTAACGCCAATAGGGACTTTCCATTGACGTCAATGGGT |
| positions 537-740 | GGAGTATTTACGGTAAACTGCCCACTTGGCAGTACATCAAGTGTATCATATG |
| Chimeric intron at | CCAAGTACGCCCCCTATTGACGTCAATGACGGTAAATGGCCCGCCTGGCAT |
| positions 793-925 | TATGCCCAGTACATGACCTTATGGGACTTTCCTACTTGGCAGTACATCTACG |
| H2B at positions | TATTAGTCATCGCTATTACCATGGTGATGCGGTTTTGGCAGTACATCAATGG |
| 948-1325 | GCGTGGATAGCGGTTTGACTCACGGGGATTTCCAAGTCTCCACCCCATTGA |
| EGFP at positions | CGTCAATGGGAGTTTGTTTTGGCACCAAAATCAACGGGACTTTCCAAAATGT |
| 1344-2063 | CGTAACAACTCCGCCCCATTGACGCAAATGGGGGGTAGGCGTGTACGGTGG |
| miR-182 target | GAGGTCTATATAAGCAGAGCTGGTTTAGTGAACCGTCAGATCCTGCAGAAGT |
| sequences (4) at | TGGTCGTGAGGCACTGGGCAGGTAAGTATCAAGGTTACAAGACAGGTTTAA |
| positions 2071- | GGAGACCAATAGAAACTGGGCTTGTCGAGACAGAGAAGACTCTTGCGTTTC |
| 2095, 2100-2124, | TGATAGGCACCTATTGGTCTTACTGACATCCACTTTGCCTTTCTCTCCACAG |
| 2129-2153, and | GTGTCCAGGCGGCCGCGCCACCATGCCAGAGCCAGCGAAGTCTGCTCCCG |
| 2158-2182 | CCCCGAAAAAGGGCTCCAAGAAGGCGGTGACTAAGGCGCAGAAGAAAGGC |
| bGH polyA signal | GGCAAGAAGCGCAAGCGCAGCCGCAAGGAGAGCTATTCCATCTATGTGTAC |
| at positions 2190-2397 | AAGGTTCTGAAGCAGGTCCACCCTGACACCGGCATTTCGTCCAAGGCCATG |
| 3′ ITR at positions | GGCATCATGAATTCGTTTGTGAACGACATTTTCGAGCGCATCGCAGGTGAG |
| 2485-2614 | GCTTCCCGCCTGGCGCATTACAACAAGCGCTCGACCATCACCTCCAGGGAG |
| | ATCCAGACGGCCGTGCGCCTGCTGCTGCCTGGGGAGTTGGCCAAGCACGC |
| | CGTGTCCGAGGGTACTAAGGCCATCACCAAGTACACCAGCGCTAAGGATCC |
| | ACCGGTCGCCACCATGGTGAGCAAGGGCGAGGAGCTGTTCACCGGGGTGG |
| | TGCCCATCCTGGTCGAGCTGGACGGCGACGTAAACGGCCACAAGTTCAGC |
| | GTGTCCGGCGAGGGCGAGGGCGATGCCACCTACGGCAAGCTGACCCTGAA |
| | GTTCATCTGCACCACCGGCAAGCTGCCCGTGCCCTGGCCCACCCTCGTGAC |
| | CACCCTGACCTACGGCGTGCAGTGCTTCAGCCGCTACCCCGACCACATGAA |
| | GCAGCACGACTTCTTCAAGTCCGCCATGCCCGAAGGCTACGTCCAGGAGCG |
| | CACCATCTTCTTCAAGGACGACGGCAACTACAAGACCCGCGCCGAGGTGAA |
| | GTTCGAGGGCGACACCCTGGTGAACCGCATCGAGCTGAAGGGCATCGACTT |
| | CAAGGAGGACGGCAACATCCTGGGGCACAAGCTGGAGTACAACTACAACAG |
| | CCACAACGTCTATATCATGGCCGACAAGCAGAAGAACGGCATCAAGGTGAA |
| | CTTCAAGATCCGCCACAACATCGAGGACGGCAGCGTGCAGCTCGCCGACCA |
| | CTACCAGCAGAACACCCCCATCGGCGACGGCCCCGTGCTGCTGCCCGACA |
| | ACCACTACCTGAGCACCCAGTCCGCCCTGAGCAAAGACCCCAACGAGAAGC |
| | GCGATCACATGGTCCTGCTGGAGTTCGTGACCGCCGCCGGGATCACTCTCG |
| | GCATGGACGAGCTGTACAAGTAATAAGCTTCGGTGTGAGTTCTACCATTGCC |
| | AAACGATCGGTGTGAGTTCTACCATTGCCAAACGATCGGTGTGAGTTCTACC |
| | ATTGCCAAACGATCGGTGTGAGTTCTACCATTGCCAAAGCCTCGACTGTGCC |
| | TTCTAGTTGCCAGCCATCTGTTGTTTGCCCCTCCCCCGTGCCTTCCTTGACC |
| | CTGGAAGGTGCCACTCCCACTGTCCTTTCCTAATAAAATGAGGAAATTGCAT |
| | CGCATTGTCTGAGTAGGTGTCATTCTATTCTGGGGGGTGGGGTGGGGCAGG |
| | ACAGCAAGGGGGAGGATTGGGAAGACAATAGCAGGCATGCTGGGGACTCG |
| | AGTTAAGGGCGAATTCCCGATAAGGATCTTCCTAGAGCATGGCTACGTAGAT |
| | AAGTAGCATGGGGGGTTAATCATTAACTACAAGGAACCCCTAGTGATGGAGT |
| | TGGCCACTCCCTCTCTGCGCGCTCGCTCGCTCACTGAGGCCGGGCGACCA |
| | AAGGTCGCCCGACGCCCGGGCTTTGCCCGGGGGGCCTCAGTGAGCGAGC |
| | GAGCGCGCAGCCTTAATTAACCTAATTCACTGGCCGTCGTTTTACAACGTCG |
| | TGACTGGGAAAACCCTGGCGTTACCCAACTTAATCGCCTTGCAGCACATCCC |
| | CCTTTCGCCAGCTGGCGTAATAGCGAAGAGGCCCGCACCGATCGCCCTTCC |
| | CAACAGTTGCGCAGCCTGAATGGCGAATGGGACGCGCCCTGTAGCGGCGC |
| | ATTAAGCGCGGCGGGTGTGGTGGTTACGCGCAGCGTGACCGCTACACTTG |
| | CCAGCGCCCTAGCGCCCGCTCCTTTCGCTTTCTTCCCTTCCTTTCTCGCCAC |
| | GTTCGCCGGCTTTCCCCGTCAAGCTCTAAATCGGGGGCTCCCTTTAGGGTT |
| | CCGATTTAGTGCTTTACGGCACCTCGACCCCAAAAAACTTGATTAGGGTGAT |
| | GGTTCACGTAGTGGGCCATCGCCCTGATAGACGGTTTTTCGCCCTTTGACG |
| | TTGGAGTCCACGTTCTTTAATAGTGGACTCTTGTTCCAAACTGGAACAACACT |
| | CAACCCTATCTCGGTCTATTCTTTTGATTTATAAGGGATTTTGCCGATTTCGG |
| | CCTATTGGTTAAAAAATGAGCTGATTTAACAAAAATTTAACGCGAATTTTAAC |
| | AAAATATTAACGCTTACAATTTAGGTGGCACTTTTCGGGGAAATGTGCGCGG |
| | AACCCCTATTTGTTTATTTTTCTAAATACATTCAAATATGTATCCGCTCATGAG |
| | ACAATAACCCTGATAAATGCTTCAATAATATTGAAAAAGGAAGAGTATGAGCC |
| | ATATTCAACGGGAAACGTCGAGGCCGCGATTAAATTCCAACATGGATGCTGA |
| | TTTATATGGGTATAAATGGGCTCGCGATAATGTCGGGCAATCAGGTGCGACA |
| | ATCTATCGCTTGTATGGGAAGCCCGATGCGCCAGAGTTGTTTCTGAAACATG |
| | GCAAAGGTAGCGTTGCCAATGATGTTACAGATGAGATGGTCAGACTAAACTG |
| | GCTGACGGAATTTATGCCTCTTCCGACCATCAAGCATTTTATCCGTACTCCT |
| | GATGATGCATGGTTACTCACCACTGCGATCCCCGGAAAAACAGCATTCCAG |
| | GTATTAGAAGAATATCCTGATTCAGGTGAAAATATTGTTGATGCGCTGGCAG |
| | TGTTCCTGCGCCGGTTGCATTCGATTCCTGTTTGTAATTGTCCTTTTAACAGC |
| | GATCGCGTATTTCGTCTTGCTCAGGCGCAATCACGAATGAATAACGGTTTGG |
| | TTGATGCGAGTGATTTTGATGACGAGCGTAATGGCTGGCCTGTTGAACAAGT |
| | CTGGAAAGAAATGCATAAACTTTTGCCATTCTCACCGGATTCAGTCGTCACT |
| | CATGGTGATTTCTCACTTGATAACCTTATTTTTGACGAGGGGAAATTAATAGG |
| | TTGTATTGATGTTGGACGAGTCGGAATCGCAGACCGATACCAGGATCTTGCC |
| | ATCCTATGGAACTGCCTCGGTGAGTTTTCTCCTTCATTACAGAAACGGCTTTT |
| | TCAAAAATATGGTATTGATAATCCTGATATGAATAAATTGCAGTTTCATTTGAT |
| | GCTCGATGAGTTTTTCTAACTGTCAGACCAAGTTTACTCATATATACTTTAGA |
| | TTGATTTAAAACTTCATTTTTAATTTAAAAGGATCTAGGTGAAGATCCTTTTTG |
| | ATAATCTCATGACCAAAATCCCTTAACGTGAGTTTTCGTTCCACTGAGCGTCA |
| | GACCCCGTAGAAAAGATCAAAGGATCTTCTTGAGATCCTTTTTTTCTGCGCG |
| | TAATCTGCTGCTTGCAAACAAAAAAACCACCGCTACCAGCGGTGGTTTGTTT |
| | GCCGGATCAAGAGCTACCAACTCTTTTTCCGAAGGTAACTGGCTTCAGCAGA |
| | GCGCAGATACCAAATACTGTTCTTCTAGTGTAGCCGTAGTTAGGCCACCACT |
| | TCAAGAACTCTGTAGCACCGCCTACATACCTCGCTCTGCTAATCCTGTTACC |
| | AGTGGCTGCTGCCAGTGGCGATAAGTCGTGTCTTACCGGGTTGGACTCAAG |
| | ACGATAGTTACCGGATAAGGCGCAGCGGTCGGGCTGAACGGGGGGTTCGT |
| | GCACACAGCCCAGCTTGGAGCGAACGACCTACACCGAACTGAGATACCTAC |
| | AGCGTGAGCTATGAGAAAGCGCCACGCTTCCCGAAGGGAGAAAGGCGGAC |
| | AGGTATCCGGTAAGCGGCAGGGTCGGAACAGGAGAGCGCACGAGGGAGCT |
| | TCCAGGGGGAAACGCCTGGTATCTTTATAGTCCTGTCGGGTTTCGCCACCT |
| | CTGACTTGAGCGTCGATTTTTGTGATGCTCGTCAGGGGGGCGGAGCCTATG |
| | GAAAAACGCCAGCAACGCGGCCTTTTTACGGTTCCTGGCCTTTTGCTGGCC |
| | TTTTGCTCACATGTTCTTTCCTGCGTTATCCCCTGATTCTGTGGATAACCGTA |
| | TTACCGCCTTTGAGTGAGCTGATACCGCTCGCCGCAGCCGAACGACCGAGC |
| | GCAGCGAGTCAGTGAGCGAGGAAGCGGAAGAGCGCCCAATACGCAAACCG |
| | CCTCTCCCCGCGCGTTGGCCGATTCATTAATGCAGCTGGCACGACAGGTTT |
| | CCCGACTGGAAAGCGGGCAGTGAGCGCAACGCAATTAATGTGAGTTAGCTC |
| | ACTCATTAGGCACCCCAGGCTTTACACTTTATGCTTCCGGCTCGTATGTTGT |
| | GTGGAATTGTGAGCGGATAACAATTTCACACAGGAAACAGCTATGACCATGA |
| | TTACGCCAGATTTAATTAAGGCCTTAATTAGG |
| SEQ ID NO: 24 | CTGCGCGCTCGCTCGCTCACTGAGGCCGCCCGGGCAAAGCCCGGGCGTCG |
| P1144 Sequence | GGCGACCTTTGGTCGCCCGGCCTCAGTGAGCGAGCGAGCGCGCAGAGAGG |
| 5′ ITR at positions | GAGTGGCCAACTCCATCACTAGGGGTTCCTTGTAGTTAATGATTAACCCGCC |
| 1-130 | ATGCTACTTATCTACGTAGCCATGCTCTAGGAAGATCGGAATTCGCCCTTAA |
| CMV enhancer at | GCTAGCGGCGCGCCACCGGTGCGATCGCCGTTACATAACTTACGGTAAATG |
| positions 233-536 | GCCCGCCTGGCTGACCGCCCAACGACCCCCGCCCATTGACGTCAATAATGA |
| CMV promoter at | CGTATGTTCCCATAGTAACGCCAATAGGGACTTTCCATTGACGTCAATGGGT |
| positions 537-740 | GGAGTATTTACGGTAAACTGCCCACTTGGCAGTACATCAAGTGTATCATATG |
| Chimeric intron at | CCAAGTACGCCCCCTATTGACGTCAATGACGGTAAATGGCCCGCCTGGCAT |
| positions 793-925 | TATGCCCAGTACATGACCTTATGGGACTTTCCTACTTGGCAGTACATCTACG |
| H2B at positions | TATTAGTCATCGCTATTACCATGGTGATGCGGTTTTGGCAGTACATCAATGG |
| 948-1325 | GCGTGGATAGCGGTTTGACTCACGGGGATTTCCAAGTCTCCACCCCATTGA |
| EGFP at positions | CGTCAATGGGAGTTTGTTTTGGCACCAAAATCAACGGGACTTTCCAAAATGT |
| 1344-2063 | CGTAACAACTCCGCCCCATTGACGCAAATGGGGGGTAGGCGTGTACGGTGG |
| miR-183 target | GAGGTCTATATAAGCAGAGCTGGTTTAGTGAACCGTCAGATCCTGCAGAAGT |
| sequences (4) at | TGGTCGTGAGGCACTGGGCAGGTAAGTATCAAGGTTACAAGACAGGTTTAA |
| positions 2071- | GGAGACCAATAGAAACTGGGCTTGTCGAGACAGAGAAGACTCTTGCGTTTC |
| 2092, 2097-2118, | TGATAGGCACCTATTGGTCTTACTGACATCCACTTTGCCTTTCTCTCCACAG |
| 2123-2144, and | GTGTCCAGGCGGCCGCGCCACCATGCCAGAGCCAGCGAAGTCTGCTCCCG |
| 2149-2170 | CCCCGAAAAAGGGCTCCAAGAAGGCGGTGACTAAGGCGCAGAAGAAAGGC |
| bGH polyA signal | GGCAAGAAGCGCAAGCGCAGCCGCAAGGAGAGCTATTCCATCTATGTGTAC |
| at positions 2178-2385 | AAGGTTCTGAAGCAGGTCCACCCTGACACCGGCATTTCGTCCAAGGCCATG |
| 3′ ITR at positions | GGCATCATGAATTCGTTTGTGAACGACATTTTCGAGCGCATCGCAGGTGAG |
| 2473-2602 | GCTTCCCGCCTGGCGCATTACAACAAGCGCTCGACCATCACCTCCAGGGAG |
| | ATCCAGACGGCCGTGCGCCTGCTGCTGCCTGGGGAGTTGGCCAAGCACGC |
| | CGTGTCCGAGGGTACTAAGGCCATCACCAAGTACACCAGCGCTAAGGATCC |
| | ACCGGTCGCCACCATGGTGAGCAAGGGCGAGGAGCTGTTCACCGGGGTGG |
| | TGCCCATCCTGGTCGAGCTGGACGGCGACGTAAACGGCCACAAGTTCAGC |
| | GTGTCCGGCGAGGGCGAGGGCGATGCCACCTACGGCAAGCTGACCCTGAA |
| | GTTCATCTGCACCACCGGCAAGCTGCCCGTGCCCTGGCCCACCCTCGTGAC |
| | CACCCTGACCTACGGCGTGCAGTGCTTCAGCCGCTACCCCGACCACATGAA |
| | GCAGCACGACTTCTTCAAGTCCGCCATGCCCGAAGGCTACGTCCAGGAGCG |
| | CACCATCTTCTTCAAGGACGACGGCAACTACAAGACCCGCGCCGAGGTGAA |
| | GTTCGAGGGCGACACCCTGGTGAACCGCATCGAGCTGAAGGGCATCGACTT |
| | CAAGGAGGACGGCAACATCCTGGGGCACAAGCTGGAGTACAACTACAACAG |
| | CCACAACGTCTATATCATGGCCGACAAGCAGAAGAACGGCATCAAGGTGAA |
| | CTTCAAGATCCGCCACAACATCGAGGACGGCAGCGTGCAGCTCGCCGACCA |
| | CTACCAGCAGAACACCCCCATCGGCGACGGCCCCGTGCTGCTGCCCGACA |
| | ACCACTACCTGAGCACCCAGTCCGCCCTGAGCAAAGACCCCAACGAGAAGC |
| | GCGATCACATGGTCCTGCTGGAGTTCGTGACCGCCGCCGGGATCACTCTCG |
| | GCATGGACGAGCTGTACAAGTAATAAGCTTAGTGAATTCTACCAGTGCCATA |
| | CGATAGTGAATTCTACCAGTGCCATACGATAGTGAATTCTACCAGTGCCATA |
| | CGATAGTGAATTCTACCAGTGCCATAGCCTCGACTGTGCCTTCTAGTTGCCA |
| | GCCATCTGTTGTTTGCCCCTCCCCCGTGCCTTCCTTGACCCTGGAAGGTGC |
| | CACTCCCACTGTCCTTTCCTAATAAAATGAGGAAATTGCATCGCATTGTCTGA |
| | GTAGGTGTCATTCTATTCTGGGGGGTGGGGTGGGGCAGGACAGCAAGGGG |
| | GAGGATTGGGAAGACAATAGCAGGCATGCTGGGGACTCGAGTTAAGGGCG |
| | AATTCCCGATAAGGATOTTCCTAGAGCATGGCTACGTAGATAAGTAGCATGG |
| | CGGGTTAATCATTAACTACAAGGAACCCCTAGTGATGGAGTTGGCCACTCCC |
| | TCTCTGCGCGCTCGCTCGCTCACTGAGGCCGGGCGACCAAAGGTCGCCCG |
| | ACGCCCGGGCTTTGCCCGGGGGGCCTCAGTGAGCGAGCGAGCGCGCAGC |
| | CTTAATTAACCTAATTCACTGGCCGTCGTTTTACAACGTCGTGACTGGGAAA |
| | ACCCTGGCGTTACCCAACTTAATCGCCTTGCAGCACATCCCCCTTTCGCCAG |
| | CTGGCGTAATAGCGAAGAGGCCCGCACCGATCGCCCTTCCCAACAGTTGCG |
| | CAGCCTGAATGGCGAATGGGACGCGCCCTGTAGCGGCGCATTAAGCGCGG |
| | CGGGTGTGGTGGTTACGCGCAGCGTGACCGCTACACTTGCCAGCGCCCTA |
| | GCGCCCGCTCCTTTCGCTTTCTTCCCTTCCTTTCTCGCCACGTTCGCCGGCT |
| | TTCCCCGTCAAGCTCTAAATCGGGGGCTCCCTTTAGGGTTCCGATTTAGTGC |
| | TTTACGGCACCTCGACCCCAAAAAACTTGATTAGGGTGATGGTTCACGTAGT |
| | GGGCCATCGCCCTGATAGACGGTTTTTCGCCCTTTGACGTTGGAGTCCACG |
| | TTCTTTAATAGTGGACTCTTGTTCCAAACTGGAACAACACTCAACCCTATCTC |
| | GGTCTATTCTTTTGATTTATAAGGGATTTTGCCGATTTCGGCCTATTGGTTAA |
| | AAAATGAGCTGATTTAACAAAAATTTAACGCGAATTTTAACAAAATATTAACG |
| | CTTACAATTTAGGTGGCACTTTTCGGGGAAATGTGCGCGGAACCCCTATTTG |
| | TTTATTTTTCTAAATACATTCAAATATGTATCCGCTCATGAGACAATAACCCTG |
| | ATAAATGCTTCAATAATATTGAAAAAGGAAGAGTATGAGCCATATTCAACGGG |
| | AAACGTCGAGGCCGCGATTAAATTCCAACATGGATGCTGATTTATATGGGTA |
| | TAAATGGGCTCGCGATAATGTCGGGCAATCAGGTGCGACAATCTATCGCTT |
| | GTATGGGAAGCCCGATGCGCCAGAGTTGTTTCTGAAACATGGCAAAGGTAG |
| | CGTTGCCAATGATGTTACAGATGAGATGGTCAGACTAAACTGGCTGACGGAA |
| | TTTATGCCTCTTCCGACCATCAAGCATTTTATCCGTACTCCTGATGATGCATG |
| | GTTACTCACCACTGCGATCCCCGGAAAAACAGCATTCCAGGTATTAGAAGAA |
| | TATCCTGATTCAGGTGAAAATATTGTTGATGCGCTGGCAGTGTTCCTGCGCC |
| | GGTTGCATTCGATTCCTGTTTGTAATTGTCCTTTTAACAGCGATCGCGTATTT |
| | CGTCTTGCTCAGGCGCAATCACGAATGAATAACGGTTTGGTTGATGCGAGT |
| | GATTTTGATGACGAGCGTAATGGCTGGCCTGTTGAACAAGTCTGGAAAGAAA |
| | TGCATAAACTTTTGCCATTCTCACCGGATTCAGTCGTCACTCATGGTGATTTC |
| | TCACTTGATAACCTTATTTTTGACGAGGGGAAATTAATAGGTTGTATTGATGT |
| | TGGACGAGTCGGAATCGCAGACCGATACCAGGATCTTGCCATCCTATGGAA |
| | CTGCCTCGGTGAGTTTTCTCCTTCATTACAGAAACGGCTTTTTCAAAAATATG |
| | GTATTGATAATCCTGATATGAATAAATTGCAGTTTCATTTGATGCTCGATGAG |
| | TTTTTCTAACTGTCAGACCAAGTTTACTCATATATACTTTAGATTGATTTAAAA |
| | CTTCATTTTTAATTTAAAAGGATCTAGGTGAAGATCCTTTTTGATAATCTCATG |
| | ACCAAAATCCCTTAACGTGAGTTTTCGTTCCACTGAGCGTCAGACCCCGTAG |
| | AAAAGATCAAAGGATCTTCTTGAGATCCTTTTTTTCTGCGCGTAATCTGCTGC |
| | TTGCAAACAAAAAAACCACCGCTACCAGCGGTGGTTTGTTTGCCGGATCAAG |
| | AGCTACCAACTCTTTTTCCGAAGGTAACTGGCTTCAGCAGAGCGCAGATACC |
| | AAATACTGTTCTTCTAGTGTAGCCGTAGTTAGGCCACCACTTCAAGAACTCT |
| | GTAGCACCGCCTACATACCTCGCTCTGCTAATCCTGTTACCAGTGGCTGCTG |
| | CCAGTGGCGATAAGTCGTGTCTTACCGGGTTGGACTCAAGACGATAGTTAC |
| | CGGATAAGGCGCAGCGGTCGGGCTGAACGGGGGGTTCGTGCACACAGCCC |
| | AGCTTGGAGCGAACGACCTACACCGAACTGAGATACCTACAGCGTGAGCTA |
| | TGAGAAAGCGCCACGCTTCCCGAAGGGAGAAAGGCGGACAGGTATCCGGT |
| | AAGCGGCAGGGTCGGAACAGGAGAGCGCACGAGGGAGCTTCCAGGGGGA |
| | AACGCCTGGTATCTTTATAGTCCTGTCGGGTTTCGCCACCTCTGACTTGAGC |
| | GTCGATTTTTGTGATGCTCGTCAGGGGGGCGGAGCCTATGGAAAAACGCCA |
| | GCAACGCGGCCTTTTTACGGTTCCTGGCCTTTTGCTGGCCTTTTGCTCACAT |
| | GTTCTTTCCTGCGTTATCCCCTGATTCTGTGGATAACCGTATTACCGCCTTTG |
| | AGTGAGCTGATACCGCTCGCCGCAGCCGAACGACCGAGCGCAGCGAGTCA |
| | GTGAGCGAGGAAGCGGAAGAGCGCCCAATACGCAAACCGCCTCTCCCCGC |
| | GCGTTGGCCGATTCATTAATGCAGCTGGCACGACAGGTTTCCCGACTGGAA |
| | AGCGGGCAGTGAGCGCAACGCAATTAATGTGAGTTAGCTCACTCATTAGGC |
| | ACCCCAGGCTTTACACTTTATGCTTCCGGCTCGTATGTTGTGTGGAATTGTG |
| | AGCGGATAACAATTTCACACAGGAAACAGCTATGACCATGATTACGCCAGAT |
| | TTAATTAAGGCCTTAATTAGG |

### Expression of Exogenous Nucleic Acids in Mammalian Cells

One platform that can be used to achieve therapeutically effective intracellular concentrations of exogenous polynucleotides in mammalian cells is via the stable expression of the polynucleotide (e.g., by integration into the nuclear or mitochondrial genome of a mammalian cell, or by episomal concatemer formation in the nucleus of a mammalian cell). In order to introduce exogenous polynucleotides into a mammalian cell, polynucleotides can be incorporated into a vector. Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

Polynucleotides can also be introduced into a mammalian cell by targeting a vector containing a polynucleotide of interest to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

The vectors described herein may be used to express one or more exogenous polynucleotides that can be transcribed to produce a desired expression product in an inner ear cell. The polynucleotide can be a polynucleotide that encodes a protein, an inhibitory RNA (e.g., an siRNA or shRNA), or a component of a gene editing system. In some embodiments, the polynucleotide is a polynucleotide that corresponds to a wild-type form of a gene implicated in hearing loss and/or vestibular dysfunction (e.g., a polynucleotide that encodes a wild-type form of the protein). Mutations in a variety of genes, such as Myosin 7A (MYO7A), POU Class 4 Homeobox 3 (POU4F3), Solute Carrier Family 17 Member 8 (SLC17A8), Gap Junction Protein Beta 2 (GJB2), Claudin 14 (CLDN14), Cochlin (COCH), Protocadherin Related 15 (PCDH15), and Transmembrane 1 (TMC1), have been linked to sensorineural hearing loss and/or deafness, and some of these mutations, such as mutations in MYO7A, POU4F3, and COCH are also associated with vestibular dysfunction. In some embodiments, the polynucleotide is a polynucleotide that is normally expressed in healthy inner ear cells, such as a polynucleotide corresponding to a gene involved in inner ear cell development, function, cell fate specification, regeneration, survival, proliferation, and/or maintenance. The polynucleotide can also encode a protein, an inhibitory RNA, or a component of a gene editing system that regulates (e.g., promotes or improves) inner ear cell development, function, cell fate specification, regeneration, survival, proliferation, and/or maintenance.

### Polynucleotides Encoding Proteins

In some embodiments, the vector described herein contains a polynucleotide corresponding to a wild-type version of a gene that is implicated in hearing loss and/or vestibular dysfunction. Examples of such genes are listed in the second column of Table 4, below. Vectors containing the wild-type version of a gene in the second (right) column can be administered to a subject to treat the associated disease or condition listed in the first (left) column.

TABLE 4
Genes implicated in sensorineural hearing
loss and vestibular dysfunction

| Condition | Gene(s) |
|---|---|
| Waardenburg syndrome (WS) | PAX3 |
| | MITF |
| | EDNRB |
| | EDN3 |
| | SOX10 |
| Branchiootorenal spectrum disorders | EYA1 |
| | SIX1 |
| | SIX5 |
| Neurofibromatosis 2 (NF2) | NF2 |
| Stickler syndrome | COL2A1 |
| | COL11A1 |
| | COL11A2 |
| | COL9A1 |
| | COL9A2 |
| | COL9A3 |
| Usher syndrome type I | MYO7A |
| | USH1C |
| | CDH23 |
| | PCDH15 |
| | USH1G |
| | CIB2 |
| Usher syndrome type II | ADGRV1 |
| | WHRN |
| | USH2A |
| Usher syndrome type III | CLRN1 |
| (OMIM 276902, 614504) | HARS1 |
| Pendred syndrome | SLC26A4 |
| Jervell and Lange-Nielsen syndrome | KCNQ1 |
| | KCNE1 |
| Biotinidase deficiency | BTD |
| Refsum disease | PHYH |
| | PEX7 |
| Alport syndrome | COL4A5 |
| | COL4A3 |
| | COL4A4 |
| Deafness-dystonia-optic neuronopathy | TIMM8A |
| syndrome | |
| (Mohr-Tranebjaerg syndrome) | |
| DFNA20/26 | ACTG1 |
| DFNA440 | CCDC50 |
| DFNA66 | CD164 |
| DFNA4B | CEACAM16 |
| DFNA9 | COCH |
| DFNA13 | COL11A2 |
| DFNA5 | GSDME |
| DFNA1 | DIAPH1 |
| — | DMXL2 |
| DFNA39 | DSPP |
| DFNA10 | EYA4 |
| DFNA3 | GJB2 |
| DFNA2B | GJB3 |
| DFNA3 | GJB6 |
| DFNA28 | GRHL2 |
| DFNA68 | HOMER2 |
| DFNA2 | KCNQ4 |
| DFNA50 | MIR96 |
| DFNA70 | MCM2 |
| DFNA4 | MYH14 |
| DFNA17 | MYH9 |
| DFNA48 | MYO1A |
| DFNA22 | MYO6 |
| DFNA11 | MYO7A |
| DFNA67 | OSBPL2 |
| DFNA41 | P2RX2 |
| DFNA15 | POU4F3 |
| DFNA23 | SIX1 |
| DFNA25 | SLC17A8 |
| DFNA65 | TBC1D24 |
| DFNA8/12 | TECTA |
| DFNA51 | TJP2 |
| | FAM189A2 |
| DFNA36 | TMC1 |
| DFNA6/14/38 | WFS1 |
| DFNB44 | ADCY1 |
| DFNB49 | BDP1 |
| DFNB73 | BSND |
| DFNB93 | CABP2 |
| DFNB105 | CDC14A |
| DFNB12 | CDH23 |
| DFNB48 | CIB2 |
| DFNB29 | CLDN14 |
| DFNB103 | CLIC5 |
| DFNB53 | COL11A2 |
| DFNB66 | DCDC2 |
| DFNB59 | PJVK |
| DFNB88 | ELMOD3 |
| DFNB102 | EPS8 |
| — | EPS8L2 |
| DFNB36 | ESPN |
| DFNB35 | ESRRB |
| DFNB15/72/95 | GIPC3 |
| DFNB1 | GJB2 |
| DFNB1 | GJB6 |
| DFNB32/82 | GPSM2 |
| DFNB25 | GRXCR1 |
| DFNB101 | GRXCR2 |
| DFNB39 | HGF |
| DFNB42 | ILDR1 |
| DFNB89 | KARS1 |
| DFNB67 | LHFPL5 |
| DFNB77 | LOXHD1 |
| DFNB63 | LRTOMT |
| DFNB49 | MARVELD2 |
| DFNB97 | MET |
| DFNB74 | MSRB3 |
| DFNB3 | MYO15A |
| DFNB30 | MYO3A |
| DFNB37 | MYO6 |
| DFNB2 | MYO7A |
| DFNB94 | NARS2 |
| DFNB18B | OTOG |
| DFNB84 | OTOGL |
| DFNB22 | OTOA |
| DFNB9 | OTOF |
| DFNB23 | PCDH15 |
| DFNB70 | PNPT1 |
| DFNB84 | PTPRQ |
| DFNB24 | RDX |
| DFNB104 | RIPOR2 |
| — | ROR1 |
| DFNB68 | S1PR2 |
| DFNB91 | SERPINB6 |
| DFNB60 | SLC22A4 |
| DFNB4 | SLC26A4 |
| DFNB61 | SLC26A5 |
| DFNB16 | STRC |
| DFNB76 | SYNE4 |
| DFNB21 | TECTA |
| DFNB86 | TBC1D24 |
| DFNB7/11 | TMC1 |
| DFNB99 | TMEM132E |
| DFNB6 | TMIE |
| DFNB8/10 | TMPRSS3 |
| DFNB79 | TPRN |
| DFNB28 | TRIOBP |
| DFNB98 | TSPEAR |
| DFNB18 | USH1C |
| — | WBP2 |
| DFNB31 | WHRN |
| DFNX1 | PRPS1 |
| DFNX2 | POU3F4 |
| DFNX4 | SMPX |
| DFNX5 | AIFM1 |
| DFNX6 | COL4A6 |
| Non-syndromic hearing loss and | MT-RNR1 |
| deafness, mitochondrial | MT-TS1 |
| | MT-CO1 |

The vectors described herein may be used to express a polynucleotide that is normally expressed in healthy inner ear cells, such as a polynucleotide corresponding to a gene involved in inner ear cell development, function, cell fate specification, regeneration, survival, proliferation, and/or maintenance. The nucleic acid can also encode a polynucleotide, an inhibitory RNA, or a component of a gene editing system that regulates (e.g., promotes or improves) inner ear cell development, function, cell fate specification, regeneration, survival, proliferation, and/or maintenance. Exemplary polynucleotides that can be expressed in an inner ear cell using a vector described herein are provided in Table 5, below, along with the inner ear cell type(s) in which they can be expressed. Accession numbers for the polynucleotides of Tables 4 and 5 are provided in Table 6.

TABLE 5
Polynucleotides that can be expressed in one or more inner ear cell types

| Cell type | Polynucleotide |
|---|---|
| Inner hair cells (IHCs) | Otoferlin (Otof), Soluble Carrier Family 17 Member 8 (Slc17a8, also |
| | known as Vglut3) |
| Outer hair cells (OHCs) | Stereocilin (Strc), Cholinergic Receptor Nicotinic Alpha 9 Subunit |
| | (Chrna9), Cholinergic Receptor Nicotinic Alpha 10 Subunit |
| | (Chrna10), Oncomodulin (Ocm) |
| IHCs and vestibular hair cells | Whirlin (Whrn) |
| Cochlear hair cells (IHCs and | Atonal BHLH Transcription Factor 1 (Atoh1), POU Class 4 |
| OHCs) | Homeobox 3 (Pou4f3), Growth Factor Independent 1 |
| | Transcriptional Repressor (Gfi1), ISL LIM Homeobox 1 (Isl1), Clarin |
| | 1 (Clrn1), Protocadherin Related 15 (Pcdh15), Cadherin Related 23 |
| | (Cdh23), Myosin 7a (Myo7a), Transmembrane Channel Like 1 |
| | (Tmc1), Harmonin (Ush1c) |
| Cells of the stria vascularis (SV) | Potassium Voltage-Gated Channel Subfamily Q Member (Kcnq1), |
| | Potassium Voltage-Gated Channel Subfamily E Regulatory Subunit |
| | 1 (Kcne1), Gap Junction Protein Beta 2 (Gjb2), Gap Junction |
| | Protein Beta 6 (Gjb6), Tyrosinase (Tyr), a nuclease (e.g., CRISPR |
| | Associated Protein 9 (Cas9), Transcription Activator-Like Effector |
| | Nuclease (TALEN), Zinc Finger Nuclease (ZFN), or gRNA) |
| Fibrocytes/mesenchyme | Collagens (e.g., Collagen Type I Alpha 1 Chain (Colla1), Collagen |
| | Type I Alpha 2 Chain (Col1a2), Collagen Type II Alpha 1 Chain |
| | (Col2a1), or other collagen genes) |
| Interdental cells | Carcinoembryonic Antigen Related Cell Adhesion Molecule 16 |
| | (Ceacam16), Otoancorin (Otoa), Gjb2, Gjb6 |
| Spiral prominence cells | Solute Carrier Family 26 Member 4 (Slc26a4) |
| Root cells | Slc26a4 |
| Cochlear and vestibular | SRY-Box 9 (Sox9), Spalt Like Transcription Factor 2 (Sall2), |
| supporting cells | Calmodulin Binding Transcription Activator 1 (Camta1), Hes |
| | Related Family BHLH Transcription Factor With YRPW Motif 2 |
| | (Hey2), Gata Binding Protein 2 (Gata2), Hes Related Family BHLH |
| | Transcription Factor With YRPW Motif 1 (Hey1), Ceramide |
| | Synthase 2 (Lass2), SRY-Box 10 (Sox10), GATA Binding Protein 3 |
| | (Gata3), Cut Like Homeobox 1 (Cux1), Nuclear Receptor Subfamily |
| | 2 Group F Member (Nr2f1), Hes Family BHLH Transcription Factor |
| | 1 (Hes1), RAR Related Orphan Receptor B (Rorb), Jun Proto- |
| | Oncogene AP-1 Transcription Factor Subunit (Jun), Zinc Finger |
| | Protein 667 (Zfp667), LIM Homeobox 3 (Lhx3), Nescient Helix- |
| | Loop-Helix 1 (Nhlh1), MAX Dimerization Protein 4 (Mxd4), Zinc |
| | Finger MIZ-Type Containing 1 (Zmiz1), Myelin Transcription Factor |
| | 1 (Myt1), Signal Transducer And Activator Of Transcription 3 |
| | (Stat3), BarH Like Homeobox 1 (Barhl1), Thymocyte Selection |
| | Associated High Mobility Group Box (Tox), Prospero Homeobox 1 |
| | (Prox1), Nuclear Factor I A (Nfia), Thyroid Hormone Receptor Beta |
| | (Thrb), MYCL Proto-Oncogene BHLH Transcription Factor (Mycl1), |
| | Lysine Demethylase 5A (Kdm5a), CAMP Responsive Element |
| | Binding Protein 3 Like 4 (Creb3l4), ETS Variant 1 (Etv1), Paternally |
| | Expressed 3 (Peg3), BTB Domain And CNC Homolog 2 (Bach2), |
| | ISL LIM Homeobox (Isl1), Zinc Finger And BTB Domain Containing |
| | 38 (Zbtb38), Limb Bud And Heart Development (Lbh), Tubby |
| | Bipartite Transcription Factor (Tub), Ubiquitin C (Hmg20), RE1 |
| | Silencing Transcription Factor (Rest), Zinc Finger Protein 827 |
| | (Zfp827), AF4/FMR2 Family Member 3 (Aff3), PBX/Knotted 1 |
| | Homeobox 2 (Pknox2), AT-Rich Interaction Domain 3B (Arid3b), |
| | MLX Interacting Protein (Mlxip), Zinc Finger Protein (Zfp532), |
| | IKAROS Family Zinc Finger 2 (Ikzf2), Spalt Like Transcription |
| | Factor 1 (Sall1), SIX Homeobox 2 (Six2), Spalt Like Transcription |
| | Factor 3 (Sall3), Lin-28 Homolog B (Lin28b), Pou4f3, Regulatory |
| | Factor X7 (Rfx7), Atoh1, a polynucleotide encoding an Atoh1 |
| | variant containing mutations at amino acids 328, 331, and/or 334 |
| | (e.g., S328A, S331A, S334A, S328A/S331A, S328A/S334A, |
| | S331A/S334A, and S328A/S331A/S334, e.g., a polynucleotide |
| | encoding a variant having the sequence of any one of SEQ ID |
| | NOs: 43-49), Gfi1, SRY-Box 4 (Sox4), Brain Derived Neurotrophic |
| | Factor (Bdnf), Neurotrophin 3 (Ntf3), SRY-Box 11 (Sox11), TEA |
| | Domain Transcription Factor 2 (Tead2), Yes Associated Protein 1 |
| | (Yap1), a nuclease (e.g., Cas9, TALEN, ZFN, or gRNA) |
| Vestibular and cochlear hair | Bdnf, Ntf3, Transmembrane and Tetratricopeptide Repeat |
| cells | Containing 4 (Tmtc4), a nuclease (e.g., Cas9, TALEN, ZFN, or |
| | gRNA) |
| Border cells (cochlear supporting | Bdnf, Ntf3, Tectorin Beta (Tectb), Tectorin Alpha (Tecta), Gjb2, |
| cell subtype) | Gjb6 |
| Inner phalangeal cells (cochlear | Bdnf, Ntf3, Tectb, Tecta, Transmembrane Protein 16A (Tmem16a), |
| supporting cell subtype) | Gjb2, Gjb6 |
| Pillar cells (cochlear supporting | Nerve Growth Factor Receptor (Ngfr), Bdnf, Ntf3, Tectb, Tecta, |
| cell subtype) | Gjb2, Gjb6 |
| Deiters cells (cochlear | Bdnf, Ntf3, Tectb, Tecta, Ikzf2, Gjb2, Gjb6 |
| supporting cell subtype) | |
| Hensen's cells (cochlear | Gjb2, Gjb6 |
| supporting cell subtype) | |
| Claudius cells (cochlear | Gjb2, Gjb6 |
| supporting cell subtype) | |
| Spiral ganglion neurons (SGN) | Bdnf, Ntf3, a nuclease (e.g., Cas9, TALEN, ZFN, or gRNA), shRNA |
| | directed to RGMA, |
| Scarpa's ganglion | Bdnf, Ntf3, shRNA directed to RGMA |
| All fibrocytes and epithelia | Gjb2, Gjb6 |
| Vestibular dark cells | Kcnq1, Kcne1, Slc26a4 |
| Glia | Peripheral Myelin Protein 22 (Pmp22), Bdnf, Ntf3, Myelin Protein |
| | Zero (Mpz) |

TABLE 6
Accession numbers for polynucleotides listed in Tables 4 and 5

| | NCBI Accession |
|---|---|
| Gene name | number |
| Otof, Otoferlin (variant 1) | NM_194248 |
| Otof, Otoferlin (variant 2) | NM_004802 |
| Otof, Otoferlin (variant 3) | NM_194322 |
| Otof, Otoferlin (variant 4) | NM_194323 |
| Otof, Otoferlin (variant 5) | NM_001287489 |
| Vglut3, Vesicular glutamate transporter 3 (variant 1) | NM_139319 |
| Vglut3, Vesicular glutamate transporter 3 (variant 2) | NM_001145288 |
| Strc, Stereocilin | NM_153700 |
| Tmc1, Transmembrane channel like 1 | NM_138691 |
| Myo7a, Myosin VIIa (variant 1) | NM_000260 |
| Myo7a, Myosin VIIa (variant 2) | NM_001127180 |
| Harmonin (USH1C, variant 1) | NM_005709 |
| Harmonin (USH1C, variant b3) | NM_153676 |
| Harmonin (USH1C, variant 3) | NM_001297764 |
| Whirlin (variant 1) | NM_015404 |
| Whirlin (variant 2) | NM_001083885 |
| Whirlin (variant 3) | NM_001173425 |
| Atoh1, Atonal BHLH transcription factor 1 | NM_005172 |
| Pou4f3, POU class 4 homeobox 3 | NM_002700 |
| Gfi1, Growth factor independent 1 transcriptional repressor (variant 1) | NM_005263 |
| Gfi1, Growth factor independent 1 transcriptional repressor (variant 2) | NM_001127215 |
| Gfi1, Growth factor independent 1 transcriptional repressor (variant 3) | NM_001127216 |
| Isl1, ISL LIM homeobox 1 | NM_00220 |
| Clrn1, Clarin 1 (variant 1) | NM_174878 |
| | NM_174879 |
| Clrn1, Clarin 1 (variant 4) | NM_052995 |
| Clrn1, Clarin 1 (variant 5) | NM_001195794 |
| Clrn1, Clarin 1 (variant 6) | NM_001256819 |
| Pcdh15, Protocadherin related 15 | NM_033056 |
| Cdh23, Cadherin related 23 (variant 1) | NM_022124 |
| Cdh23, Cadherin related 23 (variant 2) | NM_052836 |
| Cdh23, Cadherin related 23 (variant 3) | NM_001171930 |
| Cdh23, Cadherin related 23 (variant 4) | NM_001171931 |
| Cdh23, Cadherin related 23 (variant 5) | NM_001171932 |
| Cdh23, Cadherin related 23 (variant 6) | NM_001171933 |
| Cdh23, Cadherin related 23 (variant 7) | NM_001171934 |
| Cdh23, Cadherin related 23 (variant 8) | NM_001171935 |
| Cdh23, Cadherin related 23 (variant 9) | NM_001171936 |
| Kcnq1, Potassium voltage-gated channel subfamily Q member 1 (variant 1) | NM_000218 |
| Kcnq1, Potassium voltage-gated channel subfamily Q member 1 (variant 2) | NM_181798 |
| Kcne1, Potassium voltage-gated channel subfamily E regulatory subunit 1 | NM_001127670 |
| (variant 1) | |
| Kcne1, Potassium voltage-gated channel subfamily E regulatory subunit 1 | NM_000219 |
| (variant 2) | |
| Kcne1, Potassium voltage-gated channel subfamily E regulatory subunit 1 | NM_001127668 |
| (variant 3) | |
| Kcne1, Potassium voltage-gated channel subfamily E regulatory subunit 1 | NM_001127669 |
| (variant 4) | |
| Kcne1, Potassium voltage-gated channel subfamily E regulatory subunit 1 | NM_001270402 |
| (variant 5) | |
| Kcne1, Potassium voltage-gated channel subfamily E regulatory subunit 1 | NM_001270403 |
| (variant 6) | |
| Kcne1, Potassium voltage-gated channel subfamily E regulatory subunit 1 | NM_001270404 |
| (variant 7) | |
| Kcne1, Potassium voltage-gated channel subfamily E regulatory subunit 1 | NM_001270405 |
| (variant 8) | |
| Col1a1, Collagen type I alpha 1 chain | NM_000088 |
| Col1a2, Collagen type I alpha 2 chain | NM_000089 |
| Col2a1, Collagen type II alpha 1 chain (variant 1) | NM_001844 |
| Col2a1, Collagen type II alpha 1 chain (variant 2) | NM_033150 |
| Col3a1, Collagen type III alpha 1 chain | NM_000090 |
| Col4a1, Collagen type IV alpha 1 chain (variant 1) | NM_001845 |
| Col4a1, Collagen type IV alpha 1 chain (variant 2) | NM_001303110 |
| Col4a2, Collagen type IV alpha 2 chain | NM_001846 |
| Col4a3, Collagen type IV alpha 3 chain | NM_000091 |
| Col4a4, Collagen type IV alpha 4 chain | NM_000092 |
| Col4a5, Collagen type IV alpha 5 chain (variant 1) | NM_000495 |
| Col4a5, Collagen type IV alpha 5 chain (variant 2) | NM_033380 |
| Col4a6, Collagen type IV alpha 6 chain (variant A) | NM_001847 |
| Col4a6, Collagen type IV alpha 6 chain (variant B) | NM_033641 |
| Col4a6, Collagen type IV alpha 6 chain (variant 3) | NM_001287758 |
| Col4a6, Collagen type IV alpha 6 chain (variant 4) | NM_001287759 |
| Col4a6, Collagen type IV alpha 6 chain (variant 5) | NM_001287760 |
| Col5a1, Collagen type V alpha 1 chain (variant 1) | NM_000093 |
| Col5a1, Collagen type V alpha 1 chain (variant 2) | NM_001278074 |
| Col5a2, Collagen type V alpha 2 chain | NM_000393 |
| Col5a3, Collagen type V alpha 3 chain | NM_015719 |
| Col6a1, Collagen type VI alpha 1 chain | NM_001848 |
| Col6a2, Collagen type VI alpha 2 chain (variant 2C2) | NM_001849 |
| Col6a2, Collagen type VI alpha 2 chain (variant 2C2a) | NM_058174 |
| Col6a2, Collagen type VI alpha 2 chain (variant 2C2a') | NM_058175 |
| Col6a3, Collagen type VI alpha 3 chain (variant 1) | NM_004369 |
| Col6a3, Collagen type VI alpha 3 chain (variant 2) | NM_057164 |
| Col6a3, Collagen type VI alpha 3 chain (variant 3) | NM_057165 |
| Col6a3, Collagen type VI alpha 3 chain (variant 4) | NM_057166 |
| Col6a3, Collagen type VI alpha 3 chain (variant 5) | NM_057167 |
| Col6a5, Collagen type VI alpha 5 chain (variant 1) | NM_001278298 |
| Col6a5, Collagen type VI alpha 5 chain (variant 2) | NM_153264 |
| Col6a6, Collagen type VI alpha 6 chain | NM_001102608 |
| Col7a1, Collagen type VII alpha 1 chain | NM_000094 |
| Col8a1, Collagen type VIII alpha 1 chain (variant 1) | NM_001850 |
| Col8a1, Collagen type VIII alpha 1 chain (variant 2) | NM_020351 |
| Col8a2, Collagen type VIII alpha 2 chain (variant 1) | NM_005202 |
| Col8a2, Collagen type VIII alpha 2 chain (variant 2) | NM_001294347 |
| Col9a1, Collagen type IX alpha 1 chain (variant 1) | NM_001851 |
| Col9a1, Collagen type IX alpha 1 chain (variant 2) | NM_078485 |
| Col9a2, Collagen type IX alpha 2 chain | NM_001852 |
| Col9a3, Collagen type IX alpha 3 chain | NM_001853 |
| Col10a1, Collagen type X alpha 1 chain | NM_000493 |
| Col11a1, Collagen type XI alpha 1 chain (variant A) | NM_001854 |
| Col11a1, Collagen type XI alpha 1 chain (variant B) | NM_080629 |
| Col11a1, Collagen type XI alpha 1 chain (variant C) | NM_080630 |
| | NM_001168249 |
| Col11a1, Collagen type XI alpha 1 chain (variant E) | NM_001190709 |
| Col11a2, Collagen type XI alpha 2 chain (variant 1) | NM_080680 |
| Col11a2, Collagen type XI alpha 2 chain (variant 2) | NM_080681 |
| Col11a2, Collagen type XI alpha 2 chain (variant 3) | NM_080679 |
| Col11a2, Collagen type XI alpha 2 chain (variant 4) | NM_001163771 |
| Col12a1, Collagen type XII alpha 1 chain (short variant) | NM_080645 |
| Col12a1, Collagen type XII alpha 1 chain (long variant) | NM_004370 |
| Col13a1, Collagen type XIII alpha 1 chain (variant 1) | NM_001130103 |
| Col13a1, Collagen type XIII alpha 1 chain (variant 5) | NM_080801 |
| Col13a1, Collagen type XIII alpha 1 chain (variant 11) | NM_080800 |
| Col13a1, Collagen type XIII alpha 1 chain (variant 15) | NM_080802 |
| Col13a1, Collagen type XIII alpha 1 chain (variant 21) | NM_080798 |
| Col13a1, Collagen type XIII alpha 1 chain (variant 22) | NM_001320951 |
| Col14a1, Collagen type XIV alpha 1 chain | NM_021110 |
| Col15a1, Collagen type XV alpha 1 chain | NM_001855 |
| Col16a1, Collagen type XVI alpha 1 chain | NM_001856 |
| Col17a1, Collagen type XVII alpha 1 chain | NM_000494 |
| Col18a1, Collagen type XVIII alpha 1 chain (variant 1) | NM_030582 |
| Col18a1, Collagen type XVIII alpha 1 chain (variant 2) | NM_130444 |
| Col18a1, Collagen type XVIII alpha 1 chain (variant 3) | NM_130445 |
| Col19a1, Collagen type XIX alpha 1 chain | NM_001858 |
| Col20a1, Collagen type XX alpha 1 chain | NM_020882 |
| Col21a1, Collagen type XXI alpha 1 chain (variant 1) | NM_030820 |
| Col21a1, Collagen type XXI alpha 1 chain (variant 2) | NM_001318751 |
| Col21a1, Collagen type XXI alpha 1 chain (variant 3) | NM_001318752 |
| Col21a1, Collagen type XXI alpha 1 chain (variant 4) | NM_001318753 |
| Col21a1, Collagen type XXI alpha 1 chain (variant 5) | NM_001318754 |
| Col22a1, Collagen type XXII alpha 1 chain | NM_152888 |
| Col23a1, Collagen type XXIII alpha 1 chain | NM_173465 |
| Col24a1, Collagen type XXIV alpha 1 chain (variant 1) | NM_152890 |
| Col24a1, Collagen type XXIV alpha 1 chain (variant 2) | NM_001349955 |
| Col25a1, Collagen type XXV alpha 1 chain (variant 1) | NM_198721 |
| Col25a1, Collagen type XXV alpha 1 chain (variant 2) | NM_032518 |
| Col25a1, Collagen type XXV alpha 1 chain (variant 3) | NM_001256074 |
| Col26a1, Collagen type XXVI alpha 1 chain (variant 1) | NM_001278563 |
| Col26a1, Collagen type XXVI alpha 1 chain (variant 2) | NM_133457 |
| Col27a1, Collagen type XXVII alpha 1 chain | NM_032888 |
| Col28a1, Collagen type XXVIII alpha 1 chain | NM_001037763 |
| Ceacam16, Carcinoembryonic antigen related cell adhesion molecule 16 | NM_001039213 |
| Otoa, Otoancorin (variant 1) | NM_144672 |
| Otoa, Otoancorin (variant 2) | NM_170664 |
| Otoa, Otoancorin (variant 3) | NM_001161683 |
| Slc26a4, Solute carrier family 26 member 4 | NM_000441 |
| Sox9, SRY-box 9 | NM_000346 |
| Sox10, SRY-box 10 | NM_006941 |
| Sall2, Spalt like transcription factor 2 (variant 1) | NM_005407 |
| Sall2, Spalt like transcription factor 2 (variant 2) | NM_001291446 |
| Sall2, Spalt like transcription factor 2 (variant 3) | NM_001291447 |
| Sall2, Spalt like transcription factor 2 (variant 6) | NM_001364564 |
| Camta1, Calmodulin binding transcription activator 1 (variant 1) | NM_015215 |
| Camta1, Calmodulin binding transcription activator 1 (variant 2) | NM_00119556 |
| Camta1, Calmodulin binding transcription activator 1 (variant 3) | NM_001242701 |
| Camta1, Calmodulin binding transcription activator 1 (variant 5) | NM_001349608 |
| Camta1, Calmodulin binding transcription activator 1 (variant 6) | NM_001349609 |
| Camta1, Calmodulin binding transcription activator 1 (variant 7) | NM_001349610 |
| Camta1, Calmodulin binding transcription activator 1 (variant 8) | NM_001349612 |
| Camta1, Calmodulin binding transcription activator 1 (variant 9) | NM_001349613 |
| Camta1, Calmodulin binding transcription activator 1 (variant 10) | NM_001349614 |
| Camta1, Calmodulin binding transcription activator 1 (variant 11) | NM_001349615 |
| Camta1, Calmodulin binding transcription activator 1 (variant 12) | NM_001349616 |
| Camta1, Calmodulin binding transcription activator 1 (variant 13) | NM_001349617 |
| Camta1, Calmodulin binding transcription activator 1 (variant 14) | NM_001349618 |
| Camta1, Calmodulin binding transcription activator 1 (variant 15) | NM_00134961 |
| Camta1, Calmodulin binding transcription activator 1 (variant 16) | NM_001349620 |
| Camta1, Calmodulin binding transcription activator 1 (variant 17) | NM_001349621 |
| Camta1, Calmodulin binding transcription activator 1 (variant 18) | NM_001349622 |
| Camta1, Calmodulin binding transcription activator 1 (variant 19) | NM_001349623 |
| Camta1, Calmodulin binding transcription activator 1 (variant 20) | NM_001349624 |
| Camta1, Calmodulin binding transcription activator 1 (variant 21) | NM_001349625 |
| Camta1, Calmodulin binding transcription activator 1 (variant 22) | NM_001349626 |
| Camta1, Calmodulin binding transcription activator 1 (variant 23) | NM_001349627 |
| Hey1, Hes related family bHLH transcription factor with YRPW motif 1 (variant 1) | NM_012258 |
| Hey1, Hes related family bHLH transcription factor with YRPW motif 1 (variant 2) | NM_001040708 |
| Hey1, Hes related family bHLH transcription factor with YRPW motif 1 (variant 3) | NM_001282851 |
| Hey2, Hes related family bHLH transcription factor with YRPW motif 2 | NM_012259 |
| Gata2, GATA binding protein 2 (variant 1) | NM_001145661 |
| Gata2, GATA binding protein 2 (variant 2) | NM_032638 |
| Gata2, GATA binding protein 2 (variant 3) | NM_001145662 |
| Gata3, GATA binding protein 3 (variant 1) | NM_001002295 |
| Gata3, GATA binding protein 3 (variant 2) | NM_002051 |
| Lass2, Ceramide synthase 2 (variant 1) | NM_181746 |
| Lass2, Ceramide synthase 2 (variant 2) | NM_022075 |
| Cux1, Cut like homeobox 1 (variant 1) | NM_181552 |
| Cux1, Cut like homeobox 1 (variant 2) | NM_001913 |
| Cux1, Cut like homeobox 1 (variant 3) | NM_181500 |
| Cux1, Cut like homeobox 1 (variant 4) | NM_001202543 |
| Cux1, Cut like homeobox 1 (variant 5) | NM_001202544 |
| Cux1, Cut like homeobox 1 (variant 6) | NM_001202545 |
| Cux1, Cut like homeobox 1 (variant 7) | NM_001202546 |
| Nr2f1, Nuclear receptor subfamily 2 group F member 1 | NM_005654 |
| Hes1, Hes family bHLH transcription factor 1 | NM_005524 |
| Rorb, RAR related orphan receptor B (variant 1) | NM_006914 |
| Rorb, RAR related orphan receptor B (variant 2) | NM_001365023 |
| Jun, Jun proto-oncogene AP-1 transcription factor subunit | NM_002228 |
| Zfp667 (human Znf667), Zinc finger protein 667 (variant 1) | NM_022103 |
| Zfp667 (human Znf667), Zinc finger protein 667 (variant 2) | NM_00132135 |
| Zfp667 (human Znf667), Zinc finger protein 667 (variant 3) | NM_001321355 |
| Lhx3, Lim homeobox 3 (variant 1) | NM_178138 |
| Lhx3, Lim homeobox 3 (variant 2) | NM_014564 |
| Lhx3, Lim homeobox 3 (variant 3) | NM_001363746 |
| Nhlh1, Nescient helix-loop-helix 1 | NM_005598 |
| Zmiz1, Zinc finger MIZ-type containing 1 | NM_020338 |
| Myt1, Myelin transcription factor 1 | NM_004535 |
| Stat3, Signal transducer and activator of transcription 3 (variant 1) | NM_139276 |
| Stat3, Signal transducer and activator of transcription 3 (variant 2) | NM_003150 |
| Stat3, Signal transducer and activator of transcription 3 (variant 3) | NM_213662 |
| Barhl1, BarH like homeobox 1 | NM_020064 |
| Tox, Thymocyte selection associated high mobility group box | NM_014729 |
| Prox1, Prospero homeobox 1 (variant 1) | NM_001270616 |
| Prox1, Prospero homeobox 1 (variant 2) | NM_002763 |
| Nfia, Nuclear factor I A (variant 1) | NM_00113467 |
| Nfia, Nuclear factor I A (variant 2) | NM_005595 |
| Nfia, Nuclear factor I A (variant 3) | NM_001145511 |
| Nfia, Nuclear factor I A (variant 4) | NM_001145512 |
| Thrb, Thyroid hormone receptor beta (variant 1) | NM_000461 |
| Thrb, Thyroid hormone receptor beta (variant 2) | NM_001128176 |
| Thrb, Thyroid hormone receptor beta (variant 3) | NM_001128177 |
| Thrb, Thyroid hormone receptor beta (variant 4) | NM_001252634 |
| Thrb, Thyroid hormone receptor beta (variant 5) | NM_001354708 |
| Thrb, Thyroid hormone receptor beta (variant 6) | NM_001354709 |
| Thrb, Thyroid hormone receptor beta (variant 7) | NM_001354710 |
| Thrb, Thyroid hormone receptor beta (variant 8) | NM_001354711 |
| Thrb, Thyroid hormone receptor beta (variant 9) | NM_001354712 |
| Thrb, Thyroid hormone receptor beta (variant 10) | NM_001354713 |
| Thrb, Thyroid hormone receptor beta (variant 11) | NM_001354714 |
| Thrb, Thyroid hormone receptor beta (variant 12) | NM_001354715 |
| Mycl1, MYCL proto-oncogene BHLH transcription factor (variant 1) | NM_001033081 |
| Mycl1, MYCL proto-oncogene BHLH transcription factor (variant 2) | NM_001033082 |
| Mycl1, MYCL proto-oncogene BHLH transcription factor (variant 3) | NM_005376 |
| Kdm5a, Lysine demethylase 5A | NM_001042603 |
| Creb314, cAMP responsive element binding protein 3 like 4 (variant 1) | NM_130898 |
| Creb314, cAMP responsive element binding protein 3 like 4 (variant 2) | NM_001255978 |
| Creb314, cAMP responsive element binding protein 3 like 4 (variant 3) | NM_001255979 |
| Creb314, cAMP responsive element binding protein 3 like 4 (variant 4) | NM_001255980 |
| Creb314, cAMP responsive element binding protein 3 like 4 (variant 5) | NM_001255981 |
| Creb314, cAMP responsive element binding protein 3 like 4 (variant 6) | NR_045658 |
| Etv1, ETS variant 1 (variant 1) | NM_004956 |
| Etv1, ETS variant 1 (variant 2) | NM_001163147 |
| Etv1, ETS variant 1 (variant 3) | NM_001163148 |
| Etv1, ETS variant 1 (variant 4) | NM_001163149 |
| Etv1, ETS variant 1 (variant 5) | NM_001163150 |
| Etv1, ETS variant 1 (variant 6) | NM_001163151 |
| Etv1, ETS variant 1 (variant 7) | NM_001163152 |
| Peg3, Paternally expressed 3 (variant 1) | NM_006210 |
| Peg3, Paternally expressed 3 (variant 2) | NM_001146184 |
| Peg3, Paternally expressed 3 (variant 3) | NM_001146185 |
| Peg3, Paternally expressed 3 (variant 4) | NM_001146186 |
| Peg3, Paternally expressed 3 (variant 5) | NM_001146187 |
| Bach2, BTB domain and CNC homolog 2 (variant 1) | NM_021813 |
| Bach2, BTB domain and CNC homolog 2 (variant 2) | NM_001170794 |
| Zbtb38, Zinc finger and BTB domain containing 38 (variant 1) | NM_001080412 |
| Zbtb38, Zinc finger and BTB domain containing 38 (variant 2) | NM_001350099 |
| Zbtb38, Zinc finger and BTB domain containing 38 (variant 3) | NM_001350100 |
| Lbh, Limb bud and heart development | NM_030915 |
| Tub, Tubby bipartite transcription factor (variant 1) | NM_003320 |
| Tub, Tubby bipartite transcription factor (variant 2) | NM_177972 |
| Hmg20, High mobility group20A (variant 1) | NM_018200 |
| Hmg20, High mobility group20A (variant 2) | NM_001304504 |
| Hmg20, High mobility group20A (variant 3) | NM_001304505 |
| Rest, RE1 silencing transcription factor (variant 1) | NM_005612 |
| Rest, RE1 silencing transcription factor (variant 2) | NM_001193508 |
| Rest, RE1 silencing transcription factor (variant 3) | NM_001363453 |
| Zfp827 (human Znf827;), Zinc finger protein 827 (variant 1) | NM_001306215 |
| Zfp827 (human Znf827;), Zinc finger protein 827 (variant 2) | NM_178835 |
| Aff3, AFR/FMR2 family member 3 (variant 1) | NM_002285 |
| Aff3, AFR/FMR2 family member 3 (variant 2) | NM_001025108 |
| Pknox2, PBX/knotted homeobox 2 | NM_022062 |
| Arid3b, AT-rich interaction domain 3B (variant 1) | NM_001307939 |
| Arid3b, AT-rich interaction domain 3B (variant 2) | NM_006465 |
| MIxip, MLX interacting protein | NM_014938 |
| Zfp532 (human Znf532), Zinc finger protein 532 (variant 1) | NM_018181 |
| Zfp532 (human Znf532), Zinc finger protein 532 (variant 2) | NM_001318726 |
| Zfp532 (human Znf532), Zinc finger protein 532 (variant 3) | NM_001318727 |
| Zfp532 (human Znf532), Zinc finger protein 532 (variant 4) | NM_001318728 |
| Zfp532 (human Znf532), Zinc finger protein 532 (variant 5) | NM_001353525 |
| Zfp532 (human Znf532), Zinc finger protein 532 (variant 6) | NM_001353526 |
| Zfp532 (human Znf532), Zinc finger protein 532 (variant 7) | NM_001353527 |
| Zfp532 (human Znf532), Zinc finger protein 532 (variant 8) | NM_001353528 |
| Zfp532 (human Znf532), Zinc finger protein 532 (variant 9) | NM_001353529 |
| Zfp532 (human Znf532), Zinc finger protein 532 (variant 10) | NM_001353530 |
| Zfp532 (human Znf532), Zinc finger protein 532 (variant 11) | NM_001353531 |
| Zfp532 (human Znf532), Zinc finger protein 532 (variant 12) | NM_001353532 |
| Zfp532 (human Znf532), Zinc finger protein 532 (variant 13) | NM_001353533 |
| Zfp532 (human Znf532), Zinc finger protein 532 (variant 14) | NM_001353534 |
| Zfp532 (human Znf532), Zinc finger protein 532 (variant 15) | NM_001353535 |
| Zfp532 (human Znf532), Zinc finger protein 532 (variant 16) | NM_001353536 |
| Zfp532 (human Znf532), Zinc finger protein 532 (variant 17) | NM_001353537 |
| Zfp532 (human Znf532), Zinc finger protein 532 (variant 18) | NM_001353538 |
| Ikzf2, IKAROS family zinc finger 2 (variant 1) | NM_016260 |
| Ikzf2, IKAROS family zinc finger 2 (variant 2) | NM_001079526 |
| Ikzf2, IKAROS family zinc finger 2 (variant 3) | NM_001371274 |
| Ikzf2, IKAROS family zinc finger 2 (variant 4) | NM_001371275 |
| Ikzf2, IKAROS family zinc finger 2 (variant 5) | NM_001371276.1 |
| Ikzf2, IKAROS family zinc finger 2 (variant 6) | NM_001371277.1 |
| Ikzf2, IKAROS family zinc finger 2 (variant 7) | NM_001387220.1 |
| Sall1, Spalt like transcription factor 1 (variant 1) | NM_00296 |
| Sall1, Spalt like transcription factor 1 (variant 2) | NM_001127892 |
| Six2, SIX homeobox 2 | NM_016932 |
| Sall3, Spalt like transcription factor 3 | NM_171999 |
| Lin28b, Lin-28 homolog B | NM_001004317 |
| Rfx7, Regulatory factor X7 | NM_022841 |
| Sox4, SRY-box 4 | NM_003107 |
| Bdnf, Brain derived neurotrophic factor (variant 1) | NM_170735 |
| Bdnf, Brain derived neurotrophic factor (variant 2) | NM_170732 |
| Bdnf, Brain derived neurotrophic factor (variant 3) | NM_170731 |
| Bdnf, Brain derived neurotrophic factor (variant 4) | NM_001709 |
| Bdnf, Brain derived neurotrophic factor (variant 5) | NM_17073 |
| Bdnf, Brain derived neurotrophic factor (variant 6) | NM_170734 |
| Bdnf, Brain derived neurotrophic factor (variant 7) | NM_001143805 |
| Bdnf, Brain derived neurotrophic factor (variant 8) | NM_001143806 |
| Bdnf, Brain derived neurotrophic factor (variant 9) | NM_001143807 |
| Bdnf, Brain derived neurotrophic factor (variant 10) | NM_001143808 |
| Bdnf, Brain derived neurotrophic factor (variant 11) | NM_001143811 |
| Bdnf, Brain derived neurotrophic factor (variant 12) | NM_001143812 |
| Bdnf, Brain derived neurotrophic factor (variant 13) | NM_001143813 |
| Bdnf, Brain derived neurotrophic factor (variant 14) | NM_001143814 |
| Bdnf, Brain derived neurotrophic factor (variant 16) | NM_001143816 |
| Bdnf, Brain derived neurotrophic factor (variant 17) | NM_001143809 |
| Bdnf, Brain derived neurotrophic factor (variant 18) | NM_001143810 |
| Ntf3, Neurotrophin 3 (variant 1) | NM_001102654 |
| Ntf3, Neurotrophin 3 (variant 2) | NM_002527 |
| Sox11, SRY-box 11 | NM_003108 |
| Tecta, Tectorin alpha | NM_005422 |
| Tectb, Tectorin beta | NM_058222 |
| Gjb2, Gap junction protein beta 2 | NM_004004 |
| Gjb6, Gap junction protein beta 6 (variant 1) | NM_001110219 |
| Gjb6, Gap junction protein beta 6 (variant 2) | NM_001110220 |
| Gjb6, Gap junction protein beta 6 (variant 3) | NM_006783 |
| Gjb6, Gap junction protein beta 6 (variant 4) | NM_001110221 |
| Tmem16a, Transmembrane protein 16A | NM_018043 |
| Ngfr, Nerve growth factor receptor | NM_002507 |
| Pmp22, peripheral myelin protein 22 (variant 1) | NM_000304 |
| Pmp22, peripheral myelin protein 22 (variant 2) | NM_153321 |
| Pmp22, peripheral myelin protein 22 (variant 3) | NM_153322 |
| Pmp22, peripheral myelin protein 22 (variant 4) | NM_001281455 |
| Pmp22, peripheral myelin protein 22 (variant 5) | NM_001281456 |
| Pmp22, peripheral myelin protein 22 (variant 8) | NM_001330143 |
| Mpz, Myelin protein zero | NM_000530 |
| | NM_001315491 |
| Mxd4, Max dimerization protein 4 | NM_006454 |
| PAX3, Paired box 3, transcript variant PAX3 | NM_181457.4 |
| PAX3, Paired box 3, transcript variant PAX3A | NM_000438.6 |
| PAX3, Paired box 3, transcript variant PAX3B | NM_013942.5 |
| PAX3, Paired box 3, transcript variant PAX3D | NM_181458.4 |
| PAX3, Paired box 3, transcript variant PAX3E | NM_181459.4 |
| PAX3, Paired box 3, transcript variant PAX3H | NM_181460.4 |
| PAX3, Paired box 3, transcript variant PAX3G | NM_181461.4 |
| PAX3, Paired box 3, transcript variant PAX31 | NM_001127366.3 |
| MITF, Melanocyte inducing transcription factor, transcript variant 9 | NM_001354604.2 |
| MITF, Melanocyte inducing transcription factor, transcript variant 4 | NM_000248.4 |
| MITF, Melanocyte inducing transcription factor, transcript variant 3 | NM_006722.3 |
| MITF, Melanocyte inducing transcription factor, transcript variant 5 | NM_198158.3 |
| MITF, Melanocyte inducing transcription factor, transcript variant 1 | NM_198159.3 |
| MITF, Melanocyte inducing transcription factor, transcript variant 2 | NM_198177.3 |
| MITF, Melanocyte inducing transcription factor, transcript variant 6 | NM_198178.3 |
| MITF, Melanocyte inducing transcription factor, transcript variant 7 | NM_001184967.2 |
| MITF, Melanocyte inducing transcription factor, transcript variant 8 | NM_001184968.2 |
| MITF, Melanocyte inducing transcription factor, transcript variant 10 | NM_001354605.2 |
| MITF, Melanocyte inducing transcription factor, transcript variant 11 | NM_001354606.2 |
| MITF, Melanocyte inducing transcription factor, transcript variant 12 | NM_001354607.2 |
| MITF, Melanocyte inducing transcription factor, transcript variant 13 | NM_001354608.2 |
| EDNRB, Endothelin receptor type B, transcript variant 3 | NM_001122659.3 |
| EDNRB, Endothelin receptor type B, transcript variant 1 | NM_000115.5 |
| EDNRB, Endothelin receptor type B, transcript variant 2 | NM_003991.4 |
| EDNRB, Endothelin receptor type B, transcript variant 4 | NM_001201397.1 |
| EDN3, endothelin 3, transcript variant 4 | NM_207034.3 |
| EDN3, endothelin 3, transcript variant 2 | NM_207032.3 |
| EDN3, endothelin 3, transcript variant 3 | NM_207033.3 |
| EDN3, endothelin 3, transcript variant 5 | NM_001302455.2 |
| EDN3, endothelin 3, transcript variant 6 | NM_001302456.2 |
| EYA1, EYA transcriptional coactivator and phosphatase 1, transcript variant | NM_000503.6 |
| EYA1C | |
| EYA1, EYA transcriptional coactivator and phosphatase 1, transcript variant | NM_172058.4 |
| EYA1B | |
| EYA1, EYA transcriptional coactivator and phosphatase 1, transcript variant | NM_172059.5 |
| EYA1D | |
| EYA1, EYA transcriptional coactivator and phosphatase 1, transcript variant 5 | NM_001288574.2 |
| EYA1, EYA transcriptional coactivator and phosphatase 1, transcript variant 6 | NM_001288575.2 |
| EYA1, EYA transcriptional coactivator and phosphatase 1, transcript variant 7 | NM_001370333.1 |
| EYA1, EYA transcriptional coactivator and phosphatase 1, transcript variant 8 | NM_001370334.1 |
| EYA1, EYA transcriptional coactivator and phosphatase 1, transcript variant 9 | NM_001370335.1 |
| EYA1, EYA transcriptional coactivator and phosphatase 1, transcript variant 10 | NM_001370336.1 |
| SIX1, SIX homeobox 1 | NM_005982.4 |
| SIX5, SIX homeobox 5 | NM_175875.5 |
| NF2, Neurofibromin 2, transcript variant 1 | NM_000268.4 |
| NF2, Neurofibromin 2, transcript variant 2 | NM_016418.5 |
| NF2, Neurofibromin 2, transcript variant 12 | NM_181825.3 |
| NF2, Neurofibromin 2, transcript variant 5 | NM_181828.3 |
| NF2, Neurofibromin 2, transcript variant 6 | NM_181829.3 |
| NF2, Neurofibromin 2, transcript variant 7 | NM_181830.3 |
| NF2, Neurofibromin 2, transcript variant 13 | NM_181831.3 |
| NF2, Neurofibromin 2, transcript variant 8 | NM_181832.3 |
| NF2, Neurofibromin 2, transcript variant 9 | NM_181833.3 |
| USH1G, USH1 protein network component sans, transcript variant 1 | NM_173477.5 |
| USH1G, USH1 protein network component sans, transcript variant 2 | NM_001282489.3 |
| CIB2, Calcium and integrin binding family member 2, transcript variant 1 | NM_006383.4 |
| CIB2, Calcium and integrin binding family member 2, transcript variant 2 | NM_001271888.2 |
| CIB2, Calcium and integrin binding family member 2, transcript variant 3 | NM_001271889.2 |
| CIB2, Calcium and integrin binding family member 2, transcript variant 4 | NM_001301224.2 |
| ADGRV1 (also known as USH2B), Adhesion G protein-coupled receptor V1 | NM_032119.4 |
| USH2A, Usherin, transcript variant 1 | NM_007123.6 |
| USH2A, Usherin, transcript variant 2 | NM_206933.4 |
| HARS1 (also known as USH3B), histidyl-tRNA synthase 1, transcript variant 1 | NM_002109.6 |
| HARS1 (also known as USH3B), histidyl-tRNA synthase 1, transcript variant 2 | NM_001258040.3 |
| HARS1 (also known as USH3B), histidyl-tRNA synthase 1, transcript variant 3 | NM_001258041.3 |
| HARS1 (also known as USH3B), histidyl-tRNA synthase 1, transcript variant 4 | NM_001258042.3 |
| HARS1 (also known as USH3B), histidyl-tRNA synthase 1, transcript variant 5 | NM_001289092.2 |
| HARS1 (also known as USH3B), histidyl-tRNA synthase 1, transcript variant 6 | NM_001289093.2 |
| HARS1 (also known as USH3B), histidyl-tRNA synthase 1, transcript variant 7 | NM_001289094.2 |
| BTD, Biotinidase, transcript variant 3 | NM_001370658.1 |
| BTD, Biotinidase, transcript variant 1 | NM_001281723.3 |
| BTD, Biotinidase, transcript variant 2 | NM_001281724.3 |
| BTD, Biotinidase, transcript variant 4 | NM_001281725.2 |
| BTD, Biotinidase, transcript variant 5 | NM_001281726.2 |
| BTD, Biotinidase, transcript variant 6 | NM_001323582.1 |
| BTD, Biotinidase, transcript variant 7 | NM_001370752.1 |
| BTD, Biotinidase, transcript variant 8 | NM_001370753.1 |
| PHYH, Phytanoyl-CoA 2-hydroxylase, transcript variant 1 | NM_006214.4 |
| PHYH, Phytanoyl-CoA 2-hydroxylase, transcript variant 2 | NM_001037537.2 |
| PHYH, Phytanoyl-CoA 2-hydroxylase, transcript variant 3 | NM_001323080.2 |
| PHYH, Phytanoyl-CoA 2-hydroxylase, transcript variant 4 | NM_001323082.2 |
| PHYH, Phytanoyl-CoA 2-hydroxylase, transcript variant 5 | NM_001323083.2 |
| PHYH, Phytanoyl-CoA 2-hydroxylase, transcript variant 6 | NM_001323084.2 |
| TIMM8A, Translocase of inner mitochondrial membrane 8A, transcript variant 1 | NM_004085.4 |
| TIMM8A, Translocase of inner mitochondrial membrane 8A, transcript variant 2 | NM_001145951.2 |
| ACTG1, Actin gamma 1, transcript variant 1 | NM_001199954.3 |
| ACTG1, Actin gamma 1, transcript variant 2 | NM_001614.5 |
| CCDC50, Coiled-coil domain containing 50, transcript variant 1 | NM_174908.4 |
| CCDC50, Coiled-coil domain containing 50, transcript variant 2 | NM_178335.3 |
| CD164, CD164 molecule, transcript variant 1 | NM_006016.6 |
| CD164, CD164 molecule, transcript variant 2 | NM_001142401.3 |
| CD164, CD164 molecule, transcript variant 3 | NM_001142402.3 |
| CD164, CD164 molecule, transcript variant 4 | NM_001142403.3 |
| CD164, CD164 molecule, transcript variant 5 | NM_001142404.3 |
| CD164, CD164 molecule, transcript variant 6 | NM_001346500.2 |
| COCH, Cochlin, transcript variant 1 | NM_001135058.2 |
| COCH, Cochlin, transcript variant 2 | NM_004086.3 |
| COCH, Cochlin, transcript variant 3 | NM_001347720.2 |
| GSDME, Gasdermin E, transcript variant 1 | NM_004403.3 |
| GSDME, Gasdermin E, transcript variant 2 | NM_001127453.2 |
| GSDME, Gasdermin E, transcript variant 3 | NM_001127454.2 |
| DIAPH1, Diaphanous related formin 1, transcript variant 1 | NM_005219.5 |
| DIAPH1, Diaphanous related formin 1, transcript variant 2 | NM_001079812.3 |
| DIAPH1, Diaphanous related formin 1, transcript variant 3 | NM_001314007.2 |
| DMXL2, Dmx like 2, transcript variant 4 | NM_001378457.1 |
| DMXL2, Dmx like 2, transcript variant 2 | NM_015263.5 |
| DMXL2, Dmx like 2, transcript variant 1 | NM_001174116.3 |
| DMXL2, Dmx like 2, transcript variant 3 | NM_001174117.3 |
| DMXL2, Dmx like 2, transcript variant 5 | NM_001378458.1 |
| DMXL2, Dmx like 2, transcript variant 6 | NM_001378459.1 |
| DMXL2, Dmx like 2, transcript variant 7 | NM_001378460.1 |
| DMXL2, Dmx like 2, transcript variant 8 | NM_001378461.1 |
| DMXL2, Dmx like 2, transcript variant 9 | NM_001378462.1 |
| DMXL2, Dmx like 2, transcript variant 10 | NM_001378463.1 |
| DMXL2, Dmx like 2, transcript variant 11 | NM_001378464.1 |
| DSPP, Dentin sialophosphoprotein | NM_014208.3 |
| EYA4, EYA transcriptional coactivator and phosphatase 4, transcript variant 1 | NM_004100.5 |
| EYA4, EYA transcriptional coactivator and phosphatase 4, transcript variant 2 | NM_172103.4 |
| EYA4, EYA transcriptional coactivator and phosphatase 4, transcript variant 4 | NM_172105.4 |
| EYA4, EYA transcriptional coactivator and phosphatase 4, transcript variant 5 | NM_001301012.2 |
| EYA4, EYA transcriptional coactivator and phosphatase 4, transcript variant 6 | NM_001301013.2 |
| EYA4, EYA transcriptional coactivator and phosphatase 4, transcript variant 7 | NM_001370458.1 |
| EYA4, EYA transcriptional coactivator and phosphatase 4, transcript variant 8 | NM_001370459.1 |
| GJB3, Gap junction protein beta 3, transcript variant 1 | NM_024009.3 |
| GJB3, Gap junction protein beta 3, transcript variant 2 | NM_001005752.2 |
| GRHL2, Grainyhead like transcription factor 2, transcript variant 1 | NM_024915.4 |
| GRHL2, Grainyhead like transcription factor 2, transcript variant 2 | NM_001330593.2 |
| HOMER2, Homer scaffold protein 2, transcript variant 1 | NM_004839.4 |
| HOMER2, Homer scaffold protein 2, transcript variant 2 | NM_199330.3 |
| KCNQ4, Potassium voltage-gated channel subfamily Q member 4, transcript | NM_004700.4 |
| variant 1 | |
| KCNQ4, Potassium voltage-gated channel subfamily Q member 4, transcript | NM_172163.3 |
| variant 2 | |
| MCM2, Minichromosome maintenance complex component 2 | NM_004526.4 |
| MYH14, Myosin heavy chain 14, transcript variant 1 | NM_001077186.2 |
| MYH14, Myosin heavy chain 14, transcript variant 2 | NM_024729.4 |
| MYH14, Myosin heavy chain 14, transcript variant 3 | NM_001145809.2 |
| MYH9, Myosin heavy chain 9 | NM_002473.6 |
| MYO1A, Myosin IA, transcript variant 1 | NM_001256041.2 |
| MYO1A, Myosin IA, transcript variant 2 | NM_005379.4 |
| MYO6, Myosin VI, transcript variant 1 | NM_004999.4 |
| MYO6, Myosin VI, transcript variant 2 | NM_001300899.2 |
| MYO6, Myosin VI, transcript variant 3 | NM_001368136.1 |
| MYO6, Myosin VI, transcript variant 4 | NM_001368137.1 |
| MYO6, Myosin VI, transcript variant 5 | NM_001368138.1 |
| MYO6, Myosin VI, transcript variant 6 | NM_001368139.1 |
| MYO6, Myosin VI, transcript variant 7 | NM_001368140.1 |
| MYO6, Myosin VI, transcript variant 10 | NM_001368865.1 |
| MYO6, Myosin VI, transcript variant 11 | NM_001368866.1 |
| OSBPL2, Oxysterol binding protein like 2, transcript variant 1 | NM_014835.5 |
| OSBPL2, Oxysterol binding protein like 2, transcript variant 2 | NM_144498.4 |
| OSBPL2, Oxysterol binding protein like 2, transcript variant 3 | NM_001278649.3 |
| OSBPL2, Oxysterol binding protein like 2, transcript variant 4 | NM_001363878.2 |
| P2RX2, Purinergic receptor P2X2, transcript variant 1 | NM_170682.4 |
| P2RX2, Purinergic receptor P2X2, transcript variant 6 | NM_012226.5 |
| P2RX2, Purinergic receptor P2X2, transcript variant 3 | NM_016318.4 |
| P2RX2, Purinergic receptor P2X2, transcript variant 4 | NM_170683.4 |
| P2RX2, Purinergic receptor P2X2, transcript variant 5 | NM_174872.3 |
| P2RX2, Purinergic receptor P2X2, transcript variant 2 | NM_174873.3 |
| P2RX2, Purinergic receptor P2X2, transcript variant 7 | NM_001282164.2 |
| P2RX2, Purinergic receptor P2X2, transcript variant 8 | NM_001282165.2 |
| TBC1D24, TBC1 domain family member 24, transcript variant 1 | NM_001199107.2 |
| TBC1D24, TBC1 domain family member 24, transcript variant 2 | NM_020705.3 |
| PEX7, Peroxisomal biogenesis factor 7 | NM_000288.4 |
| TJP2, Tight junction protein 2, transcript variant 1 | NM_004817.4 |
| TJP2, Tight junction protein 2, transcript variant 2 | NM_201629.3 |
| TJP2, Tight junction protein 2, transcript variant 5 | NM_001170414.2 |
| TJP2, Tight junction protein 2, transcript variant 4 | NM_001170415.1 |
| TJP2, Tight junction protein 2, transcript variant 3 | NM_001170416.2 |
| TJP2, Tight junction protein 2, transcript variant 6 | NM_001369870.1 |
| TJP2, Tight junction protein 2, transcript variant 7 | NM_001369871.1 |
| TJP2, Tight junction protein 2, transcript variant 8 | NM_001369872.1 |
| TJP2, Tight junction protein 2, transcript variant 9 | NM_001369873.1 |
| TJP2, Tight junction protein 2, transcript variant 10 | NM_001369874.1 |
| TJP2, Tight junction protein 2, transcript variant 11 | NM_001369875.1 |
| FAM189A2, Family with sequence similarity 189 member A2, transcript variant 1 | NM_004816.5 |
| FAM189A2, Family with sequence similarity 189 member A2, transcript variant 2 | NM_001127608.3 |
| FAM189A2, Family with sequence similarity 189 member A2, transcript variant 3 | NM_001347995.2 |
| WFS1, Wolframin ER transmembrane glycoprotein, transcript variant 1 | NM_006005.3 |
| WFS1, Wolframin ER transmembrane glycoprotein, transcript variant 1 | NM_001145853.1 |
| ADCY1, Adenylate cyclase 1, transcript variant 1 | NM_021116.4 |
| ADCY1, Adenylate cyclase 1, transcript variant 2 | NM_001281768.2 |
| BDP1, B double prime 1, subunit of RNA polymerase III transcription initiation | NM_018429.3 |
| factor IIIB | |
| BSND, barttin CLCNK type accessory subunit beta | NM_057176.3 |
| CABP2, Calcium binding protein 2, transcript variant 1 | NM_016366.3 |
| CABP2, Calcium binding protein 2, transcript variant 3 | NM_001318496.2 |
| CDC14A, Cell division cycle 14A, transcript variant 1 | NM_003672.4 |
| CDC14A, Cell division cycle 14A, transcript variant 2 | NM_033312.3 |
| CDC14A, Cell division cycle 14A, transcript variant 3 | NM_033313.3 |
| CDC14A, Cell division cycle 14A, transcript variant 4 | NM_001319210.2 |
| CDC14A, Cell division cycle 14A, transcript variant 5 | NM_001319211.2 |
| CDC14A, Cell division cycle 14A, transcript variant 6 | NM_001319212.2 |
| CLDN14, Claudin 14, transcript variant 5 | NM_001146079.2 |
| CLDN14, Claudin 14, transcript variant epsilon | NM_012130.4 |
| CLDN14, Claudin 14, transcript variant 1 | NM_144492.3 |
| CLDN14, Claudin 14, transcript variant 3 | NM_001146077.2 |
| CLDN14, Claudin 14, transcript variant gamma | NM_001146078.3 |
| CLIC5, Chloride intracellular channel 5, transcript variant 2 | NM_016929.5 |
| CLIC5, Chloride intracellular channel 5, transcript variant 1 | NM_001114086.2 |
| CLIC5, Chloride intracellular channel 5, transcript variant 3 | NM_001256023.2 |
| CLIC5, Chloride intracellular channel 5, transcript variant 7 | NM_001370649.1 |
| CLIC5, Chloride intracellular channel 5, transcript variant 8 | NM_001370650.1 |
| DCDC2, Doublecortin domain containing 2, transcript variant 1 | NM_016356.5 |
| DCDC2, Doublecortin domain containing 2, transcript variant 2 | NM_001195610.2 |
| PJVK, Pejvakin, transcript variant 1 | NM_001042702.5 |
| PJVK, Pejvakin, transcript variant 2 | NM_001353775.2 |
| PJVK, Pejvakin, transcript variant 3 | NM_001353776.2 |
| PJVK, Pejvakin, transcript variant 4 | NM_001353777.1 |
| PJVK, Pejvakin, transcript variant 5 | NM_001353778.2 |
| PJVK, Pejvakin, transcript variant 6 | NM_001369912.1 |
| ELMOD3, ELMO domain containing 3, transcript variant 3 | NM_001135022.2 |
| ELMOD3, ELMO domain containing 3, transcript variant 2 | NM_001135021.2 |
| ELMOD3, ELMO domain containing 3, transcript variant 4 | NM_001135023.2 |
| ELMOD3, ELMO domain containing 3, transcript variant 5 | NM_001329791.2 |
| ELMOD3, ELMO domain containing 3, transcript variant 6 | NM_001329792.2 |
| ELMOD3, ELMO domain containing 3, transcript variant 7 | NM_001329793.2 |
| EPS8, Epidermal growth factor receptor pathway substrate 8 | NM_004447.6 |
| EPS8L2, EPS8 like 2 | NM_022772.4 |
| ESPN, Espin, transcript variant 1 | NM_031475.3 |
| ESPN, Espin, transcript variant 2 | NM_001367473.1 |
| ESPN, Espin, transcript variant 3 | NM_001367474.1 |
| ESRRB, Estrogen related receptor beta, transcript variant 1 | NM_004452.4 |
| ESRRB, Estrogen related receptor beta, transcript variant 2 | NM_001379180.1 |
| GIPC3, GIPC PDZ domain containing family member 3 | NM_133261.3 |
| GPSM2, G protein signaling modulator 2, transcript variant 1 | NM_001321039.3 |
| GPSM2, G protein signaling modulator 2, transcript variant 2 | NM_001321038.2 |
| GPSM2, G protein signaling modulator 2, transcript variant 3 | NM_013296.5 |
| GRXCR1, Glutaredoxin and cysteine rich domain containing 1 | NM_001080476.3 |
| GRXCR2, Glutaredoxin and cysteine rich domain containing 2 | NM_001080516.2 |
| HGF, Hepatocyte growth factor, transcript variant 1 | NM_000601.6 |
| HGF, Hepatocyte growth factor, transcript variant 2 | NM_001010931.3 |
| HGF, Hepatocyte growth factor, transcript variant 3 | NM_001010932.3 |
| HGF, Hepatocyte growth factor, transcript variant 4 | NM_001010933.3 |
| HGF, Hepatocyte growth factor, transcript variant 5 | NM_001010934.3 |
| ILDR1, Immunoglobulin like domain containing receptor 1, transcript variant 1 | NM_001199799.2 |
| ILDR1, Immunoglobulin like domain containing receptor 1, transcript variant 2 | NM_175924.4 |
| ILDR1, Immunoglobulin like domain containing receptor 1, transcript variant 3 | NM_001199800.2 |
| KARS1, Lysyl-tRNA synthase 1, transcript variant 1 | NM_001130089.2 |
| KARS1, Lysyl-tRNA synthase 1, transcript variant 2 | NM_005548.3 |
| KARS1, Lysyl-tRNA synthase 1, transcript variant 3 | NM_001378148.1 |
| LHFPL5, LHFPL tetraspan subfamily member 5 | NM_182548.4 |
| LOXHD1, Lipoxygenase homology PLAT domains 1, transcript variant 6 | NM_001384474.1 |
| LOXHD1, Lipoxygenase homology PLAT domains 1, transcript variant 1 | NM_144612.7 |
| LOXHD1, Lipoxygenase homology PLAT domains 1, transcript variant 2 | NM_001145472.3 |
| LOXHD1, Lipoxygenase homology PLAT domains 1, transcript variant 3 | NM_001145473.3 |
| LOXHD1, Lipoxygenase homology PLAT domains 1, transcript variant 4 | NM_001173129.2 |
| LOXHD1, Lipoxygenase homology PLAT domains 1, transcript variant 5 | NM_001308013.2 |
| LRTOMT, Leucine rich transmembrane and O-methyltransferase domain | NM_001145309.4 |
| containing, transcript variant 5 | |
| LRTOMT, Leucine rich transmembrane and O-methyltransferase domain | NM_001145308.5 |
| containing, transcript variant 4 | |
| LRTOMT, Leucine rich transmembrane and O-methyltransferase domain | NM_001145310.4 |
| containing, transcript variant 6 | |
| MARVELD2, MARVEL domain containing 2, transcript variant 1 | NM_001038603.3 |
| MARVELD2, MARVEL domain containing 2, transcript variant 2 | NM_001244734.2 |
| MET, MET proto-oncogene, receptor tyrosine kinase, transcript variant 1 | NM_001127500.3 |
| MET, MET proto-oncogene, receptor tyrosine kinase, transcript variant 2 | NM_000245.4 |
| MET, MET proto-oncogene, receptor tyrosine kinase, transcript variant 3 | NM_001324401.3 |
| MET, MET proto-oncogene, receptor tyrosine kinase, transcript variant 4 | NM_001324402.2 |
| MSRB3, Methionine sulfoxide reductase B3, transcript variant 1 | NM_198080.4 |
| MSRB3, Methionine sulfoxide reductase B3, transcript variant 2 | NM_001031679.3 |
| MSRB3, Methionine sulfoxide reductase B3, transcript variant 3 | NM_001193460.2 |
| MSRB3, Methionine sulfoxide reductase B3, transcript variant 4 | NM_001193461.2 |
| MYO15A, Myosin XVA | NM_016239.4 |
| MYO3A, Myosin IIIA, transcript variant 1 | NM_017433.5 |
| MYO3A, Myosin IIIA, transcript variant 2 | NM_001368265.1 |
| NARS2, Asparaginyl-tRNA synthetase 2, mitochondrial, transcript variant 1 | NM_024678.6 |
| NARS2, Asparaginyl-tRNA synthetase 2, mitochondrial, transcript variant 2 | NM_001243251.2 |
| OTOG, Otogelin, transcript variant 1 | NM_001277269.2 |
| OTOG, Otogelin, transcript variant 2 | NM_001292063.2 |
| OTOGL, Otogelin like, transcript variant 1 | NM_173591.7 |
| OTOGL, Otogelin like, transcript variant 2 | NM_001368062.3 |
| OTOGL, Otogelin like, transcript variant 3 | NM_001378609.3 |
| OTOGL, Otogelin like, transcript variant 4 | NM_001378610.3 |
| PNPT1, Polyribonucleotide nucleotidyltransferase 1 | NM_033109.5 |
| PTPRQ, Protein tyrosine phosphatase receptor type Q | NM_001145026.2 |
| RDX, Radixin, transcript variant 1 | NM_001260492.2 |
| RDX, Radixin, transcript variant 2 | NM_001260493.2 |
| RDX, Radixin, transcript variant 3 | NM_002906.4 |
| RDX, Radixin, transcript variant 4 | NM_001260494.2 |
| RDX, Radixin, transcript variant 5 | NM_001260495.2 |
| RDX, Radixin, transcript variant 6 | NM_001260496.2 |
| RIPOR2, RHO family interacting cell polarization regulator 2, transcript variant 1 | NM_014722.5 |
| RIPOR2, RHO family interacting cell polarization regulator 2, transcript variant 2 | NM_015864.5 |
| RIPOR2, RHO family interacting cell polarization regulator 2, transcript variant 3 | NM_001286445.3 |
| RIPOR2, RHO family interacting cell polarization regulator 2, transcript variant 4 | NM_001286446.3 |
| RIPOR2, RHO family interacting cell polarization regulator 2, transcript variant 5 | NM_001286447.2 |
| RIPOR2, RHO family interacting cell polarization regulator 2, transcript variant 6 | NM_001346031.2 |
| RIPOR2, RHO family interacting cell polarization regulator 2, transcript variant 7 | NM_001346032.2 |
| ROR1, Receptor tyrosine kinase like orphan receptor 1, transcript variant 1 | NM_005012.4 |
| ROR1, Receptor tyrosine kinase like orphan receptor 1, transcript variant 2 | NM_001083592.2 |
| S1PR2, Sphingosine-1-phosphate receptor 2 | NM_004230.4 |
| SERPINB6, Serpin family B member 6, transcript variant 1 | NM_004568.6 |
| SERPINB6, Serpin family B member 6, transcript variant 2 | NM_001195291.3 |
| SERPINB6, Serpin family B member 6, transcript variant 3 | NM_001271822.2 |
| SERPINB6, Serpin family B member 6, transcript variant 4 | NM_001271823.2 |
| SERPINB6, Serpin family B member 6, transcript variant 5 | NM_001271824.2 |
| SERPINB6, Serpin family B member 6, transcript variant 6 | NM_001271825.2 |
| SERPINB6, Serpin family B member 6, transcript variant 7 | NM_001297699.2 |
| SERPINB6, Serpin family B member 6, transcript variant 8 | NM_001297700.2 |
| SERPINB6, Serpin family B member 6, transcript variant 9 | NM_001374515.1 |
| SERPINB6, Serpin family B member 6, transcript variant 10 | NM_001374516.1 |
| SERPINB6, Serpin family B member 6, transcript variant 11 | NM_001374517.1 |
| SLC22A4, Solute carrier family 22 member 4 | NM_003059.3 |
| SLC26A5, Solute carrier family 26 member 5, transcript variant a | NM_198999.3 |
| SLC26A5, Solute carrier family 26 member 5, transcript variant b | NM_206883.3 |
| SLC26A5, Solute carrier family 26 member 5, transcript variant c | NM_206884.3 |
| SLC26A5, Solute carrier family 26 member 5, transcript variant d | NM_206885.3 |
| SLC26A5, Solute carrier family 26 member 5, transcript variant e | NM_001167962.2 |
| SLC26A5, Solute carrier family 26 member 5, transcript variant i | NM_001321787.2 |
| SYNE4, Spectrin repeat containing nuclear envelope family member 4, transcript | NM_001039876.3 |
| variant 1 | |
| SYNE4, Spectrin repeat containing nuclear envelope family member 4, transcript | NM_001297735.3 |
| variant 2 | |
| TMEM132E, Transmembrane protein 132E | NM_001304438.2 |
| TMIE, Transmembrane inner ear, transcript variant 1 | NM_147196.3 |
| TMIE, Transmembrane inner ear, transcript variant 2 | NM_001370524.1 |
| TMIE, Transmembrane inner ear, transcript variant 3 | NM_001370525.1 |
| TMPRSS3, Transmembrane serine protease 3, transcript variant F | NM_001256317.3 |
| TMPRSS3, Transmembrane serine protease 3, transcript variant A | NM_024022.4 |
| TMPRSS3, Transmembrane serine protease 3, transcript variant C | NM_032404.3 |
| TMPRSS3, Transmembrane serine protease 3, transcript variant D | NM_032405.2 |
| TPRN, Taperin | NM_001128228.3 |
| TRIOBP, TRIO and F-actin binding protein, transcript variant 1 | NM_007032.5 |
| TRIOBP, TRIO and F-actin binding protein, transcript variant 2 | NM_138632.2 |
| TRIOBP, TRIO and F-actin binding protein, transcript variant 6 | NM_001039141.3 |
| TSPEAR, Thrombospondin type laminin G domain and EAR repeats, variant 1 | NM_144991.3 |
| TSPEAR, Thrombospondin type laminin G domain and EAR repeats, variant 2 | NM_001272037.2 |
| WBP2, WW domain binding protein 2, transcript variant 1 | NM_012478.4 |
| WBP2, WW domain binding protein 2, transcript variant 2 | NM_001330499.2 |
| WBP2, WW domain binding protein 2, transcript variant 3 | NM_001348170.1 |
| PRPS1, Phosphoribosyl pyrophosphate synthetase 1, transcript variant 1 | NM_002764.4 |
| PRPS1, Phosphoribosyl pyrophosphate synthetase 1, transcript variant 2 | NM_001204402.2 |
| POU3F4, POU class 3 homeobox 4 | NM_000307.5 |
| SMPX, Small muscle protein X-linked | NM_014332.3 |
| AIFM1, Apoptosis inducing factor mitochondria associated 1, transcript variant 1 | NM_004208.4 |
| AIFM1, Apoptosis inducing factor mitochondria associated 1, transcript variant 2 | NM_145812.3 |
| AIFM1, Apoptosis inducing factor mitochondria associated 1, transcript variant 4 | NM_001130846.4 |
| AIFM1, Apoptosis inducing factor mitochondria associated 1, transcript variant 5 | NM_001130847.4 |

TABLE 7
Amino acid sequences of Atoh1 variants

| Variant | Amino acid sequence |
|---|---|
| Atoh1 variant | MSRLLHAEEWAEVKELGDHHRQPQPHHLPQP |
| S328A amino | PPPPQPPATLQAREHPVYPPELSLLDSTDPR |
| acid sequence | AWLAPTLQGICTARAAQYLLHSPELGASEAA |
| | APRDEVDGRGELVRRSSGGASSSKSPGPVKV |
| | REQLCKLKGGVVVDELGCSRQRAPSSKQVNG |
| | VQKQRRLAANARERRRMHGLNHAFDQLRNVI |
| | PSFNNDKKLSKYETLQMAQIYINALSELLQT |
| | PSGGEQPPPPPASCKSDHHHLRTAASYEGGA |
| | GNATAAGAQQASGGSQRPTPPGSCRTRFSAP |
| | ASAGGYSVQLDALHFSTFEDSALTAMMAQKN |
| | LSPSLPGSILQPVQEENAKTSPRSHRSDGEF |
| | SPHSHYSDSDEAS (SEQ ID NO: 43) |
| Atoh1 variant | MSRLLHAEEWAEVKELGDHHRQPQPHHLPQP |
| S331A | PPPPQPPATLQAREHPVYPPELSLLDSTDPR |
| amino acid | AWLAPTLQGICTARAAQYLLHSPELGASEAA |
| sequence | APRDEVDGRGELVRRSSGGASSSKSPGPVKV |
| | REQLCKLKGGVVVDELGCSRQRAPSSKQVNG |
| | VQKQRRLAANARERRRMHGLNHAFDQLRNVI |
| | PSFNNDKKLSKYETLQMAQIYINALSELLQT |
| | PSGGEQPPPPPASCKSDHHHLRTAASYEGGA |
| | GNATAAGAQQASGGSQRPTPPGSCRTRFSAP |
| | ASAGGYSVQLDALHFSTFEDSALTAMMAQKN |
| | LSPSLPGSILQPVQEENSKTAPRSHRSDGEF |
| | SPHSHYSDSDEAS (SEQ ID NO: 44) |
| Atoh1 variant | MSRLLHAEEWAEVKELGDHHRQPQPHHLPQP |
| S334A | PPPPQPPATLQAREHPVYPPELSLLDSTDPR |
| amino acid | AWLAPTLQGICTARAAQYLLHSPELGASEAA |
| sequence | APRDEVDGRGELVRRSSGGASSSKSPGPVKV |
| | REQLCKLKGGVVVDELGCSRQRAPSSKQVNG |
| | VQKQRRLAANARERRRMHGLNHAFDQLRNVI |
| | PSFNNDKKLSKYETLQMAQIYINALSELLQT |
| | PSGGEQPPPPPASCKSDHHHLRTAASYEGGA |
| | GNATAAGAQQASGGSQRPTPPGSCRTRFSAP |
| | ASAGGYSVQLDALHFSTFEDSALTAMMAQKN |
| | LSPSLPGSILQPVQEENSKTSPRAHRSDGEF |
| | SPHSHYSDSDEAS (SEQ ID NO: 45) |
| Atoh1 variant | MSRLLHAEEWAEVKELGDHHRQPQPHHLPQP |
| S328A/S331A | PPPPQPPATLQAREHPVYPPELSLLDSTDPR |
| amino acid | AWLAPTLQGICTARAAQYLLHSPELGASEAA |
| sequence | APRDEVDGRGELVRRSSGGASSSKSPGPVKV |
| | REQLCKLKGGVVVDELGCSRQRAPSSKQVNG |
| | VQKQRRLAANARERRRMHGLNHAFDQLRNVI |
| | PSFNNDKKLSKYETLQMAQIYINALSELLQT |
| | PSGGEQPPPPPASCKSDHHHLRTAASYEGGA |
| | GNATAAGAQQASGGSQRPTPPGSCRTRFSAP |
| | ASAGGYSVQLDALHFSTFEDSALTAMMAQKN |
| | LSPSLPGSILQPVQEENAKTAPRSHRSDGEF |
| | SPHSHYSDSDEAS (SEQ ID NO: 46) |
| Atoh1 variant | MSRLLHAEEWAEVKELGDHHRQPQPHHLPQP |
| S328A/S334A | PPPPQPPATLQAREHPVYPPELSLLDSTDPR |
| amino acid | AWLAPTLQGICTARAAQYLLHSPELGASEAÅ |
| sequence | APRDEVDGRGELVRRSSGGASSSKSPGPVKV |
| | REQLCKLKGGVVVDELGCSRQRAPSSKQVNG |
| | VQKQRRLAANARERRRMHGLNHAFDQLRNVI |
| | PSFNNDKKLSKYETLQMAQIYINALSELLQT |
| | PSGGEQPPPPPASCKSDHHHLRTAASYEGGA |
| | GNATAAGAQQASGGSQRPTPPGSCRTRFSAP |
| | ASAGGYSVQLDALHFSTFEDSALTAMMAQKN |
| | LSPSLPGSILQPVQEENAKTSPRAHRSDGEF |
| | SPHSHYSDSDEAS (SEQ ID NO: 47) |
| Atoh1 variant | MSRLLHAEEWAEVKELGDHHRQPQPHHLPQP |
| S331A/S334A | PPPPQPPATLQAREHPVYPPELSLLDSTDPR |
| amino acid | AWLAPTLQGICTARAAQYLLHSPELGASEAA |
| sequence | APRDEVDGRGELVRRSSGGASSSKSPGPVKV |
| | REQLCKLKGGVVVDELGCSRQRAPSSKQVNG |
| | VQKQRRLAANARERRRMHGLNHAFDQLRNVI |
| | PSFNNDKKLSKYETLQMAQIYINALSELLQT |
| | PSGGEQPPPPPASCKSDHHHLRTAASYEGGA |
| | GNATAAGAQQASGGSQRPTPPGSCRTRFSAP |
| | ASAGGYSVQLDALHFSTFEDSALTAMMAQKN |
| | LSPSLPGSILQPVQEENSKTAPRAHRSDGEF |
| | SPHSHYSDSDEAS (SEQ ID NO: 48) |
| Atoh1 variant | MSRLLHAEEWAEVKELGDHHRQPQPHHLPQP |
| S328A/S331A/ | PPPPQPPATLQAREHPVYPPELSLLDSTDPR |
| S334 | AWLAPTLQGICTARAAQYLLHSPELGASEAA |
| amino acid | APRDEVDGRGELVRRSSGGASSSKSPGPVKV |
| sequence | REQLCKLKGGVVVDELGCSRQRAPSSKQVNG |
| | VQKQRRLAANARERRRMHGLNHAFDQLRNVI |
| | PSFNNDKKLSKYETLQMAQIYINALSELLQT |
| | PSGGEQPPPPPASCKSDHHHLRTAASYEGGA |
| | GNATAAGAQQASGGSQRPTPPGSCRTRFSAP |
| | ASAGGYSVQLDALHFSTFEDSALTAMMAQKN |
| | LSPSLPGSILQPVQEENAKTAPRAHRSDGEF |
| | SPHSHYSDSDEAS (SEQ ID NO: 49) |

In some embodiments, the vector contains a polynucleotide that encodes a dominant negative protein, such as a dominant negative Sox2 (dnSox2) protein. The dominant negative Sox2 protein may be produced by mutating the two nuclear localization signals in the high mobility group domain of Sox2 (as described in Li et al., J Biol Chem 282:19481-92 (2007)), by generating a Sox2 polynucleotide that lacks all or most of the high mobility group domain (as described in Kishi et al., Development 127:791-800 (2000)), by generating a Sox2 polynucleotide in which the high mobility group domain is fused with the engrailed repressor domain (as described in Kishi et al., Development 127:791-800 (2000)), or by generating a Sox2 polynucleotide that only encodes the Sox2 DNA binding domain (e.g., a C-terminally truncated version of Sox2 that can compete with wild-type Sox2 by binding to Sox2 recognition sites on DNA but that lacks a transactivation domain, e.g., as described in Pan and Schultz, Biology of Reproduction 85:409-416 (2011), Hutz et al., Carcinogenesis 35:942-950 (2013), and Gaete et al., Neural Development 7:13 (2012)). In some embodiments, the dominant negative Sox2 protein is encoded by the sequence:

(SEQ ID NO: 50)
ATGTATAACATGATGGAGACGGAGCTGAAGCCGCCGGGCCCGCAGC
AAGCTTCGGGGGGCGGCGGCGGAGGAGGCAACGCCACGGGGGGGGC
GACCGGCGGCAACCAGAAGAACAGCCCGGACCGCGTCACGGGGCCC
ATGAACGCCTTCATGGTATGGTCCCGGGGGCAGCTGGGTAAGATGG
CCCAGGAGAACCCCAAGATGCACAACTCGGAGATCAGCAAGCGCCT
GGGCGCGGAGTGGAAACTTTTGTCCGAGACCGAGAAGCGGCCGTTC
ATCGACGAGGCCAAGCGGCTGCGCGCTCTGCACATGAAGGAGCACC
CGGATTATAAATACCGGCCGCTGGGGAAAACCAAGACGCTCATGAA
GAAGGATAAGTACACGCTTCCCGGAGGCTTGCTGGCCCCCGGGGGG
AACAGCATGGCGAGCGGGGTTGGGGTGGGCGCCGGCCTGGGTGCGG
GCGTGAACCAGCGCATGGACAGCTACGCGCACATGAACGGCTGGAG
CAACGGCAGCTACAGCATGATGCAGGAGCAGCTGGGCTACCCGCAG
CACCCGGGCCTCAACGCTCACGGCGCGGCACAGATGCAACCGATGC
ACCGCTACGACGTCAGCGCCCTGCAGTACAACTCCATGACCAGCTC
GCAGACCTACATGAACGGCTCGCCCACCTACAGCATGTCCTACTCG
CAGCAGGGCACCCCCGGTATGGCGCTGGGCTCCATGGGCTCTGTGG
TCAAGTCCGAGGCCAGCTCCAGCCCCCCCGTGGTTACCTCTTCCTC
CCACTCCAGGGCGCCCTGCCAGGCCGGGGACCTCCGGGACATGATC
AGCATGTACCTCCCCGGCGCCGAGGTGCCGGAGCCCGCTGCGCCCA
GTAGACTGCACATGGCCCAGCACTACCAGAGCGGCCCGGTGCCCGG
CACGGCCATTAACGGCACACTGCCCCTGTCGCAC;

or the sequence:

(SEQ ID NO: 51)
ATGTATAACATGATGGAGACGGAGCTGAAGCCGCCGGGCCCGCAGC
AAGCTTCGGGGGGCGGCGGCGGAGGAGGCAACGCCACGGCGGCGGC
GACCGGCGGCAACCAGAAGAACAGCCCGGACCGCGTCACGGGGCCC
ATGAACGCCTTCATGGTATGGTCCCGGGGGCAGCTGGGTAAGATGG
CCCAGGAGAACCCCAAGATGCACAACTCGGAGATCAGCAAGCGCCT
GGGCGCGGAGTGGAAACTTTTGTCCGAGACCGAGAAGCGGCCGTTC
ATCGACGAGGCCAAGCGGCTGCGCGCTCTGCACATGAAGGAGCACC
CGGATTATAAATACCGGCCGCTGGGGAAAACCAAGACGCTCATGAA
GAAGGATAAGTACACGCTTCCCGGAGGCTTGCTGGCCCCCGGGGGG
AACAGCATGGCGAGCGGGGTTGGGGTGGGCGCCGGCCTGGGTGCGG
GCGTGAACCAGCGCATGGACAGCTACGCGCACATGAACGGCTGGAG
CAACGGCAGCTACAGCATGATGCAGGAGCAGCTGGGCTACCCGCAG
CACCCGGGCCTCAACGCTCACGGCGCGGCACAGATGCAACCGATGC
ACCGCTACGACGTCAGCGCCCTGCAGTACAACTCCATGACCAGCTC
GCAGACCTACATGAACGGCTCGCCCACCTACAGCATGTCCTACTCG
CAGCAGGGCACCCCCGGTATGGCGCTGGGCTCCATGGGCTCTGTGG
TCAAGTCCGAGGCCAGCTCCAGCCCCCCCGTGGTTACCTCTTCCTC
CCACTCCAGGGCGCCCTGCCAGGCCGGGGACCTCCGGGACATGATC
AGCATGTACCTCCCCGGCGCCGAGGTGCCGGAGCCCGCTGCGCCCA
GTAGACTGCACATGGCCCAGCACTACCAGAGCGGCCCGGTGCCCGG
CACGGCCATTAACGGCACACTGCCCCTGTCGCACATG.

### Inhibitory RINA

In some embodiments, the polynucleotide can be transcribed to produce an inhibitory RNA molecule, such as a short interfering RNA (siRNA) molecule or a short hairpin RNA (shRNA) molecule, e.g., a molecule that acts by way of the RNA interference (RNAI) pathway. In some embodiments, the inhibitory RNA molecule is directed to Sox2 (e.g., is a molecule that can decrease the expression level (e.g., protein level or mRNA level) of Sox2). Inhibitory RNA molecules directed to Sox2 include siRNA molecules and shRNA molecules that target full-length Sox2. An siRNA is a double-stranded RNA molecule that typically has a length of about 19-25 base pairs. An shRNA is an RNA molecule containing a hairpin turn that decreases expression of target genes via RNAi. An shRNA can also be embedded into the backbone of a miRNA (e.g., miRNA-30 or mir-E, e.g., to produce an shRNA-mir), as described in Silva et al., Nature Genetics 37:1281-1288 (2005) and Fellmann et al., Cell Reports 5:1704-1713 (2013), to achieve highly efficient target gene knockdown. Exemplary Sox2 shRNA and siRNA target sequences are provided in Tables 8 and 9, below. Sequences for plasmids containing exemplary Sox2 shRNAs that are embedded in miRNA backbones are provided in Table 10, below. Exemplary Sox2 siRNA sequences are provided in Table 11, below.

TABLE 8
Human Sox2 shRNA and siRNA targets

| SEQ ID NO: | Target sequence |
|---|---|
| 52 | CTGCCGAGAATCCATGTATAT |
| 53 | GTACAGTATTTATCGAGATAA |
| 54 | AGGAGCACCCGGATTATAAAT |
| 55 | TGGACAGTTACGCGCACATGA |
| 56 | TCCCATCACCCACAGCAAATG |
| 57 | CGAGATAAACATGGCAATCAA |
| 58 | CGCTCATGAAGAAGGATAAGT |
| 59 | CAGCTCGCAGACCTACATGAA |
| 60 | CAACGGCAGCTACAGCATGAT |
| 61 | CCACCTACAGCATGTCCTACT |
| 62 | CCCTGCAGTACAACTCCATGA |
| 63 | ACATGTCCCAGCACTACCAGA |
| 64 | GCACATGAACGGCTGGAGCAA |
| 65 | GCCCACCTACAGCATGTCCTA |
| 66 | GAAGAAGGATAAGTACACGCT |
| 71 | CCAGTAATATTTAGAGCTA |
| 72 | TTGTGATATTTTAAGGTTT |
| 73 | CTTATGGTTTGTAATATTT |
| 74 | TTGATTGCCATGTTTATCTCGA |
| 75 | TTATCTCGATAAATACTGTACA |

TABLE 9
Mouse Sox2 shRNA and siRNA targets

| SEQ ID NO: | Target sequence |
|---|---|
| 52 | CTGCCGAGAATCCATGTATAT |
| 53 | GTACAGTATTTATCGAGATAA |
| 54 | AGGAGCACCCGGATTATAAAT |
| 57 | CGAGATAAACATGGCAATCAA |
| 58 | CGCTCATGAAGAAGGATAAGT |
| 59 | CAGCTCGCAGACCTACATGAA |
| 60 | CAACGGCAGCTACAGCATGAT |
| 61 | CCACCTACAGCATGTCCTACT |
| 62 | CCCTGCAGTACAACTCCATGA |
| 64 | GCACATGAACGGCTGGAGCAA |
| 65 | GCCCACCTACAGCATGTCCTA |
| 66 | GAAGAAGGATAAGTACACGCT |
| 67 | ACCAATCCCATCCAAATTAAC |
| 68 | CAAAGAGATACAAGGGAATTG |
| 69 | TGCGCCCAGTAGACTGCACAT |
| 70 | CGCGGCACAGATGCAACCGAT |

TABLE 10
Exemplary plasmid sequences containing Sox2 shRNAs in a miRNA scaffold

| SEQ ID NO: | Plasmid sequence |
|---|---|
| 76 | ccttaattaggctgcgcgctcgctcgctcactgaggccgcccgggcaaagcccgggcgtcgggcgacctttggtcg |
| (P797) | cccggcctcagtgagcgagcgagcgcgcagagagggagtggccaactccatcactaggggttccttgtagttaat |
| 5′-mir 30 | gattaacccgccatgctacttatctacgtagccatgctctaggaagatcggaattcgcccttaagctagcggcgcgcc |
| sequence at | accggtgcgatcgccgttacataacttacggtaaatggcccgcctggctgaccgcccaacgacccccgcccattga |
| positions | cgtcaataatgacgtatgttcccatagtaacgccaatagggactttccattgacgtcaatgggtggagtatttacggta |
| 2109-2233 | aactgcccacttggcagtacatcaagtgtatcatatgccaagtacgccccctattgacgtcaatgacggtaaatggcc |
| shRNA_Sox2_2 | cgcctggcattatgcccagtacatgaccttatgggactttcctacttggcagtacatctacgtattagtcatcgctattacc |
| sequence at | atggtgatgcggttttggcagtacatcaatgggcgtggatagcggtttgactcacggggatttccaagtctccacccca |
| positions | ttgacgtcaatgggagtttgttttggcaccaaaatcaacgggactttccaaaatgtcgtaacaactccgccccattgac |
| 2234-2296 | gcaaatgggcggtaggcgtgtacggtgggaggtctatataagcagagctggtttagtgaaccgtcagatcctgcag |
| 3′-mir 30 | aagttggtcgtgaggcactgggcaggtaagtatcaaggttacaagacaggtttaaggagaccaatagaaactggg |
| sequence at | cttgtcgagacagagaagactcttgcgtttctgataggcacctattggtcttactgacatccactttgcctttctctccaca |
| positions | ggtgtccaggcggccgcgccaccatgccagagccagcgaagtctgctcccgccccgaaaaagggctccaagaa |
| 2297-2426 | ggcggtgactaaggcgcagaagaaaggcggcaagaagcgcaagcgcagccgcaaggagagctattccatcta |
| | tgtgtacaaggttctgaagcaggtccaccctgacaccggcatttcgtccaaggccatgggcatcatgaattcgtttgtg |
| | aacgacattttcgagcgcatcgcaggtgaggcttcccgcctggcgcattacaacaagcgctcgaccatcacctcca |
| | gggagatccagacggccgtgcgcctgctgctgcctggggagttggccaagcacgccgtgtccgagggtactaag |
| | gccatcaccaagtacaccagcgctaaggatccaccggtcgccaccatggtgagcaagggcgaggagctgttcac |
| | cggggtggtgcccatcctggtcgagctggacggcgacgtaaacggccacaagttcagcgtgtccggcgagggcg |
| | agggcgatgccacctacggcaagctgaccctgaagttcatctgcaccaccggcaagctgcccgtgccctggccca |
| | ccctcgtgaccaccctgacctacggcgtgcagtgcttcagccgctaccccgaccacatgaagcagcacgacttcttc |
| | aagtccgccatgcccgaaggctacgtccaggagcgcaccatcttcttcaaggacgacggcaactacaagacccg |
| | cgccgaggtgaagttcgagggcgacaccctggtgaaccgcatcgagctgaagggcatcgacttcaaggaggac |
| | ggcaacatcctggggcacaagctggagtacaactacaacagccacaacgtctatatcatggccgacaagcaga |
| | agaacggcatcaaggtgaacttcaagatccgccacaacatcgaggacggcagcgtgcagctcgccgaccacta |
| | ccagcagaacacccccatcggcgacggccccgtgctgctgcccgacaaccactacctgagcacccagtccgcc |
| | ctgagcaaagaccccaacgagaagcgcgatcacatggtcctgctggagttcgtgaccgccgccgggatcactctc |
| | ggcatggacgagctgtacaagtaataagcttctcgactagggataacagggtaattgtttgaatgaggcttcagtactt |
| | tacagaatcgttgcctgcacatcttggaaacacttgctgggattacttcttcaggttaacccaacagaaggctcgaga |
| | aggtatattgctgttgAcagtgAgcgCcgagataaacatggcaatcaatagtgaagccacagatgtattgattgcc |
| | atgtttatctcgatgcCtactgCctcgcaattgaaggggctactttaggagcaattatcttgtttactaaaactgaatacc |
| | ttgctatctctttgatacatttttacaaagctgaattaaaatggtataaattaaatcacttttataaattaaatcacttttttacg |
| | cgtggatccaatcaacctctggattacaaaatttgtgaaagattgactggtattcttaactatgttgctccttttacgctatg |
| | tggatacgctgctttaatgcctttgtatcatgctattgcttcccgtatggctttcattttctcctccttgtataaatcctggttgct |
| | gtctctttatgaggagttgtggcccgttgtcaggcaacgtggcgtggtgtgcactgtgtttgctgacgcaacccccactg |
| | gttggggcattgccaccacctgtcagctcctttccgggactttcgctttccccctccctattgccacggcggaactcatc |
| | gccgcctgccttgcccgctgctggacaggggctcggctgttgggcactgacaattccgtggtgttgtcggggaaatca |
| | tcgtcctttccttggctgctcgcctgtgttgccacctggattctgcgcgggacgtccttctgctacgtcccttcggccctca |
| | atccagcggaccttccttcccgcggcctgctgccggctctgcggcctcttccgcgtcttcgagatctgcctcgactgtgc |
| | cttctagttgccagccatctgttgtttgcccctcccccgtgccttccttgaccctggaaggtgccactcccactgtcctttcc |
| | taataaaatgaggaaattgcatcgcattgtctgagtaggtgtcattctattctggggggtggggggggcaggacagc |
| | aagggggaggattgggaagacaatagcaggcatgctggggagagctcttaagggcgaattcccgataaggatct |
| | tcctagagcatggctacgtagataagtagcatggcgggttaatcattaactacaaggaacccctagtgatggagttg |
| | gccactccctctctgcgcgctcgctcgctcactgaggccgggcgaccaaaggtcgcccgacgcccgggctttgccc |
| | gggcggcctcagtgagcgagcgagcgcgcagccttaattaacctaattcactggccgtcgttttacaacgtcgtgact |
| | gggaaaaccctggcgttacccaacttaatcgccttgcagcacatccccctttcgccagctggcgtaatagcgaaga |
| | ggcccgcaccgatcgcccttcccaacagttgcgcagcctgaatggcgaatgggacgcgccctgtagcggcgcatt |
| | aagcgcggcgggtgtggtggttacgcgcagcgtgaccgctacacttgccagcgccctagcgcccgctcctttcgcttt |
| | cttcccttcctttctcgccacgttcgccggctttccccgtcaagctctaaatcgggggctccctttagggttccgatttagtg |
| | ctttacggcacctcgaccccaaaaaacttgattagggtgatggttcacgtagtgggccatcgccctgatagacggtttt |
| | tcgccctttgacgttggagtccacgttctttaatagtggactcttgttccaaactggaacaacactcaaccctatctcggt |
| | ctattcttttgatttataagggattttgccgatttcggcctattggttaaaaaatgagctgatttaacaaaaatttaacgcga |
| | attttaacaaaatattaacgcttacaatttaggtggcacttttcggggaaatgtgcgcggaacccctatttgtttatttttcta |
| | aatacattcaaatatgtatccgctcatgagacaataaccctgataaatgcttcaataatattgaaaaaggaagagtat |
| | gagccatattcaacgggaaacgtcgaggccgcgattaaattccaacatggatgctgatttatatgggtataaatggg |
| | ctcgcgataatgtcgggcaatcaggtgcgacaatctatcgcttgtatgggaagcccgatgcgccagagttgtttctga |
| | aacatggcaaaggtagcgttgccaatgatgttacagatgagatggtcagactaaactggctgacggaatttatgcct |
| | cttccgaccatcaagcattttatccgtactcctgatgatgcatggttactcaccactgcgatccccggaaaaacagcat |
| | tccaggtattagaagaatatcctgattcaggtgaaaatattgttgatgcgctggcagtgttcctgcgccggttgcattcg |
| | attcctgtttgtaattgtccttttaacagcgatcgcgtatttcgtcttgctcaggcgcaatcacgaatgaataacggtttggtt |
| | gatgcgagtgattttgatgacgagcgtaatggctggcctgttgaacaagtctggaaagaaatgcataaacttttgccat |
| | tctcaccggattcagtcgtcactcatggtgatttctcacttgataaccttatttttgacgaggggaaattaataggttgtatt |
| | gatgttggacgagtcggaatcgcagaccgataccaggatcttgccatcctatggaactgcctcggtgagttttctcctt |
| | cattacagaaacggctttttcaaaaatatggtattgataatcctgatatgaataaattgcagtttcatttgatgctcgatga |
| | gtttttctaactgtcagaccaagtttactcatatatactttagattgatttaaaacttcatttttaatttaaaaggatctaggtg |
| | aagatcctttttgataatctcatgaccaaaatcccttaacgtgagttttcgttccactgagcgtcagaccccgtagaaaa |
| | gatcaaaggatcttcttgagatcctttttttctgcgcgtaatctgctgcttgcaaacaaaaaaaccaccgctaccagcg |
| | gtggtttgtttgccggatcaagagctaccaactctttttccgaaggtaactggcttcagcagagcgcagataccaaata |
| | ctgttcttctagtgtagccgtagttaggccaccacttcaagaactctgtagcaccgcctacatacctcgctctgctaatcc |
| | tgttaccagtggctgctgccagtggcgataagtcgtgtcttaccgggttggactcaagacgatagttaccggataagg |
| | cgcagcggtcgggctgaacggggggttcgtgcacacagcccagcttggagcgaacgacctacaccgaactgag |
| | atacctacagcgtgagctatgagaaagcgccacgcttcccgaagggagaaaggcggacaggtatccggtaagc |
| | ggcagggtcggaacaggagagcgcacgagggagcttccagggggaaacgcctggtatctttatagtcctgtcgg |
| | gtttcgccacctctgacttgagcgtcgatttttgtgatgctcgtcaggggggcggagcctatggaaaaacgccagcaa |
| | cgcggcctttttacggttcctggccttttgctggccttttgctcacatgttctttcctgcgttatcccctgattctgtggataacc |
| | gtattaccgcctttgagtgagctgataccgctcgccgcagccgaacgaccgagcgcagcgagtcagtgagcgagg |
| | aagcggaagagcgcccaatacgcaaaccgcctctccccgcgcgttggccgattcattaatgcagctggcacgac |
| | aggtttcccgactggaaagcgggcagtgagcgcaacgcaattaatgtgagttagctcactcattaggcaccccagg |
| | ctttacactttatgcttccggctcgtatgttgtgtggaattgtgagcggataacaatttcacacaggaaacagctatgacc |
| | atgattacgccagatttaattaagg |
| 77 | ccttaattaggctgcgcgctcgctcgctcactgaggccgcccgggcaaagcccgggcgtcgggcgacctttggtcg |
| (P900) | cccggcctcagtgagcgagcgagcgcgcagagagggagtggccaactccatcactaggggttccttgtagttaat |
| 5′-mirE | gattaacccgccatgctacttatctacgtagccatgctctaggaagatcggaattcgcccttaagctagcggcgcgcc |
| sequence at | accggtgcgatcgccgttacataacttacggtaaatggcccgcctggctgaccgcccaacgacccccgcccattga |
| positions | cgtcaataatgacgtatgttcccatagtaacgccaatagggactttccattgacgtcaatgggtggagtatttacggta |
| 2109-2233 | aactgcccacttggcagtacatcaagtgtatcatatgccaagtacgccccctattgacgtcaatgacggtaaatggcc |
| shRNA_Sox2_2 | cgcctggcattatgcccagtacatgaccttatgggactttcctacttggcagtacatctacgtattagtcatcgctattacc |
| sequence at | atggtgatgcggttttggcagtacatcaatgggcgtggatagcggtttgactcacggggatttccaagtctccacccca |
| positions | ttgacgtcaatgggagtttgttttggcaccaaaatcaacgggactttccaaaatgtcgtaacaactccgccccattgac |
| 2234-2296 | gcaaatgggcggtaggcgtgtacggtgggaggtctatataagcagagctggtttagtgaaccgtcagatcctgcag |
| 3′-mirE | aagttggtcgtgaggcactgggcaggtaagtatcaaggttacaagacaggtttaaggagaccaatagaaactggg |
| sequence at | cttgtcgagacagagaagactcttgcgtttctgataggcacctattggtcttactgacatccactttgcctttctctccaca |
| positions | ggtgtccaggcggccgcgccaccatgccagagccagcgaagtctgctcccgccccgaaaaagggctccaagaa |
| 2297-2408 | ggcggtgactaaggcgcagaagaaaggcggcaagaagcgcaagcgcagccgcaaggagagctattccatcta |
| | tgtgtacaaggttctgaagcaggtccaccctgacaccggcatttcgtccaaggccatgggcatcatgaattcgtttgtg |
| | aacgacattttcgagcgcatcgcaggtgaggcttcccgcctggcgcattacaacaagcgctcgaccatcacctcca |
| | gggagatccagacggccgtgcgcctgctgctgcctggggagttggccaagcacgccgtgtccgagggtactaag |
| | gccatcaccaagtacaccagcgctaaggatccaccggtcgccaccatggtgagcaagggcgaggagctgttcac |
| | cggggtggtgcccatcctggtcgagctggacggcgacgtaaacggccacaagttcagcgtgtccggcgagggcg |
| | agggcgatgccacctacggcaagctgaccctgaagttcatctgcaccaccggcaagctgcccgtgccctggccca |
| | ccctcgtgaccaccctgacctacggcgtgcagtgcttcagccgctaccccgaccacatgaagcagcacgacttcttc |
| | aagtccgccatgcccgaaggctacgtccaggagcgcaccatcttcttcaaggacgacggcaactacaagacccg |
| | cgccgaggtgaagttcgagggcgacaccctggtgaaccgcatcgagctgaagggcatcgacttcaaggaggac |
| | ggcaacatcctggggcacaagctggagtacaactacaacagccacaacgtctatatcatggccgacaagcaga |
| | agaacggcatcaaggtgaacttcaagatccgccacaacatcgaggacggcagcgtgcagctcgccgaccacta |
| | ccagcagaacacccccatcggcgacggccccgtgctgctgcccgacaaccactacctgagcacccagtccgcc |
| | ctgagcaaagaccccaacgagaagcgcgatcacatggtcctgctggagttcgtgaccgccgccgggatcactctc |
| | ggcatggacgagctgtacaagtaataagcttctcgactagggataacagggtaattgtttgaatgaggcttcagtactt |
| | tacagaatcgttgcctgcacatcttggaaacacttgctgggattacttcgacttcttaacccaacagaaggctcgaga |
| | aggtatattgctgttgacagtgagcgccgagataaacatggcaatcaatagtgaagccacagatgtattgattgccat |
| | gtttatctcgatgcctactgcctcggacttcaaggggctagaattcgagcaattatcttgtttactaaaactgaataccttg |
| | ctatctctttgatacatttttacaaagctgaattaaaatggtataaattaaatcacttttttcaattgacgcgtaattctaccg |
| | gatccaatcaacctctggattacaaaatttgtgaaagattgactggtattcttaactatgttgctccttttacgctatgtgga |
| | tacgctgctttaatgcctttgtatcatgctattgcttcccgtatggctttcattttctcctccttgtataaatcctggttgctgtctct |
| | ttatgaggagttgtggcccgttgtcaggcaacgtggcgtggtgtgcactgtgtttgctgacgcaacccccactggttgg |
| | ggcattgccaccacctgtcagctcctttccgggactttcgctttccccctccctattgccacggcggaactcatcgccgc |
| | ctgccttgcccgctgctggacaggggctcggctgttgggcactgacaattccgtggtgttgtcggggaaatcatcgtcc |
| | tttccttggctgctcgcctgtgttgccacctggattctgcgcgggacgtccttctgctacgtcccttcggccctcaatccag |
| | cggaccttccttcccgcggcctgctgccggctctgcggcctcttccgcgtcttcgagatctgcctcgactgtgccttctag |
| | ttgccagccatctgttgtttgcccctcccccgtgccttccttgaccctggaaggtgccactcccactgtcctttcctaataa |
| | aatgaggaaattgcatcgcattgtctgagtaggtgtcattctattctggggggtggggggggcaggacagcaaggg |
| | ggaggattgggaagacaatagcaggcatgctggggagagctcttaagggcgaattcccgataaggatcttcctag |
| | agcatggctacgtagataagtagcatggcgggttaatcattaactacaaggaacccctagtgatggagttggccact |
| | ccctctctgcgcgctcgctcgctcactgaggccgggcgaccaaaggtcgcccgacgcccgggctttgcccgggcg |
| | gcctcagtgagcgagcgagcgcgcagccttaattaacctaattcactggccgtcgttttacaacgtcgtgactggga |
| | aaaccctggcgttacccaacttaatcgccttgcagcacatccccctttcgccagctggcgtaatagcgaagaggccc |
| | gcaccgatcgcccttcccaacagttgcgcagcctgaatggcgaatgggacgcgccctgtagcggcgcattaagcg |
| | cggcgggtgtggtggttacgcgcagcgtgaccgctacacttgccagcgccctagcgcccgctcctttcgctttcttccc |
| | ttcctttctcgccacgttcgccggctttccccgtcaagctctaaatcgggggctccctttagggttccgatttagtgctttac |
| | ggcacctcgaccccaaaaaacttgattagggtgatggttcacgtagtgggccatcgccctgatagacggtttttcgcc |
| | ctttgacgttggagtccacgttctttaatagtggactcttgttccaaactggaacaacactcaaccctatctcggtctattct |
| | tttgatttataagggattttgccgatttcggcctattggttaaaaaatgagctgatttaacaaaaatttaacgcgaattttaa |
| | caaaatattaacgcttacaatttaggtggcacttttcggggaaatgtgcgcggaacccctatttgtttatttttctaaatac |
| | attcaaatatgtatccgctcatgagacaataaccctgataaatgcttcaataatattgaaaaaggaagagtatgagcc |
| | atattcaacgggaaacgtcgaggccgcgattaaattccaacatggatgctgatttatatgggtataaatgggctcgcg |
| | ataatgtcgggcaatcaggtgcgacaatctatcgcttgtatgggaagcccgatgcgccagagttgtttctgaaacatg |
| | gcaaaggtagcgttgccaatgatgttacagatgagatggtcagactaaactggctgacggaatttatgcctcttccga |
| | ccatcaagcattttatccgtactcctgatgatgcatggttactcaccactgcgatccccggaaaaacagcattccaggt |
| | attagaagaatatcctgattcaggtgaaaatattgttgatgcgctggcagtgttcctgcgccggttgcattcgattcctgtt |
| | tgtaattgtccttttaacagcgatcgcgtatttcgtcttgctcaggcgcaatcacgaatgaataacggtttggttgatgcg |
| | agtgattttgatgacgagcgtaatggctggcctgttgaacaagtctggaaagaaatgcataaacttttgccattctcac |
| | cggattcagtcgtcactcatggtgatttctcacttgataaccttatttttgacgaggggaaattaataggttgtattgatgttg |
| | gacgagtcggaatcgcagaccgataccaggatcttgccatcctatggaactgcctcggtgagttttctccttcattaca |
| | gaaacggctttttcaaaaatatggtattgataatcctgatatgaataaattgcagtttcatttgatgctcgatgagtttttcta |
| | actgtcagaccaagtttactcatatatactttagattgatttaaaacttcatttttaatttaaaaggatctaggtgaagatcct |
| | ttttgataatctcatgaccaaaatcccttaacgtgagttttcgttccactgagcgtcagaccccgtagaaaagatcaaa |
| | ggatcttcttgagatcctttttttctgcgcgtaatctgctgcttgcaaacaaaaaaaccaccgctaccagcggtggtttgtt |
| | tgccggatcaagagctaccaactctttttccgaaggtaactggcttcagcagagcgcagataccaaatactgttcttct |
| | agtgtagccgtagttaggccaccacttcaagaactctgtagcaccgcctacatacctcgctctgctaatcctgttacca |
| | gtggctgctgccagtggcgataagtcgtgtcttaccgggttggactcaagacgatagttaccggataaggcgcagcg |
| | gtcgggctgaacggggggttcgtgcacacagcccagcttggagcgaacgacctacaccgaactgagatacctac |
| | agcgtgagctatgagaaagcgccacgcttcccgaagggagaaaggcggacaggtatccggtaagcggcaggg |
| | tcggaacaggagagcgcacgagggagcttccagggggaaacgcctggtatctttatagtcctgtcgggtttcgcca |
| | cctctgacttgagcgtcgatttttgtgatgctcgtcaggggggcggagcctatggaaaaacgccagcaacgcggcct |
| | ttttacggttcctggccttttgctggccttttgctcacatgttctttcctgcgttatcccctgattctgtggataaccgtattaccg |
| | cctttgagtgagctgataccgctcgccgcagccgaacgaccgagcgcagcgagtcagtgagcgaggaagcgga |
| | agagcgcccaatacgcaaaccgcctctccccgcgcgttggccgattcattaatgcagctggcacgacaggtttccc |
| | gactggaaagcgggcagtgagcgcaacgcaattaatgtgagttagctcactcattaggcaccccaggctttacactt |
| | tatgcttccggctcgtatgttgtgtggaattgtgagcggataacaatttcacacaggaaacagctatgaccatgattac |
| | gccagatttaattaagg |
| 78 | ccttaattaggctgcgcgctcgctcgctcactgaggccgcccgggcaaagcccgggcgtcgggcgacctttggtcg |
| (P799) | cccggcctcagtgagcgagcgagcgcgcagagagggagtggccaactccatcactaggggttccttgtagttaat |
| 5′-mir30 | gattaacccgccatgctacttatctacgtagccatgctctaggaagatcggaattcgcccttaagctagcggcgcgcc |
| sequence at | accggtgcgatcgccgttacataacttacggtaaatggcccgcctggctgaccgcccaacgacccccgcccattga |
| positions | cgtcaataatgacgtatgttcccatagtaacgccaatagggactttccattgacgtcaatgggtggagtatttacggta |
| 2109-2233 | aactgcccacttggcagtacatcaagtgtatcatatgccaagtacgccccctattgacgtcaatgacggtaaatggcc |
| shRNA_Sox2_4 | cgcctggcattatgcccagtacatgaccttatgggactttcctacttggcagtacatctacgtattagtcatcgctattacc |
| sequence at | atggtgatgcggttttggcagtacatcaatgggcgtggatagcggtttgactcacggggatttccaagtctccacccca |
| positions | ttgacgtcaatgggagtttgttttggcaccaaaatcaacgggactttccaaaatgtcgtaacaactccgccccattgac |
| 2234-2296 | gcaaatgggcggtaggcgtgtacggtgggaggtctatataagcagagctggtttagtgaaccgtcagatcctgcag |
| 3′-mir 30 | aagttggtcgtgaggcactgggcaggtaagtatcaaggttacaagacaggtttaaggagaccaatagaaactggg |
| sequence at | cttgtcgagacagagaagactcttgcgtttctgataggcacctattggtcttactgacatccactttgcctttctctccaca |
| positions | ggtgtccaggcggccgcgccaccatgccagagccagcgaagtctgctcccgccccgaaaaagggctccaagaa |
| 2297-2426 | ggcggtgactaaggcgcagaagaaaggcggcaagaagcgcaagcgcagccgcaaggagagctattccatcta |
| | tgtgtacaaggttctgaagcaggtccaccctgacaccggcatttcgtccaaggccatgggcatcatgaattcgtttgtg |
| | aacgacattttcgagcgcatcgcaggtgaggcttcccgcctggcgcattacaacaagcgctcgaccatcacctcca |
| | gggagatccagacggccgtgcgcctgctgctgcctggggagttggccaagcacgccgtgtccgagggtactaag |
| | gccatcaccaagtacaccagcgctaaggatccaccggtcgccaccatggtgagcaagggcgaggagctgttcac |
| | cggggtggtgcccatcctggtcgagctggacggcgacgtaaacggccacaagttcagcgtgtccggcgagggcg |
| | agggcgatgccacctacggcaagctgaccctgaagttcatctgcaccaccggcaagctgcccgtgccctggccca |
| | ccctcgtgaccaccctgacctacggcgtgcagtgcttcagccgctaccccgaccacatgaagcagcacgacttcttc |
| | aagtccgccatgcccgaaggctacgtccaggagcgcaccatcttcttcaaggacgacggcaactacaagacccg |
| | cgccgaggtgaagttcgagggcgacaccctggtgaaccgcatcgagctgaagggcatcgacttcaaggaggac |
| | ggcaacatcctggggcacaagctggagtacaactacaacagccacaacgtctatatcatggccgacaagcaga |
| | agaacggcatcaaggtgaacttcaagatccgccacaacatcgaggacggcagcgtgcagctcgccgaccacta |
| | ccagcagaacacccccatcggcgacggccccgtgctgctgcccgacaaccactacctgagcacccagtccgcc |
| | ctgagcaaagaccccaacgagaagcgcgatcacatggtcctgctggagttcgtgaccgccgccgggatcactctc |
| | ggcatggacgagctgtacaagtaataagcttctcgactagggataacagggtaattgtttgaatgaggcttcagtactt |
| | tacagaatcgttgcctgcacatcttggaaacacttgctgggattacttcttcaggttaacccaacagaaggctcgaga |
| | aggtatattgctgttgacagtgagcgcgtacagtatttatcgagataatagtgaagccacagatgtattatctcgataa |
| | atactgtacatgcctactgcctcgcaattgaaggggctactttaggagcaattatcttgtttactaaaactgaataccttg |
| | ctatctctttgatacatttttacaaagctgaattaaaatggtataaattaaatcacttttataaattaaatcacttttttacgcgt |
| | ggatccaatcaacctctggattacaaaatttgtgaaagattgactggtattcttaactatgttgctccttttacgctatgtgg |
| | atacgctgctttaatgcctttgtatcatgctattgcttcccgtatggctttcattttctcctccttgtataaatcctggttgctgtct |
| | ctttatgaggagttgtggcccgttgtcaggcaacgtggcgtggtgtgcactgtgtttgctgacgcaacccccactggttg |
| | gggcattgccaccacctgtcagctcctttccgggactttcgctttccccctccctattgccacggcggaactcatcgccg |
| | cctgccttgcccgctgctggacaggggctcggctgttgggcactgacaattccgtggtgttgtcggggaaatcatcgtc |
| | ctttccttggctgctcgcctgtgttgccacctggattctgcgcgggacgtccttctgctacgtcccttcggccctcaatcca |
| | gcggaccttccttcccgcggcctgctgccggctctgcggcctcttccgcgtcttcgagatctgcctcgactgtgccttcta |
| | gttgccagccatctgttgtttgcccctcccccgtgccttccttgaccctggaaggtgccactcccactgtcctttcctaata |
| | aaatgaggaaattgcatcgcattgtctgagtaggtgtcattctattctggggggtggggggggcaggacagcaagg |
| | gggaggattgggaagacaatagcaggcatgctggggagagctcttaagggcgaattcccgataaggatcttccta |
| | gagcatggctacgtagataagtagcatggcgggttaatcattaactacaaggaacccctagtgatggagttggcca |
| | ctccctctctgcgcgctcgctcgctcactgaggccgggcgaccaaaggtcgcccgacgcccgggctttgcccgggc |
| | ggcctcagtgagcgagcgagcgcgcagccttaattaacctaattcactggccgtcgttttacaacgtcgtgactggg |
| | aaaaccctggcgttacccaacttaatcgccttgcagcacatccccctttcgccagctggcgtaatagcgaagaggcc |
| | cgcaccgatcgcccttcccaacagttgcgcagcctgaatggcgaatgggacgcgccctgtagcggcgcattaagc |
| | gcggcgggtgtggtggttacgcgcagcgtgaccgctacacttgccagcgccctagcgcccgctcctttcgctttcttcc |
| | cttcctttctcgccacgttcgccggctttccccgtcaagctctaaatcgggggctccctttagggttccgatttagtgcttta |
| | cggcacctcgaccccaaaaaacttgattagggtgatggttcacgtagtgggccatcgccctgatagacggtttttcgc |
| | cctttgacgttggagtccacgttctttaatagtggactcttgttccaaactggaacaacactcaaccctatctcggtctatt |
| | cttttgatttataagggattttgccgatttcggcctattggttaaaaaatgagctgatttaacaaaaatttaacgcgaatttt |
| | aacaaaatattaacgcttacaatttaggtggcacttttcggggaaatgtgcgcggaacccctatttgtttatttttctaaat |
| | acattcaaatatgtatccgctcatgagacaataaccctgataaatgcttcaataatattgaaaaaggaagagtatgag |
| | ccatattcaacgggaaacgtcgaggccgcgattaaattccaacatggatgctgatttatatgggtataaatgggctcg |
| | cgataatgtcgggcaatcaggtgcgacaatctatcgcttgtatgggaagcccgatgcgccagagttgtttctgaaaca |
| | tggcaaaggtagcgttgccaatgatgttacagatgagatggtcagactaaactggctgacggaatttatgcctcttcc |
| | gaccatcaagcattttatccgtactcctgatgatgcatggttactcaccactgcgatccccggaaaaacagcattcca |
| | ggtattagaagaatatcctgattcaggtgaaaatattgttgatgcgctggcagtgttcctgcgccggttgcattcgattcc |
| | tgtttgtaattgtccttttaacagcgatcgcgtatttcgtcttgctcaggcgcaatcacgaatgaataacggtttggttgatg |
| | cgagtgattttgatgacgagcgtaatggctggcctgttgaacaagtctggaaagaaatgcataaacttttgccattctc |
| | accggattcagtcgtcactcatggtgatttctcacttgataaccttatttttgacgaggggaaattaataggttgtattgatg |
| | ttggacgagtcggaatcgcagaccgataccaggatcttgccatcctatggaactgcctcggtgagttttctccttcatta |
| | cagaaacggctttttcaaaaatatggtattgataatcctgatatgaataaattgcagtttcatttgatgctcgatgagttttt |
| | ctaactgtcagaccaagtttactcatatatactttagattgatttaaaacttcatttttaatttaaaaggatctaggtgaaga |
| | tcctttttgataatctcatgaccaaaatcccttaacgtgagttttcgttccactgagcgtcagaccccgtagaaaagatc |
| | aaaggatcttcttgagatcctttttttctgcgcgtaatctgctgcttgcaaacaaaaaaaccaccgctaccagcggtggt |
| | ttgtttgccggatcaagagctaccaactctttttccgaaggtaactggcttcagcagagcgcagataccaaatactgtt |
| | cttctagtgtagccgtagttaggccaccacttcaagaactctgtagcaccgcctacatacctcgctctgctaatcctgtt |
| | accagtggctgctgccagtggcgataagtcgtgtcttaccgggttggactcaagacgatagttaccggataaggcgc |
| | agcggtcgggctgaacggggggttcgtgcacacagcccagcttggagcgaacgacctacaccgaactgagata |
| | cctacagcgtgagctatgagaaagcgccacgcttcccgaagggagaaaggcggacaggtatccggtaagcggc |
| | agggtcggaacaggagagcgcacgagggagcttccagggggaaacgcctggtatctttatagtcctgtcgggtttc |
| | gccacctctgacttgagcgtcgatttttgtgatgctcgtcaggggggcggagcctatggaaaaacgccagcaacgc |
| | ggcctttttacggttcctggccttttgctggccttttgctcacatgttctttcctgcgttatcccctgattctgtggataaccgtat |
| | taccgcctttgagtgagctgataccgctcgccgcagccgaacgaccgagcgcagcgagtcagtgagcgaggaag |
| | cggaagagcgcccaatacgcaaaccgcctctccccgcgcgttggccgattcattaatgcagctggcacgacaggt |
| | ttcccgactggaaagcgggcagtgagcgcaacgcaattaatgtgagttagctcactcattaggcaccccaggcttta |
| | cactttatgcttccggctcgtatgttgtgtggaattgtgagcggataacaatttcacacaggaaacagctatgaccatg |
| | attacgccagatttaattaagg |
| 79 | ccttaattaggctgcgcgctcgctcgctcactgaggccgcccgggcaaagcccgggcgtcgggcgacctttggtcg |
| (P901) | cccggcctcagtgagcgagcgagcgcgcagagagggagtggccaactccatcactaggggttccttgtagttaat |
| 5′-mirE | gattaacccgccatgctacttatctacgtagccatgctctaggaagatcggaattcgcccttaagctagcggcgcgcc |
| sequence at | accggtgcgatcgccgttacataacttacggtaaatggcccgcctggctgaccgcccaacgacccccgcccattga |
| positions | cgtcaataatgacgtatgttcccatagtaacgccaatagggactttccattgacgtcaatgggtggagtatttacggta |
| 2109-2233 | aactgcccacttggcagtacatcaagtgtatcatatgccaagtacgccccctattgacgtcaatgacggtaaatggcc |
| shRNA_Sox2_4 | cgcctggcattatgcccagtacatgaccttatgggactttcctacttggcagtacatctacgtattagtcatcgctattacc |
| sequence at | atggtgatgcggttttggcagtacatcaatgggcgtggatagcggtttgactcacggggatttccaagtctccacccca |
| positions | ttgacgtcaatgggagtttgttttggcaccaaaatcaacgggactttccaaaatgtcgtaacaactccgccccattgac |
| 2234-2296 | gcaaatgggcggtaggcgtgtacggtgggaggtctatataagcagagctggtttagtgaaccgtcagatcctgcag |
| 3′-mirE | aagttggtcgtgaggcactgggcaggtaagtatcaaggttacaagacaggtttaaggagaccaatagaaactggg |
| sequence at | cttgtcgagacagagaagactcttgcgtttctgataggcacctattggtcttactgacatccactttgcctttctctccaca |
| positions | ggtgtccaggcggccgcgccaccatgccagagccagcgaagtctgctcccgccccgaaaaagggctccaagaa |
| 2297-2408 | ggcggtgactaaggcgcagaagaaaggcggcaagaagcgcaagcgcagccgcaaggagagctattccatcta |
| | tgtgtacaaggttctgaagcaggtccaccctgacaccggcatttcgtccaaggccatgggcatcatgaattcgtttgtg |
| | aacgacattttcgagcgcatcgcaggtgaggcttcccgcctggcgcattacaacaagcgctcgaccatcacctcca |
| | gggagatccagacggccgtgcgcctgctgctgcctggggagttggccaagcacgccgtgtccgagggtactaag |
| | gccatcaccaagtacaccagcgctaaggatccaccggtcgccaccatggtgagcaagggcgaggagctgttcac |
| | cggggtggtgcccatcctggtcgagctggacggcgacgtaaacggccacaagttcagcgtgtccggcgagggcg |
| | agggcgatgccacctacggcaagctgaccctgaagttcatctgcaccaccggcaagctgcccgtgccctggccca |
| | ccctcgtgaccaccctgacctacggcgtgcagtgcttcagccgctaccccgaccacatgaagcagcacgacttcttc |
| | aagtccgccatgcccgaaggctacgtccaggagcgcaccatcttcttcaaggacgacggcaactacaagacccg |
| | cgccgaggtgaagttcgagggcgacaccctggtgaaccgcatcgagctgaagggcatcgacttcaaggaggac |
| | ggcaacatcctggggcacaagctggagtacaactacaacagccacaacgtctatatcatggccgacaagcaga |
| | agaacggcatcaaggtgaacttcaagatccgccacaacatcgaggacggcagcgtgcagctcgccgaccacta |
| | ccagcagaacacccccatcggcgacggccccgtgctgctgcccgacaaccactacctgagcacccagtccgcc |
| | ctgagcaaagaccccaacgagaagcgcgatcacatggtcctgctggagttcgtgaccgccgccgggatcactctc |
| | ggcatggacgagctgtacaagtaataagcttctcgactagggataacagggtaattgtttgaatgaggcttcagtactt |
| | tacagaatcgttgcctgcacatcttggaaacacttgctgggattacttcgacttcttaacccaacagaaggctcgaga |
| | aggtatattgctgttgacagtgagcgCgtacagtatttatcgagataatagtgaagccacagatgtattatctcgataa |
| | atactgtacAtgcctactgcctcggacttcaaggggctagaattcgagcaattatcttgtttactaaaactgaatacctt |
| | gctatctctttgatacatttttacaaagctgaattaaaatggtataaattaaatcacttttttcaattgacgcgtaattctacc |
| | ggatccaatcaacctctggattacaaaatttgtgaaagattgactggtattcttaactatgttgctccttttacgctatgtgg |
| | atacgctgctttaatgcctttgtatcatgctattgcttcccgtatggctttcattttctcctccttgtataaatcctggttgctgtct |
| | ctttatgaggagttgtggcccgttgtcaggcaacgtggcgtggtgtgcactgtgtttgctgacgcaacccccactggttg |
| | gggcattgccaccacctgtcagctcctttccgggactttcgctttccccctccctattgccacggcggaactcatcgccg |
| | cctgccttgcccgctgctggacaggggctcggctgttgggcactgacaattccgtggtgttgtcggggaaatcatcgtc |
| | ctttccttggctgctcgcctgtgttgccacctggattctgcgcgggacgtccttctgctacgtcccttcggccctcaatcca |
| | gcggaccttccttcccgcggcctgctgccggctctgcggcctcttccgcgtcttcgagatctgcctcgactgtgccttcta |
| | gttgccagccatctgttgtttgcccctcccccgtgccttccttgaccctggaaggtgccactcccactgtcctttcctaata |
| | aaatgaggaaattgcatcgcattgtctgagtaggtgtcattctattctggggggtggggggggcaggacagcaagg |
| | gggaggattgggaagacaatagcaggcatgctggggagagctcttaagggcgaattcccgataaggatcttccta |
| | gagcatggctacgtagataagtagcatggcgggttaatcattaactacaaggaacccctagtgatggagttggcca |
| | ctccctctctgcgcgctcgctcgctcactgaggccgggcgaccaaaggtcgcccgacgcccgggctttgcccgggc |
| | ggcctcagtgagcgagcgagcgcgcagccttaattaacctaattcactggccgtcgttttacaacgtcgtgactggg |
| | aaaaccctggcgttacccaacttaatcgccttgcagcacatccccctttcgccagctggcgtaatagcgaagaggcc |
| | cgcaccgatcgcccttcccaacagttgcgcagcctgaatggcgaatgggacgcgccctgtagcggcgcattaagc |
| | gcggcgggtgtggtggttacgcgcagcgtgaccgctacacttgccagcgccctagcgcccgctcctttcgctttcttcc |
| | cttcctttctcgccacgttcgccggctttccccgtcaagctctaaatcgggggctccctttagggttccgatttagtgcttta |
| | cggcacctcgaccccaaaaaacttgattagggtgatggttcacgtagtgggccatcgccctgatagacggtttttcgc |
| | cctttgacgttggagtccacgttctttaatagtggactcttgttccaaactggaacaacactcaaccctatctcggtctatt |
| | cttttgatttataagggattttgccgatttcggcctattggttaaaaaatgagctgatttaacaaaaatttaacgcgaatttt |
| | aacaaaatattaacgcttacaatttaggtggcacttttcggggaaatgtgcgcggaacccctatttgtttatttttctaaat |
| | acattcaaatatgtatccgctcatgagacaataaccctgataaatgcttcaataatattgaaaaaggaagagtatgag |
| | ccatattcaacgggaaacgtcgaggccgcgattaaattccaacatggatgctgatttatatgggtataaatgggctcg |
| | cgataatgtcgggcaatcaggtgcgacaatctatcgcttgtatgggaagcccgatgcgccagagttgtttctgaaaca |
| | tggcaaaggtagcgttgccaatgatgttacagatgagatggtcagactaaactggctgacggaatttatgcctcttcc |
| | gaccatcaagcattttatccgtactcctgatgatgcatggttactcaccactgcgatccccggaaaaacagcattcca |
| | ggtattagaagaatatcctgattcaggtgaaaatattgttgatgcgctggcagtgttcctgcgccggttgcattcgattcc |
| | tgtttgtaattgtccttttaacagcgatcgcgtatttcgtcttgctcaggcgcaatcacgaatgaataacggtttggttgatg |
| | cgagtgattttgatgacgagcgtaatggctggcctgttgaacaagtctggaaagaaatgcataaacttttgccattctc |
| | accggattcagtcgtcactcatggtgatttctcacttgataaccttatttttgacgaggggaaattaataggttgtattgatg |
| | ttggacgagtcggaatcgcagaccgataccaggatcttgccatcctatggaactgcctcggtgagttttctccttcatta |
| | cagaaacggctttttcaaaaatatggtattgataatcctgatatgaataaattgcagtttcatttgatgctcgatgagttttt |
| | ctaactgtcagaccaagtttactcatatatactttagattgatttaaaacttcatttttaatttaaaaggatctaggtgaaga |
| | tcctttttgataatctcatgaccaaaatcccttaacgtgagttttcgttccactgagcgtcagaccccgtagaaaagatc |
| | aaaggatcttcttgagatcctttttttctgcgcgtaatctgctgcttgcaaacaaaaaaaccaccgctaccagcggtggt |
| | ttgtttgccggatcaagagctaccaactctttttccgaaggtaactggcttcagcagagcgcagataccaaatactgtt |
| | cttctagtgtagccgtagttaggccaccacttcaagaactctgtagcaccgcctacatacctcgctctgctaatcctgtt |
| | accagtggctgctgccagtggcgataagtcgtgtcttaccgggttggactcaagacgatagttaccggataaggcgc |
| | agcggtcgggctgaacggggggttcgtgcacacagcccagcttggagcgaacgacctacaccgaactgagata |
| | cctacagcgtgagctatgagaaagcgccacgcttcccgaagggagaaaggcggacaggtatccggtaagcggc |
| | agggtcggaacaggagagcgcacgagggagcttccagggggaaacgcctggtatctttatagtcctgtcgggtttc |
| | gccacctctgacttgagcgtcgatttttgtgatgctcgtcaggggggcggagcctatggaaaaacgccagcaacgc |
| | ggcctttttacggttcctggccttttgctggccttttgctcacatgttctttcctgcgttatcccctgattctgtggataaccgtat |
| | taccgcctttgagtgagctgataccgctcgccgcagccgaacgaccgagcgcagcgagtcagtgagcgaggaag |
| | cggaagagcgcccaatacgcaaaccgcctctccccgcgcgttggccgattcattaatgcagctggcacgacaggt |
| | ttcccgactggaaagcgggcagtgagcgcaacgcaattaatgtgagttagctcactcattaggcaccccaggcttta |
| | cactttatgcttccggctcgtatgttgtgtggaattgtgagcggataacaatttcacacaggaaacagctatgaccatg |
| | attacgccagatttaattaagg |

TABLE 11
Exemplary siRNA sequences

| SEQ ID NO: | Sequence |
|---|---|
| 80 | CCAGUAAUAUUUAGAGCUAUU |
| Sox2 siRNA A-058489-13 | |
| sense strand | |
| 81 | UAGCUCUAAAUAUUACUGGUU |
| Sox2 siRNA A-058489-13 | |
| antisense strand | |
| 82 | CGCUCAUGAAGAAGGAUAAUU |
| Sox2siRNAA-058489-14 | |
| sense strand | |
| 83 | UUAUCCUUCUUCAUGAGCGUU |
| Sox2siRNAA-058489-14 | |
| antisense strand | |
| 84 | UUGUGAUAUUUUAAGGUUUUU |
| Sox2siRNAA-058489-15 | |
| sense strand | |
| 85 | AAACCUUAAAAUAUCACAAUU |
| Sox2siRNAA-058489-15 | |
| antisense strand | |
| 86 | CUUAUGGUUUGUAAUAUUUUU |
| Sox2siRNAA-058489-16 | |
| sense strand | |
| 87 | AAAUAUUACAAACCAUAAGUU |
| Sox2siRNAA-058489-16 | |
| antisense strand | |

In some embodiments, the siRNA or shRNA targeting Sox2 has a nucleobase sequence containing a portion of at least 8 contiguous nucleobases (e.g., 8, 9, 10, 11, 12, 13, 14, 15, 16, 17, 18, 19, 20, or more nucleobases) having at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) to an equal length portion of a target region of an mRNA transcript of a human (e.g., the human Sox2 mRNA of NCBI Reference Sequence: NM_003106.4) or a murine (e.g., the murine Sox2 mRNA of NCBI Reference Sequence: NM_011443.4) SOX2 gene. In some embodiments the target region is at least 8 to 21 (e.g., 8 to 21, 9 to 21, 10 to 21, 11 to 21, 12 to 21, 13 to 21, 14 to 21, 15 to 21, 16 to 21, 17 to 21, 18 to 21, 19 to 21, 20 to 21, or all 21) contiguous nucleobases of any one or more of SEQ ID NOs: 52-70. In some embodiments the target region is at least 8 to 19 (e.g., 8 to 19, 9 to 19, 10 to 19, 11 to 19, 12 to 19, 13 to 19, 14 to 19, 15 to 19, 16 to 19, 17 to 19, 18 to 19, or all 19) contiguous nucleobases of any one of SEQ ID NOs: 71-73. In some embodiments the target region is at least 8 to 22 (e.g., 8 to 22, 9 to 22, 10 to 22, 11 to 22, 12 to 22, 13 to 22, 14 to 22, 15 to 22, 16 to 22, 17 to 22, 18 to 22, 19 to 22, 20 to 22, 21 to 22, or all 22) contiguous nucleobases of SEQ ID NOs: 74 or 75.

In some embodiments, the siRNA or shRNA targets SEQ ID NO: 58, SEQ ID NO: 71, SEQ ID NO: 72, or SEQ ID NO: 73, SEQ ID NO: 74, or SEQ ID NO: 75.

In some embodiments, the shRNA has at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) to the entire length of SEQ ID NO: 58, SEQ ID NO: 71, SEQ ID NO: 72, SEQ ID NO: 73, SEQ ID NO: 74, or SEQ ID NO: 75. In some embodiments, the shRNA has 100% complementarity to the entire length of SEQ ID NO: 58, SEQ ID NO: 71, SEQ ID NO: 72, SEQ ID NO: 73, SEQ ID NO: 74, or SEQ ID NO: 75.

In some embodiments, the polynucleotide that can be transcribed to produce an shRNA includes the sequence of nucleotides 2234-2296 of SEQ ID NO: 76 or nucleotides 2234-2296 of SEQ ID NO: 78.

In some embodiments, the polynucleotide that can be transcribed to produce an shRNA has the sequence of nucleotides 2234-2296 of SEQ ID NO: 76 or nucleotides 2234-2296 of SEQ ID NO: 78. In some embodiments, the shRNA is embedded into the backbone of a miRNA. In some embodiments, the miRNA backbone and the shRNA include the sequence of nucleotides 2109-2426 of SEQ ID NO: 76, nucleotides 2109-2408 of SEQ ID NO: 77, nucleotides 2109-2426 of SEQ ID NO: 78, or nucleotides 2109-2408 of SEQ ID NO: 79. In some embodiments, the miRNA backbone and the shRNA have the sequence of nucleotides 2109-2426 of SEQ ID NO: 76, nucleotides 2109-2408 of SEQ ID NO: 77, nucleotides 2109-2426 of SEQ ID NO: 78, or nucleotides 2109-2408 of SEQ ID NO: 79. These polynucleotide sequences can be operably linked to a promoter in a vector described herein and, optionally, regulated by one or more miRNA target sequences to improve cell-type specific expression.

In some embodiments, the siRNA is a pair of nucleotide sequences (sense and anti-sense strands) selected from SEQ ID NO: 80 and SEQ ID NO: 81; SEQ ID NO: 82 and SEQ ID NO: 83; SEQ ID NO: 84 and SEQ ID NO: 85; and SEQ ID NO: 86 and SEQ ID NO: 87.

siRNA and shRNA molecules for use in the methods and compositions described herein can target the mRNA sequence of Sox2 (e.g., human Sox2 mRNA or murine Sox2 mRNA). siRNA and shRNA molecules may be delivered using a vector described herein, such as a viral vector (e.g., an AAV vector), and they may be expressed using a cell type-specific promoter (e.g., a hair cell-specific promoter or a supporting cell-specific promoter) or using a ubiquitous promoter (e.g., a ubiquitous pol II or pol III promoter).

An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without wishing to be bound by theory, it is believed that certain modifications can increase nuclease resistance and/or serum stability or decrease immunogenicity.

In some embodiments, the inhibitory RNA molecule decreases the level and/or activity or function of Sox2. In some embodiments, the inhibitory RNA molecule inhibits expression of Sox2. In other embodiments, the inhibitory RNA molecule increases degradation of Sox2 and/or decreases the stability (i.e., half-life) of Sox2. The inhibitory RNA molecule can be chemically synthesized or transcribed in vitro.

The making and use of inhibitory therapeutic agents based on non-coding RNA such as ribozymes, RNase P, siRNAs, and miRNAs are also known in the art, for example, as described in Sioud, RNA Therapeutics: Function, Design, and Delivery (Methods in Molecular Biology). Humana Press 2010.

### Gene Editing Components

In some embodiments, the vector contains a polynucleotide that is or encodes a component of a gene editing system. For example, the component of a gene editing system can be used to introduce an alteration (e.g., insertion, deletion (e.g., knockout), translocation, inversion, single point mutation, or other mutation) in a gene expressed in an inner ear cell. Exemplary gene editing systems include zinc finger nucleases (ZFNs), Transcription Activator-Like Effector-based Nucleases (TALENs), and the clustered regulatory interspaced short palindromic repeat (CRISPR) system. ZFNs, TALENs, and CRISPR-based methods are described, e.g., in Gaj et al., Trends Biotechnol. 31:397-405, 2013.

CRISPR refers to a set of (or system including a set of) clustered regularly interspaced short palindromic repeats. A CRISPR system refers to a system derived from CRISPR and Cas (a CRISPR-associated protein) or another nuclease that can be used to silence or mutate a gene expressed in an inner ear cell. The CRISPR system is a naturally occurring system found in bacterial and archaeal genomes. The CRISPR locus is made up of alternating repeat and spacer sequences. In naturally occurring CRISPR systems, the spacers are typically sequences that are foreign to the bacterium (e.g., plasmid or phage sequences). The CRISPR system has been modified for use in gene editing (e.g., changing, silencing, and/or enhancing certain genes) in eukaryotes. See, e.g., Wiedenheft et al., Nature 482: 331, 2012. For example, such modification of the system includes introducing into a eukaryotic cell a plasmid containing a specifically designed CRISPR and one or more appropriate Cas proteins. The CRISPR locus is transcribed into RNA and processed by Cas proteins into small RNAs that comprise a repeat sequence flanked by a spacer. The RNAs serve as guides to direct Cas proteins to silence specific DNA/RNA sequences, depending on the spacer sequence. See, e.g., Horvath et al., Science 327: 167, 2010; Makarova et al., Biology Direct 1:7, 2006; Pennisi, Science 341:833, 2013. In some examples, the CRISPR system includes the Cas9 protein, a nuclease that cuts on both strands of the DNA. See, e.g., Id.

In some embodiments, in a CRISPR system for use described herein, e.g., in accordance with one or more methods described herein, the spacers of the CRISPR are derived from a target gene sequence, e.g., from a gene expressed in an inner ear cell.

In some embodiments, the polynucleotide includes a guide RNA (gRNA) for use in a clustered regulatory interspaced short palindromic repeat (CRISPR) system for gene editing. In some embodiments, the polynucleotide includes or encodes a zinc finger nuclease (ZFN), or an mRNA encoding a ZFN, that targets (e.g., cleaves) a nucleic acid sequence (e.g., DNA sequence) of a gene expressed in an inner ear cell. In some embodiments, the polynucleotide includes or encodes a TALEN, or an mRNA encoding a TALEN, that targets (e.g., cleaves) a nucleic acid sequence (e.g., DNA sequence) of a gene expressed in an inner ear cell.

For example, the gRNA can be used in a CRISPR system to engineer an alteration in a gene (e.g., a gene expressed in an inner ear cell). In other examples, the ZFN and/or TALEN can be used to engineer an alteration in a gene (e.g., a gene expressed in an inner ear cell). Exemplary alterations include insertions, deletions (e.g., knockouts), translocations, inversions, single point mutations, or other mutations. The alteration can be introduced in the gene in a cell, e.g., in vitro, ex vivo, or in vivo. In some embodiments, the alteration decreases the level and/or activity of (e.g., knocks down or knocks out) a gene expressed in an inner ear cell, e.g., the alteration is a negative regulator of function. In yet another example, the alteration corrects a defect (e.g., a mutation causing a defect) in a gene expressed in an inner ear cell, such as a gene that is implicated in sensorineural hearing loss or vestibular dysfunction, such as a gene listed in Table 4.

In certain embodiments, the CRISPR system is used to edit (e.g., to add or delete a base pair) a target gene, e.g., a gene expressed in an inner ear cell. In other embodiments, the CRISPR system is used to introduce a premature stop codon, e.g., thereby decreasing the expression of a target gene. In yet other embodiments, the CRISPR system is used to turn off a target gene in a reversible manner, e.g., similarly to RNA interference. In some embodiments, the CRISPR system is used to direct Cas to a promoter of a target gene, e.g., a gene expressed in an inner ear cell, thereby blocking an RNA polymerase sterically.

In some embodiments, a CRISPR system can be generated to edit a gene expressed in an inner ear cell, such as a gene that is implicated in sensorineural hearing loss or vestibular dysfunction, using technology described in, e.g., U.S. Publication No. 20140068797; Cong, Science 339: 819, 2013; Tsai, Nature Biotechnol., 32:569, 2014; and U.S. Pat. Nos. 8,871,445; 8,865,406; 8,795,965; 8,771,945; and 8,697,359.

In some embodiments, the CRISPR interference (CRISPRi) technique can be used for transcriptional repression of specific genes, e.g., a gene expressed in an inner ear cell, such as a mutant form of a gene that is implicated in sensorineural hearing loss or vestibular dysfunction. In CRISPRi, an engineered Cas9 protein (e.g., nuclease-null dCas9, or dCas9 fusion protein, e.g., dCas9-KRAB or dCas9-SID4X fusion) can pair with a sequence specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

In some embodiments, CRISPR-mediated gene activation (CRISPRa) can be used for transcriptional activation, e.g., of one or more genes described herein, e.g., a gene expressed in an inner ear cell, such as a gene that is implicated in sensorineural hearing loss or vestibular dysfunction. In the CRISPRa technique, dCas9 fusion proteins recruit transcriptional activators. For example, dCas9 can be used to recruit polypeptides (e.g., activation domains) such as VP64 or the p65 activation domain (p65D) and used with sgRNA (e.g., a single sgRNA or multiple sgRNAs), to activate a gene or genes, e.g., endogenous gene(s). Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains) such as VP64. In some examples, the synergistic activation mediator (SAM) system can be used for transcriptional activation. In SAM, MS2 aptamers are added to the sgRNA. MS2 recruits the MS2 coat protein (MCP) fused to p65AD and heat shock factor 1 (HSF1). The CRISPRi and CRISPRa techniques are described in greater detail, e.g., in Dominguez et al., Nat. Rev. Mol. Cell Biol. 17:5, 2016, incorporated herein by reference.

### Promoters

Recognition and binding of a polynucleotide by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Promoter sequences are typically located upstream of the translation start site (e.g., within two kilobases upstream of the translation start site). Examples of mammalian promoters have been described in Smith, et al., Mol. Sys. Biol., 3:73, online publication, the disclosure of which is incorporated herein by reference. The promoter used in the methods and compositions described herein can be a ubiquitous promoter or a cell type-specific promoter (e.g., a promoter that induces or increases expression of a polynucleotide in one or more specific cell types, such as hair cells or supporting cells). Ubiquitous promoters include the CAG promoter, cytomegalovirus (CMV) promoter, smCBA promoter (described in Haire et al., Invest. Opthalmol. Vis. Sci. 47:3745-3753, 2006), dihydrofolate reductase (DHFR) promoter, human β-actin promoter, phosphoglycerate I kinase (PGK) promoter, EF1α promoter, apolipoprotein E-human α1-antitrypsin promoter (hAAT), CK8 promoter, murine U1 promoter (mU1a), early growth response 1 (EGR1) promoter, thyroxine binding globulin (TBG) promoter, chicken β-actin (CBA) promoter, hybrid CMV enhancer/chicken β-actin promoter, SV40 early promoter, eukaryotic translation initiation factor 4A1 (EIF4A1) promoter, ferritin heavy (FerH) promoter, ferritin light (FerL) promoter, glyceraldehyde-3-phosphate dehydrogenase (GAPDH) promoter, heat shock protein family A member 5 (HSPA5) gene, heat shock protein family A member 4 (HSPA4) promoter, and ubiquitin B (UBB) promoter. Alternatively, promoters derived from viral genomes can also be used for the stable expression of polynucleotides in primate (e.g., human) cells. Examples of functional viral promoters that can be used for the expression of polynucleotides in primate (e.g., human) cells include adenovirus late promoter, vaccinia virus 7.5K promoter, tk promoter of HSV, mouse mammary tumor virus (MMTV) promoter, LTR promoter of HIV, promoter of moloney virus, Epstein barr virus (EBV) promoter, and the Rous sarcoma virus (RSV) promoter. A pol II promoter, such as a ubiquitous promoter described above or a cell type-specific promoter described in Table 12, below, can be used to express any protein-coding transgene described herein. A pol III promoter, including ubiquitous pol Ill promoters U6, H1, and 7SK, can be used to express a polynucleotide that is an shRNA or an siRNA.

Cell type-specific promoters that can be included in the vectors described herein to express a polynucleotide that can be transcribed to produce a desired expression product and a polynucleotide that can be transcribed to produce a miRNA target sequence in one or more inner ear cell types include hair cell-specific promoters and supporting cell-specific promoters. Exemplary inner ear cell type-specific promoters are provided in Table 12, below.

TABLE 12
Inner ear cell type-specific promoters

| Cell Type | Promoter |
|---|---|
| Supporting cells | Glial Fibrillary Acidic Protein (GFAP), Solute |
| | Carrier Family 1 Member 3 (SLC1A3, also known |
| | as GLAST, an exemplary promoter is described in |
| | Mizutani et al., Nature, 449: 351-355, 2007), LFNG |
| | O-Fucosylpeptide 3-Beta-N- |
| | Acetylglucosaminyltransferase (LFNG, an |
| | exemplary promoter is described in Morales et al., |
| | Developmental Cell 3: 63-74, 2002), Solute Carrier |
| | Family 6 Member 14 (SLC6A14), Fibroblast |
| | Growth Factor Receptor 3 (FGFR3), PROX1, |
| | Neuropeptide Y (NPY), Anterior Gradient 3, Protein |
| | Disulphide Isomerase Family Member (AGR3), |
| | Sprouty RTK Signaling Antagonist 2 (SPRY2), |
| | SOX2, HES1, Jagged 1 (JAG1), Notch 1 |
| | (NOTCH1, an exemplary promoter is described in |
| | Lambertini et al., PLoS ONE, 5: 1-13, 2010), |
| | Leucine Rich Repeat Containing G Protein- |
| | Coupled Receptor 5 (LGR5), Hes Family BHLH |
| | Transcription Factor 5 (HES5), SOX9, Kringle |
| | Containing Transmembrane Protein 1 (KREMEN1) |
| Hair cells | Myosin 15A (MYO15), MYO7A, MYO6, SLC17A8 |
| | (also known as VGLUT3), OTOF, SLC26A5 (also |
| | known as PRESTIN), OCM, CABP2, Fibroblast |
| | Growth Factor 8 (FGF8), STRC, ATPase Plasma |
| | Membrane Ca2+ Transporting 2 (ATP2B2) |
| Supporting cell progenitors | LGR5 |
| Type I vestibular HCs | ATP2B2 |
| Type II vestibular HCs | Calbindin 2 (CALB2) Microtubule associated |
| | protein tau (MAPT), Annexin A4 (ANXA4), Otoferlin |
| | (OTOF) |
| Border cells (cochlear supporting cell | GLAST, GJB2 |
| subtype) | |
| Inner phalangeal cells (cochlear supporting | GLAST, GJB2 |
| cell subtype) | |
| Pillar cells (cochlear supporting cell | CD44 Molecule (CD44), GJB2 |
| subtype) | |
| Deiters' cells (cochlear supporting cell | Fibroblast Growth Factor Receptor 3 (FGFR3), |
| subtype) | GJB2 |
| Hensen's cells (cochlear supporting cell | Frizzled Related Protein (FRZB), GJB2 |
| subtype) | |
| Claudius cells (cochlear supporting cell | FRZB, GJB2 |
| subtype) | |
| Spiral prominence cells | SLC26A4 |
| Root cells | SLC26A4 |
| Interdental cells | CEACAM16, GJB2 |
| Basal cells of the SV | Claudin 11 (CLDN11), GJB2 |
| Intermediate cells of the SV | Tyrosinase (TYR), Potassium Voltage-Gated |
| | Channel Subfamily J Member 10 (KCNJ10), GJB2 |
| Marginal cells of the SV | KCNE1, KCNQ1, GJB2 |
| SGNs | Basic Helix-Loop-Helix Family Member E22 |
| | (BHLHE22), Synapsin (SYN) |
| SGNs with a high rate of spontaneous firing | CALB2 |
| Glia | PMP22 |
| Vestibular dark cells | KCNE1 |
| Fibrocytes/mesenchyme | POU3F4, GJB2 |
| Scarpa's ganglion (Vestibular ganglion) | TUBB3, SYN |

Exemplary Myo15 promoters are described in International Application Publication Nos. WO2019210181 and WO2020163761A1 and U.S. Patent Application Publication No. US20210236654, exemplary SLC6A14 promoters are described in International Application Publication No. WO2021091950 and in International Application No. PCT/US2022/027679, exemplary OCM promoters are described in International Application Publication No. WO2021091938, exemplary CABP2 promoters are described in International Application Publication No. WO2021091940, exemplary GJB2 promoters are described in International Application Publication No. WO2021067448, exemplary SLC26A4, LGR5, and SYN1 promoters are described in International Application Publication No. WO2021231567, and exemplary GFAP promoters are described in International Application Publication Nos. WO2021231885, WO2021067448, and WO2021231567, the disclosures of which are incorporated herein by reference.

Once a polynucleotide has been incorporated into the nuclear DNA or into the nucleus of a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available (Life Technologies, Carlsbad, CA) and can be administered to a mammalian cell in order to promote gene expression according to established protocols. Further control of expression of a polynucleotide described herein can be achieved using conditional regulation elements, such as Cre recombinase systems, including FLEx-Cre, as described in Saunders et al., Front Neural Circuits 6:47 (2012).

Other DNA sequence elements that may be included in polynucleotides (e.g., polynucleotides containing a promoter operably linked to a polynucleotide that can be transcribed to produce a desired expression product and to a polynucleotide that can be transcribed to produce a miRNA target sequence) for use in the compositions and methods described herein include enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that contain a polynucleotide of interest and a polynucleotide that can be transcribed to produce a miRNA target sequence and additionally include a mammalian enhancer sequence. Many enhancer sequences are now known from mammalian genes, and examples include enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples include the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription include the CMV enhancer and RSV enhancer. An enhancer may be spliced into a vector containing a polynucleotide encoding a protein of interest, for example, at a position 5′ or 3′ to this gene. In a preferred orientation, the enhancer is positioned at the 5′ side of the promoter, which in turn is located 5′ relative to the polynucleotide encoding a protein of interest.

The nucleic acid vectors containing a promoter operably linked to a polynucleotide that can be transcribed to produce a desired expression product and to a polynucleotide that can be transcribed to produce a miRNA target sequence described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cell. The addition of the WPRE to a vector can result in a substantial improvement in the level of transgene expression from several different promoters, both in vitro and in vivo. In some embodiments of the compositions and methods described herein, the WPRE has the sequence:

(SEQ ID NO: 88)
GATCCAATCAACCTCTGGATTACAAAATTTGTGAAAGATTGACTGGT
ATTCTTAACTATGTTGCTCCTTTTACGCTATGTGGATACGCTGCTTT
AATGCCTTTGTATCATGCTATTGCTTCCCGTATGGCTTTCATTTTCT
CCTCCTTGTATAAATCCTGGTTGCTGTCTCTTTATGAGGAGTTGTGG
CCCGTTGTCAGGCAACGTGGCGTGGTGTGCACTGTGTTTGCTGACGC
AACCCCCACTGGTTGGGGCATTGCCACCACCTGTCAGCTCCTTTCCG
GGACTTTCGCTTTCCCCCTCCCTATTGCCACGGCGGAACTCATCGCC
GCCTGCCTTGCCCGCTGCTGGACAGGGGCTCGGCTGTTGGGCACTGA
CAATTCCGTGGTGTTGTCGGGGAAATCATCGTCCTTTCCTTGGCTGC
TCGCCTGTGTTGCCACCTGGATTCTGCGCGGGACGTCCTTCTGCTAC
GTCCCTTCGGCCCTCAATCCAGCGGACCTTCCTTCCCGCGGCCTGCT
GCCGGCTCTGCGGCCTCTTCCGCGTCTTCGA.

In other embodiments, the WPRE has the sequence:

(SEQ ID NO: 89)
AATCAACCTCTGGATTACAAAATTTGTGAAAGATTGACTGGTATTCT
TAACTATGTTGCTCCTTTTACGCTATGTGGATACGCTGCTTTAATGC
CTTTGTATCATGCTATTGCTTCCCGTATGGCTTTCATTTTCTCCTCC
TTGTATAAATCCTGGTTAGTTCTTGCCACGGCGGAACTCATCGCCGC
CTGCCTTGCCCGCTGCTGGACAGGGGCTCGGCTGTTGGGCACTGACA
ATTCCGTGGTGTTTATTTGTGAAATTTGTGATGCTATTGCTTTATTT
GTAACCATCTAGCTTTATTTGTGAAATTTGTGATGCTATTGCTTTAT
TTGTAACCATTATAAGCTGCAATAAACAAGTTAACAACAACAATTGC
ATTCATTTTATGTTTCAGGTTCAGGGGGAGATGTGGGAGGTTTTTTA
AA

In some embodiments, the nucleic acid vectors containing a promoter operably linked to a polynucleotide that can be transcribed to produce a desired expression product and to a polynucleotide that can be transcribed to produce a miRNA target sequence described herein include a reporter sequence, which can be useful in verifying the expression of the polynucleotide or a protein encoded by the polynucleotide, for example, in cells and tissues (e.g., in inner ear cells). Reporter sequences that may be provided in a transgene and incorporated into a vector described herein include DNA sequences encoding β-lactamase, β-galactosidase (LacZ), alkaline phosphatase, thymidine kinase, green fluorescent protein (GFP), chloramphenicol acetyltransferase (CAT), luciferase, and others well known in the art. When associated with regulatory elements that drive their expression, such as a promoter, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for-galactosidase activity. Where the transgene is green fluorescent protein or luciferase, the vector carrying the signal may be measured visually by color or light production in a luminometer.

### Methods for the Delivery of Exogenous Nucleic Acids to Target Cells

Techniques that can be used to introduce a polynucleotide, such as a polynucleotide that can be transcribed to produce a desired expression product associated with a polynucleotide that can be transcribed to produce a miRNA target sequence, into a target cell (e.g., a mammalian cell) are well known in the art. For instance, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for instance, by direct fusion of the liposome with the cell membrane or by endocytosis of the complex. Lipofection is described in detail, for instance, in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane include activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for instance, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for instance, in US 2010/0227406, the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107: 1870 (2010), the disclosure of which is incorporated herein by reference.

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane to permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Vectors for Delivery of Exogenous Nucleic Acids to Target Cells

In addition to achieving high rates of transcription and translation, stable expression of an exogenous polynucleotide in a mammalian cell can be achieved by integration of the polynucleotide into the nuclear genome of the mammalian cell. A variety of vectors for the delivery and integration of polynucleotides into the nuclear DNA of a mammalian cell have been developed. Examples of expression vectors are described in, e.g., Gellissen, Production of Recombinant Proteins: Novel Microbial and Eukaryotic Expression Systems (John Wiley & Sons, Marblehead, M A, 2006). Expression vectors for use in the compositions and methods described herein contain a promoter operably linked to a polynucleotide that can be transcribed to produce a desired expression product and to a polynucleotide that can be transcribed to produce a miRNA target sequence, as well as, e.g., additional sequence elements used for the expression of these agents and/or the integration of these polynucleotide sequences into the genome of a mammalian cell. Vectors that can contain a promoter operably linked to a polynucleotide that can be transcribed to produce a desired expression product and to a polynucleotide that can be transcribed to produce a miRNA target sequence include plasmids (e.g., circular DNA molecules that can autonomously replicate inside a cell), cosmids (e.g., pWE or sCos vectors), artificial chromosomes (e.g., a human artificial chromosome (HAC), a yeast artificial chromosome (YAC), a bacterial artificial chromosome (BAC), or a P1-derived artificial chromosome (PAC)), and viral vectors. Certain vectors that can be used for the expression of a polynucleotide associated with a miRNA target sequence include plasmids that contain regulatory sequences, such as enhancer regions, which direct gene transcription. Other useful vectors for expression of a polynucleotide associated with a miRNA target sequence contain polynucleotide sequences that enhance the rate of translation or improve the stability or nuclear export of the mRNA that results from transcription. These sequence elements include, e.g., 5′ and 3′ untranslated regions, an internal ribosomal entry site (IRES), and polyadenylation signal site in order to direct efficient transcription of the polynucleotide carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### Viral Vectors for Nucleic Acid Delivery

Viral genomes provide a rich source of vectors that can be used for the efficient delivery of a polynucleotide of interest into the genome of a target cell (e.g., a mammalian cell, such as a human cell). Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, 1996)). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, U.S. Pat. No. 5,801,030, the disclosure of which is incorporated herein by reference as it pertains to viral vectors for use in gene therapy.

### AAV Vectors for Nucleic Acid Delivery

In some embodiments, polynucleotides of the compositions and methods described herein are incorporated into rAAV vectors and/or virions in order to facilitate their introduction into a cell. rAAV vectors useful in the compositions and methods described herein are recombinant nucleic acid constructs that include (1) a promoter, (2) a heterologous polynucleotide associated with a polynucleotide that can be transcribed to produce a miRNA target sequence, and (3) viral sequences that facilitate stability and expression of the heterologous polynucleotides. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. For use in the methods and compositions described herein, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The polynucleotides and vectors described herein (e.g., a polynucleotide containing a promoter operably linked to a polynucleotide that can be transcribed to produce a desired expression product and to a polynucleotide that can be transcribed to produce a miRNA target sequence) can be incorporated into a rAAV virion in order to facilitate introduction of the polynucleotide or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10, 11, rh10, rh39, rh43, rh74, AAV2-QuadYF, Anc80, Anc80L65, DJ, DJ/8, DJ/9, 7m8, and PHP (PHP.B, PHP.B2, PHP.B3, PHP.eb, PHP.S, PHP.A). For targeting inner ear cells, AAV1, AAV2, AAV8, AAV9, Anc80, 7m8, DJ, DJ/9, PHP.B, PHP.B2, PHP.B3, PHP.eB, PHP.S, and PHP.A serotypes may be particularly useful. Serotypes evolved for transduction of the retina may also be used in the methods and compositions described herein. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

### Pharmaceutical Compositions

The vectors described herein may be incorporated into a vehicle for administration into a patient, such as a human patient suffering from hearing loss, deafness, auditory neuropathy, tinnitus, or vestibular dysfunction (e.g., dizziness, vertigo, loss of balance or imbalance, bilateral vestibulopathy, oscillopsia, or a balance disorder). Pharmaceutical compositions containing a vector described herein can be prepared using methods known in the art. For example, such compositions can be prepared using, e.g., physiologically acceptable carriers, excipients, or stabilizers (Remington: The Science and Practice of Pharmacology 22nd edition, Allen, L. Ed. (2013); incorporated herein by reference), and in a desired form, e.g., in the form of lyophilized formulations or aqueous solutions.

Mixtures of a vector described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the ear (e.g., the middle or inner ear), the composition may be formulated to contain a synthetic perilymph solution. An exemplary synthetic perilymph solution includes 20-200 mM NaCl, 1-5 mM KCl, 0.1-10 mM CaCl₂), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

### Methods of Treatment

The compositions described herein may be administered to a subject having or at risk of developing sensorineural hearing loss, deafness, auditory neuropathy, tinnitus, and/or vestibular dysfunction by a variety of routes, such as local administration to the middle or inner ear (e.g., administration into the perilymph or endolymph, such as to or through the oval window, round window, or semicircular canal (e.g., the horizontal canal), or by transtympanic or intratympanic injection, e.g., administration to an inner ear cell), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, monthly, or bi-weekly).

Subjects that may be treated as described herein are subjects having or at risk of developing sensorineural hearing loss and/or vestibular dysfunction (e.g., subjects having or at risk of developing hearing loss, vestibular dysfunction, or both). The compositions and methods described herein can be used to treat subjects having or at risk of developing damage to inner ear cells, such as hair cells (e.g., damage related to acoustic trauma, disease or infection, head trauma, ototoxic drugs, or aging), subjects having or at risk of developing sensorineural hearing loss, deafness, or auditory neuropathy, subjects having or at risk of developing vestibular dysfunction (e.g., dizziness, vertigo, imbalance, bilateral vestibulopathy, oscillopsia, or a balance disorder), subjects having tinnitus (e.g., tinnitus alone, or tinnitus that is associated with sensorineural hearing loss or vestibular dysfunction), subjects having a genetic mutation associated with hearing loss and/or vestibular dysfunction (e.g., a mutation in a gene listed in Table 4), or subjects with a family history of hereditary hearing loss, deafness, auditory neuropathy, tinnitus, or vestibular dysfunction. In some embodiments, the disease associated with damage to or loss of inner ear cells (e.g., hair cells, such as cochlear and/or vestibular hair cells) is an autoimmune disease or condition in which an autoimmune response contributes to inner ear cell damage or death. Autoimmune diseases linked to sensorineural hearing loss and vestibular dysfunction include autoimmune inner ear disease (AIED), polyarteritis nodosa (PAN), Cogan's syndrome, relapsing polychondritis, systemic lupus erythematosus (SLE), Wegener's granulomatosis, Sjögren's syndrome, and Behçet's disease. Some infectious conditions, such as Lyme disease and syphilis can also cause hearing loss and vestibular dysfunction (e.g., by triggering autoantibody production). Viral infections, such as rubella, cytomegalovirus (CMV), lymphocytic choriomeningitis virus (LCMV), HSV types 1&2, West Nile virus (WNV), human immunodeficiency virus (HIV) varicella zoster virus (VZV), measles, and mumps, can also cause hearing loss and vestibular dysfunction. In some embodiments, the subject has or is at risk of developing hearing loss and/or vestibular dysfunction that is associated with or results from loss of hair cells (e.g., cochlear or vestibular hair cells). In some embodiments, compositions and methods described herein can be used to treat a subject having or at risk of developing oscillopsia. In some embodiments, compositions and methods described herein can be used to treat a subject having or at risk of developing bilateral vestibulopathy. In some embodiments, the compositions and methods described herein can be used to treat a subject having or at risk of developing a balance disorder. The methods described herein may include a step of screening a subject for one or more mutations in genes known to be associated with hearing loss and/or vestibular dysfunction prior to treatment with or administration of the compositions described herein. A subject can be screened for a genetic mutation using standard methods known to those of skill in the art (e.g., genetic testing). The methods described herein may also include a step of assessing hearing and/or vestibular function in a subject prior to treatment with or administration of the compositions described herein. Hearing can be assessed using standard tests, such as audiometry, auditory brainstem response (ABR), electrocochleography (ECOG), and otoacoustic emissions. Vestibular function may be assessed using standard tests, such as eye movement testing (e.g., electronystagmogram (ENG) or videonystagmogram (VNG)), tests of the vestibulo-ocular reflex (VOR) (e.g., the head impulse test (Halmagyi-Curthoys test), which can be performed at the bedside or using a video-head impulse test (VHIT), or the caloric reflex test), posturography, rotary-chair testing, ECOG, vestibular evoked myogenic potentials (VEMP), and specialized clinical balance tests, such as those described in Mancini and Horak, Eur J Phys Rehabil Med, 46:239 (2010). These tests can also be used to assess hearing and/or vestibular function in a subject after treatment with or administration of the compositions described herein. The compositions and methods described herein may also be administered as a preventative treatment to patients at risk of developing hearing loss and/or vestibular dysfunction, e.g., patients who have a family history of hearing loss or vestibular dysfunction (e.g., inherited hearing loss or vestibular dysfunction), patients carrying a genetic mutation associated with hearing loss or vestibular dysfunction who do not yet exhibit hearing impairment or vestibular dysfunction, or patients exposed to one or more risk factors for acquired hearing loss (e.g., acoustic trauma, disease or infection, head trauma, ototoxic drugs, or aging) or vestibular dysfunction (e.g., disease or infection, head trauma, ototoxic drugs, or aging). The compositions and methods described herein can also be used to treat a subject with idiopathic vestibular dysfunction.

The compositions and methods described herein can be used to convert a first inner ear cell type into a second inner ear cell type. For example, the compositions and methods described herein can be used to convert supporting cells (e.g., cochlear or vestibular supporting cells) into hair cells, and can, therefore, be used to induce or increase hair cell regeneration in a subject (e.g., cochlear and/or vestibular hair cell regeneration). Vectors containing a nucleic acid encoding Atoh1 can be used to convert supporting cells to hair cells. Such vectors can further include nucleic acids encoding Gfi1, Pou4f3, and/or Ikzf2 or can be administered in combination with one or more additional vectors containing nucleic acids encoding Gfi1, Pou4f3, and/or Ikzf2. Subjects that may benefit from compositions that induce or increase hair cell regeneration include subjects suffering from hearing loss or vestibular dysfunction as a result of loss of hair cells (e.g., loss of hair cells related to trauma (e.g., acoustic trauma or head trauma), disease or infection, ototoxic drugs, or aging), and subjects with abnormal hair cells (e.g., hair cells that do not function properly when compared to normal hair cells), damaged hair cells (e.g., hair cell damage related to trauma (e.g., acoustic trauma or head trauma), disease or infection, ototoxic drugs, or aging), or reduced hair cell numbers due to genetic mutations or congenital abnormalities. The compositions and methods described herein can also be used to promote or increase cochlear and/or vestibular hair cell maturation, which can lead to improved hearing and/or vestibular function, respectively.

In some embodiments, the compositions and methods described herein are used to convert a Type II vestibular hair cell into a Type I vestibular hair cell, which can increase the generation of Type I vestibular hair cells and/or increase the number of Type I vestibular hair cells (e.g., the total number of Type I vestibular hair cells in the vestibular system) and improve vestibular function. Vectors containing a polynucleotide that encodes or that can be transcribed to produce a Sox2 inhibitor can be used to convert Type II vestibular hair cells into Type I vestibular hair cells. Exemplary Sox2 inhibitors that can be included a vector described herein include a polynucleotide encoding a dnSox2 protein and a polynucleotide that can be transcribed to produce an inhibitory RNA molecule directed to Sox2 (e.g., an shRNA, siRNA, or shRNA-mir molecule directed to Sox2). Subjects that may benefit from compositions that promote or increase generation of Type I vestibular hair cells or increase Type I vestibular hair cell numbers include subjects having or at risk of developing vestibular dysfunction as a result of loss of hair cells (e.g., loss of vestibular hair cells related to trauma (e.g., head trauma), disease or infection, ototoxic drugs, or aging), subjects with abnormal vestibular hair cells (e.g., vestibular hair cells that do not function properly compared to normal vestibular hair cells), subjects with damaged vestibular hair cells (e.g., vestibular hair cell damage related to trauma (e.g., head trauma), disease or infection, ototoxic drugs, or aging), or subjects with reduced vestibular hair cell numbers due to genetic mutations or congenital abnormalities. By promoting the generation of hair cells (e.g., cochlear and/or vestibular hair cells) and/or Type I vestibular hair cells, the compositions and methods described herein can treat sensorineural hearing loss, deafness, auditory neuropathy, tinnitus, or vestibular dysfunction associated with loss of hair cells or with a lack of functional hair cells.

The compositions and methods described herein can also be used to prevent or reduce hearing loss and/or vestibular dysfunction caused by ototoxic drug-induced hair cell damage or death (e.g., cochlear hair cell and/or vestibular hair cell damage or death) in subjects who have been treated with ototoxic drugs, or who are currently undergoing or soon to begin treatment with ototoxic drugs. Ototoxic drugs are toxic to the cells of the inner ear, and can cause sensorineural hearing loss, vestibular dysfunction (e.g., vertigo, dizziness, imbalance, bilateral vestibulopathy, or oscillopsia), tinnitus, or a combination of these symptoms. Drugs that have been found to be ototoxic include aminoglycoside antibiotics (e.g., gentamycin, neomycin, streptomycin, tobramycin, kanamycin, vancomycin, and amikacin), viomycin, antineoplastic drugs (e.g., platinum-containing chemotherapeutic agents, such as cisplatin, carboplatin, and oxaliplatin), loop diuretics (e.g., ethacrynic acid and furosemide), salicylates (e.g., aspirin, particularly at high doses), and quinine. In some embodiments, the methods and compositions described herein can be used to treat bilateral vestibulopathy or oscillopsia due to aminoglycoside ototoxicity (e.g., generate additional Type I vestibular hair cells to replace damaged or dead cells and/or promote or increase hair cell regeneration in a subject with aminoglycoside-induced bilateral vestibulopathy or oscillopsia).

In some embodiments, the compositions and methods described herein are used to treat a subject having a genetic form of hearing loss and/or vestibular dysfunction. In such embodiments, the vector can contain a promoter operably linked to a polynucleotide encoding a wild-type form of a gene that is mutated in the subject (e.g., a gene listed in Table 4) and to a polynucleotide that can be transcribed to produce a miRNA target sequence recognized by a miRNA that is not expressed in the inner ear cell type that normally expresses the gene (e.g., a miRNA target sequence for a miRNA that is expressed in one or more inner ear cell types that do not normally express the gene, which would prevent or reduce off-target expression of the polynucleotide in the one or more inner ear cell types that do not normally express it). The compositions and methods described herein can also be used to deliver a polynucleotide listed in Table 5 to the corresponding inner ear cell type listed in Table 5, e.g., using a vector containing a promoter operably linked to a polynucleotide listed in Table 5 and to one or more polynucleotides that can be transcribed to produce a miRNA target sequence for one or more miRNAs expressed in one or more inner ear cell types other than the corresponding inner ear cell type for the polynucleotide listed in Table 5. If the polynucleotide delivered using a vector described herein corresponds to a gene that regulates inner ear cell development, function, cell fate specification, regeneration, survival, proliferation, and/or maintenance, then administration of the vector to a subject can regulate inner ear cell development, function, cell fate specification, regeneration, survival, proliferation, and/or maintenance in the subject's inner ear.

Treatment may include administration of a composition containing a nucleic acid vector described herein in various unit doses. Each unit dose will ordinarily contain a predetermined quantity of the therapeutic composition. The quantity to be administered, and the particular route of administration and formulation, are within the skill of those in the clinical arts. A unit dose need not be administered as a single injection but may comprise continuous infusion over a set period of time. Dosing may be performed using a syringe pump to control infusion rate in order to minimize damage to the inner ear. In cases in which the nucleic acid vector is an AAV vector (e.g., an AAV1, AAV2, AAV3, AAV4, AAV5, AAV6, AAV7, AAV8, AAV9, AAV10, AAV11, rh10, rh39, rh43, rh74, AAV2-QuadYF, Anc80, Anc80L65, DJ, DJ/8, DJ/9, 7m8, PHP.B, PHP.B2, PBP.B3, PHP.A, PHP.eb, or PHP.S vector), the viral vector may be administered to the patient at a dose of, for example, from about 1×10⁹ vector genomes (VG)/mL to about 1×10¹⁶ VG/mL (e.g., 1×10⁹ VG/mL, 2×10⁹ VG/mL, 3×10⁹ VG/mL, 4×10⁹ VG/mL, 5×10⁹ VG/mL, 6×10⁹ VG/mL, 7×10⁹ VG/mL, 8×10⁹ VG/mL, 9×10⁹ VG/mL, 1×10¹⁰ VG/mL, 2×10¹⁰ VG/mL, 3×10¹⁰ VG/mL, 4×10¹⁰ VG/mL, 5×10¹⁰ VG/mL, 6×10¹⁰ VG/mL, 7×10¹⁰ VG/mL, 8×10¹⁰ VG/mL, 9×10¹⁰ VG/mL, 1×10¹¹ VG/mL, 2×10¹¹ VG/mL, 3×10¹¹ VG/mL, 4×10¹¹ VG/mL, 5×10¹¹ VG/mL, 6×10¹¹ VG/mL, 7×10¹¹ VG/mL, 8×10¹¹ VG/mL, 9×10¹¹ VG/mL, 1×10¹² VG/mL, 2×10¹² VG/mL, 3×10¹² VG/mL, 4×10¹² VG/mL, 5×10¹² VG/mL, 6×10¹² VG/mL, 7×10¹² VG/mL, 8×10¹² VG/mL, 9×10¹² VG/mL, 1×10¹³ VG/mL, 2×10¹³ VG/mL, 3×10¹³ VG/mL, 4×10¹³ VG/mL, 5×10¹³ VG/mL, 6×10¹³ VG/mL, 7×10¹³ VG/mL, 8×10¹³ VG/mL, 9×10¹³ VG/mL, 1×10¹⁴ VG/mL, 2×10¹⁴ VG/mL, 3×10¹⁴ VG/mL, 4×10¹⁴ VG/mL, 5×10¹⁴ VG/mL, 6×10¹⁴ VG/mL, 7×10¹⁴ VG/mL, 8×10¹⁴ VG/mL, 9×10¹⁴ VG/mL, 1×10¹⁵ VG/mL, 2×10¹⁵ VG/mL, 3×10¹⁵ VG/mL, 4×10¹⁵ VG/mL, 5×10¹⁵ VG/mL, 6×10¹⁵ VG/mL, 7×10¹⁵ VG/mL, 8×10¹⁵ VG/mL, 9×10¹⁵ VG/mL, or 1×10¹⁶ VG/mL) in a volume of 1 μL to 200 μL (e.g., 1, 2, 3, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200 μL). The AAV vector may be administered to the subject at a dose of about 1×10⁷ VG/ear to about 2×10¹⁵ VG/ear (e.g., 1×10⁷ VG/ear, 2×10⁷ VG/ear, 3×10⁷ VG/ear, 4×10⁷ VG/ear, 5×10⁷ VG/ear, 6×10⁷ VG/ear, 7×10⁷ VG/ear, 8×10⁷ VG/ear, 9×10⁷ VG/ear, 1×10⁸ VG/ear, 2×10⁸ VG/ear, 3×10⁸ VG/ear, 4×10⁸ VG/ear, 5×10⁸ VG/ear, 6×10⁸ VG/ear, 7×10⁸ VG/ear, 8×10⁸ VG/ear, 9×10⁸ VG/ear, 1×10⁹ VG/ear, 2×10⁹ VG/ear, 3×10⁹ VG/ear, 4×10⁹ VG/ear, 5×10⁹ VG/ear, 6×10⁹ VG/ear, 7×10⁹ VG/ear, 8×10⁹ VG/ear, 9×10⁹ VG/ear, 1×10¹⁰ VG/ear, 2×10¹⁰ VG/ear, 3×10¹⁰ VG/ear, 4×10¹⁰ VG/ear, 5×10¹⁰ VG/ear, 6×10¹⁰ VG/ear, 7×10¹⁰ VG/ear, 8×10¹⁰ VG/ear, 9×10¹⁰ VG/ear, 1×10¹¹ VG/ear, 2×10¹¹ VG/ear, 3×10¹¹ VG/ear, 4×10¹¹ VG/ear, 5×10¹¹ VG/ear, 6×10¹¹ VG/ear, 7×10¹¹ VG/ear, 8×10¹¹ VG/ear, 9×10¹¹ VG/ear, 1×10¹² VG/ear, 2×10¹² VG/ear, 3×10¹² VG/ear, 4×10¹² VG/ear, 5×10¹² VG/ear, 6×10¹² VG/ear, 7×10¹² VG/ear, 8×10¹² VG/ear, 9×10¹² VG/ear, 1×10¹³ VG/ear, 2×10¹³ VG/ear, 3×10¹³ VG/ear, 4×10¹³ VG/ear, 5×10¹³ VG/ear, 6×10¹³ VG/ear, 7×10¹³ VG/ear, 8×10¹³ VG/ear, 9×10¹³ VG/ear, 1×10¹⁴ VG/ear, 2×10¹⁴ VG/ear, 3×10¹⁴ VG/ear, 4×10¹⁴ VG/ear, 5×10¹⁴ VG/ear, 6×10¹⁴ VG/ear, 7×10¹⁴ VG/ear, 8×10¹⁴ VG/ear, 9×10¹⁴ VG/ear, 1×10¹⁵ VG/ear, or 2×10¹⁵ VG/ear).

The compositions described herein can be administered in an amount sufficient to improve hearing, improve vestibular function (e.g., improve balance or reduce dizziness or vertigo), reduce tinnitus, treat bilateral vestibulopathy, treat oscillopsia, treat a balance disorder, treat genetic hearing loss, deafness, or vestibular dysfunction, increase or induce hair cell regeneration (e.g., cochlear and/or vestibular hair cell regeneration), increase hair cell numbers, increase hair cell maturation (e.g., maturation of regenerated hair cells), improve the function of one or more inner ear cell types, improve inner ear cell survival (e.g., in a subject exposed to an ototoxic drug, acoustic trauma or head trauma, or a disease or infection that affects inner ear cells, or in a subject of advanced age), increase inner ear cell proliferation, increase the generation of Type I vestibular hair cells, or increase the number of Type I vestibular hair cells. Hearing may be evaluated using standard hearing tests (e.g., audiometry, ABR, electrocochleography (ECOG), and otoacoustic emissions) and may be improved by 5% or more (e.g., 5%, 10%, 15%, 20%, 30%, 40%, 50%, 60%, 70%, 80%, 90%, 100%, 125%, 150%, 200% or more) compared to hearing measurements obtained prior to treatment. Vestibular function may be evaluated using standard tests for balance and vertigo (e.g., eye movement testing (e.g., ENG or VNG), posturography, VOR testing (e.g., head impulse testing (Halmagyi-Curthoys testing, e.g., VHIT), or caloric reflex testing), rotary-chair testing, ECOG, VEMP, and specialized clinical balance tests) and may be improved by 5% or more (e.g., 5%, 10%, 15%, 20%, 30%, 40%, 50%, 60%, 70%, 80%, 90%, 100%, 125%, 150%, 200% or more) compared to measurements obtained prior to treatment. In some embodiments, the compositions are administered in an amount sufficient to improve the subject's ability to understand speech. The compositions described herein may also be administered in an amount sufficient to slow or prevent the development or progression of sensorineural hearing loss and/or vestibular dysfunction (e.g., in subjects who carry a genetic mutation associated with hearing loss or vestibular dysfunction, who have a family history of hearing loss or vestibular dysfunction (e.g., hereditary hearing loss or vestibular dysfunction), or who have been exposed to risk factors associated with hearing loss or vestibular dysfunction (e.g., ototoxic drugs, head trauma, disease or infection, or acoustic trauma) but do not yet exhibit hearing impairment or vestibular dysfunction (e.g., vertigo, dizziness, or imbalance), or in subjects exhibiting mild to moderate hearing loss or vestibular dysfunction). Hair cell regeneration, maturation, or survival or Type I vestibular hair cell generation or numbers may be evaluated indirectly based on hearing tests or tests of vestibular function, and may be increased by 5% or more (e.g., 5%, 10%, 15%, 20%, 30%, 40%, 50%, 60%, 70%, 80%, 90%, 100%, 125%, 150%, 200% or more) compared to hair cell regeneration or maturation or Type I vestibular hair cell generation or numbers prior to administration of the compositions described herein. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months, or more following administration of the composition depending on the dose and route of administration used for treatment. Depending on the outcome of the evaluation, the patient may receive additional treatments.

### Kits

The compositions described herein can be provided in a kit for use in promoting hair cell regeneration (e.g., cochlear and/or vestibular hair cell regeneration), generating Type I vestibular hair cells, improving inner ear function, and/or treating hearing loss (e.g., sensorineural hearing loss), auditory neuropathy, deafness, tinnitus, or vestibular dysfunction (e.g., dizziness, imbalance, vertigo, bilateral vestibulopathy, a balance disorder, or oscillopsia). The kit may include a nucleic acid vector containing a promoter operably linked to a polynucleotide that can be transcribed to produce a desired expression product and to a polynucleotide that can be transcribed to produce a miRNA target sequence (e.g., a target sequence for a miRNA that is differentially expressed among different inner ear cell types) The nucleic acid vectors may be packaged in an AAV virus capsid (e.g., AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S). The kit can further include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1—Effect of miRNA Target Sequences on Expression of AAV Vector-Encoded acGFP in HEK293-T Cells

HEK293-T cells are known to express the three miRNAs in the miR-183 cluster (mir-183, -96, and -182) to varying degrees. AAVs containing an acGFP transgene and target sequences for one or more of these miRNAs were used to infect HEK293-T cells to determine if they would induce GFP expression, and if that GFP expression would be modulated by the presence of the miRNA target sequences.

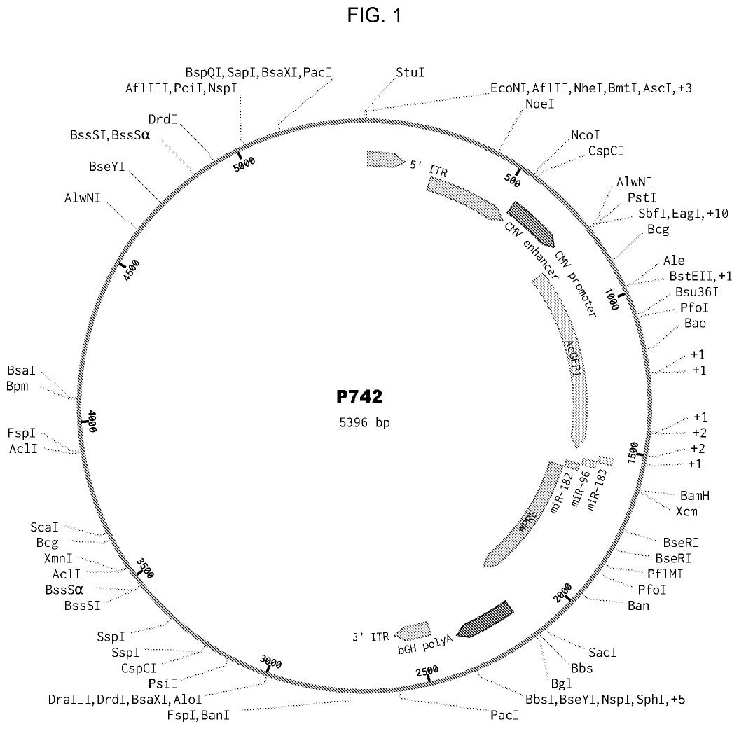

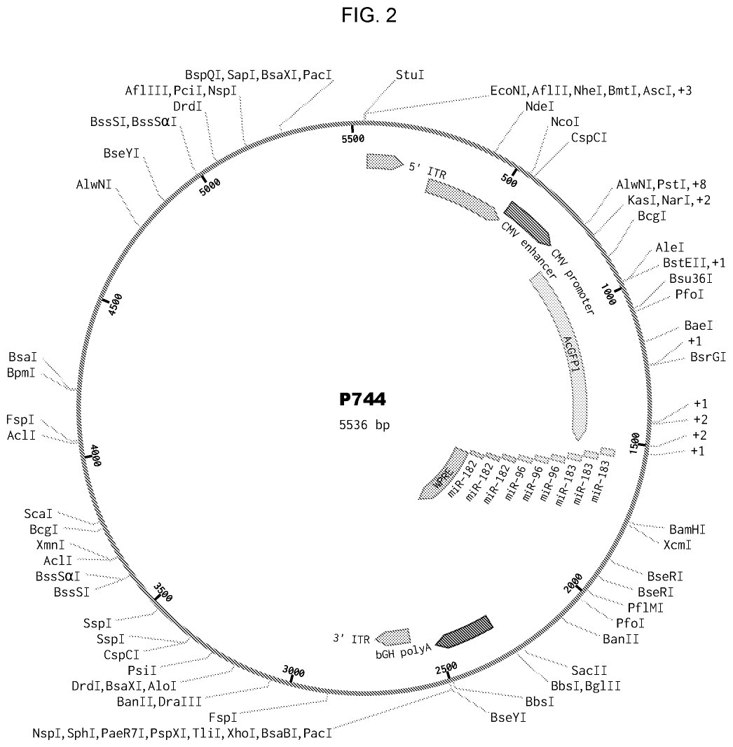

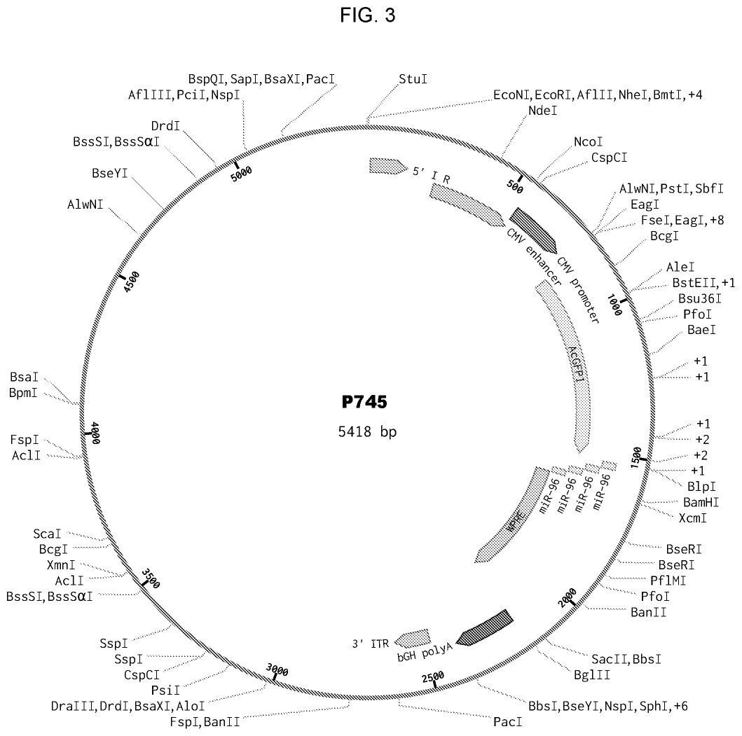

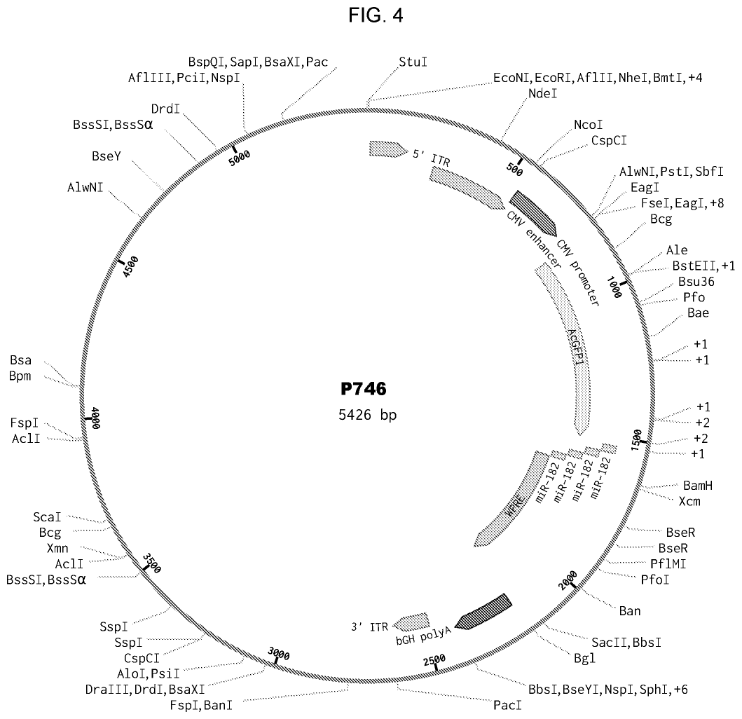

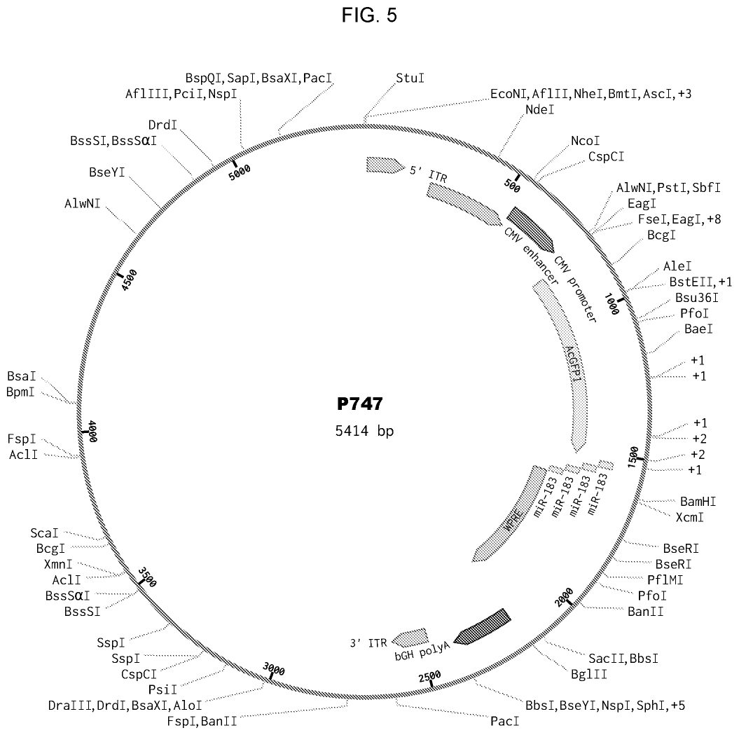

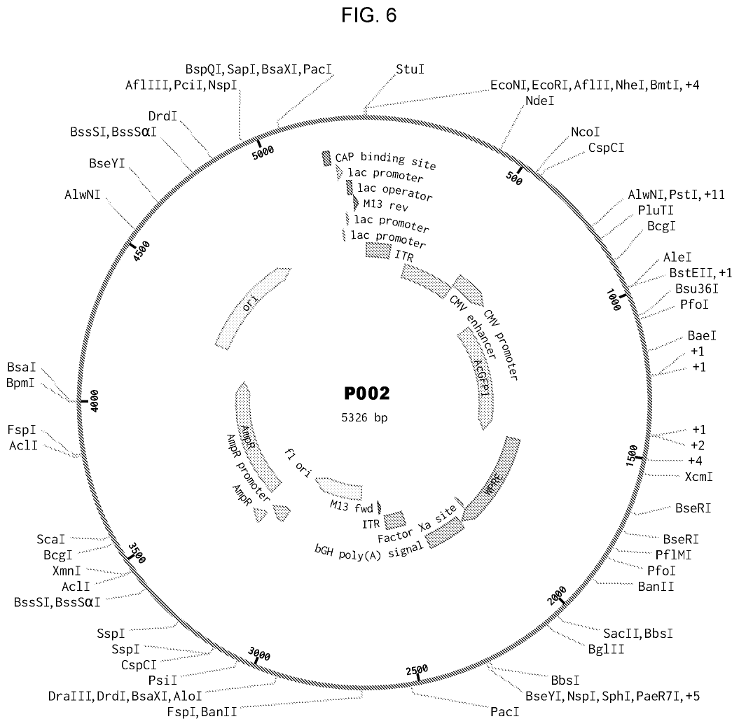

The AAV viral vectors used in this experiment were synthesized as follows. HEK293-T cells (obtained from ATCC, Manassas, VA) were seeded into cell culture-treated dishes (15 cm) and grown until they reached 70-80% confluence in the vessel. GFP-encoding plasmids containing various miRNA target sequences (plasmids P742, P744, P745, P746, P747; FIGS. 1-5), or a transgene plasmid lacking any miRNA target sequence (plasmid P002; FIG. 6) were individually combined with the plasmid pXR8 containing AAV2 rep/AAV8 cap (Addgene #112864) and the adenoviral helper plasmid pXX6-80 (X Xiao et al., J Virol 72(3), pp. 2224-32 (1998)) at a 1:1:1 molar ratio and 52.3 μg of that mixture was combined with PEIMax (Polysciences). A total of 52.3 μg of that plasmid mixture was delivered onto each 15 cm plate containing the cells. The cell culture medium and the cells were subsequently collected to extract and purify the AAV. AAV from the cells was released from cells through three cycles of freeze thaw, and the cell culture medium was collected to obtain secreted AAV. AAV from the cell culture medium was concentrated by adding PEG8000 to the solution, incubating at 4° C., and centrifuging to collect the AAV particles. All AAV was passed through iodixanol density gradient centrifugation to purify the AAV particles, and the buffer was exchanged to PBS with 0.01% pluronic F68 by passing the purified AAV and the buffer over a centrifugation column with a 100 kDa molecular weight cutoff. The other AAV viral vectors described in this and further examples herein were synthesized in a similar fashion using the appropriate transgene plasmid (which provides the promoter, the transgene(s), and other elements required for transgene expression).

HEK293-T cells were then seeded in a 96-well plate at a density of 10,000 cells/well in DMEM+GlutaMAX+10% PenStrep. At the time of seeding, wells were treated with the following AAVs, in triplicate, at an MOI of 106 viral genomes (vg)/cell. Table 13 below lists the transgene plasmids used for the individual AAV vectors and the titer of the virus.

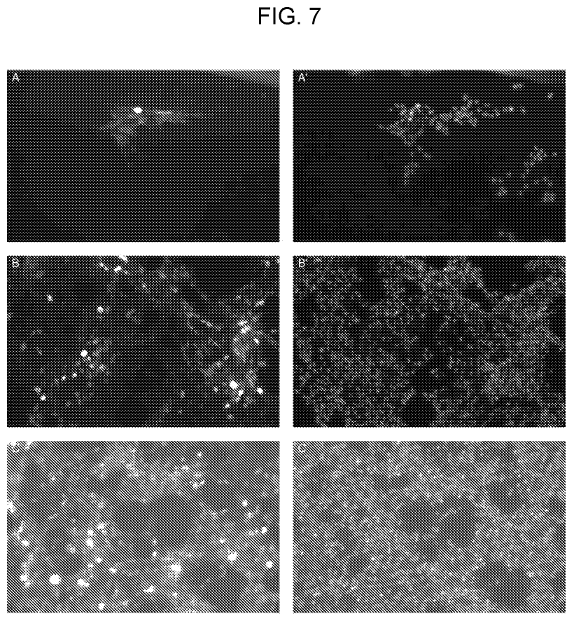

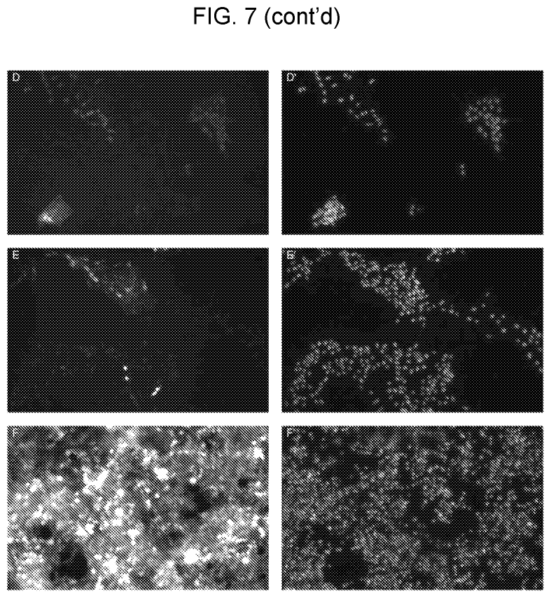

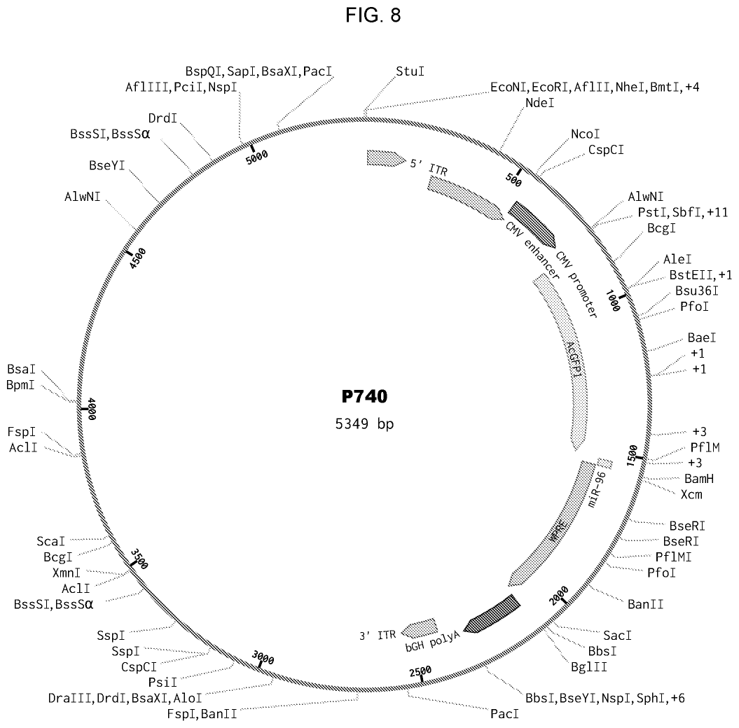

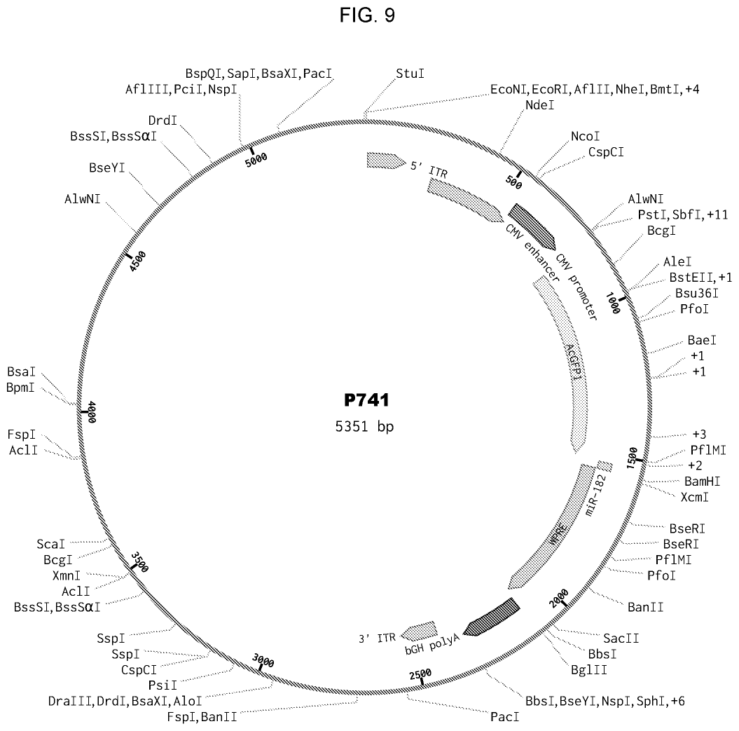

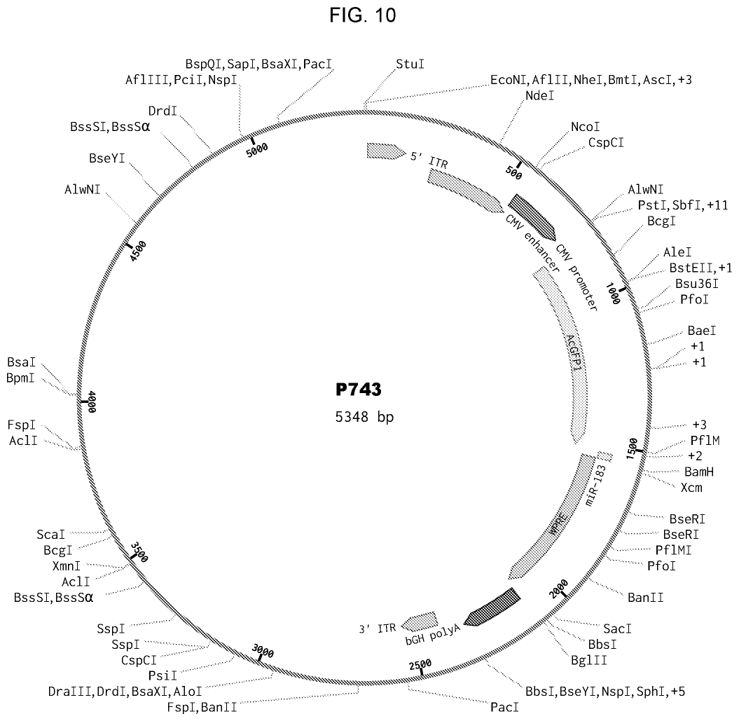

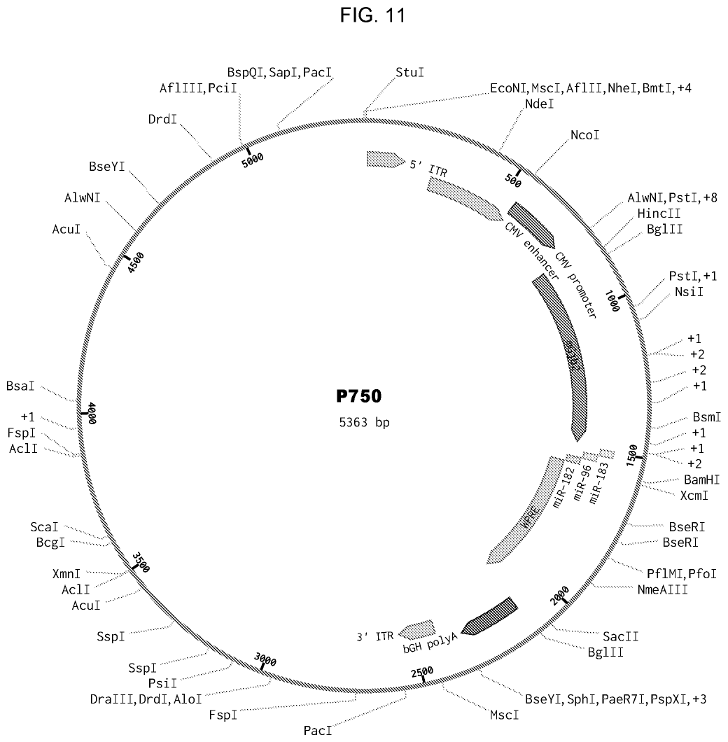

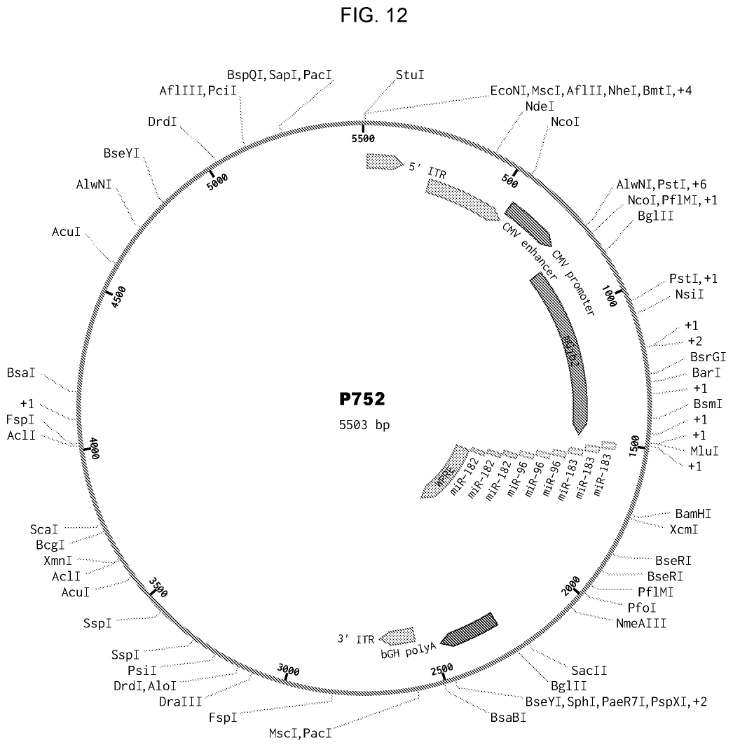

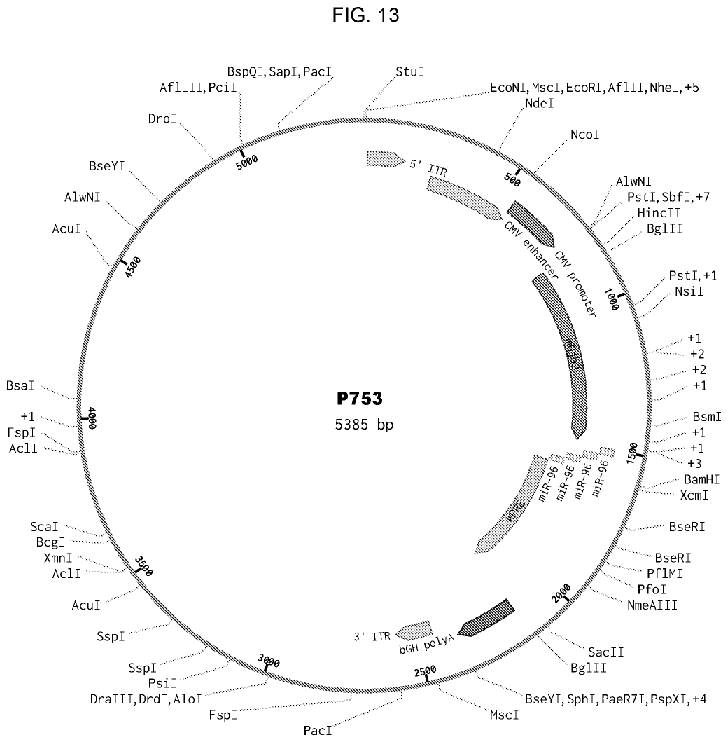

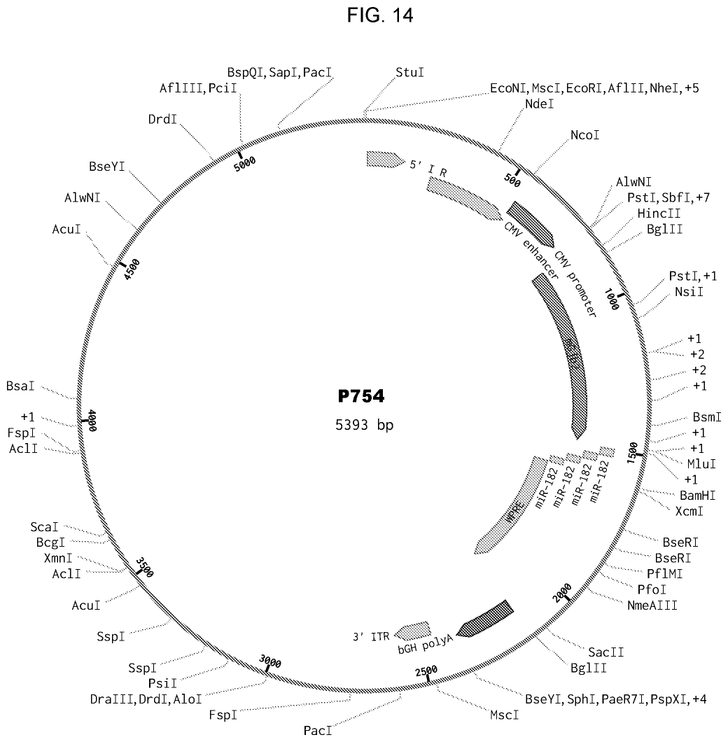

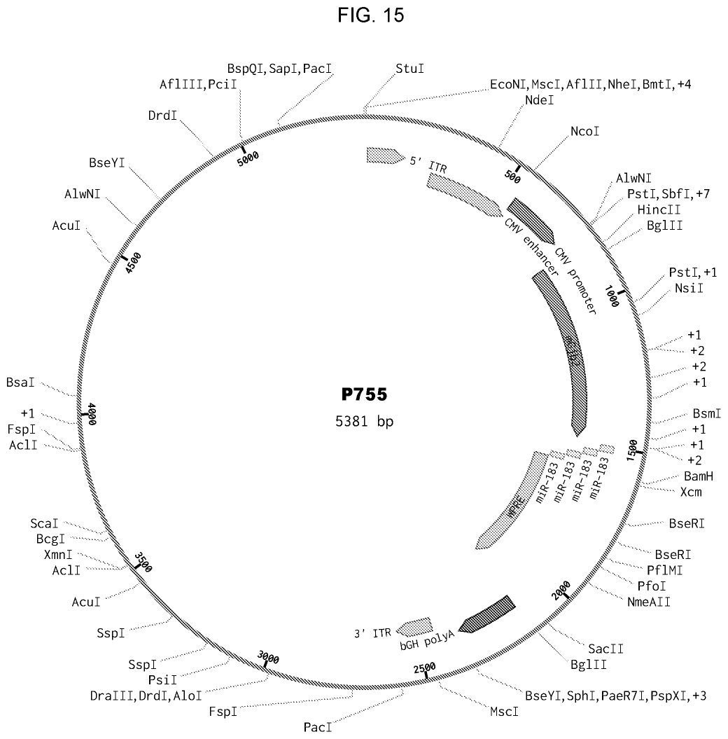

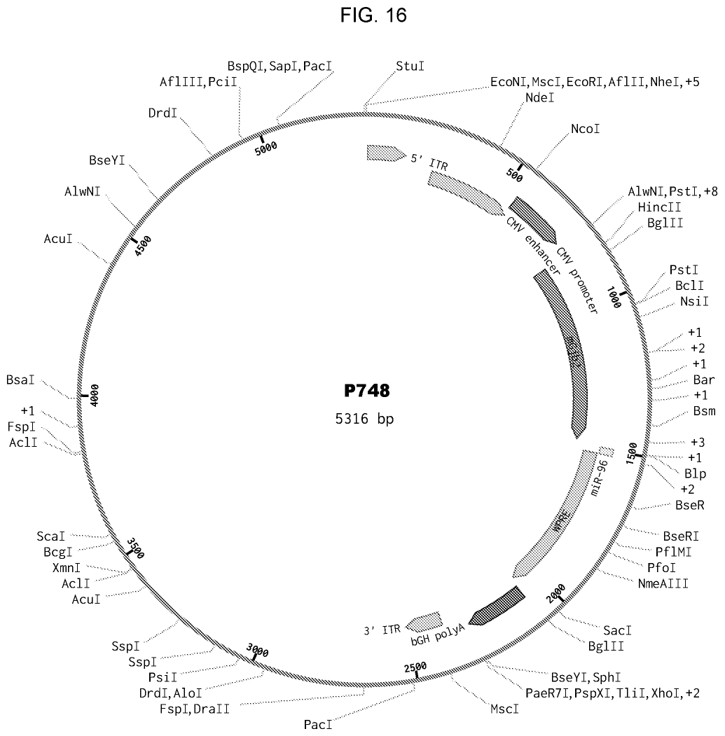

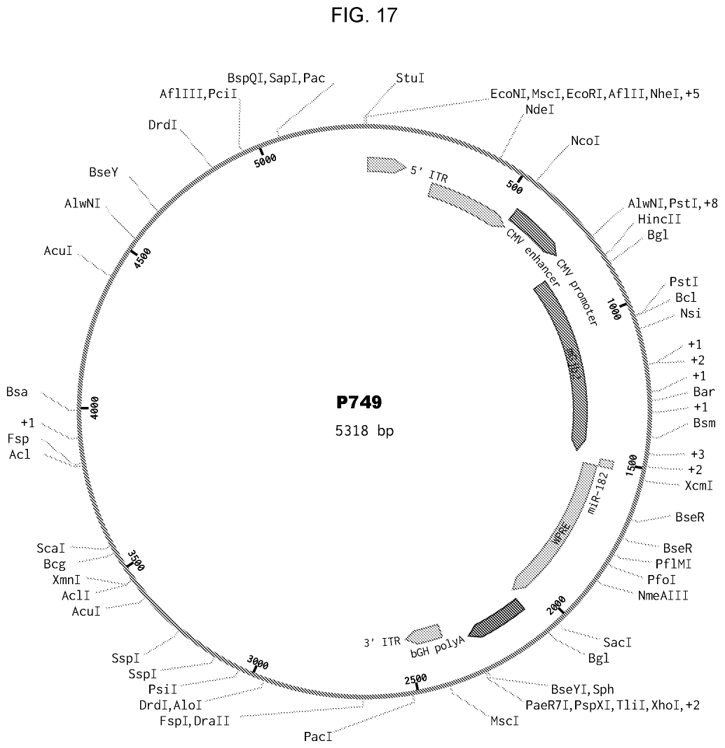

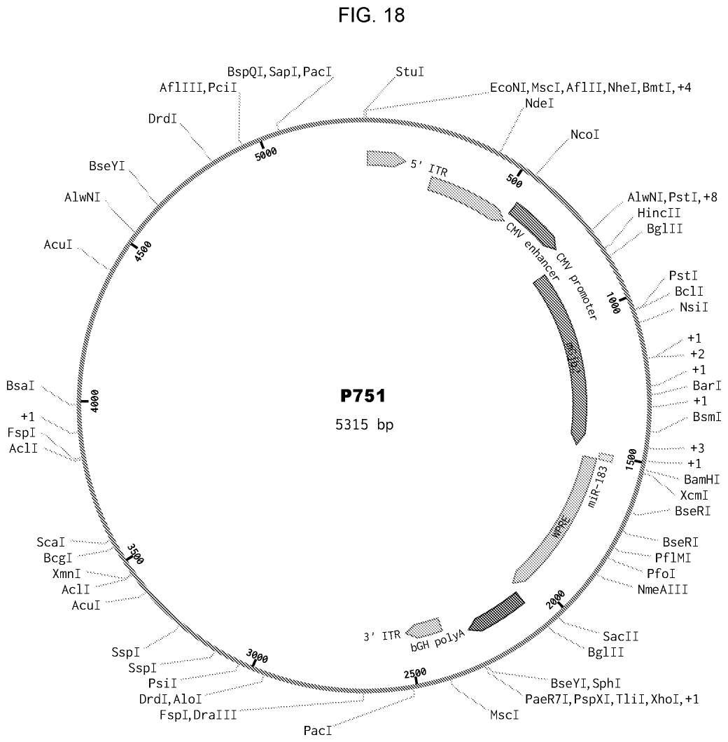

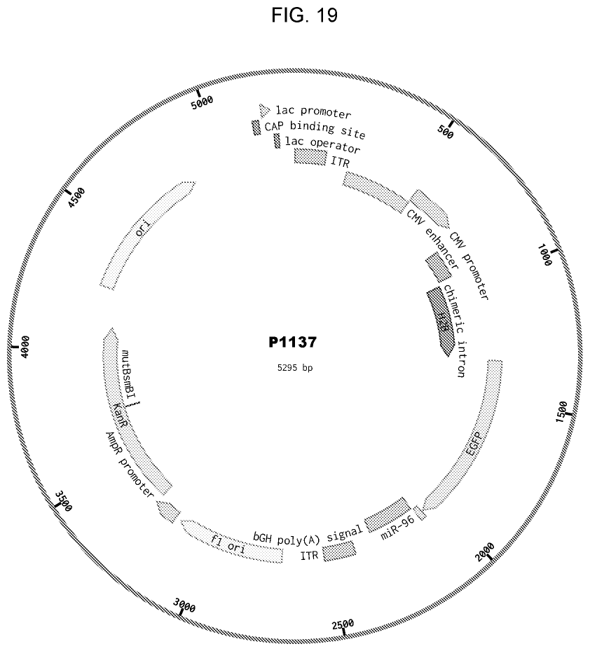

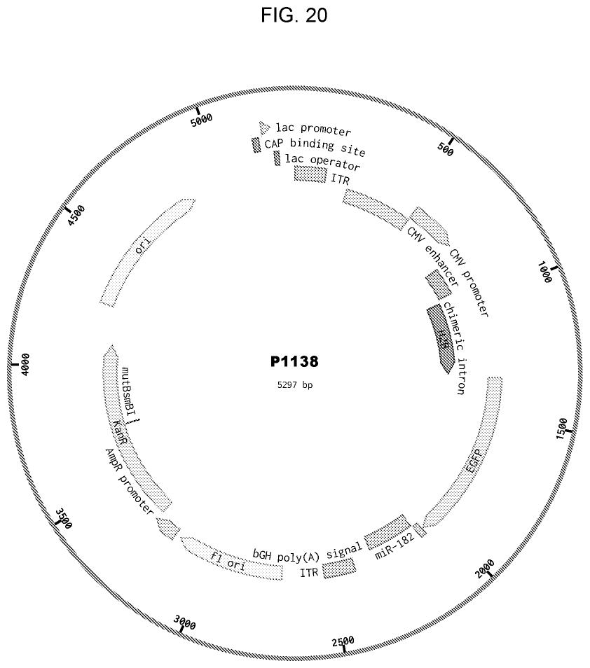

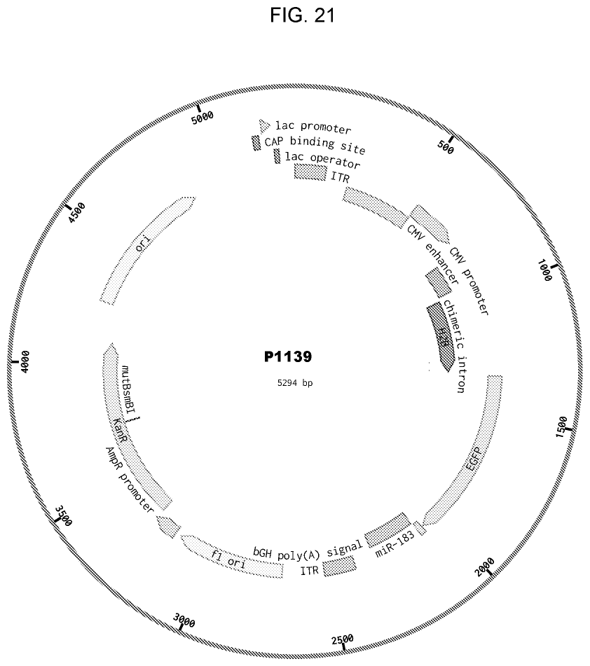

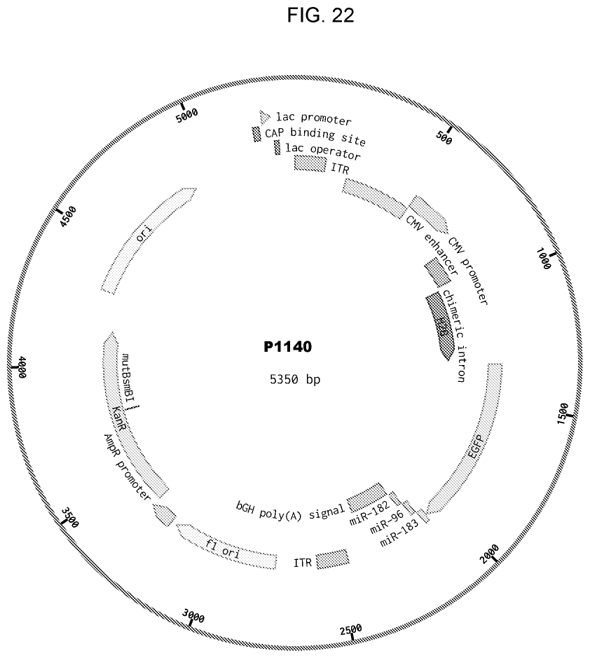

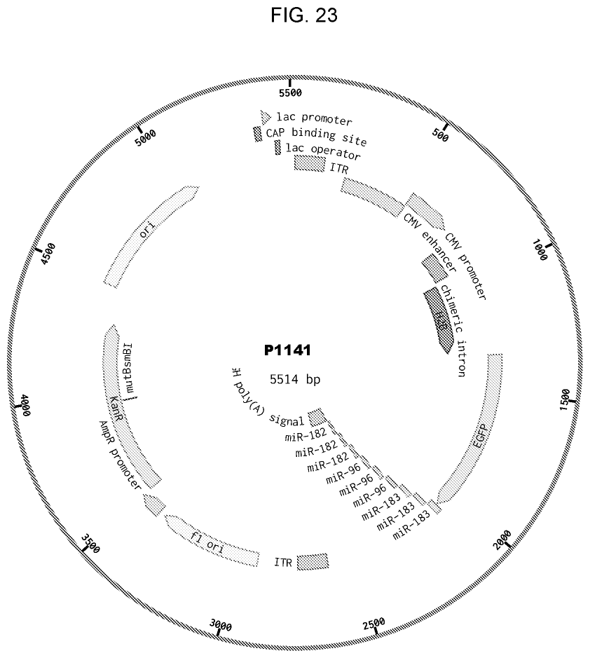

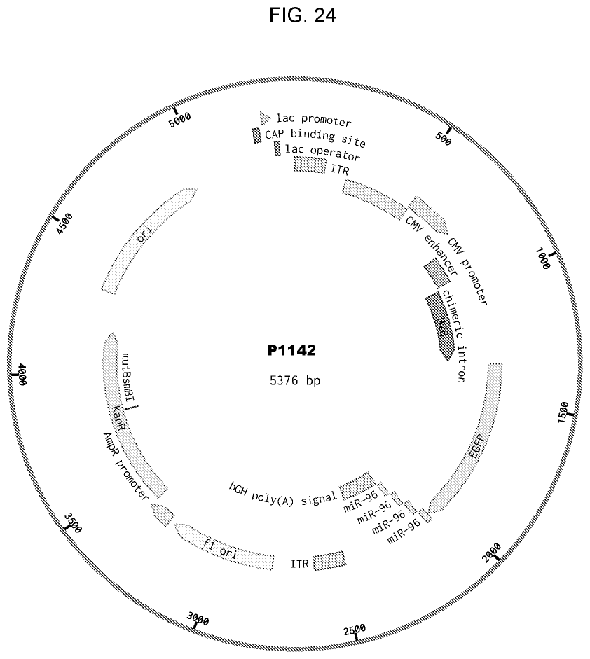

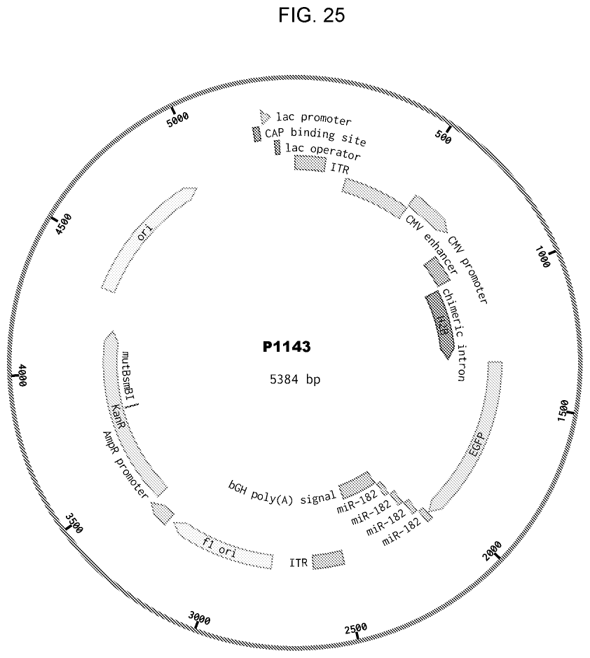

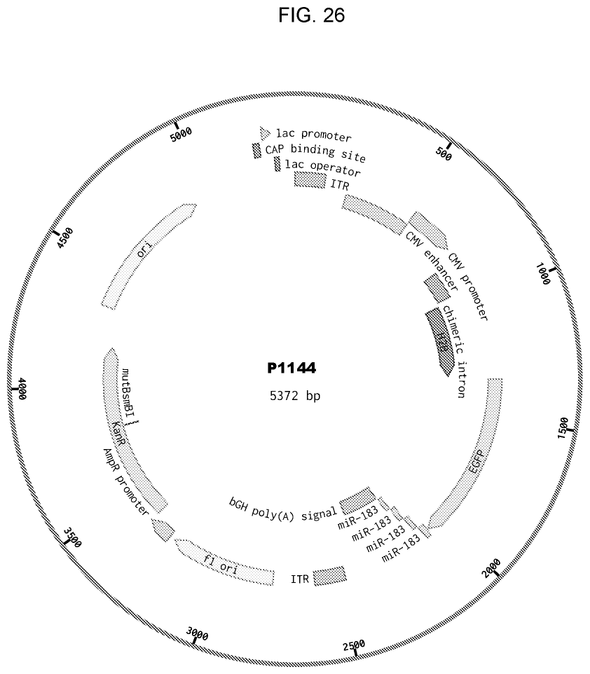

TABLE 13
Transgene plasmid sources and titers of
AAV vectors used to infect HEK293-T cells

| Corresponding Panels | Transgene Source of | |
|---|---|---|
| in FIG. 7 | AAV Vector | Titer |
| A/A′ | P742 (SEQ ID NO: 1) | 4.5703125 × 10¹³ |
| B/B′ | P744 (SEQ ID NO: 2) | 4.9453125 × 10¹³ |
| C/C′ | P745 (SEQ ID NO: 3) | 5.8046875 × 10¹³ |
| D/D′ | P746 (SEQ ID NO: 4) | 5.2578125 × 10¹³ |
| E/E′ | P747 (SEQ ID NO: 5) | 5.515625 × 10¹³ |
| F/F′ | P002 (control) | 4.5546875 × 10¹³ |

The cells were incubated for four days in the virus-containing media at 37° C. and 5% CO₂. After four days, the cells were fixed by aspirating the media+virus and incubating the wells in 4% formaldehyde at room temperature for 20 minutes, then staining with DAPI to label cell nuclei. Cells were imaged with the Zeiss Inverted Apotome microscope to look at DAPI and endogenous GFP expression. The results are shown in FIG. 7.

The positive control, which contained no miRNA target sequences, produced very strong GFP expression in HEK293-T cells, indicating that the vector transduced the cells very well and expression was not downregulated. The lower level of expression shown from the other viral vectors compared to the control suggests that the mir-183 cluster target sequences were indeed being bound by endogenous HEK293-T miRNAs to downregulate GFP expression.

### Example 2—Effect of miRNA Target Sequences on Expression of AAV Vector-Encoded EGFP in HEK293T Cells Co-Transfected with miRNA Target Sequences and Complementary Synthetic miRNAs

Plasmids containing a polynucleotide encoding a nuclear GFP together with one or more polynucleotides that can be transcribed to produce a miRNA target sequence (P1137, P1138, P1139, P1140, P1141, P1142, P1143, or P1144) were transfected into HEK293T cells with or without co-transfection with their complementary synthetic miRNAs (miR-96, miR-182, or miR-183) from the Invitrogen miRVana product line as follows. Two 24-well plates were seeded at 40,000 cells/well. After 24 hours, the confluency of seeded plates was checked. Once cells reached ≥70% confluency, the transfection was carried out. For cells that were transfected with both plasmid DNA and miRNA, a solution containing 8 ng/μl plasmid DNA and 0.2 pMol/μL miRNA in Opti-MEM was prepared. For plasmid-only transfections, a solution containing 8 ng/μl plasmid DNA in Opti-MEM was prepared. These solutions were incubated for five minutes at room temperature following preparation and then diluted with an equal volume of 4% Lipofectamine 3000 in Opti-MEM. The solution was then mixed gently and incubated for another 10-15 minutes at room temperature. Fifty μL of the appropriate DNA/miRNA/Lipo or DNA/Lipo complex was added to the cells in each well and the plate was rocked to ensure even mixing. The plates were incubated in an IncuCyte apparatus for 48 hours, with imaging occurring every six hours. After 48 hours, each sample was run through a Sony Fluorescence-Activated Cell Sorter to calculate the ratio of GFP-positive cells in each sample.

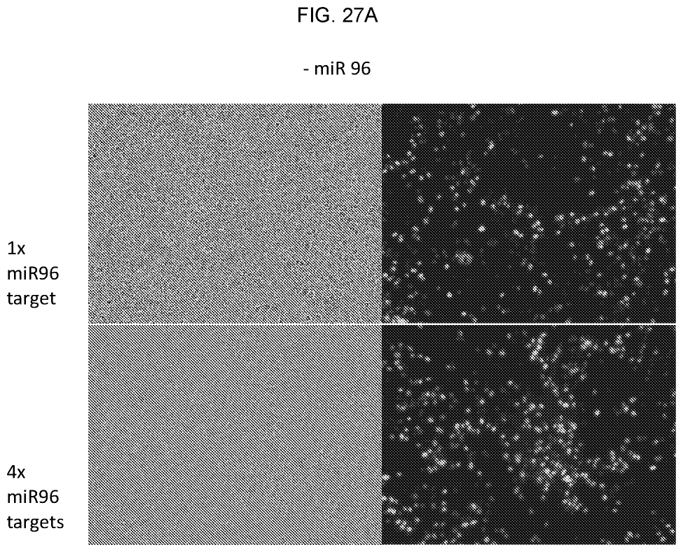

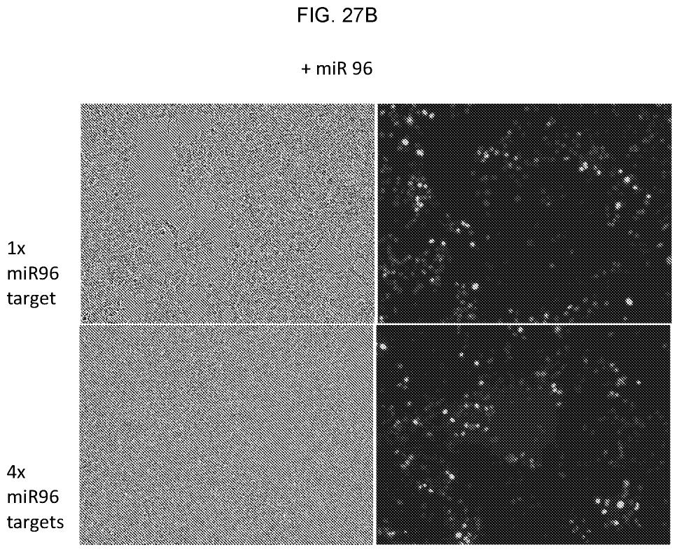

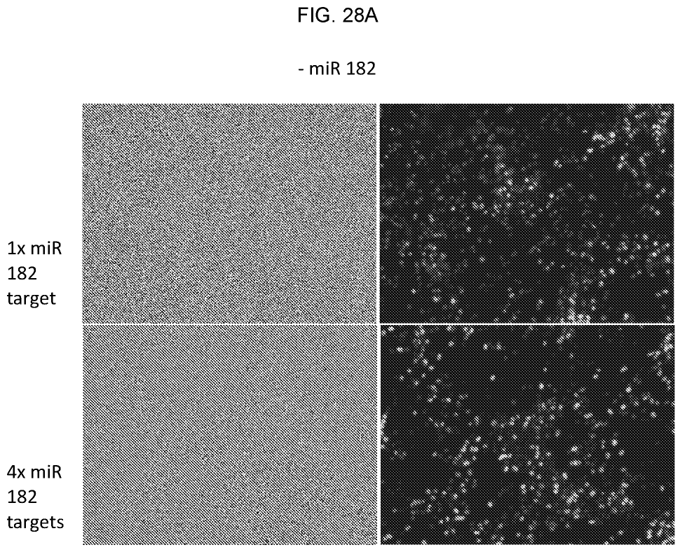

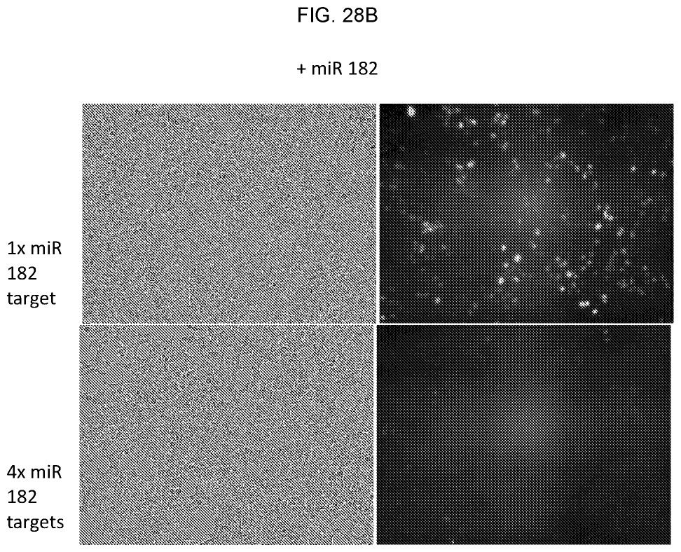

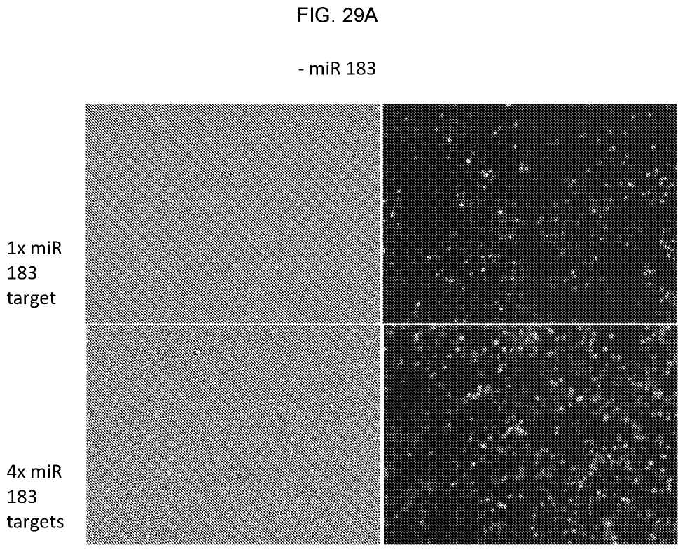

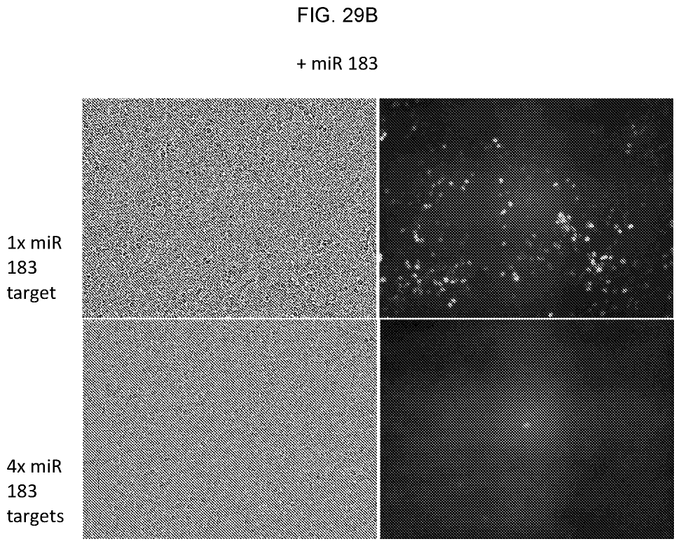

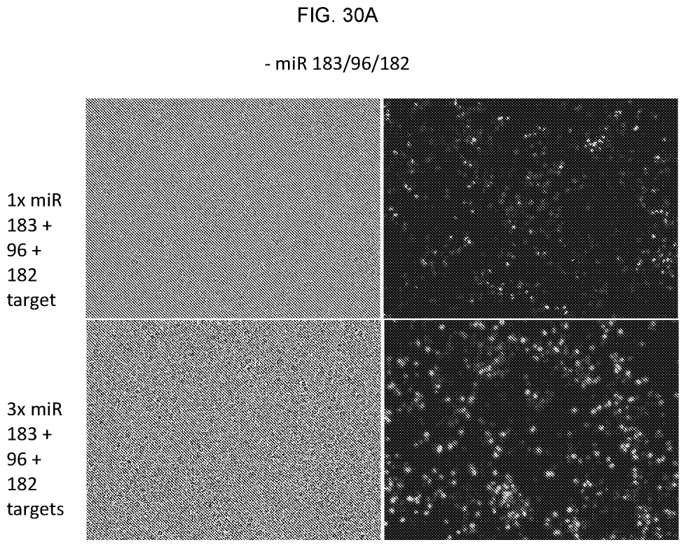

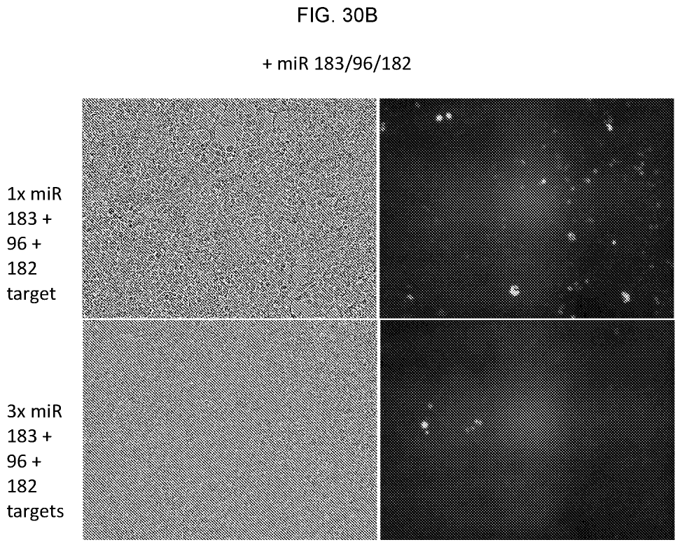

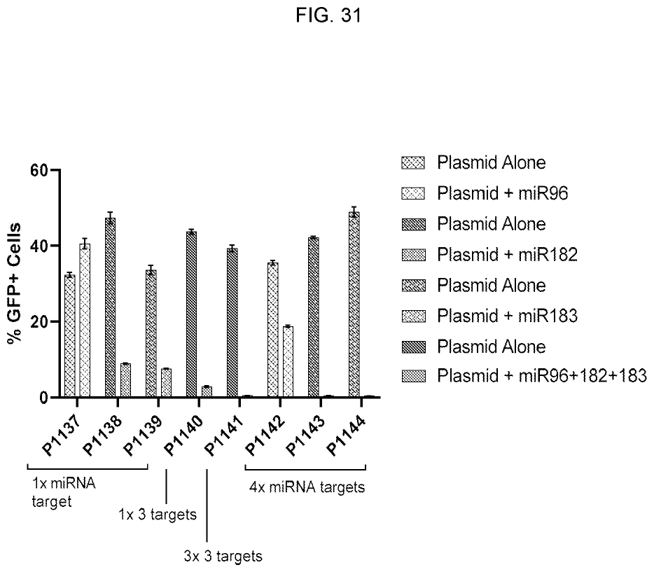

Micrographs of cells treated with different plasmids containing polynucleotides that can be transcribed to produce various miRNA targeting sequences with and without co-transfection with an appropriate miRNA are shown in FIGS. 27A-27B, 28A-28B, 29A-29B, and 30A-30B, with the bright field and GFP channels shown separately. While miR-96 did not appear to reduce GFP expression in cells transfected with a plasmid containing one copy of a polynucleotide that can be transcribed to produce an miR-96 target sequence and only moderately reduced expression in cells transfected with a plasmid containing four copies of a polynucleotide that can be transcribed to produce an miR-96 target sequence (FIGS. 27A and 27B), both miR-182 and miR-183 resulted in greatly reduced GFP expression in cells transfected with plasmids containing one or four copies of a polynucleotide that can be transcribed to produce the corresponding miRNA targeting sequence (FIGS. 28A, 28B, 29A and 29B). One copy of either a polynucleotide that can be transcribed to produce an miR-182 or miR-183 target sequence resulted in an approximately 6-fold reduction in GFP expression in cells co-transfected with the appropriate miRNA. Four copies of a polynucleotide that can be transcribed to produce these target sequences resulted in almost complete inhibition (˜100-fold reduction) of GFP expression. A plasmid harboring one copy of each polynucleotide that can be transcribed to produce a miRNA-96, miRNA-182 and miRNA-183 target sequence showed approximately 15-fold reduction in GFP expression in the presence of all three of the corresponding miRNAs. A plasmid harboring three copies of each polynucleotide that can be transcribed to produce a miRNA-96, miRNA-182 and miRNA-183 target sequence showed approximately 78-fold reduction in GFP expression in the presence of all three of the corresponding miRNAs. See FIGS. 30A and 30B. These results are summarized in FIG. 31.

### Example 3—Effect of miRNA Target Sequences on Expression of AAV Vector-Encoded eGFP in Murine Cochlear Explants

The microRNAs mir-96, mir-182, and mir-183 are highly expressed in cochlear HCs. AAV viral vectors containing an H2B-eGFP transgene and target sequences for one or more of these miRNAs were used to infect neonatal murine cochlear explants to determine if they induce GFP expression, and if that GFP expression was modulated by the presence of the miRNA target sequences.

Sensory epithelia were dissected from P1 mice and plated two to a dish on Matrigel-treated MatTek 35 mm dishes with a #0 10 mm coverslip. 150-200 μL of DMEM+10% FBS+10 μg/mL ciprofloxacin was added to each dish. After a 1-hour incubation at 37° C./5% CO₂, 1×10¹¹ viral genomes of an AAV viral vector as indicated in Table 14, below were added to each dish.

TABLE 14
Transgene plasmid sources of AAV vectors used to infect
different groups of murine cochlear explants

| Group | Transgene Source of AAV Vector |
|---|---|
| 1 | P1142 (SEQ ID NO: 22) |
| 2 | P1143 (SEQ ID NO: 23) |
| 3 | P1144 (SEQ ID NO: 24) |
| 4 | P1141 (SEQ ID NO: 21) |
| 5 | P707 (a control vector containing an H2B-eGFP transgene and |
| | no miRNA recognition sequences) |

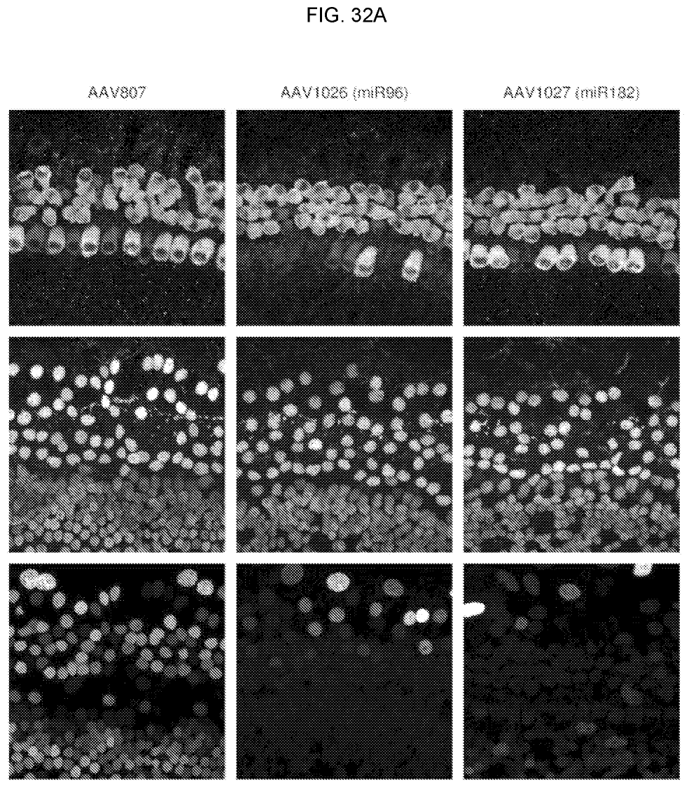

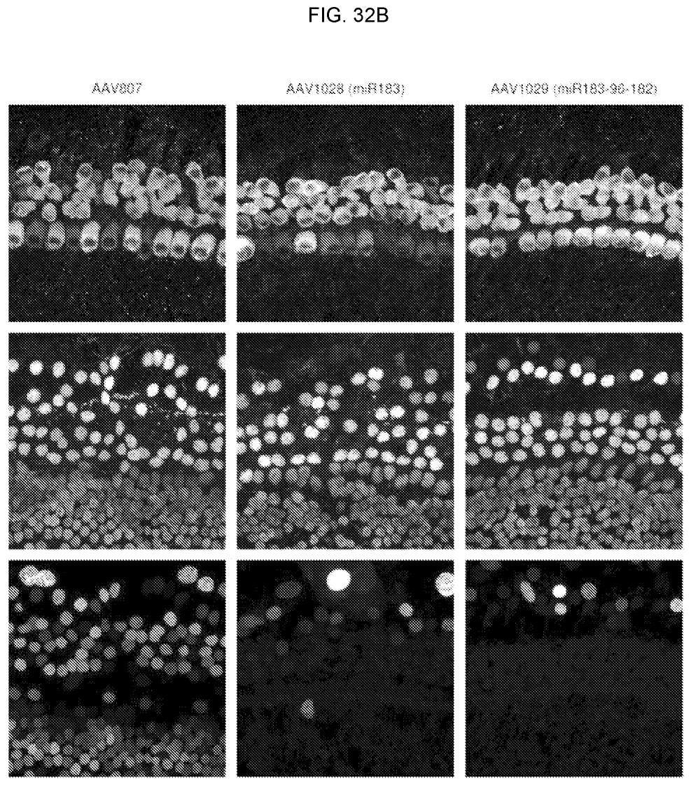

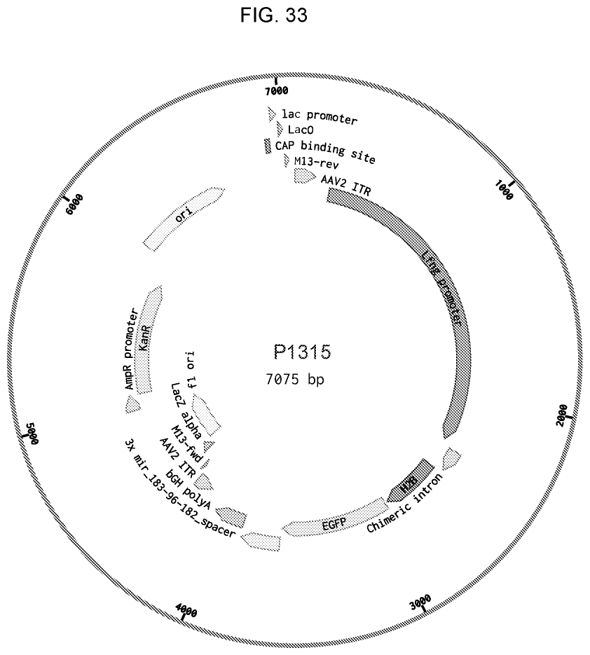

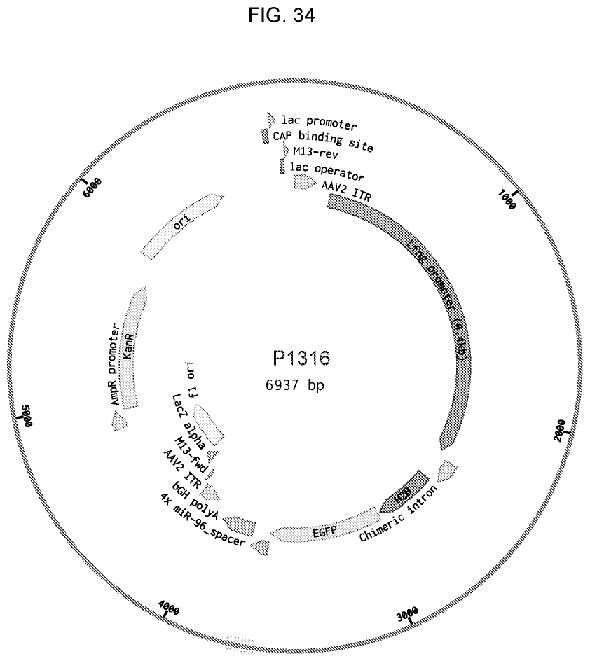

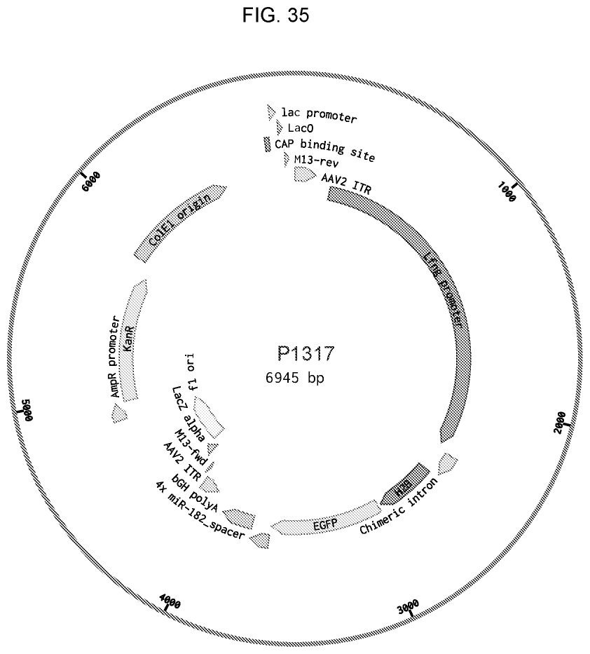

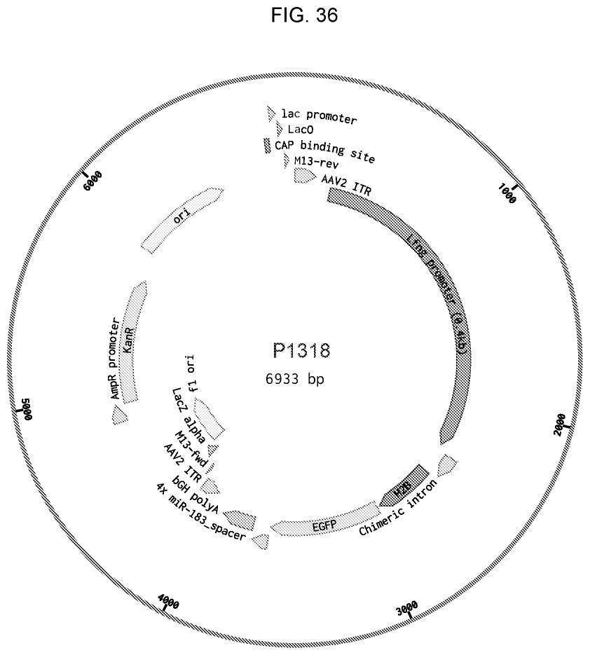

The explants are then incubated at 37° C./5% CO₂ for two days. After two days, the media and virus were removed and replaced with fresh media without virus. The explants were then incubated for an additional three days and then fixed with 4% formaldehyde (PFA) at room temperature for 20 minutes. The explants were washed 3× with PBS, then incubated in 10% normal donkey serum (NDS) in PBS+0.1% TritonX for 20 minutes. The NDS was removed and the explants were incubated with primary antibodies that are specific for hair cells (e.g., antibodies to Myosin VIIa) and that are specific for supporting cells (e.g., antibodies to Sox2), each diluted 1:1000 in PBS+0.1% TritonX, overnight at 4° C. The following day, the explants were washed 3× with PBS, then incubated with labeled secondary antibodies that enabled differentiation between the various primary antibodies, each diluted 1:1000 in PBS+0.1% TritonX, for 2-3 hours at room temperature. After incubating in secondary antibodies, the explants were washed 5× with PBS and mounted onto microscope slides using Fluoromount mounting medium. Slides were then imaged using a Zeiss LSM880 confocal microscope to differentially visualize hair cells and supporting cells, as well as to detect GFP fluorescence. The results are shown in the FIGS. 32A-32B. In tissues infected with AAV1026 and AAV1027, which contain four copies a polynucleotide that can be transcribed to produce a miR-96 or miR-182 target site, respectively, FIG. 32A demonstrates that GFP expression was restricted to supporting cells, but overall was greatly reduced compared to AAV807. The same was true for tissues infected with AAV1028 or AAV1029, as shown in FIG. 32B.

### Example 4—Effect of miRNA Target Sequences on Expression of AAV Vector-Encoded eGFP Under Control of a Supporting Cell Promoter in Murine Cochlear Explants

In order to further increase supporting cell expression, the supporting cell-specific LFNG promoter and its associated upstream enhancer sequences were employed to drive expression of a nuclear-targeted H2B-eGFP fusion protein in the presence of various miRNA target sequences in murine cochlear explants. Although the LFNG promoter primarily drives expression in supporting cells, it does promote some sporadic hair cell expression.

Sensory epithelia were dissected from P0-P2 mice and plated two to a dish on Matrigel-treated MatTek 35 mm dishes with a #0 10 mm coverslip. 150-200 μL of DMEM+10% FBS+10 μg/mL ciprofloxacin was added to each dish. After a 1-hour incubation at 37° C./5% CO₂, 1×10¹¹ viral genomes of an AAV viral vector as indicated in Table 15, below were added to each dish.

TABLE 15
Transgene plasmid sources of AAV vectors containing
the LFNG promoter used to infect different
groups of murine cochlear explants

| Group | Transgene Source of AAV Vector |
|---|---|
| 1 | P812 (a control vector containing an H2B-eGFP transgene under |
| | control of an LFNG promoter and no miRNA recognition |
| | sequences) |
| 2 | P1316 |
| 3 | P1317 |
| 4 | P1318 |
| 5 | P1315 |

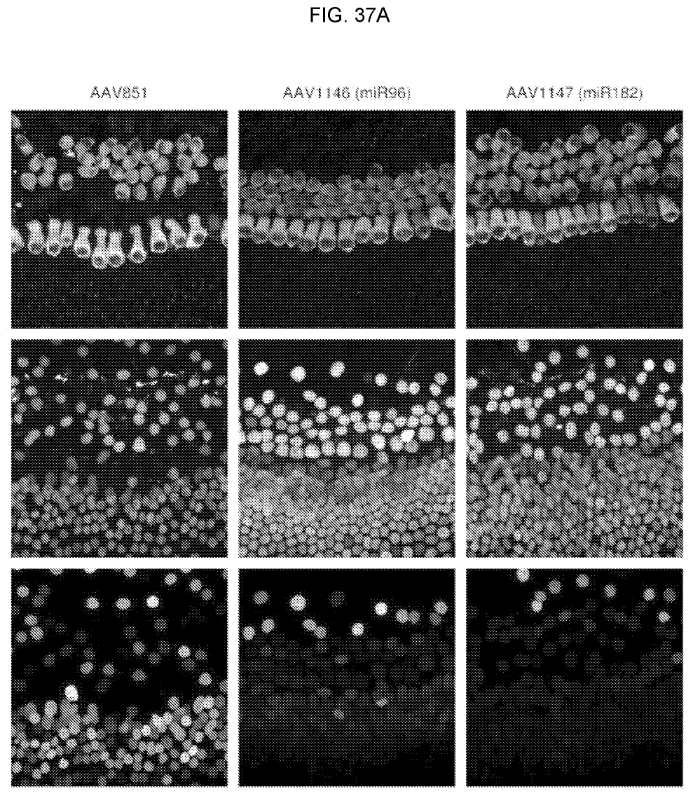

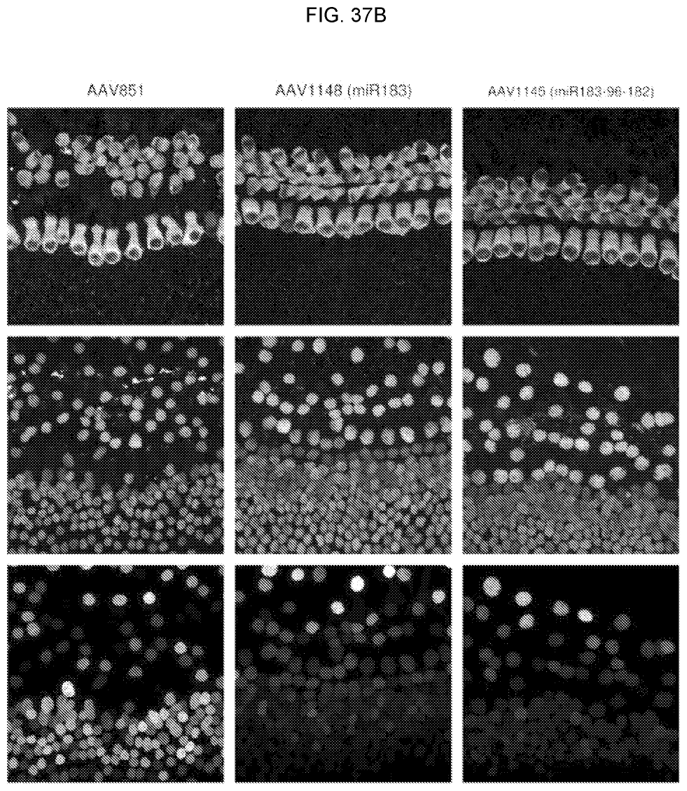

The explants are then incubated at 37° C./5% CO₂ for two days. Two days after first administration of a vector, the media and virus were removed and replaced with fresh media without virus. The explants were then incubated for an additional three days and then fixed with 4% formaldehyde at room temperature for 20 minutes. The explants were washed 3× with PBS, then incubated in 10% normal donkey serum (NDS) in PBS+0.1% TritonX for 20 minutes. The NDS was removed and the explants were incubated with primary antibodies that are specific for hair cells (e.g., antibodies to Myosin VIIa) and that are specific for supporting cells (e.g., antibodies to Sox2), each diluted 1:1000 in PBS+0.1% TritonX, overnight at 4° C. The following day, the explants were washed 3× with PBS, then incubated with labeled secondary antibodies that enabled differentiation between the various primary antibodies, each diluted 1:1000 in PBS+0.1% TritonX, for 2-3 hours at room temperature. After incubating in secondary antibodies, the explants were washed 5× with PBS and mounted onto microscope slides using Fluoromount mounting medium. Slides were then imaged using a Zeiss LSM 880 confocal microscope to differentially visualize hair cells and supporting cells, as well as to detect GFP fluorescence. The results are shown in FIGS. 37A-37B. As shown in FIG. 37A, GFP was expressed in the nuclei of both hair cells and supporting cells in tissue infected with AAV851, which contained no miRNA target sites. In tissues infected with AAV1146 and AAV1147, which contained four copies of a polynucleotide that can be transcribed to produce the miR-96 or miR-182 target site, respectively, GFP expression was restricted to supporting cells, including supporting cells in the sensory epithelium (interdigitated with hair cells) as well as strong expression lateral to the sensory epithelium and moderate expression medial to the sensory epithelium. As shown in FIG. 37B, tissues infected with AAV1148 and AAV1145, which contained four copies of a polynucleotide that can be transcribed to produce the miR-183 target site or three copies of a polynucleotide that can be transcribed to produce each of the miR-182, miR-96, and miR-183 target sites, respectively, GFP expression was also restricted to supporting cells, including supporting cells in the sensory epithelium (interdigitated with hair cells) as well as strong expression lateral to the sensory epithelium and moderate expression medial to the sensory epithelium.

### Example 5—Effect of miRNA Target Sequences on Expression of AAV Vector-Encoded eGFP Under Control of a Ubiquitous CMV Promoter in Murine Utricle Explants

Utricles were dissected from 8-week-old C57BI/6 mice and plated in 35 mm Matsunami glass bottom dishes with a 14 mm well, three to a dish. 250 μL of DMEM/F12+5% FBS+2.5 μg/mL ciprofloxacin was added to each dish, and 1×10¹¹ viral genomes of an AAV vector as indicated in Table 14, above, were added to each dish.

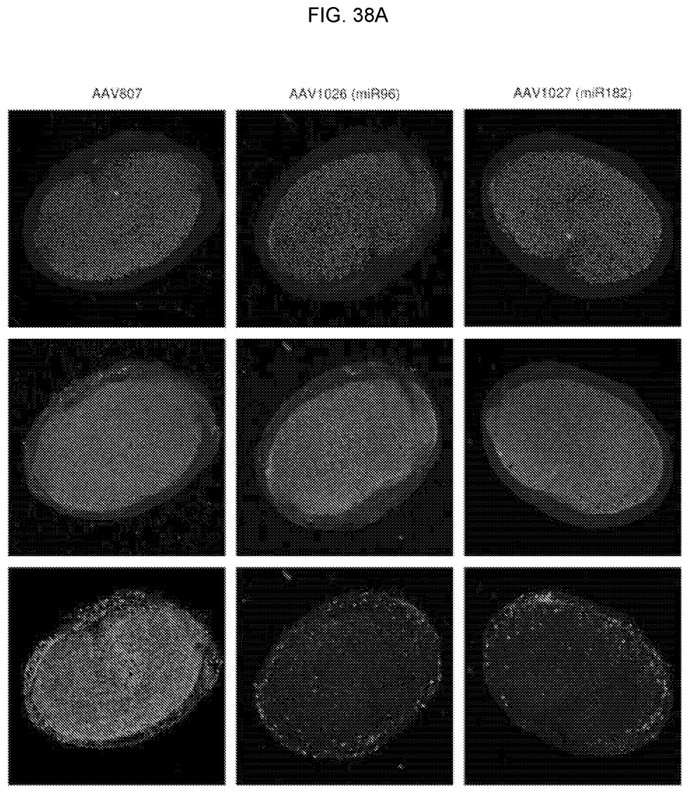

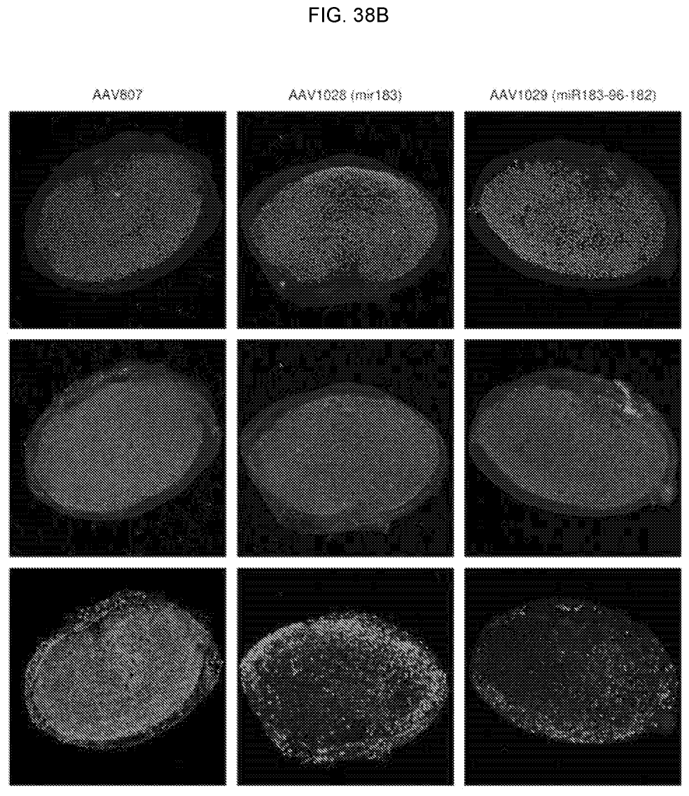

The explants were then incubated at 37° C./5% CO₂ for two days. After two days, the media and virus were removed and 2 mL of fresh media without virus was added to each dish. The explants were then incubated for an additional three days and then fixed with 4% formaldehyde at room temperature for 1 hour. The explants were washed 3× with PBS, then incubated in 10% normal donkey serum (NDS) in PBS+0.5% TritonX for 1 hour. The NDS/PBS was removed, and the explants were incubated with primary antibodies that are specific for hair cells (e.g., antibodies to Pou4f3) and that are specific for supporting cells (e.g., antibodies to Sox2), each diluted 1:500 in PBS+0.5% TritonX, overnight at 4° C. The following day, the explants were washed 3× with PBS, then incubated with labeled secondary antibodies that enabled differentiation between the various primary antibodies, each diluted 1:500 in PBS+0.5% TritonX, for 2-3 hours at room temperature. After incubating in secondary antibodies, the explants were washed 2× with PBS, 1× with DAPI, and 2× more with PBS, and mounted onto microscope slides using Diamond Anti-Fade mounting medium. Slides were then imaged using a Zeiss LSM880 confocal microscope to differentially visualize hair cells and supporting cells, as well as to detect GFP fluorescence. The results are shown in the FIGS. 38A-38B.

In utricles, the hair cell layer sits on top of the supporting cell layer. As shown in FIG. 38A, GFP was expressed in the nuclei of both hair cells (compare bottom row to top row) and supporting cells (compare bottom row to middle row) in tissue infected with AAV807, which contains no miRNA target sites. In tissues infected with AAV1026 and AAV1027, which contain 4 copies of a polynucleotide that can be transcribed to produce a miR-96 or a miR-182 target site, respectively, GFP expression was restricted to supporting cells and cells outside the sensory epithelium, but overall was greatly reduced compared to AAV807. As shown in FIG. 38B, in tissues infected with AAV1028 and AAV1029, which contain four copies of a polynucleotide that can be transcribed to produce the miR-183 target site and three copies of a polynucleotide that can be transcribed to produce each of the miR-182, miR-96, and miR-183 target sites, respectively, GFP expression remained strong but was restricted to supporting cells and cells outside of the sensory epithelium.

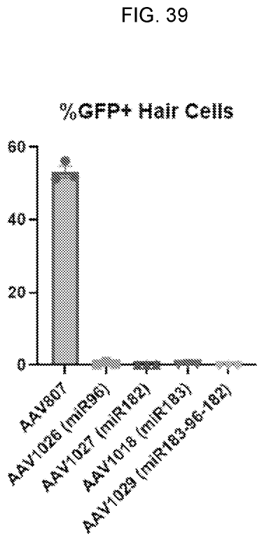

Hair cells and GFP were quantified using Imaris 9.9.1 software. Hair cells were counted by creating Spots using the Pou4f3 channel, setting a quality threshold, and manually removing any false positives. A mask encompassing the hair cells was created from these Spots. GFP positive nuclei were counted by creating Spots in the same manner with GFP channel. The GFP Spots were then filtered by the mean or median intensity of the hair cell mask to identify nuclei that were both Pou4f3 positive and GFP positive. The percentage of hair cells in each tissue that were GFP positive was then calculated. The data were then plotted using GraphPad Prism 9.3.1 software and are shown in FIG. 39.

### Example 6—Effect of miRNA Target Sequences on Expression of AAV Vector-Encoded Gjb2 in Murine Cochlear Explants

Once both expression of acGFP or eGFP and a miRNA-driven decrease of that expression are demonstrated in cochlear explants, similar AAV vectors are used that contain murine GJB2 (mGJB2) as the transgene. Defects in this gene in mice and the corresponding gene in humans (hGJB2) result in the loss of a critical gap junction protein in the cochlear sensory epithelium, which leads to improperly functioning supporting cells and, ultimately, loss of hair cells. It is important that a gene therapy vector designed to restore proper expression of this protein primarily drives expression of GJB2 in supporting cells but not in hair cells. We believe that including various types and arrays of miRNA target sequences in the Gjb2 transcript encoded by the AAV transgene vectors will achieve this cell-specific expression. This is because the miRNAs that bind the AAV vector-encoded miRNA target sequences are present in hair cells, but not supporting cells. The AAV vectors disclosed in Table 15 are used to transfect neonatal cochlear explants to confirm that mGJB2 expression in hair cells is reduced or eliminated by placing 1-4 copies of target sequences complementary to these microRNAs in the 3′ UTR of the transgene.

Sensory epithelia are dissected from P0-P2 mice and plated two to a dish on Matrigel-treated MatTek 35 mm dishes with a #0 10 mm coverslip. 150-200 μL of DMEM+10% FBS+10 μg/mL ciprofloxacin is added to each dish. After a one-hour incubation at 37° C./5% CO₂, 1×10¹¹ viral genomes of an AAV viral vector as indicated in Table 16, below is added to each dish.

TABLE 16
Transgene plasmids sources of AAV Vectors used to
infect different groups of murine cochlear explants

| Group | Transgene Source of AAV Vector |
|---|---|
| 1 | P750 (SEQ ID NO: 9) |
| 2 | P752 (SEQ ID NO: 10) |
| 3 | P753 (SEQ ID NO: 11) |
| 4 | P754 (SEQ ID NO: 12) |
| 5 | P755 (SEQ ID NO: 13) |
| 6 | P748 (SEQ ID NO: 14) |
| 7 | P749 (SEQ ID NO: 15) |
| 8 | P751 (SEQ ID NO: 16) |
| 9 | Control plasmid for expression of Gjb2 |
| | without any miRNA target sequences |

After fixation with formaldehyde, the explants are washed 3× with PBS, then incubated in 10% normal donkey serum (NDS) in PBS for 20 minutes. The NDS is removed and the explants are incubated with primary antibodies that are specific for hair cells (e.g., antibodies to Myosin VIIa), that are specific for supporting cells (e.g., antibodies to Sox2), and that are specific for GJB2, each diluted 1:1000 in PBS, overnight at 4° C. The following day, the explants are washed 3× with PBS, then incubated with labeled secondary antibodies that enable differentiation between the various primary antibodies, each diluted 1:1000 in PBS, for 2-3 hours at room temperature. After incubating in secondary antibodies, the explants are washed 5× with PBS and mounted onto microscope slides using Fluoromount mounting medium. Slides are then imaged using a Zeiss Upright Apotome light microscope to differentially visualize hair cells and supporting cells, as well as to detect GJB2.

### Example 7—Administration of a Composition Containing a Nucleic Acid Vector Containing a Promoter Operably Linked to a Polynucleotide Encoding Gjb2 and to One or More Polynucleotides that can be Transcribed to Produce a miRNA Target Sequence for a miRNA Expressed in Cochlear Hair Cells and/or Spiral Ganglion Neurons but not in Cochlear Supporting Cells

According to the methods disclosed herein, a physician of skill in the art can treat a patient, such as a human patient, with hearing loss associated with a mutation in GJB2 (e.g., DFNB1 or DFNA3) so as to improve or restore hearing. To this end, a physician of skill in the art can administer to the human patient a composition containing an AAV vector (e.g., AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eB, or PHP.S) containing a ubiquitous promoter (e.g., CMV), a GJB2 promoter, or a supporting cell-specific promoter (e.g., a FGFR3 promoter, a LFNG promoter, or a SLC1A3 promoter) operably linked to a polynucleotide encoding Gjb2 (e.g., human Gjb2) and to one or more miRNA target sequences for one or more miRNAs expressed in cochlear hair cells and/or spiral ganglion neurons but not in cochlear supporting cells (e.g., one or more target sequences for miR-183, miR-96, miR-182, miR-18a, miR-140, miR-124a, and/or miR-194). The composition containing the AAV vector may be administered to the patient, for example, by local administration to the inner ear (e.g., injection into the perilymph or to or through the round window membrane), to treat hearing loss associated with a mutation in GJB2.

Following administration of the composition to a patient, a practitioner of skill in the art can monitor the patient's improvement in response to the therapy by a variety of methods. For example, a physician can monitor the patient's hearing by performing standard tests, such as audiometry, ABR, electrocochleography (ECOG), and otoacoustic emissions following administration of the composition. A finding that the patient exhibits improved hearing in one or more of the tests following administration of the composition compared to hearing test results prior to administration of the composition indicates that the patient is responding favorably to the treatment. Subsequent doses can be determined and administered as needed.

Exemplary embodiments of the invention are described in the enumerated paragraphs below.

E1. A nucleic acid vector comprising a first promoter operably linked to:
i. a first polynucleotide that can be transcribed to produce an expression product (e.g., a polynucleotide that can be transcribed to produce a protein or inhibitory RNA); and
ii. at least one polynucleotide that can be transcribed to produce a microRNA (miRNA) target sequence (e.g., 1, 2, 3, 4, 5, 6, 7, 8, 9, 10, or more polynucleotides that can be transcribed to produce miRNA target sequences), wherein:
   the first polynucleotide is suitable for expression in a first inner ear cell type, but not in a different, second inner ear cell type; and
   the miRNA target sequence transcribed from the at least one polynucleotide operably linked to the first promoter is recognized by a miRNA expressed in the second inner ear cell type but not in the first inner ear cell type.

E2. The nucleic acid vector of E1, wherein the expression product transcribed from the first polynucleotide promotes conversion of the first inner ear cell type to the second inner ear cell type.

E3. The nucleic acid vector of E1 or E2, wherein the first polynucleotide is expressed in the first inner ear cell type but not in the second inner ear cell type.

E4. The nucleic acid vector of any one of E1-E3, comprising at least two (e.g., 2, 3, 4, 5, 6, 7, 8, 9, 10, or more) polynucleotides that can be transcribed to produce miRNA target sequences.

E5. The nucleic acid vector of E4, comprising a polynucleotide that can be transcribed to produce a first miRNA target sequence and a polynucleotide that can be transcribed to produce a second miRNA target sequence, wherein each miRNA target sequence is recognized by a different miRNA.

E6. The nucleic acid vector of E5, further comprising a polynucleotide that can be transcribed to produce a third miRNA target sequence, wherein each of the first, second, and third miRNA target sequences are recognized by different miRNAs.

E7. The nucleic acid vector of any one of E1-E5, comprising at least two copies (e.g., 2, 3, 4, 5, 6, 7, 8, 9, 10, or more copies) of a polynucleotide that can be transcribed to produce the same miRNA target sequence.

E8. The nucleic acid vector of E7, comprising at least three copies (e.g., 3, 4, 5, 6, 7, 8, 9, 10, or more copies) of the polynucleotide that can be transcribed to produce the same miRNA target sequence.

E9. The nucleic acid vector of any one of E1-E4, E7 and E8, wherein each polynucleotide that can be transcribed to produce a miRNA target sequence operably linked to the first promoter is the same.

E10. The nucleic acid vector of any one of E1-E9, wherein each polynucleotide that can be transcribed to produce a miRNA target sequence is located 3′ of the first polynucleotide.

E11. The nucleic acid vector of E10, wherein the vector further comprises a WPRE sequence located 3′ of the first polynucleotide, and wherein each polynucleotide that can be transcribed to produce a miRNA target sequence is located between the first polynucleotide and the WPRE sequence.

E12. The nucleic acid vector of E10 or E11, wherein each polynucleotide that can be transcribed to produce a miRNA target sequence is in the 3′ UTR of the first polynucleotide.

E13. The nucleic acid vector of any one of E1-E9, wherein each polynucleotide that can be transcribed to produce a miRNA target sequence is in the 5′ UTR of the first polynucleotide.

E14. The nucleic acid vector of any one of E1-E13, wherein each polynucleotide that can be transcribed to produce a miRNA target sequence operably linked to the first promoter is independently targeted by a miRNA listed in Table 2.

E15. The nucleic acid vector of any one of E1-E14, wherein each polynucleotide that can be transcribed to produce a miRNA target sequence operably linked to the first promoter is independently targeted by one of: miR-183, miR-96, miR-182, miR-18a, miR-100, miR-124a, miR-140, miR-194, miR-135, or miR-135b.

E16. The nucleic acid vector of any one of E1-E15, wherein the first inner ear cell type is a cochlear supporting cell and the second inner ear cell type is at least one of a cochlear hair cell or a spiral ganglion neuron.

E17. The nucleic acid vector of E16, wherein the second inner ear cell type is a cochlear hair cell.

E18. The nucleic acid vector of E16, wherein the second inner ear cell type is a spiral ganglion neuron.

E19. The nucleic acid vector of any one of E1-E15, wherein the first inner ear cell type is a vestibular supporting cell and the second inner ear cell type is at least one of a vestibular hair cell or a vestibular ganglion neuron.

E20. The nucleic acid vector of E19, wherein the second inner ear cell type is a vestibular hair cell.

E21. The nucleic acid vector of E20, wherein the second inner ear cell type is a vestibular type I hair cell.

E22. The nucleic acid vector of E19, wherein the second inner ear cell type is a vestibular ganglion neuron.

E23. The nucleic acid vector of any one of E1-E15, wherein the first inner ear cell type is a vestibular type II hair cell and the second inner ear cell type is a vestibular type I hair cell.

E24. The nucleic acid vector of any one of E1-E15, wherein the first inner ear cell type is a vestibular type II hair cell and the second inner ear cell type is a vestibular ganglion neuron.

E25. The nucleic acid vector of any one of E1-E15, wherein the first polynucleotide is a transgene encoding a protein, is a polynucleotide that can be transcribed to produce an inhibitory RNA, or encodes a component of a gene editing system.

E26. The nucleic acid vector of E25, wherein the first polynucleotide is a transgene encoding a protein.

E27. The nucleic acid vector of E26, wherein the transgene is a wild-type version of a gene listed in Table 4.

E28. The nucleic acid vector of E26, wherein the transgene is a polynucleotide listed in Table 5.

E29. The nucleic acid vector of E25, wherein the first polynucleotide can be transcribed to produce an inhibitory RNA.

E30. The nucleic acid vector of E29, wherein the inhibitory RNA is an siRNA, shRNA, or shRNA-mir.

E31. The nucleic acid vector of E29, wherein the inhibitory RNA is an inhibitory RNA targeting Sox2 (e.g., an inhibitory RNA described herein).

E32. The nucleic acid vector of E25, wherein the first polynucleotide encodes a component of a gene editing system.

E33. The nucleic acid vector of E32, wherein the first polynucleotide can be transcribed to produce a guide RNA.

E34. The nucleic acid vector of E32, wherein the first polynucleotide encodes a nuclease.

E35. The nucleic acid vector of any one of E1-E15, wherein the first polynucleotide encodes Atoh1, Gfi1, Pou4f3, Ikzf2, dnSox2, or Gjb2.

E36. The nucleic acid vector of any one of E1-E15, wherein the first promoter is supporting cell-specific promoter, a hair cell-specific promoter, or a ubiquitous promoter.

E37. The nucleic acid vector of any one of E1-E15, wherein the first promoter is a CMV promoter, a MYO15 promoter, an LFNG promoter, an FGFR3 promoter, a SLC1A3 promoter, a GFAP promoter, or a SLC6A14 promoter.

E38. The nucleic acid vector of any one of E1-E37, further comprising a second polynucleotide that can be transcribed to produce an expression product, wherein the second polynucleotide is different from the first polynucleotide.

E39. The nucleic acid vector of E38, wherein the vector comprises in 5′ to 3′ order: the first promoter, the first polynucleotide, the second polynucleotide, and the at least one polynucleotide that can be transcribed to produce a miRNA target sequence, wherein the second polynucleotide is suitable for expression in the first inner ear cell type, but not in the second inner ear cell type.

E40. The nucleic acid vector of E38, wherein the second polynucleotide is operably linked to a second promoter.

E41. The nucleic acid vector of E40, wherein the vector comprises in 5′ to 3′ order: the first promoter, the first polynucleotide, the at least one polynucleotide that can be transcribed to produce a miRNA target sequence, the second promoter, and the second polynucleotide.

E42. The nucleic acid vector of E41, wherein expression of the second polynucleotide is not regulated by a miRNA target sequence.

E43. The nucleic acid vector of E41, wherein the vector further comprises at least one polynucleotide that can be transcribed to produce a miRNA target sequence 3′ of the second polynucleotide that is operably linked to the second promoter, wherein the second polynucleotide is suitable for expression in a third inner ear cell type, but not in a different, fourth inner ear cell type, and wherein the miRNA target sequence transcribed from the at least one polynucleotide operably linked to the second promoter is recognized by a miRNA expressed in the fourth inner ear cell type, but not in the third inner ear cell type.

E44. The nucleic acid vector of any one of E38-E43, further comprising a third polynucleotide that can be transcribed to produce an expression product, wherein the third polynucleotide is different from the first polynucleotide and the second polynucleotide.

E45. The nucleic acid vector of E44, wherein the vector comprises in 5′ to 3′ order: the first promoter, the first polynucleotide, the second polynucleotide, the third polynucleotide, and the at least one polynucleotide that can be transcribed to produce a miRNA target sequence, wherein the third polynucleotide is suitable for expression in the first inner ear cell type, but not in the second inner ear cell type.

E46. The nucleic acid vector of E44, wherein the first polynucleotide is operably linked to the first promoter and the second and third polynucleotides are operably linked to the second promoter.

E47. The nucleic acid vector of E45, wherein the vector comprises in 5′ to 3′ order: the first promoter, the first polynucleotide, the at least one polynucleotide that can be transcribed to produce a miRNA target sequence, the second promoter, the second polynucleotide, and the third polynucleotide.

E48. The nucleic acid vector of E47, wherein expression of the second and third polynucleotides is not regulated by a miRNA target sequence.

E49. The nucleic acid vector of E47, wherein the vector further comprises at least one polynucleotide that can be transcribed to produce a miRNA target sequence 3′ of the third polynucleotide that is operably linked to the second promoter, wherein the second and third polynucleotides are suitable for expression in a third inner ear cell type, but not in a different, fourth inner ear cell type, and wherein the miRNA target sequence transcribed from the at least one polynucleotide operably linked to the second promoter is recognized by a miRNA expressed in the fourth inner ear cell type, but not in the third inner ear cell type.

E50. The nucleic acid vector of E44, wherein the first polynucleotide and the second polynucleotide are operably linked to the first promoter and the third nucleic acid is operably linked to a second promoter.

E51. The nucleic acid vector of E50, wherein the vector comprises in 5′ to 3′ order: the first promoter, the first polynucleotide, the second polynucleotide, the at least one polynucleotide that can be transcribed to produce a miRNA target sequence, the second promoter, and the third polynucleotide.

E52. The nucleic acid vector of E51, wherein expression of the third polynucleotide is not regulated by a miRNA target sequence.

E53. The nucleic acid vector of E51, wherein the vector further comprises at least one polynucleotide that can be transcribed to produce a miRNA target sequence 3′ of the third polynucleotide that is operably linked to the second promoter, wherein the third polynucleotide is suitable for expression in a third inner ear cell type, but not in a different, fourth inner ear cell type, and wherein the miRNA target sequence transcribed from the at least one polynucleotide operably linked to the second promoter is recognized by a miRNA expressed in the fourth inner ear cell type, but not in the third inner ear cell type.

E54. The nucleic acid vector of E44, wherein the first polynucleotide is operably linked to the first promoter, the second polynucleotide is operably linked to the second promoter, and the third polynucleotide is operably linked to a third promoter.

E55. The nucleic acid vector of E54, wherein the vector comprises in 5′ to 3′ order: the first promoter, the first polynucleotide, at least one polynucleotide that can be transcribed to produce a miRNA target sequence, the second promoter, the second polynucleotide, the third promoter, and the third polynucleotide.

E56. The nucleic acid vector of E55, wherein expression of the second and third polynucleotides is not regulated by a miRNA target sequence.

E57. The nucleic acid vector of E54, wherein the vector comprises in 5′ to 3′ order: the first promoter, the first polynucleotide, at least one polynucleotide that can be transcribed to produce a miRNA target sequence, the second promoter, the second polynucleotide, at least one polynucleotide that can be transcribed to produce a miRNA target sequence, the third promoter, and the third polynucleotide, wherein the second polynucleotide is suitable for expression in a third inner ear cell type, but not in a different, fourth inner ear cell type, and wherein the miRNA target sequence transcribed from the at least one polynucleotide operably linked to the second promoter is recognized by a miRNA expressed in the fourth inner ear cell type, but not in the third inner ear cell type.

E58. The nucleic acid vector of E57, wherein expression of the third polynucleotide is not regulated by a miRNA target sequence.

E59. The nucleic acid vector of E57, wherein the vector further comprises at least one polynucleotide that can be transcribed to produce a miRNA target sequence 3′ of the third polynucleotide that is operably linked to the third promoter, wherein the third polynucleotide is suitable for expression in a fifth inner ear cell type, but not in a different, sixth inner ear cell type, and wherein the miRNA target sequence transcribed from the at least one polynucleotide operably linked to the third promoter is recognized by a miRNA expressed in the sixth inner ear cell type, but not in the fifth inner ear cell type.

E60. The nucleic acid vector of any one of E43, E49, E53, and E57, wherein the fourth inner ear cell type is different from the second inner ear cell type.

E61. The nucleic acid vector of any one of E43, E49, E53, and E57, wherein the fourth inner ear cell type is the same as the second inner ear cell type.

E62. The nucleic acid vector of any one of E43, E49, E53, E57, E60, and E61, wherein the third inner ear cell type is different from the first inner ear cell type.

E63. The nucleic acid vector of any one of E43, E49, E53, E57, E60, and E62, wherein the first inner ear cell type is the same as the fourth inner ear cell type.

E64. The nucleic acid vector of any one of E43, E49, E53, E57, and E60-E62, wherein the first inner ear cell type is different than the fourth inner ear cell type.

E65. The nucleic acid vector of any one of E43, E49, E53, E57, E60, and E62, wherein the third inner ear cell type is the same as the second inner ear cell type.

E66. The nucleic acid vector of any one of E43, E49, E53, E57, E60-E62, and E64, wherein the third inner ear cell type is different than the second inner ear cell type.

E67. The nucleic acid vector of any one of E43, E49, E53, E57, and E60, wherein the third inner ear cell type is the same as the first inner ear cell type.

E68. The nucleic acid vector of any one of E59-E67, wherein the sixth inner ear cell type is different from the fourth and the second inner ear cell types.

E69. The nucleic acid vector of any one of E59, E60, and E62-E67, wherein the sixth inner ear cell type is the same as either the fourth inner ear cell type or the second inner ear cell type.

E70. The nucleic acid vector of any one of E59, E61, E62, E64, and E66, wherein the sixth inner ear cell type is the same as the fourth and the second inner ear cell types.

E71. The nucleic acid vector of any one of E59-E70, wherein the fifth inner ear cell type is different from the first and third inner ear cell types.

E72. The nucleic acid vector of any one of E59-E66 and E68-E70, wherein the fifth inner ear cell type is the same as either the first inner ear cell type or the third inner ear cell type.

E73. The nucleic acid vector of any one of E59, E60, and E67-E69, wherein the fifth inner ear cell type is the same as the first and the third inner ear cell types.

E74. The nucleic acid vector of any one of E40-E73, wherein the second promoter is a supporting cell-specific promoter, a hair cell-specific promoter, or a ubiquitous promoter.

E75. The nucleic acid vector of any one of E40-E74, wherein the second promoter is a CMV promoter, a MYO15 promoter, an LFNG promoter, an FGFR3 promoter, a SLC1A3 promoter, a GFAP promoter, or a SLC6A14 promoter.

E76. The nucleic acid vector of any one of E38-E75, wherein the second polynucleotide is a transgene encoding a protein, is a polynucleotide that can be transcribed to produce an inhibitory RNA, or encodes a component of a gene editing system.

E77. The nucleic acid vector of E76, wherein the second polynucleotide is a transgene encoding a protein.

E78. The nucleic acid vector of E77, wherein the transgene is a wild-type version of a gene listed in Table 4.

E79. The nucleic acid vector of E77, wherein the transgene is a polynucleotide listed in Table 5.

E80. The nucleic acid vector of E76, wherein the second polynucleotide can be transcribed to produce an inhibitory RNA.

E81. The nucleic acid vector of E79, wherein the inhibitory RNA is an siRNA, shRNA, or shRNA-mir.

E82. The nucleic acid vector of E79, wherein the inhibitory RNA is an inhibitory RNA targeting Sox2 (e.g., an inhibitory RNA described herein).

E83. The nucleic acid vector of E76, wherein the second polynucleotide encodes a component of a gene editing system.

E84. The nucleic acid vector of E83, wherein the second polynucleotide can be transcribed to produce a guide RNA.

E85. The nucleic acid vector of E83, wherein the second polynucleotide encodes a nuclease.

E86. The nucleic acid vector of any one of E38-E75, wherein the second polynucleotide encodes Atoh1, Gfi1, Pou4f3, Ikzf2, dnSox2, or Gjb2.

E87. The nucleic acid vector of any one of E43-E86, wherein one or more (e.g., 1, 2, 3, 4, 5, 6, 7, 8, 9, 10, or more) polynucleotides that can be transcribed to produce a miRNA target sequence are operably linked to the second promoter.

E88. The nucleic acid vector of any one of E43-E87, wherein each polynucleotide that can be transcribed to produce a miRNA target sequence that is operably linked to the second promoter is independently targeted by a miRNA listed in Table 2.

E89. The nucleic acid vector of any one of E43-E88, wherein each polynucleotide that can be transcribed to produce a miRNA target sequence that is operably linked to the second promoter is independently targeted by one of: miR-183, miR-96, miR-182, miR-18a, miR-100, miR-124a, miR-140, miR-194, miR-135, or miR-135b.

E90. The nucleic acid vector of any one of E43-E89, wherein each polynucleotide that can be transcribed to produce a miRNA target sequence that is operably linked to the second promoter is the same.

E91. The nucleic acid vector of any one of E54-E90, wherein the third promoter is a supporting cell-specific promoter, a hair cell-specific promoter, or a ubiquitous promoter.

E92. The nucleic acid vector of any one of E54-E91, wherein the third promoter is a CMV promoter, a MYO15 promoter, a LFNG promoter, a FGFR3 promoter, a SLC1A3 promoter, a GFAP promoter, or a SLC6A14 promoter.

E93. The nucleic acid vector of any one of E44-E92, wherein the third polynucleotide is a transgene encoding a protein, is a polynucleotide that can be transcribed to produce an inhibitory RNA, or encodes a component of a gene editing system.

E94. The nucleic acid vector of E93, wherein the third polynucleotide is a transgene encoding a protein.

E95. The nucleic acid vector of E94, wherein the transgene is a wild-type version of a gene listed in Table 4.

E96. The nucleic acid vector of E94, wherein the transgene is a polynucleotide listed in Table 5.

E97. The nucleic acid vector of E93, wherein the third polynucleotide can be transcribed to produce an inhibitory RNA.

E98. The nucleic acid vector of E97, wherein the inhibitory RNA is an siRNA, shRNA, or shRNA-mir.

E99. The nucleic acid vector of E97, wherein the inhibitory RNA is an inhibitory RNA targeting Sox2 (e.g., an inhibitory RNA described herein).

E100. The nucleic acid vector of E93, wherein the third polynucleotide encodes a component of a gene editing system.

E101. The nucleic acid vector of E100, wherein the third polynucleotide can be transcribed to produce a guide RNA.

E102. The nucleic acid vector of E100, wherein the third polynucleotide encodes a nuclease.

E103. The nucleic acid vector of any one of E44-E92, wherein the third polynucleotide encodes Atoh1, Gfi1, Pou4f3, Ikzf2, dnSox2, or Gjb2.

E104. The nucleic acid vector of any one of E59-E103, wherein one or more (e.g., 1, 2, 3, 4, 5, 6, 7, 8, 9, 10, or more) polynucleotides that can be transcribed to produce a miRNA target sequence are operably linked to the third promoter.

E105. The nucleic acid vector of any one of E59-E104, wherein each polynucleotide that can be transcribed to produce a miRNA target sequence that is operably linked to the third promoter is independently targeted by a miRNA listed in Table 2.

E106. The nucleic acid vector of any one of E59-E105, wherein each polynucleotide that can be transcribed to produce a miRNA target sequence that is operably linked to the third promoter is independently targeted by one of: miR-183, miR-96, miR-182, miR-18a, miR-100, miR-124a, miR-140, miR-194, miR-135, or miR-135b.

E107. The nucleic acid vector of any one of E59-E106, wherein each polynucleotide that can be transcribed to produce a miRNA target sequence that is operably linked to the third promoter is the same.

E108. The nucleic acid vector of any one of E1-E15, E26-E29, and E35-E107, wherein:
a. the first polynucleotide encodes Atoh1, Gfi1, Pou4f3, Ikzf2, dnSox2, or Gjb2 or can be transcribed to produce an inhibitory RNA targeting Sox2;
b. the first promoter is a CMV promoter, an FGFR3 promoter, an LFNG promoter, or a SLC1A3 promoter;
c. each miRNA target sequence transcribed from a polynucleotide operably linked to the first promoter is independently targeted by one of: miR-183, miR-96, miR-182, miR-18a, miR-140, or miR-194;
d. the first inner ear cell type is a cochlear supporting cell; and
e. the second inner ear cell type is cochlear hair cell.

E109. The nucleic acid vector of E108, wherein the first polynucleotide encodes Atoh1 and the second polynucleotide encodes is Ikzf2.

E110. The nucleic acid vector of E108, wherein the first polynucleotide encodes Atoh1, the second polynucleotide encodes Gfi1, and the third polynucleotide encodes Pou4f3.

E111. The nucleic acid vector of any one of E1-E15, E26-E29, and E35-E107, wherein:
a. the first polynucleotide encodes GJB2;
b. the first promoter is a GJB2 promoter, a CMV promoter, an FGFR3 promoter, an LFNG promoter, or a SLC1A3 promoter;
c. each miRNA target sequence transcribed from a polynucleotide operably linked to the first promoter is independently targeted by one of: miR-183, miR-96, miR-182, miR-18a, miR-124, or miR-194;
d. the first inner ear cell type is a cochlear supporting cell; and
e. the second inner ear cell type is spiral ganglion neuron.

E112. The nucleic acid vector of any one of E1-E15, E26-E29, and E35-E107, wherein:
a. the first polynucleotide encodes Atoh1 or dnSox2 or can be transcribed to produce an inhibitory RNA targeting Sox2;
b. the first promoter is a CMV promoter, a GFAP promoter, a SLC6A14 promoter, or a SLC1A3 promoter;
c. each miRNA target sequence transcribed from a polynucleotide operably linked to the first promoter is independently targeted by one of: miR-183, miR-96, miR-182, miR-18a, miR-140, or miR-135b;
d. the first inner ear cell type is a vestibular supporting cell; and
e. the second inner ear cell type is vestibular hair cell.

E113. The nucleic acid vector of any one of E1-E15, E26-E29, and E35-E107, wherein:
a. the first polynucleotide encodes Atoh1 or dnSox2 or can be transcribed to produce an inhibitory RNA targeting Sox2;
b. the first promoter is a CMV promoter, a GFAP promoter, a SLC6A14 promoter, or a SLC1A3 promoter;
c. each miRNA target sequence transcribed from a polynucleotide operably linked to the first promoter is independently targeted by one of: miR-183, miR-96, miR-182, miR-18a, miR-124a, miR-100, or miR-135;
d. the first inner ear cell type is a vestibular supporting cell; and
e. the second inner ear cell type is vestibular ganglion neuron

E114. The nucleic acid vector of any one of E1-E15, E26-E29, and E35-E107, wherein:
a. the first polynucleotide encodes dnSox2 or can be transcribed to produce an inhibitory RNA targeting Sox2;
b. the first promoter is a MYO15 promoter;
c. each miRNA target sequence transcribed from a polynucleotide operably linked to the first promoter is independently targeted by one of: miR-183, miR-96, miR-182, miR-18a, miR-124a, miR-100, or miR-135;
d. the first inner ear cell type is a type II hair cell; and
e. the second inner ear cell type is vestibular ganglion neuron.

E115. The nucleic acid vector of E114, wherein each miRNA target sequence present is independently targeted by one of: miR-18a, miR-124a, miR-100, or miR-135.

E116. The method of any one of E31, E108, and E112-E114, wherein the inhibitory RNA targeting Sox2 is an siRNA.

E117. The method of any one of E31, E108, and E112-E114, wherein the inhibitory RNA targeting Sox2 is an shRNA.

E118. The method of E116 or E117, wherein the siRNA or shRNA targeting Sox2 has a nucleobase sequence containing a portion of at least 8 contiguous nucleobases having at least 80% complementarity to an equal length portion of a target region of an mRNA transcript of a human or murine SOX2 gene.

E119. The method of E118, wherein the target region is an mRNA transcript of the human SOX2 gene.

E120. The method of E118, wherein the target region is at least 8 to 21 contiguous nucleobases of any one of SEQ ID NOs: 52-70, at least 8 to 22 contiguous nucleobases of SEQ ID NO: 74 or SEQ ID NO: 75, or at least 8 to 19 contiguous nucleobases of any one of SEQ ID NOs: 71-73.

E121. The method of E118, wherein the siRNA or shRNA has a nucleobase sequence containing a portion of at least 8 contiguous nucleobases having at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) complementarity to an equal length portion of any one of SEQ ID NOs: 52-75.

E122. The method of E121, wherein the siRNA or shRNA has a nucleobase sequence having at least 70% complementarity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity) complementarity to any one of SEQ ID NO: 58, SEQ ID NO: 71, SEQ ID NO: 72, SEQ ID NO: 73, SEQ ID NO: 74, and SEQ ID NO: 75.

E123. The method of E117, wherein the shRNA comprises the sequence of nucleotides 2234-2296 of SEQ ID NO: 76 or nucleotides 2234-2296 of SEQ ID NO: 78.

E124. The method of any one of E117-E123, wherein the shRNA is embedded in a microRNA (miRNA) backbone.

E125. The method of E124, wherein the shRNA is embedded in a miR-30 or mir-E backbone.

E126. The method of E125, wherein the shRNA comprises the sequence of nucleotides 2109-2426 of SEQ ID NO: 76, nucleotides 2109-2408 of SEQ ID NO: 66, nucleotides 2109-2426 of SEQ ID NO: 78, or nucleotides 2109-2408 of SEQ ID NO: 79.

E127. The method of any one of E116 and E118-E120, wherein the siRNA comprises a sense strand and an antisense strand selected from the following pairs: SEQ ID NO: 80 and SEQ ID NO: 81; SEQ ID NO: 82 and SEQ ID NO: 83; SEQ ID NO: 84 and SEQ ID NO: 85; and SEQ ID NO: 86 and SEQ ID NO: 87.

E128. The method of any one of E35, E108, and E112-E115, wherein the polynucleotide encoding the dnSox2 protein has the sequence of SEQ ID NO: 50 or SEQ ID NO: 51.

E129. The method of any one of E35, E108, and E112-E115, wherein the dnSox2 protein is a Sox2 protein that lacks most or all of the high mobility group domain (HMGD), a Sox2 protein in which the nuclear localization signals in the HMGD are mutated, a Sox2 protein in which the HMGD is fused to an engrailed repressor domain, or a c-terminally truncated Sox2 protein comprising only the DNA binding domain.

E130. The method of any one of E1-E129, wherein the nucleic acid vector is a plasmid, cosmid, artificial chromosome, or viral vector.

E131. The method of E130, wherein the nucleic acid vector is a viral vector.

E132. The method of E131, wherein the viral vector is selected from the group consisting of an adeno-associated virus (AAV), an adenovirus, and a lentivirus.

E133. The method of E132, wherein the viral vector is an AAV vector.

E134. The method of E133, wherein the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ, DJ/8, DJ/9, 7m8, PHP.B, PHP.B2, PBP.B3, PHP.A, PHP.eb, or PHP.S capsid.

E135. A pharmaceutical composition comprising the nucleic acid vector of any one of E1-E134 and a pharmaceutically acceptable carrier, excipient, or diluent.

E136. A kit comprising the nucleic acid vector of any one of E1-E134 or the pharmaceutical composition of E135.

E137. A method of expressing a polynucleotide in a first inner ear cell type and not in a second inner ear cell type in a subject in need thereof, comprising locally administering to the middle or inner ear of the subject an effective amount of the vector of any one of E1-E134 or the pharmaceutical composition of E135.

E138. A method of reducing off-target expression of a polynucleotide in an inner ear of a subject (e.g., reducing off target expression in a particular inner ear cell type), comprising locally administering to the middle or inner ear of the subject an effective amount of the vector of any one of E1-E134 or the pharmaceutical composition of E135.

E139. The method of E137 or E138, wherein the subject has or is at risk of developing hearing loss, vestibular dysfunction, or tinnitus.

E140. A method of treating a subject having or at risk of developing hearing loss, vestibular dysfunction, or tinnitus, comprising administering to the subject an effective amount of the vector of any one of E1-E134 or the pharmaceutical composition of E135.

E141. The method of E139 or E140, wherein the subject has or is at risk of developing vestibular dysfunction.

E142. The method any one of E139-E141, wherein the vestibular dysfunction comprises vertigo, dizziness, imbalance, bilateral vestibulopathy, oscillopsia, or a balance disorder.

E143. The method of any one of E139-E142, wherein the vestibular dysfunction is age-related vestibular dysfunction, head trauma-related vestibular dysfunction, disease or infection-related vestibular dysfunction, or ototoxic drug-induced vestibular dysfunction.

E144. The method of any one of E139-E1413, wherein the vestibular dysfunction is associated with a genetic mutation.

E145. The method of E1144, wherein the genetic mutation is a mutation in a gene listed in Table 4.

E146. The method of E139 or E140, wherein the vestibular dysfunction is idiopathic vestibular dysfunction.

E147. The method of E139 or E140, wherein the subject has or is at risk of developing hearing loss (e.g., sensorineural hearing loss, including auditory neuropathy and deafness).

E148. The method of any one of E139, E140, and E147, wherein the hearing loss is genetic hearing loss.

E149. The method of E148, wherein the genetic hearing loss is autosomal dominant hearing loss, autosomal recessive hearing loss, or X-linked hearing loss.

E150. The method of E148 or E1149, wherein the genetic hearing loss is a condition associated with a mutation in a gene listed in Table 4.

E151. The method of any one of E139, E140, and E147, wherein the hearing loss is acquired hearing loss.

E152. The method of E151, wherein the acquired hearing loss is noise-induced hearing loss, age-related hearing loss, disease or infection-related hearing loss, head trauma-related hearing loss, or ototoxic drug-induced hearing loss.

E153. The method of E143 or E152, wherein the ototoxic drug is an aminoglycoside, an antineoplastic drug, ethacrynic acid, furosemide, a salicylate, or quinine.

E154. The method of E139 or E140, wherein the hearing loss or vestibular dysfunction is or is associated with age-related hearing loss, noise-induced hearing loss, DFNB61, DFNB1, DFNB7/11, DFNA2, DFNB77, DFNB28, DFNA41, DFNB8, DFNB37, DFNA22, DFNB3, Usher syndrome type 1, Usher syndrome type 2, or bilateral vestibulopathy.

E155. The method of E154, wherein the hearing loss is or is associated with age-related hearing loss, noise-induced hearing loss, DFNB61, DFNB1, DFNB7/11, DFNA2, DFNB77, DFNB28, DFNA41, DFNB8, DFNB37, DFNA22, DFNB3, Usher syndrome type 1, or Usher syndrome type 2 and the first polynucleotide encodes Atoh1.

E156. The method of E155, wherein the second polynucleotide encodes Ikzf2.

E157. The method of E155, wherein the second polynucleotide encodes Pou4f3 and the third polynucleotide encodes Gfi1.

E158. The method of any one of E137-E157, wherein the method further comprises administering to the subject one or more (e.g., 1, 2, 3, 4, 5, or more) additional nucleic acid vectors.

E159. The method of E155, wherein the subject is additionally administered a vector comprising a polynucleotide encoding Ikzf2.

E160. The method of E155, wherein the subject is additionally administered a vector comprising a polynucleotide encoding Pou4f3 and a vector comprising a polynucleotide encoding Gfi1.

E161. The method of E154, wherein the hearing loss or vestibular dysfunction is or is associated with DFNB1, DFNB7/11, DFNA2, DFNB77, DFNB28, DFNA41, DFNB8, DFNB37, DFNA22, DFNB3, Usher syndrome type 1, Usher syndrome type 2, or bilateral vestibulopathy and the first polynucleotide encodes dnSox2.

E162. The method of E161, wherein the second polynucleotide encodes Atoh1.

E163. The method of E161, wherein subject is additionally administered a vector comprising a polynucleotide encoding Atoh1.

E164. The method of any one of E158-E160 and E163, wherein at least one of the one or more additional nucleic acid vectors comprises a promoter operably linked to a polynucleotide that can be transcribed to produce an expression product (e.g., Ikzf2, Pou4f3, Gfi1, or Atoh1) and to a polynucleotide that can be transcribed to produce a miRNA target sequence.

E165. The method of any one of E158-E160 and E163, wherein none of the additional nucleic acid vectors comprise a polynucleotide that can be transcribed to produce a miRNA target sequence.

E166. A method of treating a condition listed in Table 4 in a subject in need thereof, comprising locally administering to the middle or inner ear of the subject an effective amount of the vector of any one of E1-E134 or the pharmaceutical composition of E135, wherein the first polynucleotide is a wild-type version of a gene associated with the condition listed in Table 4 that is mutated in the subject.

E167. The method of any one of E137-E166, wherein the method further comprises evaluating the vestibular function of the subject prior to administering the nucleic acid vector or pharmaceutical composition.

E168. The method of any one of claims E137-E167, wherein the method further comprises evaluating the vestibular function of the subject after administering the nucleic acid vector or pharmaceutical composition.

E169. The method of any one of E137-E168, wherein the method further comprises evaluating the hearing of the subject prior to administering the nucleic acid vector or pharmaceutical composition.

E170. The method of any one of E137-E169, wherein the method further comprises evaluating the hearing of the subject after administering the nucleic acid vector or pharmaceutical composition.

E171. The method of any one of E137-E170, wherein the nucleic acid vector or pharmaceutical composition is administered to the inner ear.

E172. The method of any one of E137-E170, wherein the nucleic acid vector or pharmaceutical composition is administered to the middle ear.

E173. The method of any one of E137-E170, wherein the nucleic acid vector or pharmaceutical composition is administered to a semicircular canal.

E174. The method of any one of E137-E170, wherein the nucleic acid vector or pharmaceutical composition is administered transtympanically or intratympanically.

E175. The method of any one of E137-E170, wherein the nucleic acid vector or pharmaceutical composition is administered into the perilymph.

E176. The method of any one of E137-E170, wherein the nucleic acid vector or pharmaceutical composition is administered into the endolymph.

E177. The method of any one of E137-E170, wherein the nucleic acid vector or pharmaceutical composition is administered to or through the oval window.

E178. The method of any one of E137-E170, wherein the nucleic acid vector or pharmaceutical composition is administered to or through the round window.

E179. The method of any one of E137-E178, wherein the nucleic acid vector or pharmaceutical composition is administered in an amount sufficient to prevent or reduce vestibular dysfunction, delay the development of vestibular dysfunction, slow the progression of vestibular dysfunction, improve vestibular function, prevent or reduce hearing loss, prevent or reduce tinnitus, delay the development of hearing loss, slow the progression of hearing loss, improve hearing, increase vestibular and/or cochlear hair cell numbers, increase vestibular and/or cochlear hair cell maturation, increase vestibular and/or cochlear hair cell regeneration, treat bilateral vestibulopathy, treat oscillopsia, treat a balance disorder, improve the function of one or more inner ear cell types, improve inner ear cell survival, increase inner ear cell proliferation, increase the generation of Type I vestibular hair cells, or increase the number of Type I vestibular hair cells.

E180. An inner ear cell comprising the vector of any one of E1-E134 or the pharmaceutical composition of E135.E181. The inner ear cell of E180, wherein the inner ear cell is a cochlear supporting cell.

E182. The inner ear cell of E180, wherein the inner ear cell is a vestibular supporting cell.

E183. The inner ear cell of E180, wherein the inner ear cell is a cochlear hair cell.

E184. The inner ear cell of E180, wherein the inner ear cell is a vestibular hair cell.

E185. The inner ear cell of E180, wherein the inner ear cell is a vestibular type I hair cell.

E186. The inner ear cell of E180, wherein the inner ear cell is a vestibular type II hair cell.

E187. The inner ear cell of E180, wherein the inner ear cell is a spiral ganglion neuron.

E188. The inner ear cell of E180, wherein the inner ear cell is a vestibular ganglion neuron.

E189. The inner ear cell of any one of E180-E188, wherein the inner ear cell is a human inner ear cell.

E190. The method of any one of E137-E179, wherein the subject is a human.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. A vector comprising a promoter operably linked to:
i. a first polynucleotide that can be transcribed to produce an expression product; and
ii. at least one polynucleotide that can be transcribed to produce a microRNA (miRNA) target sequence, wherein:
the first polynucleotide is suitable for expression in a first inner ear cell type, but not in a different, second inner ear cell type; and
the miRNA target sequence is recognized by a miRNA expressed in the second inner ear cell type, but not in the first inner ear cell type.

2. The vector of claim 1, wherein the expression product transcribed from the first polynucleotide promotes conversion of the first inner ear cell type to the second inner ear cell type.

3. The vector of claim 1 or 2, wherein the first polynucleotide is expressed in the first inner ear cell type but not in the second inner ear cell type.

4. The vector of any one of claims 1-3, comprising at least two polynucleotides that can be transcribed to produce miRNA target sequences.

5. The vector of claim 4, comprising a polynucleotide that can be transcribed to produce a first miRNA target sequence and a polynucleotide that can be transcribed to produce a second miRNA target sequence, wherein each miRNA target sequence is recognized by a different miRNA.

6. The vector of claim 5, further comprising a polynucleotide that can be transcribed to produce a third miRNA target sequence, wherein each of the first, second, and third miRNA target sequences are recognized by different miRNAs.

7. The vector of any one of claims 1-5, comprising at least two copies of a polynucleotide that can be transcribed to produce the same miRNA target sequence.

8. The vector of claim 7, comprising at least three copies of the polynucleotide that can be transcribed to produce the same miRNA target sequence.

9. The vector of any one of claims 1-4, wherein each polynucleotide that can be transcribed to produce a miRNA target sequence operably linked to the promoter is the same.

10. The vector of any one of claims 1-9, wherein each polynucleotide that can be transcribed to produce a miRNA target sequence is located 3′ of the first polynucleotide.

11. The vector of claim 10, wherein the vector further comprises a WPRE sequence located 3′ of the first polynucleotide, and wherein each polynucleotide that can be transcribed to produce a miRNA target sequence is located between the first polynucleotide and the WPRE sequence.

12. The vector of any one of claims 1-11, wherein each miRNA target sequence transcribed from a polynucleotide operably linked to the promoter is independently targeted by one of: miR-183, miR-96, miR-182, miR-18a, miR-100, miR-124a, miR-140, miR-194, miR-135, or miR-135b.

13. The vector of any one of claims 1-12, wherein the first inner ear cell type is a cochlear supporting cell and the second inner ear cell type is at least one of a cochlear hair cell or a spiral ganglion neuron.

14. The vector of claim 13, wherein the second inner ear cell type is a cochlear hair cell.

15. The vector of any one of claims 1-12, wherein the first inner ear cell type is a vestibular supporting cell and the second inner ear cell type is at least one of a vestibular hair cell or a vestibular ganglion neuron.

16. The vector of claim 15, wherein the second inner ear cell type is a vestibular hair cell.

17. The vector of claim 16, wherein the second inner ear cell type is a vestibular type I hair cell.

18. The vector of any one of claims 1-12, wherein the first inner ear cell type is a vestibular type II hair cell and the second inner ear cell type is a vestibular type I hair cell.

19. The vector of any one of claims 1-12, wherein the first inner ear cell type is a vestibular type II hair cell and the second inner ear cell type is a vestibular ganglion neuron.

20. The vector of any one of claims 1-12, wherein the polynucleotide encodes Atonal BHLH Transcription Factor 1 (Atoh1), Growth Factor Independent 1 Transcriptional Repressor (Gfi1), POU Class 4 Homeobox 3 (Pou4f3), IKAROS Family Zinc Finger 2 (Ikzf2), dominant negative Sox2 (dnSox2), or Gap Junction Protein Beta 2 (Gjb2).

21. The vector of any one of claims 1-12, wherein the promoter is a supporting cell-specific promoter, a hair cell-specific promoter, or a ubiquitous promoter.

22. The vector of any one of claims 1-12, wherein the promoter is a cytomegalovirus (CMV) promoter, a Myosin 15 (MYO15) promoter, a LFNG O-Fucosylpeptide 3-Beta-N-Acetylglucosaminyltransferase (LFNG) promoter, a Fibroblast Growth Factor Receptor 3 (FGFR3) promoter, a Solute Carrier Family 1 Member 3 (SLC1A3) promoter, a Glial Fibrillary Acidic Protein (GFAP) promoter, or a Solute Carrier Family 6 Member 14 (SLC6A14) promoter.

23. The vector of any one of claims 1-22, further comprising a second polynucleotide that can be transcribed to produce an expression product, wherein the second polynucleotide is different from the first polynucleotide.

24. The vector of claim 23, wherein the second polynucleotide is operably linked to the promoter, the second polynucleotide is located 3′ of the first polynucleotide, the at least one polynucleotide that can be transcribed to produce a miRNA target sequence is located 3′ of the second polynucleotide, and the second polynucleotide is suitable for expression in the first inner ear cell type, but not in the second inner ear cell type.

25. The vector of claim 23 or 24, further comprising a third polynucleotide that can be transcribed to produce an expression product, wherein the third polynucleotide is different from the first polynucleotide and the second polynucleotide.

26. The vector of claim 25, wherein the third polynucleotide is operably linked to the promoter, the third polynucleotide is located 3′ of the second polynucleotide, the at least one polynucleotide that can be transcribed to produce a miRNA target sequence is located 3′ of the third polynucleotide, and the third polynucleotide is suitable for expression in the first inner ear cell type, but not in the second inner ear cell type.

27. The vector of any one of claims 1-12 and 20-26, wherein:
a. the first polynucleotide encodes Atoh1, Gfi1, Pou4f3, Ikzf2, dnSox2, or Gjb2;
b. the promoter is a CMV promoter, an FGFR3 promoter, an LFNG promoter, or a SLC1A3 promoter;
c. each miRNA target sequence transcribed from a polynucleotide operably linked to the promoter is independently targeted by one of: miR-183, miR-96, miR-182, miR-18a, miR-140, or miR-194;
d. the first inner ear cell type is a cochlear supporting cell; and
e. the second inner ear cell type is cochlear hair cell.

28. The vector of claim 27, wherein the first polynucleotide encodes Atoh1 and the second polynucleotide encodes Ikzf2.

29. The vector of claim 27, wherein the first polynucleotide encodes Atoh1, the second polynucleotide encodes Gfi1, and the third polynucleotide encodes Pou4f3.

30. The vector of any one of claims 1-12 and 20-26, wherein:
a. the first polynucleotide encodes GJB2;
b. the promoter is a GJB2 promoter, a CMV promoter, an FGFR3 promoter, an LFNG promoter, or a SLC1A3 promoter;
c. each miRNA target sequence transcribed from a polynucleotide operably linked to the promoter is independently targeted by one of: miR-183, miR-96, miR-182, miR-18a, miR-124, or miR-194;
d. the first inner ear cell type is a cochlear supporting cell; and
e. the second inner ear cell type is spiral ganglion neuron.

31. The vector of any one of claims 1-12 and 20-26, wherein:
a. the first polynucleotide encodes Atoh1 or dnSox2;
b. the promoter is a CMV promoter, a GFAP promoter, a SLC6A14 promoter, or a SLC1A3 promoter;
c. each miRNA target sequence transcribed from a polynucleotide operably linked to the promoter is independently targeted by one of: miR-183, miR-96, miR-182, miR-18a, miR-140, or miR-135b;
d. the first inner ear cell type is a vestibular supporting cell; and
e. the second inner ear cell type is vestibular hair cell.

32. The vector of any one of claims 1-12 and 20-26, wherein:
a. the first polynucleotide encodes Atoh1 or dnSox2;
b. the promoter is a CMV promoter, a GFAP promoter, a SLC6A14 promoter, or a SLC1A3 promoter;
c. each miRNA target sequence transcribed from a polynucleotide operably linked to the promoter is independently targeted by one of: miR-183, miR-96, miR-182, miR-18a, miR-124a, miR-100, or miR-135;
d. the first inner ear cell type is a vestibular supporting cell; and
e. the second inner ear cell type is vestibular ganglion neuron.

33. The vector of any one of claims 1-12 and 20-26, wherein:
a. the first polynucleotide encodes dnSox2;
b. the promoter is a MYO15 promoter;
c. each miRNA target sequence transcribed from a polynucleotide operably linked to the promoter is independently targeted by one of: miR-183, miR-96, miR-182, miR-18a, miR-124a, miR-100, or miR-135;
d. the first inner ear cell type is a vestibular type II hair cell; and
e. the second inner ear cell type is vestibular ganglion neuron.

34. The vector of claim 33, wherein each miRNA target sequence is independently targeted by one of: miR-18a, miR-124a, miR-100, or miR-135.

35. The vector of any one of claims 1-34, wherein the vector is an AAV vector.

36. A pharmaceutical composition comprising the vector of any one of claims 1-35 and a pharmaceutically acceptable carrier, excipient, or diluent.

37. A method of expressing a polynucleotide in a first inner ear cell type and not in a second inner ear cell type in a subject in need thereof, comprising locally administering to the middle or inner ear of the subject an effective amount of the vector of any one of claims 1-35 or the pharmaceutical composition of claim 36.

38. A method of reducing off-target expression of a polynucleotide in an inner ear of a subject, comprising locally administering to the middle or inner ear of the subject an effective amount of the vector of any one of claims 1-35 or the pharmaceutical composition of claim 36.

39. A method of treating a subject having or at risk of developing hearing loss, vestibular dysfunction, or tinnitus, comprising administering to the subject an effective amount of the vector of any one of claims 1-35 or the pharmaceutical composition of claim 36.

40. The method of claim 39, wherein the vestibular dysfunction comprises vertigo, dizziness, imbalance, bilateral vestibulopathy, oscillopsia, or a balance disorder.

41. The method of claim 39 or 40, wherein the vestibular dysfunction is age-related vestibular dysfunction, head trauma-related vestibular dysfunction, disease or infection-related vestibular dysfunction, or ototoxic drug-induced vestibular dysfunction.

42. The method of claim 39 or 40, wherein the vestibular dysfunction is idiopathic vestibular dysfunction.

43. The method of any one of claims 39-41, wherein the vestibular dysfunction is associated with a genetic mutation.

44. The method of claim 43, wherein the genetic mutation is a mutation in a gene listed in Table 4.

45. The method of claim 39, wherein the hearing loss is genetic hearing loss.

46. The method of claim 45, wherein the genetic hearing loss is autosomal dominant hearing loss, autosomal recessive hearing loss, or X-linked hearing loss.

47. The method of claim 45 or 46, wherein the genetic hearing loss is a condition associated with a mutation in a gene listed in Table 4.

48. The method of claim 39, wherein the hearing loss is acquired hearing loss.

49. The method of claim 48, wherein the acquired hearing loss is noise-induced hearing loss, age-related hearing loss, disease or infection-related hearing loss, head trauma-related hearing loss, or ototoxic drug-induced hearing loss.

50. The method of claim 41 or 49, wherein the ototoxic drug is an aminoglycoside, an antineoplastic drug, ethacrynic acid, furosemide, a salicylate, or quinine.

51. The method of claim 39, wherein the hearing loss or vestibular dysfunction is associated with age-related hearing loss, noise-induced hearing loss, DFNB61, DFNB1, DFNB7/11, DFNA2, DFNB77, DFNB28, DFNA41, DFNB8, DFNB37, DFNA22, DFNB3, Usher syndrome type 1, Usher syndrome type 2, or bilateral vestibulopathy.

52. The method of claim 51, wherein the hearing loss is associated with age-related hearing loss, noise-induced hearing loss, DFNB61, DFNB1, DFNB7/11, DFNA2, DFNB77, DFNB28, DFNA41, DFNB8, DFNB37, DFNA22, DFNB3, Usher syndrome type 1, or Usher syndrome type 2 and the first polynucleotide encodes Atoh1.

53. The method of claim 52, wherein the second polynucleotide encodes Ikzf2.

54. The method of claim 52, wherein the second polynucleotide encodes Pou4f3 and the third polynucleotide encodes Gfi1.

55. The method of claim 52, wherein the subject is additionally administered a vector comprising a polynucleotide encoding Ikzf2.

56. The method of claim 52, wherein the subject is additionally administered a vector comprising a polynucleotide encoding Pou4f3 and a vector comprising a polynucleotide encoding Gfi1.

57. The method of claim 51, wherein the hearing loss or vestibular dysfunction is associated with DFNB1, DFNB7/11, DFNA2, DFNB77, DFNB28, DFNA41, DFNB8, DFNB37, DFNA22, DFNB3, Usher syndrome type 1, Usher syndrome type 2, or bilateral vestibulopathy and the first polynucleotide encodes dnSox2.

58. The method of claim 57, wherein the second polynucleotide encodes Atoh1.

59. The method of claim 57, wherein subject is additionally administered a vector comprising a polynucleotide encoding Atoh1.


SEQUENCE LISTING (89 sequences)

SEQ ID NO: 1 - DNA, 5396 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcacgcgtatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtaataataagcttagtgaattctaccagtgccataagcaaaaatgtgctagtgccaaacggtgtgagttctaccattgccaaaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 2 - DNA, 5536 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcacgcgtatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtaataataagcttagtgaattctaccagtgccataagtgaattctaccagtgccataagtgaattctaccagtgccataagcaaaaatgtgctagtgccaaaagcaaaaatgtgctagtgccaaaagcaaaaatgtgctagtgccaaacggtgtgagttctaccattgccaaacggtgtgagttctaccattgccaaacggtgtgagttctaccattgccaaaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 3 - DNA, 5418 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcacgcgtatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtaataataagcttagcaaaaatgtgctagtgccaaaagcaaaaatgtgctagtgccaaaagcaaaaatgtgctagtgccaaaagcaaaaatgtgctagtgccaaaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 4 - DNA, 5426 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcacgcgtatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtaataataagcttcggtgtgagttctaccattgccaaacggtgtgagttctaccattgccaaacggtgtgagttctaccattgccaaacggtgtgagttctaccattgccaaaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 5 - DNA, 5414 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcacgcgtatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtaataataagcttagtgaattctaccagtgccataagtgaattctaccagtgccataagtgaattctaccagtgccataagtgaattctaccagtgccataggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 6 - DNA, 5349 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcacgcgtatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtaataataagcttagcaaaaatgtgctagtgccaaaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 7 - DNA, 5351 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcacgcgtatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtaataataagcttcggtgtgagttctaccattgccaaaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 8 - DNA, 5348 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcacgcgtatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtaataataagcttagtgaattctaccagtgccataggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 9 - DNA, 5363 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcgccgccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctaaacgcgttaataagcttagtgaattctaccagtgccataagcaaaaatgtgctagtgccaaacggtgtgagttctaccattgccaaaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 10 - DNA, 5503 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcgccgccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctaaacgcgttaataagcttagtgaattctaccagtgccataagtgaattctaccagtgccataagtgaattctaccagtgccataagcaaaaatgtgctagtgccaaaagcaaaaatgtgctagtgccaaaagcaaaaatgtgctagtgccaaacggtgtgagttctaccattgccaaacggtgtgagttctaccattgccaaacggtgtgagttctaccattgccaaaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 11 - DNA, 5385 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcgccgccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctaaacgcgttaataagcttagcaaaaatgtgctagtgccaaaagcaaaaatgtgctagtgccaaaagcaaaaatgtgctagtgccaaaagcaaaaatgtgctagtgccaaaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 12 - DNA, 5393 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcgccgccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctaaacgcgttaataagcttcggtgtgagttctaccattgccaaacggtgtgagttctaccattgccaaacggtgtgagttctaccattgccaaacggtgtgagttctaccattgccaaaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 13 - DNA, 5381 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcgccgccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctaaacgcgttaataagcttagtgaattctaccagtgccataagtgaattctaccagtgccataagtgaattctaccagtgccataagtgaattctaccagtgccataggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 14 - DNA, 5316 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcgccgccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctaaacgcgttaataagcttagcaaaaatgtgctagtgccaaaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 15 - DNA, 5318 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcgccgccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctaaacgcgttaataagcttcggtgtgagttctaccattgccaaaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 16 - DNA, 5315 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagggccggccgcggccgcgccgccatggattggggcacactccagagcatcctcgggggtgtcaacaaacactccaccagcattggaaagatctggctcacggtcctcttcatcttccgcatcatgatcctcgtggtggctgcaaaggaggtgtggggagatgagcaagccgattttgtctgcaacacgctccagcctggctgcaagaatgtatgctacgaccaccacttccccatctctcacatccggctctgggctctgcagctgatcatggtgtccacgccagccctcctggtagctatgcatgtggcctaccggagacatgaaaagaaacggaagttcatgaagggagagataaagaacgagtttaaggacatcgaagagatcaaaacccagaaggtccgtatcgaagggtccctgtggtggacctacaccaccagcatcttcttccgggtcatctttgaagccgtcttcatgtacgtcttttacatcatgtacaatggcttcttcatgcaacgtctggtgaaatgcaacgcttggccctgccccaatacagtggactgcttcatttccaggcccacagaaaagactgtcttcaccgtgtttatgatttctgtgtctggaatttgcattctgctaaatatcacagagctgtgctatttgttcgttaggtattgctcaggaaagtccaaaagaccagtctaaacgcgttaataagcttagtgaattctaccagtgccataggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 17 - DNA, 5295 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttagcaaaaatgtgctagtgccaaagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 18 - DNA, 5297 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttcggtgtgagttctaccattgccaaagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 19 - DNA, 5294 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttagtgaattctaccagtgccatagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 20 - DNA, 5350 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttagtgaattctaccagtgccatacgatagcaaaaatgtgctagtgccaaacgatcggtgtgagttctaccattgccaaagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 21 - DNA, 5514 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttagtgaattctaccagtgccatacgatagtgaattctaccagtgccatacgatagtgaattctaccagtgccatacgatagcaaaaatgtgctagtgccaaacgatagcaaaaatgtgctagtgccaaacgatagcaaaaatgtgctagtgccaaacgatcggtgtgagttctaccattgccaaacgatcggtgtgagttctaccattgccaaacgatcggtgtgagttctaccattgccaaagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 22 - DNA, 5376 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttagcaaaaatgtgctagtgccaaacgatagcaaaaatgtgctagtgccaaacgatagcaaaaatgtgctagtgccaaacgatagcaaaaatgtgctagtgccaaagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 23 - DNA, 5384 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttcggtgtgagttctaccattgccaaacgatcggtgtgagttctaccattgccaaacgatcggtgtgagttctaccattgccaaacgatcggtgtgagttctaccattgccaaagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 24 - DNA, 5372 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttagtgaattctaccagtgccatacgatagtgaattctaccagtgccatacgatagtgaattctaccagtgccatacgatagtgaattctaccagtgccatagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 25 - RNA, 22 nt, Mus musculus
uauggcacugguagaauucacu

SEQ ID NO: 26 - RNA, 23 nt, Mus musculus
uuuggcacuagcacauuuuugcu

SEQ ID NO: 27 - RNA, 25 nt, Mus musculus
uuuggcaaugguagaacucacaccg

SEQ ID NO: 28 - RNA, 23 nt, Mus musculus
uaaggugcaucuagugcagauag

SEQ ID NO: 29 - RNA, 22 nt, Mus musculus
cagugguuuuacccuaugguag

SEQ ID NO: 30 - RNA, 22 nt, Mus musculus
uguaacagcaacuccaugugga

SEQ ID NO: 31 - RNA, 22 nt, Mus musculus
uagcagcacauaaugguuugug

SEQ ID NO: 32 - RNA, 22 nt, Mus musculus
uguaaacauccuacacucagcu

SEQ ID NO: 33 - RNA, 22 nt, Mus musculus
aacccguagauccgaucuugug

SEQ ID NO: 34 - RNA, 20 nt, Mus musculus
uaaggcacgcggugaaugcc

SEQ ID NO: 35 - RNA, 22 nt, Mus musculus
uccuucauuccaccggagucug

SEQ ID NO: 36 - RNA, 21 nt, Mus musculus
aucguagaggaaaauccacgu

SEQ ID NO: 37 - RNA, 21 nt, Mus musculus
aucauagaggaacauccacuu

SEQ ID NO: 38 - RNA, 23 nt, Mus musculus
uauggcuuuucauuccuauguga

SEQ ID NO: 39 - RNA, 22 nt, Mus musculus
aacccguagauccgaacuugug

SEQ ID NO: 40 - RNA, 23 nt, Mus musculus
uauggcuuuuuauuccuauguga

SEQ ID NO: 41 - RNA, 21 nt, Mus musculus
aucauagaggaacauccacuu

SEQ ID NO: 42 - RNA, 21 nt, Mus musculus
aucguagaggaaaauccacgu

SEQ ID NO: 43 - protein, 354 aa, Artificial Sequence
  Synthetic Construct
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENAKTSPRSHRSDGEFSPHSHYSDSDEAS

SEQ ID NO: 44 - protein, 354 aa, Artificial Sequence
  Synthetic Construct
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTAPRSHRSDGEFSPHSHYSDSDEAS

SEQ ID NO: 45 - protein, 354 aa, Artificial Sequence
  Synthetic Construct
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTSPRAHRSDGEFSPHSHYSDSDEAS

SEQ ID NO: 46 - protein, 354 aa, Artificial Sequence
  Synthetic Construct
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENAKTAPRSHRSDGEFSPHSHYSDSDEAS

SEQ ID NO: 47 - protein, 354 aa, Artificial Sequence
  Synthetic Construct
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENAKTSPRAHRSDGEFSPHSHYSDSDEAS

SEQ ID NO: 48 - protein, 354 aa, Artificial Sequence
  Synthetic Construct
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTAPRAHRSDGEFSPHSHYSDSDEAS

SEQ ID NO: 49 - protein, 354 aa, Artificial Sequence
  Synthetic Construct
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENAKTAPRAHRSDGEFSPHSHYSDSDEAS

SEQ ID NO: 50 - DNA, 954 bp, Artificial Sequence
  Synthetic Construct
atgtataacatgatggagacggagctgaagccgccgggcccgcagcaagcttcggggggcggcggcggaggaggcaacgccacggcggcggcgaccggcggcaaccagaagaacagcccggaccgcgtcacggggcccatgaacgccttcatggtatggtcccgggggcagctgggtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgctggggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcac

SEQ ID NO: 51 - DNA, 957 bp, Artificial Sequence
  Synthetic Construct
atgtataacatgatggagacggagctgaagccgccgggcccgcagcaagcttcggggggcggcggcggaggaggcaacgccacggcggcggcgaccggcggcaaccagaagaacagcccggaccgcgtcacggggcccatgaacgccttcatggtatggtcccgggggcagctgggtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgctggggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatg

SEQ ID NO: 52 - DNA, 21 bp, Artificial Sequence
  Synthetic Construct
ctgccgagaatccatgtatat

SEQ ID NO: 53 - DNA, 21 bp, Artificial Sequence
  Synthetic Construct
gtacagtatttatcgagataa

SEQ ID NO: 54 - DNA, 21 bp, Artificial Sequence
  Synthetic Construct
aggagcacccggattataaat

SEQ ID NO: 55 - DNA, 21 bp, Artificial Sequence
  Synthetic Construct
tggacagttacgcgcacatga

SEQ ID NO: 56 - DNA, 21 bp, Artificial Sequence
  Synthetic Construct
tcccatcacccacagcaaatg

SEQ ID NO: 57 - DNA, 21 bp, Artificial Sequence
  Synthetic Construct
cgagataaacatggcaatcaa

SEQ ID NO: 58 - DNA, 21 bp, Artificial Sequence
  Synthetic Construct
cgctcatgaagaaggataagt

SEQ ID NO: 59 - DNA, 21 bp, Artificial Sequence
  Synthetic Construct
cagctcgcagacctacatgaa

SEQ ID NO: 60 - DNA, 21 bp, Artificial Sequence
  Synthetic Construct
caacggcagctacagcatgat

SEQ ID NO: 61 - DNA, 21 bp, Artificial Sequence
  Synthetic Construct
ccacctacagcatgtcctact

SEQ ID NO: 62 - DNA, 21 bp, Artificial Sequence
  Synthetic Construct
ccctgcagtacaactccatga

SEQ ID NO: 63 - DNA, 21 bp, Artificial Sequence
  Synthetic Construct
acatgtcccagcactaccaga

SEQ ID NO: 64 - DNA, 21 bp, Artificial Sequence
  Synthetic Construct
gcacatgaacggctggagcaa

SEQ ID NO: 65 - DNA, 21 bp, Artificial Sequence
  Synthetic Construct
gcccacctacagcatgtccta

SEQ ID NO: 66 - DNA, 21 bp, Artificial Sequence
  Synthetic Construct
gaagaaggataagtacacgct

SEQ ID NO: 67 - DNA, 21 bp, Artificial Sequence
  Synthetic Construct
accaatcccatccaaattaac

SEQ ID NO: 68 - DNA, 21 bp, Artificial Sequence
  Synthetic Construct
caaagagatacaagggaattg

SEQ ID NO: 69 - DNA, 21 bp, Artificial Sequence
  Synthetic Construct
tgcgcccagtagactgcacat

SEQ ID NO: 70 - DNA, 21 bp, Artificial Sequence
  Synthetic Construct
cgcggcacagatgcaaccgat

SEQ ID NO: 71 - DNA, 19 bp, Artificial Sequence
  Synthetic Construct
ccagtaatatttagagcta

SEQ ID NO: 72 - DNA, 19 bp, Artificial Sequence
  Synthetic Construct
ttgtgatattttaaggttt

SEQ ID NO: 73 - DNA, 19 bp, Artificial Sequence
  Synthetic Construct
cttatggtttgtaatattt

SEQ ID NO: 74 - DNA, 22 bp, Artificial Sequence
  Synthetic Construct
ttgattgccatgtttatctcga

SEQ ID NO: 75 - DNA, 22 bp, Artificial Sequence
  Synthetic Construct
ttatctcgataaatactgtaca

SEQ ID NO: 76 - DNA, 6198 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttctcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgccgagataaacatggcaatcaatagtgaagccacagatgtattgattgccatgtttatctcgatgcctactgcctcgcaattgaaggggctactttaggagcaattatcttgtttactaaaactgaataccttgctatctctttgatacatttttacaaagctgaattaaaatggtataaattaaatcacttttataaattaaatcacttttttacgcgtggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagctcttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 77 - DNA, 6195 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttctcgactagggataacagggtaattgtttgaatgaggcttcagtactttacagaatcgttgcctgcacatcttggaaacacttgctgggattacttcgacttcttaacccaacagaaggctcgagaaggtatattgctgttgacagtgagcgccgagataaacatggcaatcaatagtgaagccacagatgtattgattgccatgtttatctcgatgcctactgcctcggacttcaaggggctagaattcgagcaattatcttgtttactaaaactgaataccttgctatctctttgatacatttttacaaagctgaattaaaatggtataaattaaatcacttttttcaattgacgcgtaattctaccggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagctcttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 78 - DNA, 6198 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttctcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgcgtacagtatttatcgagataatagtgaagccacagatgtattatctcgataaatactgtacatgcctactgcctcgcaattgaaggggctactttaggagcaattatcttgtttactaaaactgaataccttgctatctctttgatacatttttacaaagctgaattaaaatggtataaattaaatcacttttataaattaaatcacttttttacgcgtggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagctcttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 79 - DNA, 6195 bp, Artificial Sequence
  Synthetic Construct
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcggcgcgccaccggtgcgatcgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcgccaccatgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcttctcgactagggataacagggtaattgtttgaatgaggcttcagtactttacagaatcgttgcctgcacatcttggaaacacttgctgggattacttcgacttcttaacccaacagaaggctcgagaaggtatattgctgttgacagtgagcgcgtacagtatttatcgagataatagtgaagccacagatgtattatctcgataaatactgtacatgcctactgcctcggacttcaaggggctagaattcgagcaattatcttgtttactaaaactgaataccttgctatctctttgatacatttttacaaagctgaattaaaatggtataaattaaatcacttttttcaattgacgcgtaattctaccggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagctcttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtcttgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg

SEQ ID NO: 80 - RNA, 21 nt, Artificial Sequence
  Synthetic Construct
ccaguaauauuuagagcuauu

SEQ ID NO: 81 - RNA, 21 nt, Artificial Sequence
  Synthetic Construct
uagcucuaaauauuacugguu

SEQ ID NO: 82 - RNA, 21 nt, Artificial Sequence
  Synthetic Construct
cgcucaugaagaaggauaauu

SEQ ID NO: 83 - RNA, 21 nt, Artificial Sequence
  Synthetic Construct
uuauccuucuucaugagcguu

SEQ ID NO: 84 - RNA, 21 nt, Artificial Sequence
  Synthetic Construct
uugugauauuuuaagguuuuu

SEQ ID NO: 85 - RNA, 21 nt, Artificial Sequence
  Synthetic Construct
aaaccuuaaaauaucacaauu

SEQ ID NO: 86 - RNA, 21 nt, Artificial Sequence
  Synthetic Construct
cuuaugguuuguaauauuuuu

SEQ ID NO: 87 - RNA, 21 nt, Artificial Sequence
  Synthetic Construct
aaauauuacaaaccauaaguu

SEQ ID NO: 88 - DNA, 548 bp, Artificial Sequence
  Synthetic Construct
gatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcga

SEQ ID NO: 89 - DNA, 425 bp, Artificial Sequence
  Synthetic Construct
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaa